(19) United States
(12) Patent Application Publication
(10) Pub. No.: US 2004/0132771 A1
(43) Pub. Date: Jul. 8, 2004

(54) Compositions of choleseteryl ester transfer protein inhibitors and HMG-CoA reductase inhibitors

(75) Inventors: Walter C. Babcock, Bend, OR (US); Dwayne T. Friesen, Bend, OR (US); Ravi M. Shankar, Groton, CT (US); Daniel T. Smithey, Bend, OR (US)

Correspondence Address: PFIZER INC., PATENT DEPARTMENT, MS8260-1611, EASTERN POINT ROAD, GROTON, CT, 06340, US

(73) Assignee: Pfizer Inc

(21) Appl. No.: 10/678,145

(22) Filed: Oct. 6, 2003

Related U.S. Application Data
(60) Provisional application No. 60/435,328, filed on Dec. 20, 2002.

(51) Int. Cl. A61K 31/47; A61K 9/14

(52) U.S. Cl. 514/311; 424/486

(57) ABSTRACT

A composition comprises (1) a solid amorphous adsorbate comprising a cholesteryl ester transfer protein (CETP) inhibitor and a substrate; and (2) an HMG-CoA reductase inhibitor. The solid amorphous adsorbate provides concentration enhancement of the CETP inhibitor relative to a control composition consisting essentially of the unadsorbed CETP inhibitor alone, resulting in improved bioavailability.

### CROSS-REFERENCE TO RELATED APPLICATION

This application claims the benefit of priority of provisional Patent Application Serial No. 60/435,328 filed Dec. 20, 2002, which is incorporated herein by reference in its entirety for all purposes.

### BACKGROUND

The present invention relates to compositions comprising: (1) a solid amorphous adsorbate comprising a cholesteryl ester transfer protein (CETP) inhibitor and a substrate; and (2) an HMG-CoA reductase inhibitor.

It is well known that inhibitors of 3-hydroxy-3-methylglutaryl-coenzyme A reductase (HMG-CoA reductase), an important enzyme catalyzing the intracellular synthesis of cholesterol, will bring about reduced levels of blood cholesterol, especially in terms of the low density lipoprotein form of cholesterol (LDL-C). Therefore, HMG-CoA reductase inhibitors are considered potentially useful as hypocholesterolemic or hypolipidemic agents.

CETP inhibitors are another class of compounds that are capable of modulating levels of blood cholesterol, such as by raising high-density lipoprotein (HDL) cholesterol and lowering low-density lipoprotein (LDL) cholesterol. It is desired to use CETP inhibitors to lower certain plasma lipid levels, such as LDL-cholesterol and triglycerides and to elevate certain other plasma lipid levels, including HDL-cholesterol and accordingly to treat diseases which are affected by low levels of HDL cholesterol and/or high levels of LDL-cholesterol and triglycerides, such as atherosclerosis and cardiovascular diseases in certain mammals (i.e., those which have CETP in their plasma), including humans.

It is well known that a combination therapy of a CETP inhibitor and an HMG-CoA reductase inhibitor may be used to treat elevated LDL cholesterol and low HDL cholesterol levels. For example, WO02/13797 A2 relates to pharmaceutical combinations of cholesteryl ester transfer protein inhibitors and atorvastatin. The application discloses that the compounds may be generally administered separately or together, with a pharmaceutically acceptable carrier, vehicle or diluent. The compounds may be administered individually or together in any conventional oral, parenteral or transdermal dosage form. For oral administration, the dosage form may take the form of solutions, suspensions, tablets, pills, capsules, powders and the like.

DeNinno et al., U.S. Pat. No. 6,310,075 B1, relates to CETP inhibitors, pharmaceutical compositions containing such inhibitors and the use of such inhibitors. DeNinno et al. disclose a pharmaceutical combination composition comprising a CETP inhibitor and an HMG-CoA reductase inhibitor. DeNinno et al. disclose that the compounds of the invention may be administered in the form of a pharmaceutical composition comprising at least one of the compounds, together with a pharmaceutically acceptable vehicle, diluent, or carrier. For oral administration a pharmaceutical composition can take the form of solutions, suspensions, tablets, pills, capsules, powders and the like. Similarly, DeNinno et al., U.S. Pat. No. 6,197,786 B1, disclose pharmaceutical combinations comprising CETP inhibitors and HMG-CoA reductase inhibitors.

U.S. Pat. No. 6,462,091 B1 discloses combinations of CETP inhibitors and HMG-CoA reductase inhibitors for cardiovascular indications. The pharmaceutical compositions include those suitable for oral, rectal, topical, buccal, and parenteral administration. The application discloses solid dosage forms for oral administration including capsules, tablets, pills, powders, gel caps and granules.

Schmeck et al., U.S. Pat. No. 5,932,587, disclose another class of CETP inhibitors. Schmeck et al. disclose that the CETP inhibitors may be used in combination with certain HMG-CoA reductase inhibitors such as statins, including atorvastatin.

CETP inhibitors, particularly those that have high binding activity, are generally hydrophobic, have extremely low aqueous solubility and have low oral bioavailability when dosed conventionally. Such compounds have generally proven to be difficult to formulate for oral administration such that high bioavailabilities are achieved. Accordingly, CETP inhibitors must be formulated so as to be capable of providing good bioavailability. Such formulations generally increase the size of the dosage form, e.g. tablet or capsule, making it more difficult to administer, e.g. swallow, particularly for elderly patients.

Designing dosage forms for combination therapy of an HMG-CoA reductase inhibitor and a CETP inhibitor presents even further challenges. Not only is it preferable that the dosage form be of a size that is easily swallowed, it is also preferable that the number of dosage forms taken per dose be low, preferably one unit, because many patients take multiple drugs.

Thus, there is a continuing need to find safe, effective methods of delivering combinations of HMG-CoA reductase inhibitors and CETP inhibitors.

### SUMMARY OF INVENTION

The present invention overcomes the drawbacks of the prior art by providing a composition comprising (1) a cholesteryl ester transfer protein (CETP) inhibitor in a solubility-improved form and (2) an HMG-CoA reductase inhibitor, wherein the solubility-improved form is a solid amorphous adsorbate, the solid amorphous adsorbate being selected from the group consisting of a solid adsorbate comprising a low-solubility CETP inhibitor adsorbed onto a substrate and adsorbates of the CETP inhibitor in a crosslinked polymer. In one embodiment, the solubility-improved form comprises a solid adsorbate comprising a low-solubility CETP inhibitor adsorbed onto a substrate, the substrate having a surface area of at least 20 m²/g, and wherein at least a major portion of the CETP inhibitor in the solid adsorbate is amorphous. The solid adsorbate may optionally comprise a concentration-enhancing polymer. The solid adsorbate may also be mixed with a concentration-enhancing polymer. The solid amorphous adsorbate comprising a CETP inhibitor and a substrate provides concentration enhancement of the CETP inhibitor relative to a control composition consisting essentially of the unadsorbed CETP inhibitor alone.

In another aspect, the compositions and dosage forms of the present invention may be used to treat any condition, which is subject to treatment by administering a CETP inhibitor and an HMG-CoA reductase inhibitor, as disclosed in commonly assigned, copending U.S. patent application Ser. No. 2002/0035125A1, the disclosure of which is herein incorporated by reference.

The foregoing and other objectives, features, and advantages of the invention will be more readily understood upon consideration of the following detailed description of the invention.

### DETAILED DESCRIPTION OF THE PREFERRED EMBODIMENTS

The present invention provides a composition comprising (1) a solid amorphous adsorbate comprising a CETP inhibitor and a substrate; and (2) an HMG-CoA reductase inhibitor. In one aspect, the solid amorphous adsorbate provides concentration-enhancement of the CETP inhibitor when administered to an aqueous environment of use relative to a control composition consisting essentially of the unadsorbed CETP inhibitor alone.

The terms “use environment” and “aqueous environment of use” are used interchangeably herein and can either mean in vivo fluids, such as the GI tract, subdermal, intranasal, buccal, intrathecal, ocular, intraaural, subcutaneous spaces, vaginal tract, arterial and venous blood vessels, pulmonary tract or intramuscular tissue of an animal, such as a mammal and particularly a human, or the in vitro environment of a test solution, such as phosphate buffered saline (PBS) or a Model Fasted Duodenal (MFD) solution. An appropriate PBS solution is an aqueous solution comprising 20 mM sodium phosphate (Na₂HPO₄), 47 mM potassium phosphate (KH₂PO₄), 87 mM NaCl, and 0.2 mM KCl, adjusted to pH 6.5 with NaOH. An appropriate MFD solution is the same PBS solution wherein additionally is present 7.3 mM sodium taurocholic acid and 1.4 mM of 1-palmitoyl-2-oleyl-sn-glycero-3-phosphocholine.

“Administration” to a use environment means, where the in vivo use environment is the GI tract, delivery by ingestion or swallowing or other such means to deliver the drugs. One skilled in the art will understand that “administration” to other in vivo use environments means contacting the use environment with the composition of the invention using methods known in the art. See for example, Remington: The Science and Practice of Pharmacy, 20^{th} Edition (2000). Where the use environment is in vitro, “administration” refers to placement or delivery of the composition or dosage form to the in vitro test medium.

CETP inhibitors, solid amorphous adsorbates, HMG-CoA reductase inhibitors, improved bioavailability obtained with the compositions of the present invention, and suitable dosage forms of the present invention are discussed in more detail below.

### CHOLESTERYL ESTER TRANSFER PROTEIN INHIBITORS

The CETP inhibitor may be any compound capable of inhibiting the cholesteryl ester transfer protein. The CETP inhibitor is typically “sparingly water-soluble,” which means that the CETP inhibitor has a minimum aqueous solubility of less than about 1 to 2 mg/mL at any physiologically relevant pH (e.g., pH 1-8) and at about 22° C. Many CETP inhibitors are “substantially water-insoluble,” which means that the CETP inhibitor has a minimum aqueous solubility of less than about 0.01 mg/mL (or 10 μg/ml) at any physiologically relevant pH (e.g., pH 1-8) and at about 22° C. (Unless otherwise specified, reference to aqueous solubility herein and in the claims is determined at about 22° C.) Compositions of the present invention find greater utility as the aqueous solubility of the CETP inhibitors decreases, and thus are preferred for CETP inhibitors with aqueous solubilities less than about 10 μg/mL, and of even more utility for CETP inhibitors with aqueous solubilities less than about 1 μg/mL. Many CETP inhibitors have even lower aqueous solubilities (some even less than 0.1 μg/mL), and require dramatic concentration enhancement to be sufficiently bioavailable upon oral dosing for effective plasma concentrations to be reached at practical doses.

In general, the CETP inhibitor has a dose-to-aqueous solubility ratio greater than about 100 mL, where the aqueous solubility (mg/mL) is the minimum value observed in any physiologically relevant aqueous solution (e.g., those with pH values from 1 to 8) including USP simulated gastric and intestinal buffers, and dose is in mg. Compositions of the present invention, as mentioned above, find greater utility as the aqueous solubility of the CETP inhibitor decreases and the dose increases. Thus, the compositions have greater utility as the dose-to-solubility ratio increases, and thus are preferred for dose-to-solubility ratios greater than 1000 mL, and have even greater utility for dose-to-solubility ratios greater than about 5000 ml. The dose-to-solubility ratio may be determined by dividing the dose (in mg) by the aqueous solubility (in mg/ml).

Oral delivery of many CETP inhibitors is particularly difficult because their aqueous solubility is usually extremely low, typically being less than about 10 μg/ml, often being less than 0.1 μg/ml. Such low solubilities are a direct consequence of the particular structural characteristics of species that bind to CETP and thus act as CETP inhibitors. This low solubility is primarily due to the hydrophobic nature of CETP inhibitors. Log P, defined as the base 10 logarithm of the ratio of the drug solubility in octanol to the drug solubility in water, is a widely accepted measure of hydrophobicity. Log P may be measured experimentally or calculated using methods known in the art. Calculated Log P values are often referred to by the calculation method, such as Clog P, Alog P and Mlog P. In general, Log P values for CETP inhibitors are greater than 4 and are often greater than 5. Thus, the hydrophobic and insoluble nature of CETP inhibitors as a class pose a particular challenge for oral delivery. Achieving therapeutic drug levels in the blood by oral dosing of practical quantities of drug generally requires a large enhancement in drug concentrations in the gastrointestinal fluid and a resulting large enhancement in bioavailability. Such enhancements in drug concentration in gastrointestinal fluid typically need to be at least about 10-fold and often at least about 50-fold or even at least about 200-fold to achieve desired blood levels.

In contrast to conventional wisdom, the relative degree of enhancement in aqueous concentration and bioavailability provided by the solid amorphous adsorbates generally improves for CETP inhibitors as solubility decreases and hydrophobicity increases. In fact, the inventors have recognized a subclass of CETP inhibitors that are essentially aqueous insoluble, highly hydrophobic, and are characterized by a set of physical properties. This subclass of CETP inhibitors, referred to herein as “hydrophobic CETP inhibitors,” exhibits dramatic enhancements in aqueous concentration and bioavailability when formulated using a solid amorphous adsorbate.

The first property of hydrophobic CETP inhibitors is extremely low aqueous solubility. By extremely low aqueous solubility is meant that the minimum aqueous solubility at physiologically relevant pH (pH of 1 to 8) is less than about 10 μg/ml and typically less than about 1 μg/ml.

A second property is a very high dose-to-solubility ratio. Extremely low aqueous solubility often leads to poor or slow absorption of the drug from the fluid of the gastrointestinal tract, when the drug is dosed orally in a conventional manner. For extremely low solubility drugs, poor absorption generally becomes progressively more difficult as the dose (mass of drug given orally) increases. Thus, a second property of hydrophobic CETP inhibitors is a very high dose (in mg) to solubility (in mg/ml) ratio (ml). By “very high dose-to-solubility ratio” is meant that the dose-to-solubility ratio may have a value of at least 1000 ml, at least 5,000 ml, or even at least 10,000 ml.

A third property of hydrophobic CETP inhibitors is that they are extremely hydrophobic. By extremely hydrophobic is meant that the Log P value of the drug may have a value of at least 4.0, a value of at least 5.0, and even a value of at least 5.5.

A fourth property of hydrophobic CETP inhibitors is that they have a low melting point. Generally, drugs of this subclass will have a melting point of about 150° C. or less, and often about 140° C. or less.

Primarily, as a consequence of some or all of these four properties, hydrophobic CETP inhibitors typically have very low absolute bioavailabilities. Specifically, the absolute bioavailability of drugs in this subclass when dosed orally in their unadsorbed state is less than about 10% and more often less than about 5%. As discussed below, when formulated as a solid amorphous adsorbate, hydrophobic CETP inhibitors often exhibit dramatic enhancements in aqueous concentration in the use environment and in bioavailability when dosed orally.

Thus, in one embodiment, the invention provides a composition comprising (a) a solid amorphous adsorbate, the solid amorphous adsorbate comprising a CETP inhibitor and a substrate, and (b) an HMG-CoA reductase inhibitor, wherein the CETP inhibitor is a hydrophobic CETP inhibitor.

In the following, by “pharmaceutically acceptable forms” thereof is meant any pharmaceutically acceptable derivative or variation, including stereoisomers, stereoisomer mixtures, enantiomers, solvates, hydrates, isomorphs, pseudomorphs, polymorphs, salt forms and prodrugs.

One class of CETP inhibitors that finds utility with the present invention consists of oxy substituted 4-carboxyamino-2-methyl-1,2,3,4-tetrahydroquinolines having the Formula I

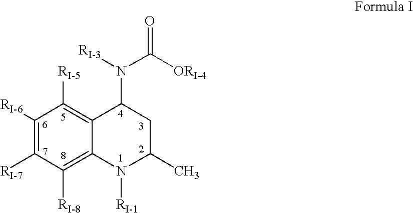

and pharmaceutically acceptable forms thereof;

wherein R_{I-1} is hydrogen, Y_{I}, W_{I}—X_{I}, W_{I}—Y_{I};

wherein W_{I} is a carbonyl, thiocarbonyl, sulfinyl or sulfonyl;

X_{I} is —O—Y_{I}, —S—Y_{I}, —N(H)—Y, or —N—(Y_{I})₂;

wherein Y_{I} for each occurrence is independently Z_{I} or a fully saturated, partially unsaturated or fully unsaturated one to ten membered straight or branched carbon chain wherein the carbons, other than the connecting carbon, may optionally be replaced with one or two heteroatoms selected independently from oxygen, sulfur and nitrogen and said carbon is optionally mono-, di- or tri-substituted independently with halo, said carbon is optionally mono-substituted with hydroxy, said carbon is optionally mono-substituted with oxo, said sulfur is optionally mono- or di-substituted with oxo, said nitrogen is optionally mono-, or di-substituted with oxo, and said carbon chain is optionally mono-substituted with Z_{I};

wherein Z_{I} is a partially saturated, fully saturated or fully unsaturated three to eight membered ring optionally having one to four heteroatoms selected independently from oxygen, sulfur and nitrogen, or, a bicyclic ring consisting of two fused partially saturated, fully saturated or fully unsaturated three to six membered rings, taken independently, optionally having one to four heteroatoms selected independently from nitrogen, sulfur and oxygen;

wherein said Z_{I} substituent is optionally mono-, di- or tri-substituted independently with halo, (C₂-C₆)alkenyl, (C₁-C₆) alkyl, hydroxy, (C₁-C₆)alkoxy, (C₁-C₄)alkylthio, amino, nitro, cyano, oxo, carboxyl, (C₁-C₆)alkyloxycarbonyl, mono-N- or di-N,N-(C₁-C₆)alkylamino wherein said (C₁-C₆)alkyl substituent is optionally mono-, di- or tri-substituted independently with halo, hydroxy, (C₁-C₆)alkoxy, (C₁-C₄)alkylthio, amino, nitro, cyano, oxo, carboxyl, (C₁-C₆)alkyloxycarbonyl, mono-N- or di-N,N-(C₁-C₆)alkylamino, said (C₁-C₆)alkyl substituent is also optionally substituted with from one to nine fluorines;

R_{I-3} is hydrogen or Q_{I};

wherein Q_{I} is a fully saturated, partially unsaturated or fully unsaturated one to six membered straight or branched carbon chain wherein the carbons, other than the connecting carbon, may optionally be replaced with one heteroatom selected from oxygen, sulfur and nitrogen and said carbon is optionally mono-, di- or tri-substituted independently with halo, said carbon is optionally mono-substituted with hydroxy, said carbon is optionally mono-substituted with oxo, said sulfur is optionally mono- or di-substituted with oxo, said nitrogen is optionally mono-, or di-substituted with oxo, and said carbon chain is optionally mono-substituted with V_{I};

wherein V_{I} is a partially saturated, fully saturated or fully unsaturated three to eight membered ring optionally having one to four heteroatoms selected independently from oxygen, sulfur and nitrogen, or a bicyclic ring consisting of two fused partially saturated, fully saturated or fully unsaturated three to six membered rings, taken independently, optionally having one to four heteroatoms selected independently from nitrogen, sulfur and oxygen;

wherein said V_{I} substituent is optionally mono-, di-, tri-, or tetra-substituted independently with halo, (C₁-C₆)alkyl, (C₂-C₆)alkenyl, hydroxy, (C₁-C₆)alkoxy, (C₁-C₄)alkylthio, amino, nitro, cyano, oxo, carbamoyl, mono-N- or di-N,N-(C₁-C₆) alkylcarbamoyl, carboxyl, (C₁-C₆)alkyloxycarbonyl, mono-N- or di-N,N-(C₁-C₆)alkylamino wherein said (C₁-C₆)alkyl or (C₂-C₆)alkenyl substituent is optionally mono-, di- or tri-substituted independently with hydroxy, (C₁-C₆)alkoxy, (C₁-C₄)alkylthio, amino, nitro, cyano, oxo, carboxyl, (C₁-C₆)alkyloxycarbonyl, mono-N- or di-N,N-(C₁-C₆)alkylamino, said (C₁-C₆)alkyl or (C₂-C₆)alkenyl substituents are also optionally substituted with from one to nine fluorines;

R_{I-4} is Q_{I-1} or V_{I-1}

wherein Q_{I-1} is a fully saturated, partially unsaturated or fully unsaturated one to six membered straight or branched carbon chain wherein the carbons, other than the connecting carbon, may optionally be replaced with one heteroatom selected from oxygen, sulfur and nitrogen and said carbon is optionally mono-, di- or tri-substituted independently with halo, said carbon is optionally mono-substituted with hydroxy, said carbon is optionally mono-substituted with oxo, said sulfur is optionally mono- or di-substituted with oxo, said nitrogen is optionally mono-, or di-substituted with oxo, and said carbon chain is optionally mono-substituted with V_{I-1};

wherein V_{I-1} is a partially saturated, fully saturated or fully unsaturated three to six membered ring optionally having one to two heteroatoms selected independently from oxygen, sulfur and nitrogen;

wherein said V_{I-1} substituent is optionally mono-, di-, tri-, or tetra-substituted independently with halo, (C₁-C₆)alkyl, (C₁-C₆)alkoxy, amino, nitro, cyano, (C₁-C₆)alkyloxycarbonyl, mono-N- or di-N,N-(C₁-C₆)alkylamino wherein said (C₁-C₆)alkyl substituent is optionally mono-substituted with oxo, said (C₁-C₆)alkyl substituent is also optionally substituted with from one to nine fluorines;

wherein either R_{I-3} must contain V_{I} or R_{I-4} must contain V_{I-1}; and R_{I-5}, R_{I-6}, R_{I-7} and R_{I-8} are each independently hydrogen, hydroxy or oxy wherein said oxy is substituted with T_{I} or a partially saturated, fully saturated or fully unsaturated one to twelve membered straight or branched carbon chain wherein the carbons, other than the connecting carbon, may optionally be replaced with one or two heteroatoms selected independently from oxygen, sulfur and nitrogen and said carbon is optionally mono-, di- or tri-substituted independently with halo, said carbon is optionally mono-substituted with hydroxy, said carbon is optionally mono-substituted with oxo, said sulfur is optionally mono- or di-substituted with oxo, said nitrogen is optionally mono- or di-substituted with oxo, and said carbon chain is optionally mono-substituted with T_{I};

wherein T_{I} is a partially saturated, fully saturated or fully unsaturated three to eight membered ring optionally having one to four heteroatoms selected independently from oxygen, sulfur and nitrogen, or a bicyclic ring consisting of two fused partially saturated, fully saturated or fully unsaturated three to six membered rings, taken independently, optionally having one to four heteroatoms selected independently from nitrogen, sulfur and oxygen;

wherein said T_{I} substituent is optionally mono-, di- or tri-substituted independently with halo, (C₁-C₆)alkyl, (C₂-C₆)alkenyl, hydroxy, (C₁-C₆)alkoxy, (C₁-C₄)alkylthio, amino, nitro, cyano, oxo, carboxy, (C₁-C₆)alkyloxycarbonyl, mono-N- or di-N,N-(C₁-C₆)alkylamino wherein said (C₁-C₆)alkyl substituent is optionally mono-, di- or tri-substituted independently with hydroxy, (C₁-C₆)alkoxy, (C₁-C₄)alkylthio, amino, nitro, cyano, oxo, carboxy, (C₁-C₆)alkyloxycarbonyl, mono-N- or di-N,N-(C₁-C₆)alkylamino, said (C₁-C₆)alkyl substituent is also optionally substituted with from one to nine fluorines.

Compounds of Formula I are disclosed in commonly assigned U.S. Pat. No. 6,140,342, the complete disclosure of which is herein incorporated by reference.

In a preferred embodiment, the CETP inhibitor is selected from one of the following compounds of Formula I:

[2R,4S] 4-[(3,5-dichloro-benzyl)-methoxycarbonyl-amino]-6,7-dimethoxy-2-methyl-3,4-dihydro-2H-quinoline-1-carboxylic acid ethyl ester;

[2R,4S] 4-[(3,5-dinitro-benzyl)-methoxycarbonyl-amino]-6,7-dimethoxy-2-methyl-3,4-dihydro-2H-quinoline-1-carboxylic acid ethyl ester;

[2R,4S] 4-[(2,6-dichloro-pyridin-4-ylmethyl)-methoxycarbonyl-amino]-6,7-dimethoxy-2-methyl-3,4-dihydro-2H-quinoline-1-carboxylic acid ethyl ester;

[2R,4S] 4-[(3,5-bis-trifluoromethyl-benzyl)-methoxycarbonyl-amino]-6,7-dimethoxy-2-methyl-3,4-dihydro-2H-quinoline-1-carboxylic acid ethyl ester;

[2R,4S] 4-[(3,5-bis-trifluoromethyl-benzyl)-methoxycarbonyl-amino]-6-methoxy-2-methyl-3,4-dihydro-2H-quinoline-1-carboxylic acid ethyl ester;

[2R,4S] 4-[(3,5-bis-trifluoromethyl-benzyl)-methoxycarbonyl-amino]-7-methoxy-2-methyl-3,4-dihydro-2H-quinoline-1-carboxylic acid ethyl ester,

[2R,4S] 4-[(3,5-bis-trifluoromethyl-benzyl)-methoxycarbonyl-amino]-6,7-dimethoxy-2-methyl-3,4-dihydro-2H-quinoline-1-carboxylic acid isopropyl ester;

[2R,4S] 4-[(3,5-bis-trifluoromethyl-benzyl)-ethoxycarbonyl-amino]-6,7-dimethoxy-2-methyl-3,4-dihydro-2H-quinoline-1-carboxylic acid ethyl ester;

[2R,4S] 4-[(3,5-bis-trifluoromethyl-benzyl)-methoxycarbonyl-amino]-6,7-dimethoxy-2-methyl-3,4-dihydro-2H-quinoline-1-carboxylic acid 2,2,2-trifluoro-ethylester;

[2R,4S] 4-[(3,5-bis-trifluoromethyl-benzyl)-methoxycarbonyl-amino]-6,7-dimethoxy-2-methyl-3,4-dihydro-2H-quinoline-1-carboxylic acid propyl ester;

[2R,4S] 4-[(3,5-bis-trifluoromethyl-benzyl)-methoxycarbonyl-amino]-6,7-dimethoxy-2-methyl-3,4-dihydro-2H-quinoline-1-carboxylic acid tert-butyl ester;

[2R,4S] 4-[(3,5-bis-trifluoromethyl-benzyl)-methoxycarbonyl-amino]-2-methyl-6-trifluoromethoxy-3,4-dihydro-2H-quinoline-1-carboxylic acid ethyl ester,

[2R,4S] (3,5-bis-trifluoromethyl-benzyl)-(1-butyryl-6,7-dimethoxy-2-methyl-1,2,3,4-tetrahydro-quinolin-4-yl)-carbamic acid methyl ester;

[2R,4S] (3,5-bis-trifluoromethyl-benzyl)-(1-butyl-6,7-dimethoxy-2-ethyl-1,2,3,4-tetrahydro-quinolin-4-yl)-carbamic acid methyl ester;

[2R,4S] (3,5-bis-trifluoromethyl-benzyl)-[1-(2-ethyl-butyl)-6,7-dimethoxy-2-methyl-1,2,3,4-tetrahydro-quinolin-4-yl]-carbamic acid methyl ester, hydrochloride

Another class of CETP inhibitors that finds utility with the present invention consists of 4-carboxyamino-2-methyl-1,2,3,4,-tetrahydroquinolines, having the Formula II

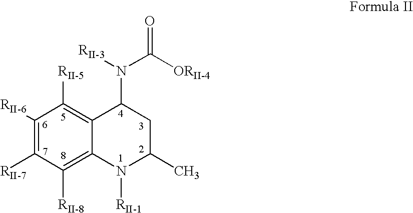

and pharmaceutically acceptable forms thereof;

wherein R_{II-1} is hydrogen, Y_{II}, W_{II}—X_{II}, W_{II}—Y_{II};

wherein W_{II} is a carbonyl, thiocarbonyl, sulfinyl or sulfonyl;

X_{II} is —O—Y_{II}, —S—Y_{II}, —N(H)—Y_{II} or —N—(Y_{II})₂;

wherein Y_{II} for each occurrence is independently Z_{II} or a fully saturated, partially unsaturated or fully unsaturated one to ten membered straight or branched carbon chain wherein the carbons, other than the connecting carbon, may optionally be replaced with one or two heteroatoms selected independently from oxygen, sulfur and nitrogen and said carbon is optionally mono-, di- or tri-substituted independently with halo, said carbon is optionally mono-substituted with hydroxy, said carbon is optionally mono-substituted with oxo, said sulfur is optionally mono- or di-substituted with oxo, said nitrogen is optionally mono-, or di-substituted with oxo, and said carbon chain is optionally mono-substituted with Z_{II};

Z_{II} is a partially saturated, fully saturated or fully unsaturated three to twelve membered ring optionally having one to four heteroatoms selected independently from oxygen, sulfur and nitrogen, or a bicyclic ring consisting of two fused partially saturated, fully saturated or fully unsaturated three to six membered rings, taken independently, optionally having one to four heteroatoms selected independently from nitrogen, sulfur and oxygen;

wherein said Z_{II} substituent is optionally mono-, di- or tri-substituted independently with halo, (C₂-C₆)alkenyl, (C₁-C₆) alkyl, hydroxy, (C₁-C₆)alkoxy, (C₁-C₄)alkylthio, amino, nitro, cyano, oxo, carboxy, (C₁-C₆)alkyloxycarbonyl, mono-N- or di-N,N-(C₁-C₆)alkylamino wherein said (C₁-C₆)alkyl substituent is optionally mono-, di- or tri-substituted independently with halo, hydroxy, (C₁-C₆)alkoxy, (C₁-C₄)alkylthio, amino, nitro, cyano, oxo, carboxy, (C₁-C₆)alkyloxycarbonyl, mono-N- or di-N,N-(C₁-C₆)alkylamino, said (C₁-C₆)alkyl is also optionally substituted with from one to nine fluorines;

R_{II-3} is hydrogen or Q_{II};

wherein Q_{II} is a fully saturated, partially unsaturated or fully unsaturated one to six membered straight or branched carbon chain wherein the carbons, other than the connecting carbon, may optionally be replaced with one heteroatom selected from oxygen, sulfur and nitrogen and said carbon is optionally mono-, di- or tri-substituted independently with halo, said carbon is optionally mono-substituted with hydroxy, said carbon is optionally mono-substituted with oxo, said sulfur is optionally mono- or di-substituted with oxo, said nitrogen is optionally mono- or di-substituted with oxo, and said carbon chain is optionally mono-substituted with V_{II};

wherein V_{II} is a partially saturated, fully saturated or fully unsaturated three to twelve membered ring optionally having one to four heteroatoms selected independently from oxygen, sulfur and nitrogen, or, a bicyclic ring consisting of two fused partially saturated, fully saturated or fully unsaturated three to six membered rings, taken independently, optionally having one to four heteroatoms selected independently from nitrogen, sulfur and oxygen;

wherein said V_{II} substituent is optionally mono-, di-, tri-, or tetra-substituted independently with halo, (C₁-C₆)alkyl, (C₂-C₆)alkenyl, hydroxy, (C₁-C₆)alkoxy, (C₁-C₄)alkylthio, amino, nitro, cyano, oxo, carboxamoyl, mono-N- or di-N,N-(C₁-C₆) alkylcarboxamoyl, carboxy, (C₁-C₆)alkyloxycarbonyl, mono-N- or di-N,N-(C₁-C₆)alkylamino wherein said (C₁-C₆)alkyl or (C₂-C₆)alkenyl substituent is optionally mono-, di- or tri-substituted independently with hydroxy, (C₁-C₆)alkoxy, (C₁-C₄)alkylthio, amino, nitro, cyano, oxo, carboxy, (C₁-C₆)alkyloxycarbonyl, mono-N- or di-N,N-(C₁-C₆)alkylamino or said (C₁-C₆)alkyl or (C₂-C₆)alkenyl substituents are optionally substituted with from one to nine fluorines;

R_{II-4} is Q_{II-1} or V_{II-1}

wherein Q_{II-1} a fully saturated, partially unsaturated or fully unsaturated one to six membered straight or branched carbon chain wherein the carbons, other than the connecting carbon, may optionally be replaced with one heteroatom selected from oxygen, sulfur and nitrogen and said carbon is optionally mono-, di- or tri-substituted independently with halo, said carbon is optionally mono-substituted with hydroxy, said carbon is optionally mono-substituted with oxo, said sulfur is optionally mono- or di-substituted with oxo, said nitrogen is optionally mono- or di-substituted with oxo, and said carbon chain is optionally mono-substituted with V_{II-1};

wherein V_{II-1} is a partially saturated, fully saturated or fully unsaturated three to six membered ring optionally having one to two heteroatoms selected independently from oxygen, sulfur and nitrogen;

wherein said V_{II-1} substituent is optionally mono-, di-, tri-, or tetra-substituted independently with halo, (C₁-C₆)alkyl, (C₁-C₆)alkoxy, amino, nitro, cyano, (C₁-C₆)alkyloxycarbonyl, mono-N- or di-N,N-(C₁-C₆)alkylamino wherein said (C₁-C₆)alkyl substituent is optionally mono-substituted with oxo, said (C₁-C₆)alkyl substituent is optionally substituted with from one to nine fluorines;

wherein either R_{II-3} must contain V_{II} or R_{II-4} must contain V_{II-1}; and

R_{II-5}, R_{II-6}, R_{II-7} and R_{II-8} are each independently hydrogen, a bond, nitro or halo wherein said bond is substituted with T_{II} or a partially saturated, fully saturated or fully unsaturated (C₁-C₁₂) straight or branched carbon chain wherein carbon may optionally be replaced with one or two heteroatoms selected independently from oxygen, sulfur and nitrogen wherein said carbon atoms are optionally mono-, di- or tri-substituted independently with halo, said carbon is optionally mono-substituted with hydroxy, said carbon is optionally mono-substituted with oxo, said sulfur is optionally mono- or di-substituted with oxo, said nitrogen is optionally mono- or di-substituted with oxo, and said carbon is optionally mono-substituted with T_{II};

wherein T_{II} is a partially saturated, fully saturated or fully unsaturated three to twelve membered ring optionally having one to four heteroatoms selected independently from oxygen, sulfur and nitrogen, or, a bicyclic ring consisting of two fused partially saturated, fully saturated or fully unsaturated three to six membered rings, taken independently, optionally having one to four heteroatoms selected independently from nitrogen, sulfur and oxygen;

wherein said T_{II} substituent is optionally mono-, di- or tri-substituted independently with halo, (C₁-C₆)alkyl, (C₂-C₆)alkenyl, hydroxy, (C₁-C₆)alkoxy, (C₁-C₄)alkylthio, amino, nitro, cyano, oxo, carboxy, (C₁-C₆)alkyloxycarbonyl, mono-N- or di-N,N-(C₁-C₆)alkylamino wherein said (C₁-C₆)alkyl substituent is optionally mono-, di- or tri-substituted independently with hydroxy, (C₁-C₆)alkoxy, (C₁-C₄)alkylthio, amino, nitro, cyano, oxo, carboxy, (C₁-C₆)alkyloxycarbonyl, mono-N- or di-N,N-(C₁-C₆)alkylamino, said (C₁-C₆)alkyl substituent is also optionally substituted with from one to nine fluorines; provided that at least one of substituents R_{II-5}, R_{II-6}, R_{II-7} and R_{II-8} is not hydrogen and is not linked to the quinoline moiety through oxy.

Compounds of Formula II are disclosed in commonly assigned U.S. Pat. No. 6,147,090, the complete disclosure of which is herein incorporated by reference.

In a preferred embodiment, the CETP inhibitor is selected from one of the following compounds of Formula II:

[2R,4S] 4-[(3,5-Bis-trifluoromethyl-benzyl)-methoxycarbonyl-amino]-2-methyl-7-trifluoromethyl-3,4-dihydro-2H-quinoline-1-carboxylic acid ethyl ester;

[2R,4S] 4-[(3,5-Bis-trifluoromethyl-benzyl)-methoxycarbonyl-amino]-7-chloro-2-methyl-3,4-dihydro-2H-quinoline-1-carboxylic acid ethyl ester;

[2R,4S] 4-[(3,5-Bis-trifluoromethyl-benzyl)-methoxycarbonyl-amino]-6-chloro-2-methyl-3,4-dihydro-2H-quinoline-1-carboxylic acid ethyl ester;

[2R,4S] 4-[(3,5-Bis-trifluoromethyl-benzyl)-methoxycarbonyl-amino]-2,6,7-trimethyl-3,4-dihydro-2H-quinoline-1-carboxylic acid ethyl ester

[2R,4S] 4-[(3,5-Bis-trifluoromethyl-benzyl)-methoxycarbonyl-amino]-6,7-diethyl-2-methyl-3,4-dihydro-2H-quinoline-1-carboxylic acid ethyl ester;

[2R,4S] 4-[(3,5-Bis-trifluoromethyl-benzyl)-methoxycarbonyl-amino]-6-ethyl-2-methyl-3,4-dihydro-2H-quinoline-1-carboxylic acid ethyl ester;

[2R,4S] 4-[(3,5-Bis-trifluoromethyl-benzyl)-methoxycarbonyl-amino]-2-methyl-6-trifluoromethyl-3,4-dihydro-2H-quinoline-1-carboxylic acid ethyl ester.

[2R,4S] 4-[(3,5-bis-trifluoromethyl-benzyl)-methoxycarbonyl-amino]-2-methyl-6-trifluoromethyl-3,4-dihydro-2H-quinoline-1-carboxylic acid isopropyl ester.

Another class of CETP inhibitors that finds utility with the present invention consists of annulated 4-carboxyamino-2-methyl-1,2,3,4,-tetrahydroquinolines, having the Formula III

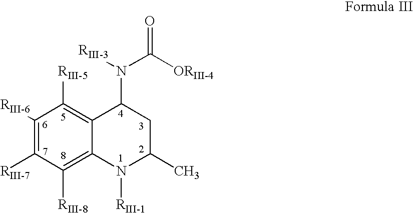

and pharmaceutically acceptable forms thereof;

wherein R_{III-1}, is hydrogen, Y_{III}, W_{III}—X_{III}, W_{III}—Y_{III};

wherein W_{III} is a carbonyl, thiocarbonyl, sulfinyl or sulfonyl;

X_{III} is —O—Y_{III}, —S—Y_{III}, —N(H)—Y_{III} or —N—(Y_{III})₂;

Y_{III} for each occurrence is independently Z_{III} or a fully saturated, partially unsaturated or fully unsaturated one to ten membered straight or branched carbon chain wherein the carbons, other than the connecting carbon, may optionally be replaced with one or two heteroatoms selected independently from oxygen, sulfur and nitrogen and said carbon is optionally mono-, di- or tri-substituted independently with halo, said carbon is optionally mono-substituted with hydroxy, said carbon is optionally mono-substituted with oxo, said sulfur is optionally mono- or di-substituted with oxo, said nitrogen is optionally mono-, or di-substituted with oxo, and said carbon chain is optionally mono-substituted with Z_{III};

wherein Z_{III} is a partially saturated, fully saturated or fully unsaturated three to twelve membered ring optionally having one to four heteroatoms selected independently from oxygen, sulfur and nitrogen, or a bicyclic ring consisting of two fused partially saturated, fully saturated or fully unsaturated three to six membered rings, taken independently, optionally having one to four heteroatoms selected independently from nitrogen, sulfur and oxygen;

wherein said Z_{III} substituent is optionally mono-, di- or tri-substituted independently with halo, (C₂-C₆)alkenyl, (C₁-C₆) alkyl, hydroxy, (C₁-C₆)alkoxy, (C₁-C₄)alkylthio, amino, nitro, cyano, oxo, carboxy, (C₁-C₆)alkyloxycarbonyl, mono-N- or di-N,N-(C₁-C₆)alkylamino wherein said (C₁-C₆)alkyl substituent is optionally mono-, di- or tri-substituted independently with halo, hydroxy, (C₁-C₆)alkoxy, (C₁-C₄)alkylthio, amino, nitro, cyano, oxo, carboxy, (C₁-C₆)alkyloxycarbonyl, mono-N- or di-N,N-(C₁-C₆)alkylamino, said (C₁-C₆)alkyl optionally substituted with from one to nine fluorines;

R_{III-3} is hydrogen or Q_{III};

wherein Q_{III} is a fully saturated, partially unsaturated or fully unsaturated one to six membered straight or branched carbon chain wherein the carbons, other than the connecting carbon, may optionally be replaced with one heteroatom selected from oxygen, sulfur and nitrogen and said carbon is optionally mono-, di- or tri-substituted independently with halo, said carbon is optionally mono-substituted with hydroxy, said carbon is optionally mono-substituted with oxo, said sulfur is optionally mono- or di-substituted with oxo, said nitrogen is optionally mono- or di-substituted with oxo, and said carbon chain is optionally mono-substituted with V_{III};

wherein V_{III} is a partially saturated, fully saturated or fully unsaturated three to twelve membered ring optionally having one to four heteroatoms selected independently from oxygen, sulfur and nitrogen, or a bicyclic ring consisting of two fused partially saturated, fully saturated or fully unsaturated three to six membered rings, taken independently, optionally having one to four heteroatoms selected independently from nitrogen, sulfur and oxygen;

wherein said V_{III} substituent is optionally mono-, di-, tri-, or tetra-substituted independently with halo, (C₁-C₆)alkyl, (C₂-C₆)alkenyl, hydroxy, (C₁-C₆)alkoxy, (C₁-C₄)alkylthio, amino, nitro, cyano, oxo, carboxamoyl, mono-N- or di-N,N-(C₁-C₆) alkylcarboxamoyl, carboxy, (C₁-C₆)alkyloxycarbonyl, mono-N- or di-N,N-(C₁-C₆)alkylamino wherein said (C₁-C₆)alkyl or (C₂-C₆)alkenyl substituent is optionally mono-, di- or tri-substituted independently with hydroxy, (C₁-C₆)alkoxy, (C₁-C₄)alkylthio, amino, nitro, cyano, oxo, carboxy, (C₁-C₆)alkyloxycarbonyl, mono-N- or di-N,N-(C₁-C₆)alkylamino or said (C₁-C₆)alkyl or (C₂-C₆)alkenyl are optionally substituted with from one to nine fluorines;

R_{III-4} is Q_{III-1} or V_{III-1};

wherein Q_{III-1} a fully saturated, partially unsaturated or fully unsaturated one to six membered straight or branched carbon chain wherein the carbons, other than the connecting carbon, may optionally be replaced with one heteroatom selected from oxygen, sulfur and nitrogen and said carbon is optionally mono-, di- or tri-substituted independently with halo, said carbon is optionally mono-substituted with hydroxy, said carbon is optionally mono-substituted with oxo, said sulfur is optionally mono- or di-substituted with oxo, said nitrogen is optionally mono- or di-substituted with oxo, and said carbon chain is optionally mono-substituted with V_{III-1};

wherein V_{III-1} is a partially saturated, fully saturated or fully unsaturated three to six membered ring optionally having one to two heteroatoms selected independently from oxygen, sulfur and nitrogen;

wherein said V_{III-1} substituent is optionally mono-, di-, tri-, or tetra-substituted independently with halo, (C₁-C₆)alkyl, (C₁-C₆)alkoxy, amino, nitro, cyano, (C₁-C₆)alkyloxycarbonyl, mono-N- or di-N,N-(C₁-C₆)alkylamino wherein said (C₁-C₆)alkyl substituent is optionally mono-substituted with oxo, said (C₁-C₆)alkyl substituent optionally having from one to nine fluorines;

wherein either R_{III-3} must contain V_{III} or R_{III-4} must contain V_{III-1}; and R_{III-5} and R_{III-6}, or R_{III-6} and R_{III-7}, and/or R_{III-7} and R_{III-8} are taken together and form a four to eight membered ring that is partially saturated or fully unsaturated optionally having one to three heteroatoms independently selected from nitrogen, sulfur and oxygen;

wherein said ring or rings formed by R_{III-5} and R_{III-6}, or R_{III-6} and R_{III-7}, and/or R_{III-7} and R_{III-8} are optionally mono-, di- or tri-substituted independently with halo, (C₁-C₆)alkyl, (C₁-C₄)alkylsulfonyl, (C₂-C₆)alkenyl, hydroxy, (C₁-C₆)alkoxy, (C₁-C₄)alkylthio, amino, nitro, cyano, oxo, carboxy, (C₁-C₆)alkyloxycarbonyl, mono-N- or di-N,N-(C₁-C₆)alkylamino wherein said (C₁-C₆)alkyl substituent is optionally mono-, di- or tri-substituted independently with hydroxy, (C₁-C₆)alkoxy, (C₁-C₄)alkylthio, amino, nitro, cyano, oxo, carboxy, (C₁-C₆)alkyloxycarbonyl, mono-N- or di-N,N-(C₁-C₆)alkylamino, said (C₁-C₆)alkyl substituent optionally having from one to nine fluorines;

provided that the R_{III-5}, R_{III-6}, R_{III-7} and/or R_{III-8}, as the case may be, that do not form at least one ring are each independently hydrogen, halo, (C₁-C₆)alkoxy or (C₁-C₆)alkyl, said (C₁-C₆)alkyl optionally having from one to nine fluorines.

Compounds of Formula III are disclosed in commonly assigned pending U.S. Pat. No. 6,147,089, the complete disclosure of which is herein incorporated by reference.

In a preferred embodiment, the CETP inhibitor is selected from one of the following compounds of Formula III:

[2R,4S] 4-[(3,5-bis-trifluoromethyl-benzyl)-methoxycarbonyl-amino]-2-methyl-2,3,4,6,7,8-hexahydro-cyclopenta[g]quinoline-1-carboxylic acid ethyl ester;

[6R,8S] 8-[(3,5-bis-trifluoromethyl-benzyl)-methoxycarbonyl-amino]-6-methyl-3,6,7,8-tetrahydro-1H-2-thia-5-aza-cyclopenta[b]naphthalene-5-carboxylic acid ethylester;

[6R,8S] 8-[(3,5-bis-trifluoromethyl-benzyl)-methoxycarbonyl-amino]-6-methyl-3,6,7,8-tetrahydro-2H-furo[2,3-g]quinoline-5-carboxylic acid ethyl ester;

[2R,4S] 4-[(3,5-bis-trifluoromethyl-benzyl)-methoxycarbonyl-amino]-2-methyl-3,4,6,8-tetrahydro-2H-furo[3,4-g]quinoline-1-carboxylic acid ethyl ester;

[2R,4S] 4-[(3,5-bis-trifluoromethyl-benzyl)-methoxycarbonyl-amino]-2-methyl-3,4,6,7,8,9-hexahydro-2H-benzo[g]quinoline-1-carboxylic acid propyl ester;

[7R,9S] 9-[(3,5-bis-trifluoromethyl-benzyl)-methoxycarbonyl-amino]-7-methyl-1,2,3,7,8,9-hexahydro-6-aza-cyclopenta[a]naphthalene-6-carboxylic acid ethyl ester; and

[6S,8R] 6-[(3,5-bis-trifluoromethyl-benzyl)-methoxycarbonyl-amino]-8-methyl-1,2,3,6,7,8-hexahydro-9-aza-cyclopenta[a]naphthalene-9-carboxylic acid ethyl ester.

Another class of CETP inhibitors that finds utility with the present invention consists of 4-carboxyamino-2-substituted-1,2,3,4,-tetrahydroquinolines, having the Formula IV

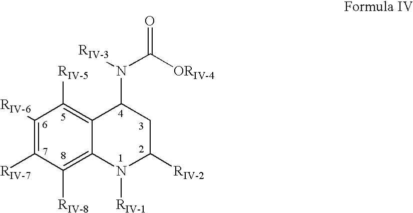

and pharmaceutically acceptable forms thereof;

wherein R_{IV-1} is hydrogen, Y_{IV}, W_{IV}—-X_{IV} or W_{IV}—Y_{IV};

wherein W_{IV} is a carbonyl, thiocarbonyl, sulfinyl or sulfonyl;

X_{IV} is —O—Y_{IV}, —S—Y_{IV}, —N(H)—Y_{IV} or —N—(Y_{IV})₂;

wherein Y_{IV} for each occurrence is independently Z_{IV} or a fully saturated, partially unsaturated or fully unsaturated one to ten membered straight or branched carbon chain wherein the carbons, other than the connecting carbon, may optionally be replaced with one or two heteroatoms selected independently from oxygen, sulfur and nitrogen and said carbon is optionally mono-, di- or tri-substituted independently with halo, said carbon is optionally mono-substituted with hydroxy, said carbon is optionally mono-substituted with oxo, said sulfur is optionally mono- or di-substituted with oxo, said nitrogen is optionally mono-, or di-substituted with oxo, and said carbon chain is optionally mono-substituted with Z_{IV};

wherein Z_{IV} is a partially saturated, fully saturated or fully unsaturated three to eight membered ring optionally having one to four heteroatoms selected independently from oxygen, sulfur and nitrogen, or a bicyclic ring consisting of two fused partially saturated, fully saturated or fully unsaturated three to six membered rings, taken independently, optionally having one to four heteroatoms selected independently from nitrogen, sulfur and oxygen;

wherein said Z_{IV} substituent is optionally mono-, di- or tri-substituted independently with halo, (C₂-C₆)alkenyl, (C₁-C₆) alkyl, hydroxy, (C₁-C₆)alkoxy, (C₁-C₄)alkylthio, amino, nitro, cyano, oxo, carboxy, (C₁-C₆)alkyloxycarbonyl, mono-N- or di-N,N-(C₁-C₆)alkylamino wherein said (C₁-C₆)alkyl substituent is optionally mono-, di- or tri-substituted independently with halo, hydroxy, (C₁-C₆)alkoxy, (C₁-C₄)alkylthio, amino, nitro, cyano, oxo, carboxy, (C₁-C₆)alkyloxycarbonyl, mono-N- or di-N,N-C₁-C₆)alkylamino, said (C₁-C₆)alkyl substituent is also optionally substituted with from one to nine fluorines;

R_{IV-2} is a partially saturated, fully saturated or fully unsaturated one to six membered straight or branched carbon chain wherein the carbons, other than the connecting carbon, may optionally be replaced with one or two heteroatoms selected independently from oxygen, sulfur and nitrogen wherein said carbon atoms are optionally mono-, di- or tri-substituted independently with halo, said carbon is optionally mono-substituted with oxo, said carbon is optionally mono-substituted with hydroxy, said sulfur is optionally mono- or di-substituted with oxo, said nitrogen is optionally mono- or di-substituted with oxo; or said R_{IV-2} is a partially saturated, fully saturated or fully unsaturated three to seven membered ring optionally having one to two heteroatoms selected independently from oxygen, sulfur and nitrogen, wherein said R_{IV-2} ring is optionally attached through (C₁-C₄)alkyl;

wherein said R_{IV-2} ring is optionally mono-, di- or tri-substituted independently with halo, (C₂-C₆)alkenyl, (C₁-C₆) alkyl, hydroxy, (C₁-C₆)alkoxy, (C_{1-C}₄)alkylthio, amino, nitro, cyano, oxo, carboxy, (C₁-C₆)alkyloxycarbonyl, mono-N- or di-N,N-(C₁-C₆)alkylamino wherein said (C₁-C₆)alkyl substituent is optionally mono-, di- or tri-substituted independently with halo, hydroxy, (C₁-C₆)alkoxy, (C₁-C₄)alkylthio, oxo or (C₁-C₆)alkyloxycarbonyl;

with the proviso that R_{IV-2} is not methyl;

R_{IV-3} is hydrogen or Q_{IV};

wherein Q_{IV} is a fully saturated, partially unsaturated or fully unsaturated one to six membered straight or branched carbon chain wherein the carbons other than the connecting carbon, may optionally be replaced with one heteroatom selected from oxygen, sulfur and nitrogen and said carbon is optionally mono-, di- or tri-substituted independently with halo, said carbon is optionally mono-substituted with hydroxy, said carbon is optionally mono-substituted with oxo, said sulfur is optionally mono- or di-substituted with oxo, said nitrogen is optionally mono- or di-substituted with oxo, and said carbon chain is optionally mono-substituted with V_{IV};

wherein V_{IV} is a partially saturated, fully saturated or fully unsaturated three to eight membered ring optionally having one to four heteroatoms selected independently from oxygen, sulfur and nitrogen, or a bicyclic ring consisting of two fused partially saturated, fully saturated or fully unsaturated three to six membered rings, taken independently, optionally having one to four heteroatoms selected independently from nitrogen, sulfur and oxygen;

wherein said V_{IV} substituent is optionally mono-, di-, tri-, or tetra-substituted independently with halo, (C₁-C₆)alkyl, (C₂-C₆)alkenyl, hydroxy, (C₁-C₆)alkoxy, (C₁-C₄)alkylthio, amino, nitro, cyano, oxo, carboxamoyl, mono-N- or di-N,N-(C₁-C₆) alkylcarboxamoyl, carboxy, (C₁-C₆)alkyloxycarbonyl, mono-N- or di-N,N-(C₁-C₆)alkylamino wherein said (C₁-C₆)alkyl or (C₂-C₆)alkenyl substituent is optionally mono-, di- or tri-substituted independently with hydroxy, (C₁-C₆)alkoxy, (C₁-C₄)alkylthio, amino, nitro, cyano, oxo, carboxy, (C₁-C₆)alkyloxycarbonyl, mono-N- or di-N,N-(C₁-C₆)alkylamino, said (C₁-C₆)alkyl or (C₂-C₆)alkenyl substituents are also optionally substituted with from one to nine fluorines;

R_{IV-4} is Q_{IV-1} or V_{IV-1};

wherein Q_{IV-1} a fully saturated, partially unsaturated or fully unsaturated one to six membered straight or branched carbon chain wherein the carbons, other than the connecting carbon, may optionally be replaced with one heteroatom selected from oxygen, sulfur and nitrogen and said carbon is optionally mono-, di- or tri-substituted independently with halo, said carbon is optionally mono-substituted with hydroxy, said carbon is optionally mono-substituted with oxo, said sulfur is optionally mono- or di-substituted with oxo, said nitrogen is optionally mono- or di-substituted with oxo, and said carbon chain is optionally mono-substituted with V_{IV-1};

wherein V_{IV-1} is a partially saturated, fully saturated or fully unsaturated three to six membered ring optionally having one to two heteroatoms selected independently from oxygen, sulfur and nitrogen;

wherein said V_{IV-1} substituent is optionally mono-, di-, tri-, or tetra-substituted independently with halo, (C₁-C₆)alkyl, (C₁-C₆)alkoxy, amino, nitro, cyano, (C₁-C₆)alkyloxycarbonyl, mono-N- or di-N,N-(C₁-C₆)alkylamino wherein said (C₁-C₆)alkyl substituent is optionally mono-substituted with oxo, said (C₁-C₆)alkyl substituent is also optionally substituted with from one to nine fluorines;

wherein either R_{IV-3} must contain V_{IV} or R_{IV-4} must contain V_{IV-1};

R_{IV-5}, R_{IV-6} R_{IV-7} and R_{IV-8} are each independently hydrogen, a bond, nitro or halo wherein said bond is substituted with T_{IV} or a partially saturated, fully saturated or fully unsaturated (C₁-C₁₂) straight or branched carbon chain wherein carbon, may optionally be replaced with one or two heteroatoms selected independently from oxygen, sulfur and nitrogen wherein said carbon atoms are optionally mono-, di- or tri-substituted independently with halo, said carbon is optionally mono-substituted with hydroxy, said carbon is optionally mono-substituted with oxo, said sulfur is optionally mono- or di-substituted with oxo, said nitrogen is optionally mono- or di-substituted with oxo, and said carbon is optionally mono-substituted with T_{IV};

wherein T_{IV} is a partially saturated, fully saturated or fully unsaturated three to eight membered ring optionally having one to four heteroatoms selected independently from oxygen, sulfur and nitrogen, or, a bicyclic ring consisting of two fused partially saturated, fully saturated or fully unsaturated three to six membered rings, taken independently, optionally having one to four heteroatoms selected independently from nitrogen, sulfur and oxygen;

wherein said T_{IV} substituent is optionally mono-, di- or tri-substituted independently with halo, (C₁-C₆)alkyl, (C₂-C₆)alkenyl, hydroxy, (C₁-C₆)alkoxy, (C₁-C₄)alkylthio, amino, nitro, cyano, oxo, carboxy, (C₁-C₆)alkyloxycarbonyl, mono-N- or di-N,N-(C₁-C₆)alkylamino wherein said (C₁-C₆)alkyl substituent is optionally mono-, di- or tri-substituted independently with hydroxy, (C₁-C₆)alkoxy, (C₁-C₄)alkylthio, amino, nitro, cyano, oxo, carboxy, (C₁-C₆)alkyloxycarbonyl, mono-N- or di-N,N-(C₁-C₆)alkylamino, said (C₁-C₆)alkyl substituent is also optionally substituted with from one to nine fluorines; and

wherein R_{IV-5} and R_{IV-6} or R_{IV-6} and R_{IV-7}, and/or R_{IV-7} and R_{IV-8} may also be taken together and can form at least one four to eight membered ring that is partially saturated or fully unsaturated optionally having one to three heteroatoms independently selected from nitrogen, sulfur and oxygen;

wherein said ring or rings formed by R_{IV-5} and R_{IV-6}, or R_{IV-6} and R_{IV-7}, and/or R_{IV-7} and R_{IV-8} are optionally mono-, di- or tri-substituted independently with halo, (C₁-C₆)alkyl, (C₁-C₄)alkylsulfonyl, (C₂-C₆)alkenyl, hydroxy, (C₁-C₆)alkoxy, (C₁-C₄)alkylthio, amino, nitro, cyano, oxo, carboxy, (C₁-C₆)alkyloxycarbonyl, mono-N- or di-N,N-(C₁-C₆)alkylamino wherein said (C₁-C₆)alkyl substituent is optionally mono-, di- or tri-substituted independently with hydroxy, (C₁-C₆)alkoxy, (C₁-C₄)alkylthio, amino, nitro, cyano, oxo, carboxy, (C₁-C₆)alkyloxycarbonyl, mono-N- or di-N,N-(C₁-C₆)alkylamino, said (C₁-C₆)alkyl substituent is also optionally substituted with from one to nine fluorines; with the proviso that when R_{IV-2} is carboxyl or (C₁-C₄) alkylcarboxyl, then R_{IV-1} is not hydrogen.

Compounds of Formula IV are disclosed in commonly assigned U.S. Pat. No. 6,197,786, the complete disclosure of which is herein incorporated by reference.

In a preferred embodiment, the CETP inhibitor is selected from one of the following compounds of Formula IV:

[2S,4S] 4-[(3,5-bis-trifluoromethyl-benzyl)-methoxycarbonyl-amino]-2-isopropyl-6-trifluoromethyl-3,4-dihydro-2H-quinoline-1-carboxylic acid isopropyl ester;

[2S,4S] 4-[(3,5-bis-trifluoromethyl-benzyl)-methoxycarbonyl-amino]-6-chloro-2-cyclopropyl-3,4-dihydro-2H-quinoline-l-carboxylic acid isopropyl ester;

[2S,4S] 2-cyclopropyl-4-[(3,5-dichloro-benzyl)-methoxycarbonyl-amino]-6-trifluoromethyl-3,4-dihydro-2H-quinoline-1-carboxylic acid isopropyl ester;

[2S,4S] 4-[(3,5-bis-trifluoromethyl-benzyl)-methoxycarbonyl-amino]-2-cyclopropyl-6-trifluoromethyl-3,4-dihydro-2H-quinoline-1-carboxylic acid tert-butyl ester;

[2R,4R] 4-[(3,5-bis-trifluoromethyl-benzyl)-methoxycarbonyl-amino]-2-cyclopropyl-6-trifluoromethyl-3,4-dihydro-2H-quinaline-1-carboxylic acid isopropyl ester;

[2S,4S] 4-[(3,5-bis-trifluoromethyl-benzyl)-methoxycarbonyl-amino]-2-cyclopropyl-6-trifluoromethyl-3,4-dihydro-2H-quinoline-1-carboxylic acid isopropyl ester;

[2S,4S] 4-[(3,5-bis-trifluoromethyl-benzyl)-methoxycarbonyl-amino]-2-cyclobutyl-6-trifluoromethyl-3,4-dihydro-2H-quinoline-1-carboxylic acid isopropyl ester,

[2R,4S] 4-[(3,5-bis-trifluoromethyl-benzyl)-methoxycarbonyl-amino]-2-ethyl-6-trifluoromethyl-3,4-dihydro-2H-quinoline-1-carboxylic acid isopropyl ester;

[2S,4S] 4-[(3,5-bis-trifluoromethyl-benzyl)-methoxycarbonyl-amino]-2-methoxymethyl-6-trifluoromethyl-3,4-dihydro-2H-quinoline-1-carboxylic acid isopropyl ester;

[2R,4S] 4-[(3,5-bis-trifluoromethyl-benzyl)-methoxycarbonyl-amino]-2-ethyl-6-trifluoromethyl-3,4-dihydro-2H-quinoline-1-carboxylic acid 2-hydroxy-ethyl ester;

[2S,4S] 4-[(3,5-bis-trifluoromethyl-benzyl)-methoxycarbonyl-amino]-2-cyclopropyl-6-trifluoromethyl-3,4-dihydro-2H-quinoline-1-carboxylic acid ethyl ester;

[2R,4S] 4-[(3,5-bis-trifluoromethyl-benzyl)-methoxycarbonyl-amino]-2-ethyl-6-trifluoromethyl-3,4-dihydro-2H-quinoline-1-carboxylic acid ethyl ester;

[2S,4S] 4-[(3,5-bis-trifluoromethyl-benzyl)-methoxycarbonyl-amino]-2-cyclopropyl-6-trifluoromethyl-3,4-dihydro-2H-quinoline-1-carboxylic acid propyl ester; and

[2R,4S] 4-[(3,5-bis-trifluoromethyl-benzyl)-methoxycarbonyl-amino]-2-ethyl-6-trifluoromethyl-3,4-dihydro-2H-quinoline-1-carboxylic acid propyl ester.

Another class of CETP inhibitors that finds utility with the present invention consists of 4-amino substituted-2-substituted-1,2,3,4,-tetrahydroquinolines, having the Formula V

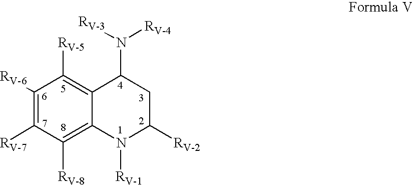

and pharmaceutically acceptable forms thereof;

wherein R_{V-1} is Y_{V}, W_{V}—X_{V} or W_{V}—Y_{V};

wherein W_{V} is a carbonyl, thiocarbonyl, sulfinyl or sulfonyl;

X_{V} is —O—Y_{V}, —S—Y_{V}, —N(H)—Y_{V} or —N—(Y_{V})₂;

wherein Y_{V} for each occurrence is independently Z_{V} or a fully saturated, partially unsaturated or fully unsaturated one to ten membered straight or branched carbon chain wherein the carbons, other than the connecting carbon, may optionally be replaced with one or two heteroatoms selected independently from oxygen, sulfur and nitrogen and said carbon is optionally mono-, di- or tri-substituted independently with halo, said carbon is optionally mono-substituted with hydroxy, said carbon is optionally mono-substituted with oxo, said sulfur is optionally mono- or di-substituted with oxo, said nitrogen is optionally mono-, or di-substituted with oxo, and said carbon chain is optionally mono-substituted with Z_{V};

wherein Z_{V} is a partially saturated, fully saturated or fully unsaturated three to eight membered ring optionally having one to four heteroatoms selected independently from oxygen, sulfur and nitrogen, or a bicyclic ring consisting of two fused partially saturated, fully saturated or fully unsaturated three to six membered rings, taken independently, optionally having one to four heteroatoms selected independently from nitrogen, sulfur and oxygen;

wherein said Z_{V} substituent is optionally mono-, di- or tri-substituted independently with halo, (C₂-C₆)alkenyl, (C₁-C₆) alkyl, hydroxy, (C₁-C₆)alkoxy, (C₁-C₄)alkylthio, amino, nitro, cyano, oxo, carboxy, (C₁-C₆)alkyloxycarbonyl, mono-N- or di-N,N-(C₁-C₆)alkylamino wherein said (C₁-C₆)alkyl substituent is optionally mono-, di- or tri-substituted independently with halo, hydroxy, (C₁-C₆)alkoxy, (C₁-C₄)alkylthio, amino, nitro, cyano, oxo, carboxy, (C₁-C₆)alkyloxycarbonyl, mono-N- or di-N,N-(C₁-C₆)alkylamino, said (C₁-C₆)alkyl substituent is also optionally substituted with from one to nine fluorines;

R_{V-2} is a partially saturated, fully saturated or fully unsaturated one to six membered straight or branched carbon chain wherein the carbons, other than the connecting carbon, may optionally be replaced with one or two heteroatoms selected independently from oxygen, sulfur and nitrogen wherein said carbon atoms are optionally mono-, di- or tri-substituted independently with halo, said carbon is optionally mono-substituted with oxo, said carbon is optionally mono-substituted with hydroxy, said sulfur is optionally mono- or di-substituted with oxo, said nitrogen is optionally mono- or di-substituted with oxo; or said R_{V-2} is a partially saturated, fully saturated or fully unsaturated three to seven membered ring optionally having one to two heteroatoms selected independently from oxygen, sulfur and nitrogen, wherein said R_{V-2} ring is optionally attached through (C₁-C₄)alkyl;

wherein said R_{V-2} ring is optionally mono-, di- or tri-substituted independently with halo, (C₂-C₆)alkenyl, (C_{1-C}₆) alkyl, hydroxy, (C₁-C₆)alkoxy, (C₁-C₄)alkylthio, amino, nitro, cyano, oxo, carboxy, (C₁-C₆)alkyloxycarbonyl, mono-N- or di-N,N-(C₁-C₆)alkylamino wherein said (C₁-C₆)alkyl substituent is optionally mono-, di- or tri-substituted independently with halo, hydroxy, (C₁-C₆)alkoxy, (C₁-C₄)alkylthio, oxo or (C₁-C₆)alkyloxycarbonyl;

R_{V-3} is hydrogen or Q_{V};

wherein Q_{V} is a fully saturated, partially unsaturated or fully unsaturated one to six membered straight or branched carbon chain wherein the carbons, other than the connecting carbon, may optionally be replaced with one heteroatom selected from oxygen, sulfur and nitrogen and said carbon is optionally mono-, di- or tri-substituted independently with halo, said carbon is optionally mono-substituted with hydroxy, said carbon is optionally mono-substituted with oxo, said sulfur is optionally mono- or di-substituted with oxo, said nitrogen is optionally mono-, or di-substituted with oxo, and said carbon chain is optionally mono-substituted with V_{V};

wherein V_{V} is a partially saturated, fully saturated or fully unsaturated three to eight membered ring optionally having one to four heteroatoms selected independently from oxygen, sulfur and nitrogen, or a bicyclic ring consisting of two fused partially saturated, fully saturated or fully unsaturated three to six membered rings, taken independently, optionally having one to four heteroatoms selected independently from nitrogen, sulfur and oxygen;

wherein said V_{V} substituent is optionally mono-, di-, tri-, or tetra-substituted independently with halo, (C₁-C₆)alkyl, (C₂-C₆)alkenyl, hydroxy, (C₁-C₆)alkoxy, (C₁-C₄)alkylthio, amino, nitro, cyano, oxo, carboxamoyl, mono-N- or di-N,N-(C₁-C₆) alkylcarboxamoyl, carboxy, (C₁-C₆)alkyloxycarbonyl, mono-N- or di-N,N-(C₁-C₆)alkylamino wherein said (C₁-C₆)alkyl or (C₂-C₆)alkenyl substituent is optionally mono-, di- or tri-substituted independently with hydroxy, (C₁-C₆)alkoxy, (C₁-C₄)alkylthio, amino, nitro, cyano, oxo, carboxy, (C₁-C₆)alkyloxycarbonyl, mono-N- or di-N,N-(C₁-C₆)alkylamino, said (C₁-C₆)alkyl or (C₂-C₆)alkenyl substituents are also optionally substituted with from one to nine fluorines;

R_{V-4} is cyano, formyl, W_{V-1}Q_{V-1}, W_{V-1}V_{V-1}, (C₁-C₄)alkylene V_{V-1} or V_{V-2};

wherein W_{V-1} is carbonyl, thiocarbonyl, SO or SO₂,

wherein Q_{V-1} a fully saturated, partially unsaturated or fully unsaturated one to six membered straight or branched carbon chain wherein the carbons may optionally be replaced with one heteroatom selected from oxygen, sulfur and nitrogen and said carbon is optionally mono-, di- or tri-substituted independently with halo, said carbon is optionally mono-substituted with hydroxy, said carbon is optionally mono-substituted with oxo, said sulfur is optionally mono- or di-substituted with oxo, said nitrogen is optionally mono-, or di-substituted with oxo, and said carbon chain is optionally mono-substituted with V_{V-1};

wherein V_{V-1} is a partially saturated, fully saturated or fully unsaturated three to six membered ring optionally having one to two heteroatoms selected independently from oxygen, sulfur and nitrogen, or a bicyclic ring consisting of two fused partially saturated, fully saturated or fully unsaturated three to six membered rings, taken independently, optionally having one to four heteroatoms selected independently from nitrogen, sulfur and oxygen;

wherein said V_{V-1} substituent is optionally mono-, di-, tri-, or tetra-substituted independently with halo, (C₁-C₆)alkyl, (C₁-C₆)alkoxy, hydroxy, oxo, amino, nitro, cyano, (C₁-C₆)alkyloxycarbonyl, mono-N- or di-N,N-(C₁-C₆)alkylamino wherein said (C₁-C₆)alkyl substituent is optionally mono-substituted with oxo, said (C₁-C₆)alkyl substituent is also optionally substituted with from one to nine fluorines;

wherein V_{V-2} is a partially saturated, fully saturated or fully unsaturated five to seven membered ring containing one to four heteroatoms selected independently from oxygen, sulfur and nitrogen;

wherein said V_{V-2} substituent is optionally mono-, di- or tri-substituted independently with halo, (C₁-C₂)alkyl, (C₁-C₂)alkoxy, hydroxy, or oxo wherein said (C₁-C₂)alkyl optionally has from one to five fluorines; and

wherein R_{V-4} does not include oxycarbonyl linked directly to the C₄ nitrogen;

wherein either R_{V-3} must contain V_{V} or R_{V-4} must contain V_{V-1};

R_{V-5}, R_{V-6}, R_{V-7} and R_{V-8} are independently hydrogen, a bond, nitro or halo wherein said bond is substituted with T_{V} or a partially saturated, fully saturated or fully unsaturated (C₁-C₁₂) straight or branched carbon chain wherein carbon may optionally be replaced with one or two heteroatoms selected independently from oxygen, sulfur and nitrogen, wherein said carbon atoms are optionally mono-, di- or tri-substituted independently with halo, said carbon is optionally mono-substituted with hydroxy, said carbon is optionally mono-substituted with oxo, said sulfur is optionally mono- or di-substituted with oxo, said nitrogen is optionally mono- or di-substituted with oxo, and said carbon chain is optionally mono-substituted with T_{V};

wherein T_{V} is a partially saturated, fully saturated or fully unsaturated three to twelve membered ring optionally having one to four heteroatoms selected independently from oxygen, sulfur and nitrogen, or a bicyclic ring consisting of two fused partially saturated, fully saturated or fully unsaturated three to six membered rings, taken independently, optionally having one to four heteroatoms selected independently from nitrogen, sulfur and oxygen;

wherein said T_{V} substituent is optionally mono-, di- or tri-substituted independently with halo, (C₁-C₆)alkyl, (C₂-C₆)alkenyl, hydroxy, (C₁-C₆)alkoxy, (C₁-C₄)alkylthio, amino, nitro, cyano, oxo, carboxy, (C₁-C₆)alkyloxycarbonyl, mono-N- or di-N,N-(C₁-C₆)alkylamino wherein said (C₁-C₆)alkyl substituent is optionally mono-, di- or tri-substituted independently with hydroxy, (C₁-C₆)alkoxy, (C₁-C₄)alkylthio, amino, nitro, cyano, oxo, carboxy, (C₁-C₆)alkyloxycarbonyl, mono-N- or di-N,N-(C₁-C₆)alkylamino, said (C₁-C₆)alkyl substituent also optionally has from one to nine fluorines;

wherein R_{V-5} and R_{V-6}, or R_{V-6} and R_{V-7}, and/or R_{V-7} and R_{V-8} may also be taken together and can form at least one ring that is a partially saturated or fully unsaturated four to eight membered ring optionally having one to three heteroatoms independently selected from nitrogen, sulfur and oxygen;

wherein said rings formed by R_{V-5} and R_{V-6}, or R_{V-6} and R_{V-7}, and/or R_{V-7} and R_{V-8} are optionally mono-, di- or tri-substituted independently with halo, (C₁-C₆)alkyl, (C₁-C₄)alkylsulfonyl, (C₂-C₆)alkenyl, hydroxy, (C₁-C₆)alkoxy, (C₁-C₄)alkylthio, amino, nitro, cyano, oxo, carboxy, (C₁-C₆)alkyloxycarbonyl, mono-N- or di-N,N-(C₁-C₆)alkylamino wherein said (C₁-C₆)alkyl substituent is optionally mono-, di- or tri-substituted independently with hydroxy, (C₁-C₆)alkoxy, (C₁-C₄)alkylthio, amino, nitro, cyano, oxo, carboxy, (C₁-C₆)alkyloxycarbonyl, mono-N- or di-N,N-(C₁-C₆)alkylamino, said (C₁-C₆)alkyl substituent also optionally has from one to nine fluorines.

Compounds of Formula V are disclosed in commonly assigned U.S. Pat. No. 6,140,343, the complete disclosure of which is herein incorporated by reference.

In a preferred embodiment, the CETP inhibitor is selected from one of the following compounds of Formula V:

[2S,4S] 4-[(3,5-bis-trifluoromethyl-benzyl)-formyl-amino]-2-cyclopropyl-6-trifluoromethyl-3,4-dihydro-2H-quinoline-1-carboxylic acid isopropyl ester;

[2S,4S] 4-[(3,5-bis-trifluoromethyl-benzyl)-formyl-amino]-2-cyclopropyl-6-trifluoromethyl-3,4-dihydro-2H-quinoline-1-carboxylic acid propyl ester;

[2S,4S] 4-[acetyl-(3,5-bis-trifluoromethyl-benzyl)-amino]-2-cyclopropyl-6-trifluoromethyl-3,4-dihydro-2H-quinoline-1-carboxylic acid tert-butyl ester;

[2R,4S] 4-[acetyl-(3,5-bis-trifluoromethyl-benzyl)-amino]-2-ethyl-6-trifluoromethyl-3,4-dihydro-2H-quinoline-1-carboxylic acid isopropyl ester;

[2R,4S] 4-[acetyl-(3,5-bis-trifluoromethyl-benzyl)-amino]-2-methyl-6-trifluoromethyl-3,4-dihydro-2H-quinoline-1-carboxylic acid ethyl ester,

[2S,4S] 4-[1-(3,5-bis-trifluoromethyl-benzyl)-ureido]-2-cyclopropyl-6-trifluoromethyl-3,4-dihydro-2H-quinoline-1-carboxylic acid isopropyl ester;

[2R,4S] 4-[acetyl-(3,5-bis-trifluoromethyl-benzyl)-amino]-2-ethyl-6-trifluoromethyl-3,4-dihydro-2H-quinoline-1-carboxylic acid ethyl ester;

[2S,4S] 4-[acetyl-(3,5-bis-trifluoromethyl-benzyl)-amino]-2-methoxymethyl-6-trifluoromethyl-3,4-dihydro-2H-quinoline-1-carboxylic acid isopropyl ester;

[2S,4S] 4-[acetyl-(3,5-bis-trifluoromethyl-benzyl)-amino]-2-cyclopropyl-6-trifluoromethyl-3,4-dihydro-2H-quinoline-1-carboxylic acid propyl ester;

[2S,4S] 4-[acetyl-(3,5-bis-trifluoromethyl-benzyl)-amino]-2-cyclopropyl-6-trifluoromethyl-3,4-dihydro-2H-quinoline-1-carboxylic acid ethyl ester;

[2R,4S] 4-[(3,5-bis-trifluoromethyl-benzyl)-formyl-amino]-2-ethyl-6-trifluoromethyl-3,4-dihydro-2H-quinoline-1-carboxylic acid isopropyl ester;

[2R,4S] 4-[(3,5-bis-trifluoromethyl-benzyl)-formyl-amino]-2-methyl-6-trifluoromethyl-3,4-dihydro-2H-quinoline-1-carboxylic acid ethyl ester;

[2S,4S] 4-[acetyl-(3,5-bis-trifluoromethyl-benzyl)-amino]-2-cyclopropyl-6-trifluoromethyl-3,4-dihydro-2H-quinoline-1-carboxylic acid isopropyl ester;

[2R,4S] 4-[(3,5-bis-trifluoromethyl-benzyl)-formyl-amino]-2-ethyl-6-trifluoromethyl-3,4-dihydro-2H-quinoline-1-carboxylic acid ethyl ester;

[2S,4S] 4-[(3,5-bis-trifluoromethyl-benzyl)-formyl-amino]-2-cyclopropyl-6-trifluoromethyl-3,4-dihydro-2H-quinoline-1-carboxylic acid ethyl ester;

[2R,4S] 4-[(3,5-bis-trifluoromethyl-benzyl)-formyl-amino]-2-methyl-6-trifluoromethyl-3,4-dihydro-2H-quinoline-1-carboxylic acid isopropyl ester; and

[2R,4S] 4-[acetyl-(3,5-bis-trifluoromethyl-benzyl)-amino]-2-methyl-6-trifluoromethyl-3,4-dihydro-2H-quinoline-1-carboxylic acid isopropyl ester.

Another class of CETP inhibitors that finds utility with the present invention consists of cycloalkano-pyridines having the Formula VI

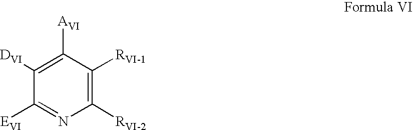

and pharmaceutically acceptable forms thereof;

in which A_{VI} denotes an aryl containing 6 to 10 carbon atoms, which is optionally substituted with up to five identical or different substituents in the form of a halogen, nitro, hydroxyl, trifluoromethyl, trifluoromethoxy or a straight-chain or branched alkyl, acyl, hydroxyalkyl or alkoxy containing up to 7 carbon atoms each, or in the form of a group according to the formula —NR_{VI-3}R_{VI-4}, wherein

R_{VI-3} and R_{VI-4} are identical or different and denote a hydrogen, phenyl or a straight-chain or branched alkyl containing up to 6 carbon atoms,

D_{VI} denotes an aryl containing 6 to 10 carbon atoms, which is optionally substituted with a phenyl, nitro, halogen, trifluoromethyl or trifluoromethoxy, or a radical according to the formula R_{VI-5}-L_{VI}-,

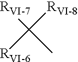

or R_{VI-9}-T_{VI}-V_{VI}—X_{VI}, wherein

R_{VI-5}, R_{VI-6} and R_{VI-9} denote, independently from one another, a cycloalkyl containing 3 to 6 carbon atoms, or an aryl containing 6 to 10 carbon atom or a 5- to 7-membered, optionally benzo-condensed, saturated or unsaturated, mono-, bi- or tricyclic heterocycle containing up to 4 heteroatoms from the series of S, N and/or O, wherein the rings are optionally substituted, in the case of the nitrogen-containing rings also via the N function, with up to five identical or different substituents in the form of a halogen, trifluoromethyl, nitro, hydroxyl, cyano, carboxyl, trifluoromethoxy, a straight-chain or branched acyl, alkyl, alkylthio, alkylalkoxy, alkoxy or alkoxycarbonyl containing up to 6 carbon atoms each, an aryl or trifluoromethyl-substituted aryl containing 6 to 10 carbon atoms each, or an optionally benzo-condensed, aromatic 5- to 7-membered heterocycle containing up to 3 heteoatoms from the series of S, N and/or O, and/or in the form of a group according to the formula —OR_{VI-10}, —SR_{VI-11}, —SO₂R_{VI-12} or —NR_{VI-13}R_{VI-14}, wherein

R_{VI-10}, R_{VI-11} and R_{VI-12} denote, independently from one another, an aryl containing 6 to 10 carbon atoms, which is in turn substituted with up to two identical or different substituents in the form of a phenyl, halogen or a straight-chain or branched alkyl containing up to 6 carbon atoms,

R_{VI-13} and R_{VI-14} are identical or different and have the meaning of R_{VI-3} and R_{VI-4} given above, or

R_{VI-5} and/or R_{VI-6} denote a radical according to the formula

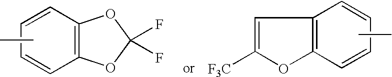

R_{VI-7} denotes a hydrogen or halogen, and

R_{VI-8} denotes a hydrogen, halogen, azido, trifluoromethyl, hydroxyl, trifluoromethoxy, a straight-chain or branched alkoxy or alkyl containing up to 6 carbon atoms each, or a radical according to the formula

—NR_{VI-15}R_{VI-16}

wherein

R_{VI-15} and R_{VI-16} are identical or different and have the meaning of R_{VI-3} and R_{VI-4} given above, or

R_{VI-7} and R_{VI-8} together form a radical according to the formula ═O or ═NR_{VI-17}, wherein

R_{VI-17} denotes a hydrogen or a straight-chain or branched alkyl, alkoxy or acyl containing up to 6 carbon atoms each,

L_{VI} denotes a straight-chain or branched alkylene or alkenylene chain containing up to 8 carbon atoms each, which are optionally substituted with up to two hydroxyl groups,

T_{VI} and X_{VI} are identical or different and denote a straight-chain or branched alkylene chain containing up to 8 carbon atoms, or

T_{VI} or X_{VI} denotes a bond,

V_{VI} denotes an oxygen or sulfur atom or an —NR_{VI-18} group, wherein

R_{VI-18} denotes a hydrogen or a straight-chain or branched alkyl containing up to 6 carbon atoms or a phenyl,

E_{VI} denotes a cycloalkyl containing 3 to 8 carbon atoms, or a straight-chain or branched alkyl containing up to 8 carbon atoms, which is optionally substituted with a cycloalkyl containing 3 to 8 carbon atoms or a hydroxyl, or a phenyl, which is optionally substituted with a halogen or trifluoromethyl,

R_{VI-1} and R_{VI-2} together form a straight-chain or branched alkylene chain containing up to 7 carbon atoms, which must be substituted with a carbonyl group and/or a radical according to the formula

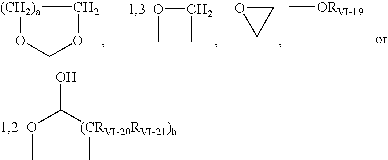

wherein

a and b are identical or different and denote a number equaling 1, 2 or 3,

R_{VI-19} denotes a hydrogen atom, a cycloalkyl containing 3 to 7 carbon atoms, a straight-chain or branched silylalkyl containing up to 8 carbon atoms, or a straight-chain or branched alkyl containing up to 8 carbon atoms, which is optionally substituted with a hydroxyl, a straight-chain or a branched alkoxy containing up to 6 carbon atoms or a phenyl, which may in turn be substituted with a halogen, nitro, trifluoromethyl, trifluoromethoxy or phenyl or tetrazole-substituted phenyl, and an alkyl that is optionally substituted with a group according to the formula —OR_{VI-22}, wherein

R_{VI-22} denotes a straight-chain or branched acyl containing up to 4 carbon atoms or benzyl, or

R_{VI-19} denotes a straight-chain or branched acyl containing up to 20 carbon atoms or benzoyl, which is optionally substituted with a halogen, trifluoromethyl, nitro or trifluoromethoxy, or a straight-chain or branched fluoroacyl containing up to 8 carbon atoms,

R_{VI-20} and R_{VI-21} are identical or different and denote a hydrogen, phenyl or a straight-chain or branched alkyl containing up to 6 carbon atoms, or

R_{VI-20} and R_{VI-21} together form a 3- to 6-membered carbocyclic ring, and a the carbocyclic rings formed are optionally substituted, optionally also geminally, with up to six identical or different substituents in the form of trifluoromethyl, hydroxyl, nitrile, halogen, carboxyl, nitro, azido, cyano, cycloalkyl or cycloalkyloxy containing 3 to 7 carbon atoms each, a straight-chain or branched alkoxycarbonyl, alkoxy or alkylthio containing up to 6 carbon atoms each, or a straight-chain or branched alkyl containing up to 6 carbon atoms, which is in turn substituted with up to two identical or different substituents in the form of a hydroxyl, benzyloxy, trifluoromethyl, benzoyl, a straight-chain or branched alkoxy, oxyacyl or carboxyl containing up to 4 carbon atoms each and/or a phenyl, which may in turn be substituted with a halogen, trifluoromethyl or trifluoromethoxy, and/or the carbocyclic rings formed are optionally substituted, also geminally, with up to five identical or different substituents in the form of a phenyl, benzoyl, thiophenyl or sulfonylbenzyl, which in turn are optionally substituted with a halogen, trifluoromethyl, trifluoromethoxy or nitro, and/or optionally in the form of a radical according to the formula

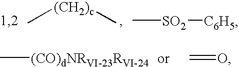

wherein

c is a number equaling 1, 2, 3 or 4,

d is a number equaling 0 or 1,

R_{VI-23} and R_{VI-24} are identical or different and denote a hydrogen, cycloalkyl containing 3 to 6 carbon atoms, a straight-chain or branched alkyl containing up to 6 carbon atoms, benzyl or phenyl, which is optionally substituted with up to two identical or different substituents in the form of halogen, trifluoromethyl, cyano, phenyl or nitro, and/or the carbocyclic rings formed are optionally substituted with a spiro-linked radical according to the formula

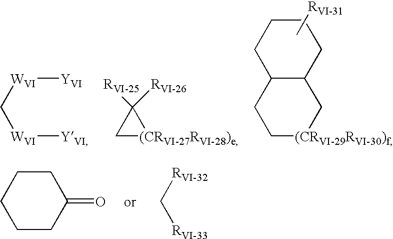

wherein

W_{VI} denotes either an oxygen atom or a sulfur atom,

Y_{VI} and Y′_{VI} together form a 2- to 6-membered straight-chain or branched alkylene chain,

e is a number equaling 1, 2, 3, 4, 5, 6 or 7,

f is a number equaling 1 or 2,

R_{VI-25}, R_{VI-26}, R_{VI-27}, R_{VI-28}, R_{VI-29}, R_{VI-30} and R_{VI-31} are identical or different and denote a hydrogen, trifluoromethyl, phenyl, halogen or a straight-chain or branched alkyl or alkoxy containing up to 6 carbon atoms each, or

R_{VI-25} and R_{VI-26} or R_{VI-27} and R_{VI-28} each together denote a straight-chain or branched alkyl chain containing up to 6 carbon atoms or

R_{VI-25} and R_{VI-26} or R_{VI-27} and R_{VI-28} each together form a radical according to the formula

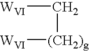

wherein

W_{VI} has the meaning given above,

g is a number equaling 1, 2, 3, 4, 5, 6 or 7,

R_{VI-32} and R_{VI-33} together form a 3- to 7-membered heterocycle, which contains an oxygen or sulfur atom or a group according to the formula SO, SO₂ or —NR_{VI-34}, wherein

R_{VI-34} denotes a hydrogen atom, a phenyl, benzyl, or a straight-chain or branched alkyl containing up to 4 carbon atoms, and salts and N oxides thereof, with the exception of 5(6H)-quinolones, 3-benzoyl-7,8-dihydro-2,7,7-trimethyl-4-phenyl.

Compounds of Formula VI are disclosed in European Patent Application No. EP 818448 A1, the complete disclosure of which is herein incorporated by reference.

In a preferred embodiment, the CETP inhibitor is selected from one of the following compounds of Formula VI:

2-cyclopentyl-4-(4-fluorophenyl)-7,7-dimethyl-3-(4-trifluoromethylbenzoyl)-4,6,7,8-tetrahydro-1H-quinolin-5-one;

2-cyclopentyl-4-(4-fluorophenyl)-7,7-dimethyl-3-(4-trifluoromethylbenzoyl)-7,8-dihydro-6H-quinolin-5-one;

[2-cyclopentyl-4-(4-fluorophenyl)-5-hydroxy-7,7-dimethyl-5,6,7,8-tetrahydroquinolin-3-yl]-(4-trifluoromethylphenyl)-methanone;

[5-(t-butyldimethylsilanyloxy)-2-cyclopentyl-4-(4-fluorophenyl)-7,7-dimethyl-5,6,7,8-tetrahydroquinolin-3-yl]-(4-trifluoromethylphenyl)-methanone;

[5-(t-butyldimethylsilanyloxy)-2-cyclopentyl-4-(4-fluorophenyl)-7,7-dimethyl-5,6,7,8-tetrahydroquinolin-3-yl]-(4-trifluoromethylphenyl)-methanol;

5-(t-butyldimethylsilanyloxy)-2-cyclopentyl-4-(4-fluorophenyl)-3-[fluoro-(4-trifluoromethylphenyl)-methyl]-7,7-dimethyl-5,6,7,8-tetrahydroquinoline;

2-cyclopentyl-4-(4-fluorophenyl)-3-[fluoro-(4-trifluoromethylphenyl)-methyl]-7,7-dimethyl-5,6,7,8-tetrahydroquinolin-5-ol.

Another class of CETP inhibitors that finds utility with the present invention consists of substituted-pyridines having the Formula VII

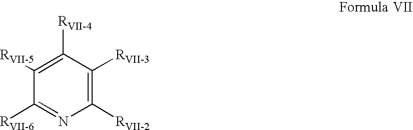

and pharmaceutically acceptable forms thereof, wherein

R_{VII-2} and R_{VII-6} are independently selected from the group consisting of hydrogen, hydroxy, alkyl, fluorinated alkyl, fluorinated aralkyl, chlorofluorinated alkyl, cycloalkyl, heterocyclyl, aryl, heteroaryl, alkoxy, alkoxyalkyl, and alkoxycarbonyl; provided that at least one of R_{VII-2} and R_{VII-6} is fluorinated alkyl, chlorofluorinated alkyl or alkoxyalkyl;

R_{VII-3} is selected from the group consisting of hydroxy, amido, arylcarbonyl, heteroarylcarbonyl, hydroxymethyl —CHO,—CO₂R_{VII-7}, wherein R_{VII-7} is selected from the group consisting of hydrogen, alkyl and cyanoalkyl; and

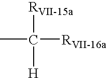

wherein R_{VII-15a} is selected from the group consisting of hydroxy, hydrogen, halogen, alkylthio, alkenylthio, alkynylthio, arylthio, heteroarylthio, heterocyclylthio, alkoxy, alkenoxy, alkynoxy, aryloxy, heteroaryloxy and heterocyclyloxy, and R_{VII-16a} is selected from the group consisting of alkyl, haloalkyl, alkenyl, haloalkenyl, alkynyl, haloalkynyl, aryl, heteroaryl, and heterocyclyl, arylalkoxy, trialkylsilyloxy;

R_{VII-4} is selected from the group consisting of hydrogen, hydroxy, halogen, alkyl, alkenyl, alkynyl, cycloalkyl, cycloalkenyl, haloalkyl, haloalkenyl, haloalkynyl, aryl, heteroaryl, heterocyclyl, cycloalkylalkyl, cycloalkenylalkyl, aralkyl, heteroarylalkyl, heterocyclylalkyl, cycloalkylalkenyl, cycloalkenylalkenyl, aralkenyl, hetereoarylalkenyl, heterocyclylalkenyl, alkoxy, alkenoxy, alkynoxy, aryloxy, heteroaryloxy, heterocyclyloxy, alkanoyloxy, alkenoyloxy, alkynoyloxy, aryloyloxy, heteroaroyloxy, heterocyclyloyloxy, alkoxycarbonyl, alkenoxycarbonyl, alkynoxycarbonyl, aryloxycarbonyl, heteroaryloxycarbonyl, heterocyclyloxycarbonyl, thio, alkylthio, alkenylthio, alkynylthio, arylthio, heteroarylthio, heterocyclylthio, cycloalkylthio, cycloalkenylthio, alkylthioalkyl, alkenylthioalkyl, alkynylthioalkyl, arylthioalkyl, heteroarylthioalkyl, heterocyclylthioalkyl, alkylthioalkenyl, alkenylthioalkenyl, alkynylthioalkenyl, arylthioalkenyl, heteroarylthioalkenyl, heterocyclythioalkenyl, alkylamino, alkenylamino, alkynylamino, arylamino, heteroarylamino, heterocyclylamino, aryldialkylamino, diarylamino, diheteroarylamino, alkylarylamino, alkylheteroarylamino, arylheteroarylamino, trialkylsilyl, trialkenylsilyl, triarylsilyl, —CO(O)N(R_{VII-8a}R_{VII-8b}), wherein R_{VII-8a} and R_{VII-8b} are independently selected from the group consisting of alkyl, alkenyl, alkynyl, aryl, heteroaryl and heterocyclyl, —SO₂R_{VII-9}, wherein R_{VII-9} is selected from the group consisting of hydroxy, alkyl, alkenyl, alkynyl, aryl, heteroaryl and heterocyclyl, —OP(O)(OR_{VII-10a}) (OR_{VII-10b}), wherein R_{VII-10a} and R_{VII-10b} are independently selected from the group consisting of hydrogen, hydroxy, alkyl, alkenyl, alkynyl, aryl, heteroaryl and heterocyclyl, and —OP(S) (OR_{VII-11a}) (OR_{VII-11b}), wherein R_{VII-11a} and R_{VII-11b} are independently selected from the group consisting of alkyl, alkenyl, alkynyl, aryl, heteroaryl and heterocyclyl;

R_{VII-5} is selected from the group consisting of hydrogen, hydroxy, halogen, alkyl, alkenyl, alkynyl, cycloalkyl, cycloalkenyl, haloalkyl, haloalkenyl, haloalkynyl, aryl, heteroaryl, heterocyclyl, alkoxy, alkenoxy, alkynoxy, aryloxy, heteroaryloxy, heterocyclyloxy, alkylcarbonyloxyalkyl, alkenylcarbonyloxyalkyl, alkynylcarbonyloxyalkyl, arylcarbonyloxyalkyl, heteroarylcarbonyloxyalkyl, heterocyclylcarbonyloxyalkyl, cycloalkylalkyl, cycloalkenylalkyl, aralkyl, heteroarylalkyl, heterocyclylalkyl, cycloalkylalkenyl, cycloalkenylalkenyl, aralkenyl, heteroarylalkenyl, heterocyclylalkenyl, alkylthioalkyl, cycloalkylthioalkyl, alkenylthioalkyl, alkynylthioalkyl, arylthioalkyl, heteroarylthioalkyl, heterocyclylthioalkyl, alkylthioalkenyl, alkenylthioalkenyl, alkynylthioalkenyl, arylthioalkenyl, heteroarylthioalkenyl, heterocyclylthioalkenyl, alkoxyalkyl, alkenoxyalkyl, alkynoxylalkyl, aryloxyalkyl, heteroaryloxyalkyl, heterocyclyloxyalkyl, alkoxyalkenyl, alkenoxyalkenyl, alkynoxyalkenyl, aryloxyalkenyl, heteroaryloxyalkenyl, heterocyclyloxyalkenyl, cyano, hydroxymethyl, —CO₂R_{VII-14}, wherein R_{VII-14} is selected from the group consisting of alkyl, alkenyl, alkynyl, aryl, heteroaryl and heterocyclyl;

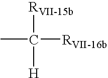

wherein R_{VII-15b} is selected from the group consisting of hydroxy, hydrogen, halogen, alkylthio, alkenylthio, alkynylthio, arylthio, heteroarylthio, heterocyclylthio, alkoxy, alkenoxy, alkynoxy, aryloxy, heteroaryloxy, heterocyclyloxy, aroyloxy, and alkylsulfonyloxy, and

R_{VII-16b} is selected form the group consisting of alkyl, alkenyl, alkynyl, aryl, heteroaryl, heterocyclyl, arylalkoxy, and trialkylsilyloxy;

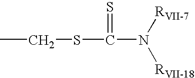

wherein R_{VII-17} and R_{VII-18} are independently selected from the group consisting of alkyl, cycloalkyl, alkenyl, alkynyl, aryl, heteroaryl and heterocyclyl;

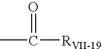

wherein R_{VII-19} is selected from the group consisting of alkyl, cycloalkyl, alkenyl, alkynyl, aryl, heteroaryl, heterocyclyl, —SR_{VII-20}, —OR_{VII-21}, and —R_{VII-22}CO₂R_{VII-23}, wherein

R_{VII-20} is selected from the group consisting of alkyl, alkenyl, alkynyl, aryl, heteroaryl, heterocyclyl, aminoalkyl, aminoalkenyl, aminoalkynyl, aminoaryl, aminoheteroaryl, aminoheterocyclyl, alkylheteroarylamino, arylheteroarylamino,

R_{VII-21} is selected from the group consisting of alkyl, alkenyl, alkynyl, aryl, heteroaryl, and heterocyclyl,

R_{VII-22} is selected from the group consisting of alkylene or arylene, and

R_{VII-23} is selected from the group consisting of alkyl, alkenyl, alkynyl, aryl, heteroaryl, and heterocyclyl;

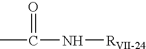

wherein R_{VII-24} is selected from the group consisting of hydrogen, alkyl, cycloalkyl, alkenyl, alkynyl, aryl, heteroaryl, heterocyclyl, aralkyl, aralkenyl, and aralkynyl;

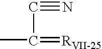

wherein R_{VII-25} is heterocyclylidenyl;

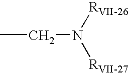

wherein R_{VII-26} and R_{VII-27} are independently selected from the group consisting of hydrogen, alkyl, cycloalkyl, alkenyl, alkynyl, aryl, heteroaryl, and heterocyclyl;

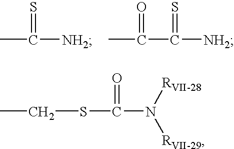

wherein R_{VII-28} and R_{VII-29} are independently selected from the group consisting of hydrogen, alkyl, cycloalkyl, alkenyl, alkynyl, aryl, heteroaryl, and heterocyclyl;

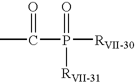

wherein R_{VII-30} and R_{VII-31} are independently alkoxy, alkenoxy, alkynoxy, aryloxy, heteroaryloxy, and heterocyclyloxy; and

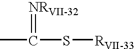

wherein R_{VII-32} and R_{VII-33} are independently selected from the group consisting of hydrogen, alkyl, cycloalkyl, alkenyl, alkynyl, aryl, heteroaryl, and heterocyclyl;

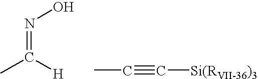

wherein R_{VII-36} is selected from the group consisting of alkyl, alkenyl, aryl, heteroaryl and heterocyclyl;

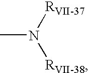

wherein R_{VII-37} and R_{VII-38} are independently selected from the group consisting of hydrogen, alkyl, cycloalkyl, alkenyl, alkynyl, aryl, heteroaryl, and heterocyclyl;

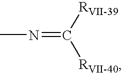

wherein R_{VII-39} is selected from the group consisting of hydrogen, alkoxy, alkenoxy, alkynoxy, aryloxy, heteroaryloxy, heterocyclyloxy, alkylthio, alkenylthio, alkynylthio, arylthio, heteroarylthio and heterocyclylthio, and

R_{VII-40} is selected from the group consisting of haloalkyl, haloalkenyl, haloalkynyl, haloaryl, haloheteroaryl, haloheterocyclyl, cycloalkyl, cycloalkenyl, heterocyclylalkoxy, heterocyclylalkenoxy, heterocyclylalkynoxy, alkylthio, alkenylthio, alkynylthio, arylthio, heteroarylthio and heterocyclylthio;

—N═R_{VII-41},

wherein R_{VII-41} is heterocyclylidenyl;

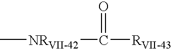

wherein R_{VII-42} is selected from the group consisting of hydrogen, alkyl, alkenyl, alkynyl, aryl, heteroaryl, and heterocyclyl, and

R_{VII-43} is selected from the group consisting of hydrogen, alkyl, alkenyl, alkynyl, aryl, heteroaryl, heterocyclyl, cycloalkyl, cycloalkenyl, haloalkyl, haloalkenyl, haloalkynyl, haloaryl, haloheteroaryl, and haloheterocyclyl;

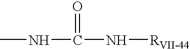

wherein R_{VII-44} is selected from the group consisting of hydrogen, alkyl, cycloalkyl, alkenyl, alkynyl, aryl, heteroaryl and heterocyclyl;

—N═S═O;

—N═C═S;

—N═C═O;

—N₃;

—SR_{VII-45}

wherein R_{VII-45} is selected from the group consisting of hydrogen, alkyl, alkenyl, alkynyl, aryl, heteroaryl, heterocyclyl, haloalkyl, haloalkenyl, haloalkynyl, haloaryl, haloheteroaryl, haloheterocyclyl, heterocyclyl, cycloalkylalkyl, cycloalkenylalkyl, aralkyl, heteroarylalkyl, heterocyclylalkyl, cycloalkylalkenyl, cycloalkenylalkenyl, aralkenyl, heteroarylalkenyl, heterocyclylalkenyl, alkylthioalkyl, alkenylthioalkyl, alkynylthioalkyl, arylthioalkyl,heteroarylthioalkyl, heterocyclylthioalkyl, alkylthioalkenyl, alkenylthioalkenyl, alkynylthioalkenyl, arylthioalkenyl, heteroarylthioalkenyl, heterocyclylthioalkenyl, aminocarbonylalkyl, aminocarbonylalkenyl, aminocarbonylalkynyl, aminocarbonylaryl, aminocarbonylheteroaryl, and aminocarbonylheterocyclyl,

—SR_{VII-46}, and —CH₂R_{VII-47},

wherein R_{VII-46} is selected from the group consisting of alkyl, alkenyl, alkynyl, aryl, heteroaryl and heterocyclyl, and

R_{VII-47} is selected from the group consisting of hydrogen, alkyl, alkenyl, alkynyl, aryl, heteroaryl and heterocyclyl; and

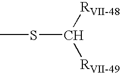

wherein R_{VII-48} is selected from the group consisting of hydrogen, alkyl, cycloalkyl, alkenyl, alkynyl, aryl, heteroaryl and heterocyclyl, and

R_{VII-49} is selected from the group consisting of alkoxy, alkenoxy, alkynoxy, aryloxy, heteroaryloxy, heterocyclyloxy, haloalkyl, haloalkenyl, haloalkynyl, haloaryl, haloheteroaryl and haloheterocyclyl;

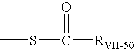

wherein R_{VII-50} is selected from the group consisting of hydrogen, alkyl, cycloalkyl, alkenyl, alkynyl, aryl, heteroaryl, heterocyclyl, alkoxy, alkenoxy, alkynoxy, aryloxy, heteroaryloxy and heterocyclyloxy;

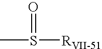

wherein R_{VII-51} is selected from the group consisting of alkyl, alkenyl, alkynyl, aryl, heteroaryl, heterocyclyl, haloalkyl, haloalkenyl, haloalkynyl, haloaryl, haloheteroaryl and haloheterocyclyl; and

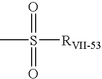

wherein R_{VII-53} is selected from the group consisting of alkyl, alkenyl, alkynyl, aryl, heteroaryl and heterocyclyl;

provided that when R_{VII-5} is selected from the group consisting of heterocyclylalkyl and heterocyclylalkenyl, the heterocyclyl radical of the corresponding heterocyclylalkyl or heterocyclylalkenyl is other than δ-lactone; and

provided that when R_{VII-4} is aryl, heteroaryl or heterocyclyl, and one of R_{VII-2} and R_{VII-6} is trifluoromethyl, then the other of R_{VII-2} and R_{VII-6} is difluoromethyl.

Compounds of Formula VII are disclosed in WO 9941237-A1, the complete disclosure of which is incorporated by reference.

In a preferred embodiment, the CETP inhibitor is selected from the following compounds of Formula VII:

dimethyl 5,5′-dithiobis[2-difluoromethyl-4-(2-methylpropyl)-6-(trifluoromethyl)-3-pyridine-carboxylate].

Another class of CETP inhibitors that finds utility with the present invention consists of substituted pyridines and biphenyls having the Formula VIII

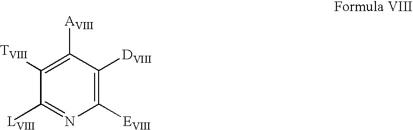

and pharmaceutically acceptable forms thereof,

in which

A_{VIII} stands for aryl with 6 to 10 carbon atoms, which is optionally substituted up to 3 times in an identical manner or differently by halogen, hydroxy, trifluoromethyl, trifluoromethoxy, or by straight-chain or branched alkyl, acyl, or alkoxy with up to 7 carbon atoms each, or by a group of the formula

—NR_{VIII-1}R_{VIII-2}, wherein

R_{VIII-1} and R_{VIII-2} are identical or different and denote hydrogen, phenyl, or straight-chain or branched alkyl with up to 6 carbon atoms,

D_{VIII} stands for straight-chain or branched alkyl with up to 8 carbon atoms, which is substituted by hydroxy,

E_{VIII} and L_{VIII} are either identical or different and stand for straight-chain or branched alkyl with up to 8 carbon atoms, which is optionally substituted by cycloalkyl with 3 to 8 carbon atoms, or stands for cycloalkyl with 3 to 8 carbon atoms, or

E_{VIII} has the above-mentioned meaning and

L_{VIII} in this case stands for aryl with 6 to 10 carbon atoms, which is optionally substituted up to 3 times in an identical manner or differently by halogen, hydroxy, trifluoromethyl, trifluoromethoxy, or by straight-chain or branched alkyl, acyl, or alkoxy with up to 7 carbon atoms each, or by a group of the formula

—NR_{VIII-3}R_{VIII-4}, wherein

R_{VIII-3} and R_{VIII-4} are identical or different and have the meaning given above for R_{VIII-1} and R_{VIII-2}, or

E_{VIII} stands for straight-chain or branched alkyl with up to 8 carbon atoms, or stands for aryl with 6 to 10 carbon atoms, which is optionally substituted up to 3 times in an identical manner or differently by halogen, hydroxy, trifluoromethyl, trifluoromethoxy, or by straight-chain or branched alkyl, acyl, or alkoxy with up to 7 carbon atoms each, or by a group of the formula

—NR_{VIII-5}R_{VIII-6}, wherein

R_{VIII-5} and R_{VIII-6} are identical or different and have the meaning given above for R_{VIII-1} and R_{VIII-2}, and

L_{VIII} in this case stands for straight-chain or branched alkoxy with up to 8 carbon atoms or for cycloalkyloxy with 3 to 8 carbon atoms,

T_{VIII} stands for a radical of the formula

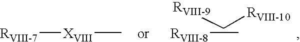

wherein

R_{VIII-7} and R_{VIII-8} are identical or different and denote cycloalkyl with 3 to 8 carbon atoms, or aryl with 6 to 10 carbon atoms, or denote a 5- to 7-member aromatic, optionally benzo-condensed, heterocyclic compound with up to 3 heteroatoms from the series S, N and/or O, which are optionally substituted up to 3 times in an identical manner or differently by trifluoromethyl, trifluoromethoxy, halogen, hydroxy, carboxyl, by straight-chain or branched alkyl, acyl, alkoxy, or alkoxycarbonyl with up to 6 carbon atoms each, or by phenyl, phenoxy, or thiophenyl, which can in turn be substituted by halogen, trifluoromethyl, or trifluoromethoxy, and/or the rings are substituted by a group of the formula

—NR_{VIII-11}R_{V}_{III-12}, wherein

R_{VIII-11} and R_{VIII-12} are identical or different and have the meaning given above for R_{VIII-1} and R_{VIII-2},

X_{VIII} denotes a straight or branched alkyl chain or alkenyl chain with 2 to 10 carbon atoms each, which are optionally substituted up to 2 times by hydroxy,

R_{VIII-9} denotes hydrogen, and

R_{VIII-10} denotes hydrogen, halogen, azido, trifluoromethyl, hydroxy, mercapto, trifluoromethoxy, straight-chain or branched alkoxy with up to 5 carbon atoms, or a radical of the formula

—NR_{VIII-13}R_{VIII-14}, wherein

R_{VIII-13} and R_{VIII-14} are identical or different and have the meaning given above for R_{VIII-1} and R_{VIII-2}, or

R_{VIII-9} and R_{VIII-10} form a carbonyl group together with the carbon atom.

Compounds of Formula VIII are disclosed in WO 9804528, the complete disclosure of which is incorporated by reference.

Another class of CETP inhibitors that finds utility with the present invention consists of substituted 1,2,4-triazoles having the Formula IX

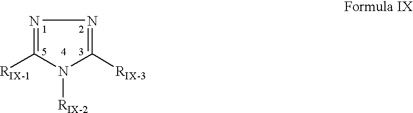

and pharmaceutically acceptable forms thereof;

wherein R_{IX-1} is selected from higher alkyl, higher alkenyl, higher alkynyl, aryl, aralkyl, aryloxyalkyl, alkoxyalkyl, alkylthioalkyl, arylthioalkyl, and cycloalkylalkyl;

wherein R_{IX-2} is selected from aryl, heteroaryl, cycloalkyl, and cycloalkenyl,

wherein R_{IX-2} is optionally substituted at a substitutable position with one or more radicals independently selected from alkyl, haloalkyl, alkylthio, alkylsulfinyl, alkylsulfonyl, alkoxy, halo, aryloxy, aralkyloxy, aryl, aralkyl, aminosulfonyl, amino, monoalkylamino and dialkylamino; and

wherein R_{IX-3} is selected from hydrido, —SH and halo;

provided R_{IX-2} cannot be phenyl or 4-methylphenyl when R_{IX-1} is higher alkyl and when R_{IX-3} is —SH.

Compounds of Formula IX are disclosed in WO 9914204, the complete disclosure of which is incorporated by reference.

In a preferred embodiment, the CETP inhibitor is selected from the following compounds of Formula IX:

2,4-dihydro-4-(3-methoxyphenyl)-5-tridecyl-3H-1,2,4-triazole-3-thione;

2,4-dihydro-4-(2-fluorophenyl)-5-tridecyl-3H-1,2,4-triazole-3-thione;

2,4-dihydro-4-(2-methylphenyl)-5-tridecyl-3H-1,2,4-triazole-3-thione;

2,4-dihydro-4-(3-chlorophenyl)-5-tridecyl-3H-1,2,4-triazole-3-thione;

2,4-dihydro-4-(2-methoxyphenyl)-5-tridecyl-3H-1,2,4-triazole-3-thione;

2,4-dihydro-4-(3-methylphenyl)-5-tridecyl-3H-1,2,4-triazole-3-thione;

4-cyclohexyl-2,4-dihydro-5-tridecyl-3H-1,2,4-triazole-3-thione;

2,4-dihydro-4-(3-pyridyl)-5-tridecyl-3H-1,2,4-triazole-3-thione;

2,4-dihydro-4-(2-ethoxyphenyl)-5-tridecyl-3H-1,2,4-triazole-3-thione;

2,4-dihydro-4-(2,6-dimethylphenyl)-5-tridecyl-3H-1,2,4-triazole-3-thione;

2,4-dihydro-4-(4-phenoxyphenyl)-5-tridecyl-3H-1,2,4-triazole-3-thione;

4-(1,3-benzodioxol-5-yl)-2,4-dihydro-5-tridecyl-3H-1,2,4-triazole-3-thione;

4-(2-chlorophenyl)-2,4-dihydro-5-tridecyl-3H-1,2,4-triazole-3-thione;

2,4-dihydro-4-(4-methoxyphenyl)-5-tridecyl-3H-1,2,4-triazole-3-thione;

2,4-dihydro-5-tridecyl-4-(3-trifluoromethylphenyl)-3H-1,2,4-triazole-3-thione;

2,4-dihydro-5-tridecyl-4-(3-fluorophenyl)-3H-1,2,4-triazole-3-thione;

4-(3-chloro-4-methylphenyl)-2,4-dihydro-5-tridecyl-3H-1,2,4-triazole-3-thione;

2,4-dihydro-4-(2-methylthiophenyl)-5-tridecyl-3H-1,2,4-triazole-3-thione;

4-(4-benzyloxyphenyl)-2,4-dihydro-5-tridecyl-3H-1,2,4-triazole-3-thione;

2,4-dihydro-4-(2-naphthyl)-5-tridecyl-3H-1,2,4-triazole-3-thione;

2,4-dihydro-5-tridecyl-4-(4-trifluoromethylphenyl)-3H-1,2,4-triazole-3-thione;

2,4-dihydro-4-(1-naphthyl)-5-tridecyl-3H-1,2,4-triazole-3-thione;

2,4-dihydro-4-(3-methylthiophenyl)-5-tridecyl-3H-1,2,4-triazole-3-thione;

2,4-dihydro-4-(4-methylthiophenyl)-5-tridecyl-3H-1,2,4-triazole-3-thione;

2,4-dihydro-4-(3,4-dimethoxyphenyl)-5-tridecyl-3H-1,2,4-triazole-3-thione;

2,4-dihydro-4-(2,5-dimethoxyphenyl)-5-tridecyl-3H-1,2,4-triazole-3-thione;

2,4-dihydro-4-(2-methoxy-5-chlorophenyl)-5-tridecyl-3H-1,2,4-triazole-3-thione;

4-(4-aminosulfonylphenyl)-2,4-dihydro-5-tridecyl-3H-1,2,4-triazole-3-thione;

2,4-dihydro-5-dodecyl-4-(3-methoxyphenyl)-3H-1,2,4-triazole-3-thione;

2,4-dihydro-4-(3-methoxyphenyl)-5-tetradecyl-3H-1,2,4-triazole-3-thione;

2,4-dihydro-4-(3-methoxyphenyl)-5-undecyl-3H-1,2,4-triazole-3-thione; and

2,4-dihydro-(4-methoxyphenyl)-5-pentadecyl-3H-1,2,4-triazole-3-thione.

Another class of CETP inhibitors that finds utility with the present invention consists of hetero-tetrahydroquinolines having the Formula X

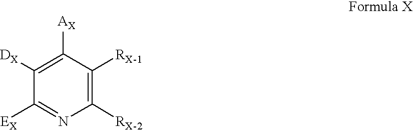

N-oxides of said compounds, and pharmaceutically acceptable forms thereof; in which

A_{X} represents cycloalkyl with 3 to 8 carbon atoms or a 5- to 7-membered, saturated, partially saturated or unsaturated, optionally benzo-condensed heterocyclic ring containing up to 3 heteroatoms from the series comprising S, N and/or O, that in case of a saturated heterocyclic ring is bonded to a nitrogen function, optionally bridged over it, and in which the aromatic systems mentioned above are optionally substituted up to 5-times in an identical or different substituents in the form of halogen, nitro, hydroxy, trifluoromethyl, trifluoromethoxy or by a straight-chain or branched alkyl, acyl, hydroxyalkyl or alkoxy each having up to 7 carbon atoms or by a group of the formula —NR_{X-3}R_{X-4},

in which

R_{X-3} and R_{X-4} are identical or different and denote hydrogen, phenyl or straight-chain or branched alkyl having up to 6 carbon atoms, or

A_{X} represents a radical of the formula

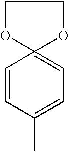

D_{X} represents an aryl having 6 to 10 carbon atoms, that is optionally substituted by phenyl, nitro, halogen, trifluormethyl or trifluormethoxy, or it represents a radical of the formula

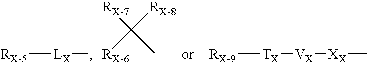

in which

R_{X-5}, R_{X-6} and R_{X-9} independently of one another denote cycloalkyl having 3 to 6 carbon atoms, or an aryl having 6 to 10 carbon atoms or a 5- to 7-membered aromatic, optionally benzo-condensed saturated or unsaturated, mono-, bi-, or tricyclic heterocyclic ring from the series consisting of S, N and/or 0, in which the rings are substituted, optionally, in case of the nitrogen containing aromatic rings via the N function, with up to 5 identical or different substituents in the form of halogen, trifluoromethyl, nitro, hydroxy, cyano, carbonyl, trifluoromethoxy, straight straight-chain or branched acyl, alkyl, alkylthio, alkylalkoxy, alkoxy, or alkoxycarbonyl each having up to 6 carbon atoms, by aryl or trifluoromethyl-substituted aryl each having 6 to 10 carbon atoms or by an, optionally benzo-condensed, aromatic 5- to 7-membered heterocyclic ring having up to 3 heteroatoms from the series consisting of S, N, and/or O, and/or substituted by a group of the formula —OR_{X-10}, —SR_{X-11}, SO₂R_{X-12} or —NR_{X-13}R_{X-14},

in which

R_{X-10}, R_{X-11} and R_{X-12} independently from each other denote aryl having 6 to 10 carbon atoms, which is in turn substituted with up to 2 identical or different substituents in the form of phenyl, halogen or a straight-chain or branched alkyl having up to 6 carbon atoms,

R_{X-13} and R_{X-14} are identical or different and have the meaning of R_{X-3} and R_{X-4} indicated above,

or

R_{X-5} and/or R_{X-6} denote a radical of the formula

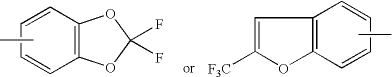

R_{X-7} denotes hydrogen or halogen, and

R_{X-8} denotes hydrogen, halogen, azido, trifluoromethyl, hydroxy, trifluoromethoxy, straight-chain or branched alkoxy or alkyl having up to 6 carbon atoms or a radical of the formula —NR_{X-15}R_{X-16}, in which

R_{X-15} and R_{X-16} are identical or different and have the meaning of R_{X-3} and R_{X-4} indicated above,

or

R_{X-7} and R_{X-8} together form a radical of the formula ═O or ═NR_{X-17},

in which

R_{X-17} denotes hydrogen or straight chain or branched alkyl, alkoxy or acyl having up to 6 carbon atoms,

L_{X} denotes a straight chain or branched alkylene or alkenylene chain having up to 8 carbon atoms, that are optionally substituted with up to 2 hydroxy groups,

T_{X} and X_{X} are identical or different and denote a straight chain or branched alkylene chain with up to 8 carbon atoms

or

T_{X} or X_{X} denotes a bond,

V_{X} represents an oxygen or sulfur atom or an —NR_{X-18}-group, in which

R_{X-18} denotes hydrogen or straight chain or branched alkyl with up to 6 carbon atoms or phenyl,

E_{X} represents cycloalkyl with 3 to 8 carbon atoms, or straight chain or branched alkyl with up to 8 carbon atoms, that is optionally substituted by cycloalkyl with 3 to 8 carbon atoms or hydroxy, or represents a phenyl, that is optionally substituted by halogen or trifluoromethyl,

R_{X-1} and R_{X-2} together form a straight-chain or branched alkylene chain with up to 7 carbon atoms, that must be substituted by carbonyl group and/or by a radical with the formula

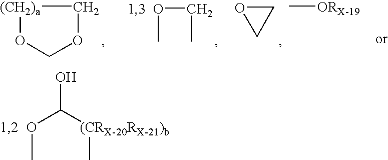

in which a and b are identical or different and denote a number equaling 1,2, or 3,

R_{X-19} denotes hydrogen, cycloalkyl with 3 up to 7 carbon atoms, straight chain or branched silylalkyl with up to 8 carbon atoms or straight chain or branched alkyl with up to 8 carbon atoms, that are optionally substituted by hydroxyl, straight chain or branched alkoxy with up to 6 carbon atoms or by phenyl, which in turn might be substituted by halogen, nitro, trifluormethyl, trifluoromethoxy or by phenyl or by tetrazole-substituted phenyl, and alkyl, optionally be substituted by a group with the formula —OR_{X-22},

in which

R_{X-22} denotes a straight chain or branched acyl with up to 4 carbon atoms or benzyl,

or

R_{X-19} denotes straight chain or branched acyl with up to 20 carbon atoms or benzoyl, that is optionally substituted by halogen, trifluoromethyl, nitro or trifluoromethoxy, or it denotes straight chain or branched fluoroacyl with up to 8 carbon atoms and 9 fluorine atoms,

R_{X-20} and R_{X-21} are identical or different and denote hydrogen, phenyl or straight chain or branched alkyl with up to 6 carbon atoms,

or

R_{X-20} and R_{X-21} together form a 3- to 6-membered carbocyclic ring, and the carbocyclic rings formed are optionally substituted, optionally also geminally, with up to six identical or different substituents in the form of triflouromethyl, hydroxy, nitrile, halogen, carboxyl, nitro, azido, cyano, cycloalkyl or cycloalkyloxy with 3 to 7 carbon atoms each, by straight chain or branched alkoxycarbonyl, alkoxy or alkylthio with up to 6 carbon atoms each or by straight chain or branched alkyl with up to 6 carbon atoms, which in turn is substituted with up to 2 identically or differently by hydroxyl, benzyloxy, trifluoromethyl, benzoyl, straight chain or branched alkoxy, oxyacyl or carbonyl with up to 4 carbon atoms each and/or phenyl, which may in turn be substituted with a halogen, trifuoromethyl or trifluoromethoxy, and/or the formed carbocyclic rings are optionally substituted, also geminally, with up to 5 identical or different substituents in the form of phenyl, benzoyl, thiophenyl or sulfonylbenzyl, which in turn are optionally substituted by halogen, trifluoromethyl, trifluoromethoxy or nitro, and/or optionally are substituted by a radical with the formula

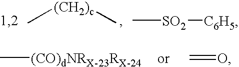

in which

c denotes a number equaling 1, 2, 3, or 4,

d denotes a number equaling 0 or 1,

R_{X-23} and R_{X-24} are identical or different and denote hydrogen, cycloalkyl with 3 to 6 carbon atoms, straight chain or branched alkyl with up to 6 carbon atoms, benzyl or phenyl, that is optionally substituted with up to 2 identically or differently by halogen, trifluoromethyl, cyano, phenyl or nitro, and/or the formed carbocyclic rings are substituted optionally by a spiro-linked radical with the formula

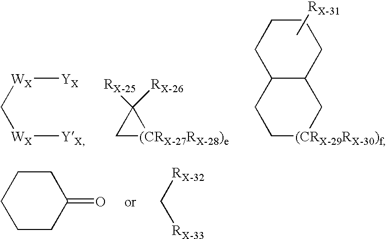

in which

W_{X} denotes either an oxygen or a sulfur atom

Y_{X} and Y′_{X} together form a 2 to 6 membered straight chain or branched alkylene chain,

e denotes a number equaling 1, 2, 3, 4, 5, 6, or 7,

f denotes a number equaling 1 or 2,

R_{X-25}, R_{X-26}, R_{X-27}, R_{X-28}, R_{X-29}, R_{X-30} and R_{X-31} are identical or different and denote hydrogen, trifluoromethyl, phenyl, halogen or straight chain or branched alkyl or alkoxy with up to 6 carbon atoms each,

or

R_{X-25} and R_{X-26} or R_{X-27} and R_{X-28} respectively form together a straight chain or branched alkyl chain with up to 6 carbon atoms,

or

R_{X-25} and R_{X-26} or R_{X-27} and R_{X-28} each together form a radical with the formula

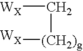

in which

W_{X} has the meaning given above,

g denotes a number equaling 1, 2, 3, 4, 5, 6, or 7,

R_{X-32} and R_{X-33} form together a 3- to 7-membered heterocycle, which contains an oxygen or sulfur atom or a group with the formula SO, SO₂ or π-NR_{X-34}, in which

R_{X-34} denotes hydrogen, phenyl, benzyl or straight or branched alkyl with up to 4 carbon atoms.

Compounds of Formula X are disclosed in WO 9914215, the complete disclosure of which is incorporated by reference.

In a preferred embodiment, the CETP inhibitor is selected from the following compounds of Formula X:

2-cyclopentyl-5-hydroxy-7,7-dimethyl-4-(3-thienyl)-3-(4-trifluoromethylbenxoyl)-5,6,7,8-tetrahydroquinoline;

2-cyclopentyl-3-[fluoro-(4-trifluoromethylphenyl)methyl]-5-hydroxy-7,7-dimethyl-4-(3-thienyl)-5,6,7,8-tetrahydroquinoline; and

2-cyclopentyl-5-hydroxy-7,7-dimethyl-4-(3-thienyl)-3-(trifluoromethylbenxyl)-5,6,7,8-tetrahydroquinoline.

Another class of CETP inhibitors that finds utility with the present invention consists of substituted tetrahydro naphthalines and analogous compounds having the Formula XI

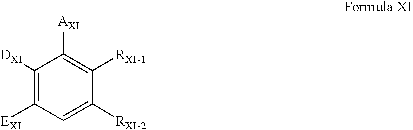

and pharmaceutically acceptable forms thereof, in which

A_{XI} stands for cycloalkyl with 3 to 8 carbon atoms, or stands for aryl with 6 to 10 carbon atoms, or stands for a 5- to 7-membered, saturated, partially unsaturated or unsaturated, possibly benzocondensated, heterocycle with up to 4 heteroatoms from the series S, N and/or O, where aryl and the heterocyclic ring systems mentioned above are substituted up to 5-fold, identical or different, by cyano, halogen, nitro, carboxyl, hydroxy, trifluoromethyl, trifluoro-methoxy, or by straight-chain or branched alkyl, acyl, hydroxyalkyl, alkylthio, alkoxycarbonyl, oxyalkoxycarbonyl or alkoxy each with up to 7 carbon atoms, or by a group of the formula

—NR_{XI-3}R_{XI-4},

in which

R_{XI-3} and R_{XI-4} are identical or different and denote hydrogen, phenyl, or straight-chain or branched alkyl with up to 6 carbon atoms

D_{XI} stands for a radical of the formula

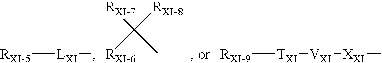

in which

R_{XI-5}, R_{XI-6} and R_{XI-9}, independent of each other, denote cycloalkyl with 3 to 6 carbon atoms, or denote aryl with 6 to 10 carbon atoms, or denote a 5- to 7-membered, possibly benzocondensated, saturated or unsaturated, mono-, bi- or tricyclic heterocycle with up to 4 heteroatoms of the series S, N and/or O, where the cycles are possibly substituted—in the case of the nitrogen-containing rings also via the N-function-up to 5-fold, identical or different, by halogen, trifluoromethyl, nitro, hydroxy, cyano, carboxyl, trifluoromethoxy, straight-chain or branched acyl, alkyl, alkylthio, alkylalkoxy, alkoxy or alkoxycarbonyl with up to 6 carbon atoms each by aryl or trifluoromethyl substituted aryl with 6 to 10 carbon atoms each, or by a possibly benzocondensated aromatic 5- to 7-membered heterocycle with up to 3 heteroatoms of the series S, N and/or O, and/or are substituted by a group of the formula

—OR_{XI-10}, —SR_{XI-11}, —SO₂R_{XI-12} or —NR_{XI-13}R_{XI-14},

in which

R_{XI-10}, R_{XI-11} and R_{XI-12}, independent of each other, denote aryl with 6 to 10 carbon atoms, which itself is substituted up to 2-fold, identical or different, by phenyl, halogen, or by straight-chain or branched alkyl with up to 6 carbon atoms,

R_{XI-13} and R_{XI-14} are identical or different and have the meaning given above for R_{XI-3} and R_{X-14},

or

R_{XI-5} and/or R_{XI-6} denote a radical of the formula

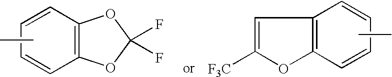

R_{XI-7} denotes hydrogen, halogen or methyl,

and

R_{XI-8} denotes hydrogen, halogen, azido, trifluoromethyl, hydroxy, trifluoromethoxy, straight-chain or branched alkoxy or alkyl with up to 6 carbon atoms each, or a radical of the formula —NR_{XI-15}R_{XI-16},

in which

R_{XI-15} and R_{XI-16} are identical or different and have the meaning given above for R_{XI-3} and R_{XI-4},

or

R_{XI-7} and R_{XI-8} together form a radical of the formula ═O or ═NR_{XI-17}, in which

R_{XI-17} denotes hydrogen or straight-chain or branched alkyl, alkoxy or acyl with up to 6 carbon atoms each,

L_{XI} denotes a straight-chain or branched alkylene- or alkenylene chain with up to 8 carbon atoms each, which is possibly substituted up to 2-fold by hydroxy,

T_{XI} and X_{XI} are identical or different and denote a straight-chain or branched alkylene chain with up to 8 carbon atoms,

or

T_{XI} and X_{XI} denotes a bond,

V_{XI} stands for an oxygen- or sulfur atom or for an —NR_{XI-18} group,

in which

R_{XI-18} denotes hydrogen or straight-chain or branched alkyl with up to 6 carbon atoms, or phenyl,

E_{XI} stands for cycloalkyl with 3 to 8 carbon atoms, or stands for straight-chain or branched alkyl with up to 8 carbon atoms, which is possibly substituted by cycloalkyl with 3 to 8 carbon atoms or hydroxy, or stands for phenyl, which is possibly substituted by halogen or trifluoromethyl,

R_{XI-1} and R_{XI-2} together form a straight-chain or branched alkylene chain with up to 7 carbon atoms, which must be substituted by a carbonyl group and/or by a radical of the formula

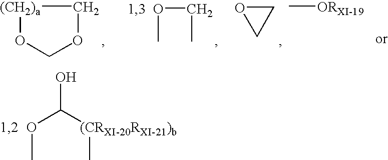

in which

a and b are identical or different and denote a number 1, 2 or 3

R_{XI-19} denotes hydrogen, cycloalkyl with 3 to 7 carbon atoms, straight-chain or branched silylalkyl with up to 8 carbon atoms, or straight-chain or branched alkyl with up to 8 carbon atoms, which is possibly substituted by hydroxy, straight-chain or branched alkoxy with up to 6 carbon atoms, or by phenyl, which itself can be substituted by halogen, nitro, trifluoromethyl, trifluoromethoxy or by phenyl substituted by phenyl or tetrazol, and alkyl is possibly substituted by a group of the formula —OR_{XI-22},

in which

R_{XI-22} denotes straight-chain or branched acyl with up to 4 carbon atoms, or benzyl,

or

R_{XI-19} denotes straight-chain or branched acyl with up to 20 carbon atoms or benzoyl, which is possibly substituted by halogen, trifluoromethyl, nitro or trifluoromethoxy, or denotes straight-chain or branched fluoroacyl with up to 8 carbon atoms and 9 fluorine atoms,

R_{XI-20} and R_{XI-21} are identical or different, denoting hydrogen, phenyl or straight-chain or branched alkyl with up to 6 carbon atoms,

or

R_{XI-20} and R_{XI-21} together form a 3- to 6-membered carbocycle, and, possibly also geminally, the alkylene chain formed by R_{XI-1} and R_{XI-2}, is possibly substituted up to 6-fold, identical or different, by trifluoromethyl, hydroxy, nitrile, halogen, carboxyl, nitro, azido, cyano, cycloalkyl or cycloalkyloxy with 3 to 7 carbon atoms each, by straight-chain or branched alkoxycarbonyl, alkoxy or alkoxythio with up to 6 carbon atoms each, or by straight- chain or branched alkyl with up to 6 carbon atoms, which itself is substituted up to 2-fold, identical or different, by hydroxyl, benzyloxy, trifluoromethyl, benzoyl, straight-chain or branched alkoxy, oxyacyl or carboxyl with up to 4 carbon atoms each, and/or phenyl- which itself can be substituted by halogen, trifluoromethyl or trifluoromethoxy, and/or the alkylene chain formed by R_{XI-1} and R_{XI-2} is substituted, also geminally, possibly up to 5-fold, identical or different, by phenyl, benzoyl, thiophenyl or sulfobenzyl -which themselves are possibly substituted by halogen, trifluoromethyl, trifluoromethoxy or nitro, and/or the alkylene chain formed by R_{XI-1} and R_{XI-2} is possibly substituted by a radical of the formula

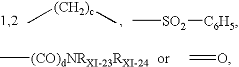

in which

c denotes a number 1,2, 3 or 4,

d denotes a number 0 or 1,

R_{XI-23} and R_{XI-24} are identical or different and denote hydrogen, cycloalkyl with 3 to 6 carbon atoms, straight-chain or branched alkyl with up to 6 carbon atoms, benzyl or phenyl, which is possibly substituted up to 2-fold identical or different, by halogen, trifluoromethyl, cyano, phenyl or nitro, and/or the alkylene chain formed by R_{XI-1} and R_{XI-2} is possibly substituted by a spiro-jointed radical of the formula

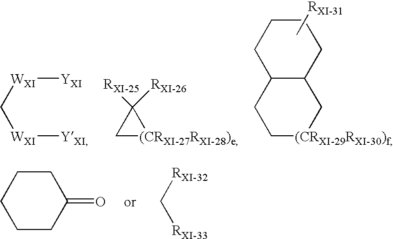

in which

W_{XI} denotes either an oxygen or a sulfur atom,

Y_{XI} and Y′_{XI} together form a 2- to 6-membered straight-chain or branched alkylene chain,

e is a number 1, 2, 3, 4, 5, 6 or 7,

f denotes a number 1 or 2,

R_{XI-25}, R_{XI-26}, R_{XI-27}, R_{XI-28}, R_{XI-29}, R_{XI-30} and R_{XI-31} are identical or different and denote hydrogen, trifluoromethyl, phenyl, halogen, or straight-chain or branched alkyl or alkoxy with up to 6 carbon atoms each,

or

R_{XI-25} and R_{XI-26} or R_{XI-27} and R_{XI-28} together form a straight-chain or branched alkyl chain with up to 6 carbon atoms,

or

R_{XI-25} and R_{XI-26} or R_{XI-27} and R_{XI-28} together form a radical of the formula

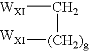

in which

W_{Xi} has the meaning given above,

g is a number 1, 2, 3, 4, 5, 6 or 7,

R_{XI-32} and R_{XI-33} together form a 3- to 7-membered heterocycle that contains an oxygen- or sulfur atom or a group of the formula SO, SO₂ or —NR_{XI-34},

in which R_{XI-34} denotes hydrogen, phenyl, benzyl, or straight-chain or branched alkyl with up to 4 carbon atoms.

Compounds of Formula XI are disclosed in WO 9914174, the complete disclosure of which is incorporated by reference.

Another class of CETP inhibitors that finds utility with the present invention consists of 2-aryl-substituted pyridines having the Formula XII

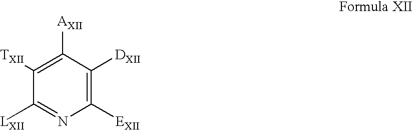

and pharmaceutically acceptable forms thereof, in which

A_{XII} and E_{XII} are identical or different and stand for aryl with 6 to 10 carbon atoms which is possibly substituted, up to 5-fold identical or different, by halogen, hydroxy, trifluoromethyl, trifluoromethoxy, nitro or by straight-chain or branched alkyl, acyl, hydroxy alkyl or alkoxy with up to 7 carbon atoms each, or by a group of the formula —NR_{XII-1}R_{XII-2},

where

R_{XII-1} and R_{XII-2} are identical or different and are meant to be hydrogen, phenyl or straight-chain or branched alkyl with up to 6 carbon atoms,

D_{XII} stands for straight-chain or branched alkyl with up to 8 carbon atoms, which is substituted by hydroxy,

L_{XII} stands for cycloalkyl with 3 to 8 carbon atoms or for straight-chain or branched alkyl with up to 8 carbon atoms, which is possibly substituted by cycloalkyl with 3 to 8 carbon atoms, or by hydroxy,

T_{XII} stands for a radical of the formula R_{XII-3}—X_{XII}— or

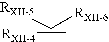

where

R_{XII-3} and R_{XII-4} are identical or different and are meant to be cycloalkyl with 3 to 8 carbon atoms, or aryl with 6 to 10 carbon atoms, or a 5- to 7-membered aromatic, possibly benzocondensated heterocycle with up to 3 heteroatoms from the series S, N and/or O, which are possibly substituted up to 3-fold identical or different, by trifluoromethyl, trifluoromethoxy, halogen, hydroxy, carboxyl, nitro, by straight-chain or branched alkyl, acyl, alkoxy or alkoxycarbonyl with up to 6 carbon atoms each or by phenyl, phenoxy or phenylthio which in turn can be substituted by halogen trifluoromethyl or trifluoromethoxy, and/or where the cycles are possibly substituted by a group of the formula —NR_{XII-7}R_{XII-8},

where

R_{XII-7} and R_{XII-8} are identical or different and have the meaning of R_{XII-1} and R_{XII-2} given above,

X_{XII} is a straight-chain or branched alkyl or alkenyl with 2 to 10 carbon atoms each, possibly substituted up to 2-fold by hydroxy or halogen,

R_{XII-5} stands for hydrogen,

and

R_{XII-6} means to be hydrogen, halogen, mercapto, azido, trifluoromethyl, hydroxy, trifluoromethoxy, straight-chain or branched alkoxy with up to 5 carbon atoms, or a radical of the formula —NR_{XII-9}R_{XII-10},

where

R_{XII-9} and R_{XII-10} are identical or different and have the meaning of R_{XII-1} and R_{XII-2} given above,

or

R_{XII-5} and R_{XII-6}, together with the carbon atom, form a carbonyl group.

Compounds of Formula XII are disclosed in EP 796846-A1, the complete disclosure of which is incorporated by reference.

In a preferred embodiment, the CETP inhibitor is selected from the following compounds of Formula XII:

4,6-bis-(p-fluorophenyl)-2-isopropyl-3-[(p-trifluoromethylphenyl)-(fluoro)-methyl]-5-(1-hydroxyethyl)pyridine;

2,4-bis-(4-fluorophenyl)-6-isopropyl-5-[4-(trifluoromethylphenyl)-fluoromethyl]-3-hydroxymethyl)pyridine; and

2,4-bis-(4-fluorophenyl)-6-isopropyl-5-[2-(3-trifluoromethylphenyl)vinyl]-3-hydroxymethyl)pyridine.

Another class of CETP inhibitors that finds utility with the present invention consists of compounds having the Formula XIII

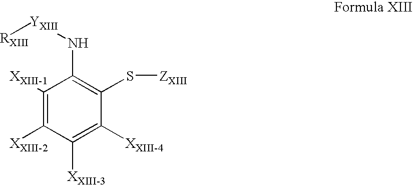

and pharmaceutically acceptable forms thereof, in which

R_{XIII} is a straight chain or branched C₁₋₁₀ alkyl; straight chain or branched C₂₋₁₀ alkenyl; halogenated C₁₋₄ lower alkyl; C₃₋₁₀ cycloalkyl that may be substituted; C₅₋₈ cycloalkenyl that may be substituted; C₃₋₁₀ cycloalkyl C₁₋₁₀ alkyl that may be substituted; aryl that may be substituted; aralkyl that may be substituted; or a 5- or 6-membered heterocyclic group having 1 to 3 nitrogen atoms, oxygen atoms or sulfur atoms that may be substituted,

X_{XIII-1}, X_{XIII-2}, X_{XIII-3}, X_{XIII-4} may be the same or different and are a hydrogen atom; halogen atom; C₁₋₄ lower alkyl; halogenated C₁₋₄ lower alkyl; C₁₋₄ lower alkoxy; cyano group; nitro group; acyl; or aryl, respectively;

Y_{XIII} is —CO—; or —SO₂—; and

Z_{XIII} is a hydrogen atom; or mercapto protective group.

Compounds of Formula XIII are disclosed in WO 98/35937, the complete disclosure of which is incorporated by reference.

In a preferred embodiment, the CETP inhibitor is selected from the following compounds of Formula XIII:

N,N′-(dithiodi-2,1-phenylene)bis[2,2-dimethyl-propanamide];

N,N′-(dithiodi-2,1-phenylene)bis[1-methyl-cyclohexanecarboxamide];

N,N′-(dithiodi-2,1-phenylene)bis[1-(3-methylbutyl)-cyclopentanecarboxamide];

N,N′-(dithiodi-2,1-phenylene)bis[1-(3-methylbutyl)-cyclohexanecarboxamide];

N,N′-(dithiodi-2,1-phenylene)bis[1-(2-ethylbutyl)-cyclohexanecarboxamide];

N,N′-(dithiodi-2,1-phenylene)bis-tricyclo[3.3.1.1^{3,7}]decane-1-carboxamide;

propanethioic acid, 2-methyl-,S-[2[[[1-(2-ethylbutyl)cyclohexyl]carbonyl]amino]phenyl] ester;

propanethioic acid, 2,2-dimethyl-,S-[2-[[[1-(2-ethylbutyl)cyclohexyl]carbonyl]amino]phenyl] ester; and

ethanethioic acid, S-[2-[[[1-(2-ethylbutyl)cyclohexyl]carbonyl]amino]phenyl] ester.

Another class of CETP inhibitors that finds utility with the present invention consists of polycyclic aryl and heteroaryl tertiary-heteroalkylamines having the Formula XIV

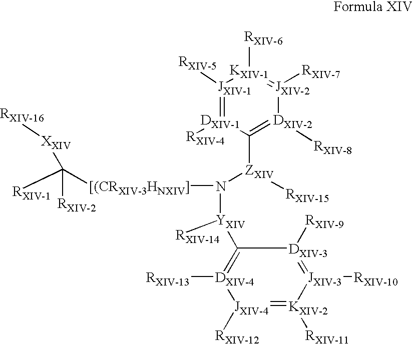

and pharmaceutically acceptable forms thereof, wherein:

n_{XIV} is an integer selected from 0 through 5;

R_{XIV-I} is selected from the group consisting of haloalkyl, haloalkenyl, haloalkoxyalkyl, and haloalkenyloxyalkyl;

X_{XIV} is selected from the group consisting of O, H, F, S, S(O), NH, N(OH), N(alkyl), and N(alkoxy);

R_{XIV-16} is selected from the group consisting of hydrido, alkyl, alkenyl, alkynyl, aryl, aralkyl, aryloxyalkyl, alkoxyalkyl, alkenyloxyalkyl, alkylthioalkyl, arylthioalkyl, aralkoxyalkyl, heteroaralkoxyalkyl, alkylsulfinylalkyl, alkylsulfonylalkyl, cycloalkyl, cycloalkylalkyl, cycloalkylalkenyl, cycloalkenyl, cycloalkenylalkyl, haloalkyl, haloalkenyl, halocycloalkyl, halocycloalkenyl, haloalkoxyalkyl, haloalkenyloxyalkyl, halocycloalkoxyalkyl, halocycloalkenyloxyalkyl, perhaloaryl, perhaloaralkyl, perhaloaryloxyalkyl, heteroaryl, heteroarylalkyl, monocarboalkoxyalkyl, monocarboalkoxy, dicarboalkoxyalkyl, monocarboxamido, monocyanoalkyl, dicyanoalkyl, carboalkoxycyanoalkyl, acyl, aroyl, heteroaroyl, heteroaryloxyalkyl, dialkoxyphosphonoalkyl, trialkylsilyl, and a spacer selected from the group consisting of a covalent single bond and a linear spacer moiety having from 1 through 4 contiguous atoms linked to the point of bonding of an aromatic substituent selected from the group consisting of R_{XIV-4}, R_{XIV-8}, R_{XIV-9}, and R_{XIV-13} to form a heterocyclyl ring having from 5 through 10 contiguous members with the provisos that said spacer moiety is other than a covalent single bond when R_{XIV-2} is alkyl and there is no R_{XIV-16} wherein X is H or F;

D_{XIV-1}, D_{XIV-2}, J_{XIV-1}, J_{XIV-2} and K_{XIV-1} are independently selected from the group consisting of C, N, O, S and a covalent bond with the provisos that no more than one of D_{XIV-1}, D_{XIV-2}, J_{XIV-1}, J_{XIV-2} and K_{XIV-1} is a covalent bond, no more than one of D_{XIV-1, D}_{XIV-2}, J_{XIV-1}, J_{XIV-2} and K_{XIV-1} is O, no more than one of D_{XIV-1}, D_{XIV-2}, J_{XIV-1}, J_{XIV-2} and K_{XIV-1} is S, one of D_{XIV-1}, D_{XIV-2}, J_{XIV-1}, J_{XIV-2} and K_{XIV-1} must be a covalent bond when two of D_{XIV-1}, D_{XIV-2}, J_{XIV-1}, J_{XIV-2} and K_{XIV-1} are O and S, and no more than four of D_{XIV-1}, D_{XIV-2}, J_{XIV-1}, J_{XIV-2} and K_{XIV-1} are N;

D_{XIV-3}, D_{XIV-4}, J_{XIV-3}, J_{XIV-4} and K_{XIV-2} are independently selected from the group consisting of C, N, O, S and a covalent bond with the provisos that no more than one of D_{XIV-3}, D_{XIV-4}, J_{XIV-3}, J_{XIV-4} and K_{XIV-2} is a covalent bond, no more than one of D_{XIV-3}, D_{XIV-4}, J_{XIV-3}, J_{XIV-4} and K_{XIV-2} is O, no more than one of D_{XIV-3}, D_{XIV-4}, J_{XIV-3}, J_{XIV-4} and K_{XIV-2} is S, one of D_{XIV-3}, D_{XIV-4}, J_{XIV-3} , J_{XIV-4} and K_{XIV-2} must be a covalent bond when two of D_{XIV-3}, D_{XIV-4}, J_{XIV-3}, J_{XIV-4} and K_{XIV-2} are O and S, and no more than four of D_{XIV-3}, D_{XIV-4}, J_{XIV-3}, J_{XIV-4} and K_{XIV-2} and K_{XIV-2} are N;

R_{XIV-2} is independently selected from the group consisting of hydrido, hydroxy, hydroxyalkyl, amino, aminoalkyl, alkylamino, dialkylamino, alkyl, alkenyl, alkynyl, aryl, aralkyl, aralkoxyalkyl, aryloxyalkyl, alkoxyalkyl, heteroaryloxyalkyl, alkenyloxyalkyl, alkylthioalkyl, aralkylthioalkyl, arylthioalkyl, cycloalkyl, cycloalkylalkyl, cycloalkylalkenyl, cycloalkenyl, cycloalkenylalkyl, haloalkyl, haloalkenyl, halocycloalkyl, halocycloalkenyl, haloalkoxy, aloalkoxyalkyl, haloalkenyloxyalkyl, halocycloalkoxy, halocycloalkoxyalkyl, halocycloalkenyloxyalkyl, perhaloaryl, perhaloaralkyl, perhaloaryloxyalkyl, heteroaryl, heteroarylalkyl, heteroarylthioalkyl, heteroaralkylthioalkyl, monocarboalkoxyalkyl, dicarboalkoxyalkyl, monocyanoalkyl, dicyanoalkyl, carboalkoxycyanoalkyl, alkylsulfinyl, alkylsulfonyl, alkylsulfinylalkyl, alkylsulfonylalkyl, haloalkylsulfinyl, haloalkylsulfonyl, arylsulfinyl, arylsulfinylalkyl, arylsulfonyl, arylsulfonylalkyl, aralkylsulfinyl, aralkylsulfonyl, cycloalkylsulfinyl, cycloalkylsulfonyl, cycloalkylsulfinylalkyl, cycloalkylsufonylalkyl, heteroarylsulfonylalkyl, heteroarylsulfinyl, heteroarylsulfonyl, heteroarylsulfinylalkyl, aralkylsulfinylalkyl, aralkylsulfonylalkyl, carboxy, carboxyalkyl, carboalkoxy, carboxamide, carboxamidoalkyl, carboaralkoxy, dialkoxyphosphono, diaralkoxyphosphono, dialkoxyphosphonoalkyl, and diaralkoxyphosphonoalkyl;

R_{XIV-2} and R_{XIV-3} are taken together to form a linear spacer moiety selected from the group consisting of a covalent single bond and a moiety having from 1 through 6 contiguous atoms to form a ring selected from the group consisting of a cycloalkyl having from 3 through 8 contiguous members, a cycloalkenyl having from 5 through 8 contiguous members, and a heterocyclyl having from 4 through 8 contiguous members;

R_{XIV-3 is} selected from the group consisting of hydrido, hydroxy, halo, cyano, aryloxy, hydroxyalkyl, amino, alkylamino, dialkylamino, acyl, sulfhydryl, acylamido, alkoxy, alkylthio, arylthio, alkyl, alkenyl, alkynyl, aryl, aralkyl, aryloxyalkyl, alkoxyalkyl, heteroarylthio, aralkylthio, aralkoxyalkyl, alkylsulfinylalkyl, alkylsulfonylalkyl, aroyl, heteroaroyl, aralkylthioalkyl, heteroaralkylthioalkyl, heteroaryloxyalkyl, alkenyloxyalkyl, alkylthioalkyl, arylthioalkyl, cycloalkyl, cycloalkylalkyl, cycloalkylalkenyl, cycloalkenyl, cycloalkenylalkyl, haloalkyl, haloalkenyl, halocycloalkyl, halocycloalkenyl, haloalkoxy, haloalkoxyalkyl, haloalkenyloxyalkyl, halocycloalkoxy, halocycloalkoxyalkyl, halocycloalkenyloxyalkyl, perhaloaryl, perhaloaralkyl, perhaloaryloxyalkyl, heteroaryl, heteroarylalkyl, heteroarylthioalkyl, monocarboalkoxyalkyl, dicarboalkoxyalkyl, monocyanoalkyl, dicyanoalkyl, carboalkoxycyanoalkyl, alkylsulfinyl, alkylsulfonyl, haloalkylsulfinyl, haloalkylsulfonyl, arylsulfinyl, arylsulfinylalkyl, arylsulfonyl, arylsulfonylalkyl, aralkylsulfinyl, aralkylsulfonyl, cycloalkylsulfinyl, cycloalkylsulfonyl, cycloalkylsulfinylalkyl, cycloalkylsufonylalkyl, heteroarylsulfonylalkyl, heteroarylsulfinyl, heteroarylsulfonyl, heteroarylsulfinylalkyl, aralkylsulfinylalkyl, aralkylsulfonylalkyl, carboxy, carboxyalkyl, carboalkoxy, carboxamide, carboxamidoalkyl, carboaralkoxy, dialkoxyphosphono, diaralkoxyphosphono, dialkoxyphosphonoalkyl, and diaralkoxyphosphonoalkyl;

Y_{XIV} is selected from a group consisting of a covalent single bond,(C(R_{XIV-14})₂)_{qXIV} wherein _{qXIV} is an integer selected from 1 and 2 and (CH(R_{XIV-14}))_{gXIV}—W_{XIV}—(CH(R_{XIV-14}))_{pXIV} wherein _{gXIV} and _{pXIV} are integers independently selected from 0 and 1;

R_{XIV-14} is independently selected from the group consisting of hydrido, hydroxy, halo, cyano, aryloxy, amino, alkylamino, dialkylamino, hydroxyalkyl, acyl, aroyl, heteroaroyl, heteroaryloxyalkyl, sulfhydryl, acylamido, alkoxy, alkylthio, arylthio, alkyl, alkenyl, alkynyl, aryl, aralkyl, aryloxyalkyl, aralkoxyalkylalkoxy, alkylsulfinylalkyl, alkylsulfonylalkyl, aralkylthioalkyl, heteroaralkoxythioalkyl, alkoxyalkyl, heteroaryloxyalkyl, alkenyloxyalkyl, alkylthioalkyl, arylthioalkyl, cycloalkyl, cycloalkylalkyl, cycloalkylalkenyl, cycloalkenyl, cycloalkenylalkyl, haloalkyl, haloalkenyl, halocycloalkyl, halocycloalkenyl, haloalkoxy, haloalkoxyalkyl, haloalkenyloxyalkyl, halocycloalkoxy, halocycloalkoxyalkyl, halocycloalkenyloxyalkyl, perhaloaryl, perhaloaralkyl, perhaloaryloxyalkyl, heteroaryl, heteroarylalkyl, heteroarylthioalkyl, heteroaralkylthioalkyl, monocarboalkoxyalkyl, dicarboalkoxyalkyl, monocyanoalkyl, dicyanoalkyl, carboalkoxycyanoalkyl, alkylsulfinyl, alkylsulfonyl, haloalkylsulfinyl, haloalkylsulfonyl, arylsulfinyl, arylsulfinylalkyl, arylsulfonyl, arylsulfonylalkyl, aralkylsulfinyl, aralkylsulfonyl, cycloalkylsulfinyl, cycloalkylsulfonyl, cycloalkylsulfinylalkyl, cycloalkylsufonylalkyl, heteroarylsulfonylalkyl, heteroarylsulfinyl, heteroarylsulfonyl, heteroarylsulfinylalkyl, aralkylsulfinylalkyl, aralkylsulfonylalkyl, carboxy, carboxyalkyl, carboalkoxy, carboxamide, carboxamidoalkyl, carboaralkoxy, dialkoxyphosphono, diaralkoxyphosphono, dialkoxyphosphonoalkyl, diaralkoxyphosphonoalkyl, a spacer selected from a moiety having a chain length of 3 to 6 atoms connected to the point of bonding selected from the group consisting of R_{XIV-9} and R_{XIV-13} to form a ring selected from the group consisting of a cycloalkenyl ring having from 5 through 8 contiguous members and a heterocyclyl ring having from 5 through 8 contiguous members and a spacer selected from a moiety having a chain length of 2 to 5 atoms connected to the point of bonding selected from the group consisting of R_{XIV-4} and R_{XIV-8} to form a heterocyclyl having from 5 through 8 contiguous members with the proviso that, when Y_{XIV} is a covalent bond, an R_{XIV-14} substituent is not attached to Y_{XIV};

R_{XIV-14} and R_{XIV-14}, when bonded to the different atoms, are taken together to form a group selected from the group consisting of a covalent bond, alkylene, haloalkylene, and a spacer selected from a group consisting of a moiety having a chain length of 2 to 5 atoms connected to form a ring selected from the group of a saturated cycloalkyl having from 5 through 8 contiguous members, a cycloalkenyl having from 5 through 8 contiguous members, and a heterocyclyl having from 5 through 8 contiguous members;

R_{XIV-14} and R_{XIV-14}, when bonded to the same atom are taken together to form a group selected from the group consisting of oxo, thiono, alkylene, haloalkylene, and a spacer selected from the group consisting of a moiety having a chain length of 3 to 7 atoms connected to form a ring selected from the group consisting of a cycloalkyl having from 4 through 8 contiguous members, a cycloalkenyl having from 4 through 8 contiguous members, and a heterocyclyl having from 4 through 8 contiguous members;

W_{XIV} is selected from the group consisting of O, C(O), C(S), C(O)N(R_{XIV-14}), C(S)N(R_{XIV-14}), (R_{XIV-14})NC(O), (R_{XIV-14})NC(S), S, S(O), S(O)₂, S(O)₂N(R_{XIV-14}), (R_{XIV-14})NS(O)₂, and N(R_{XIV-14}) with the proviso that R_{XIV-14} is selected from other than halo and cyano;

Z_{XIV} is independently selected from a group consisting of a covalent single bond, (C(R_{XIV-15})₂)_{qXIV-2} wherein _{qXIV-2} is an integer selected from 1 and 2, (CH(R_{XIV-15}))_{jXIV}—W—(CH(R_{XIV-15}))_{kXIV} wherein _{jXIV} and _{kXIV} are integers independently selected from 0 and 1 with the proviso that, when Z_{XIV} is a covalent single bond, an R_{XIV-15} substituent is not attached to Z_{XIV};

R_{XIV-15} is independently selected, when Z_{XIV} is (C(R_{XIV-15})₂)_{qXIV} wherein qXIV is an integer selected from 1 and 2, from the group consisting of hydrido, hydroxy, halo, cyano, aryloxy, amino, alkylamino, dialkylamino, hydroxyalkyl, acyl, aroyl, heteroaroyl, heteroaryloxyalkyl, sulfhydryl, acylamido, alkoxy, alkylthio, arylthio, alkyl, alkenyl, alkynyl, aryl, aralkyl, aryloxyalkyl, aralkoxyalkyl, alkylsulfinylalkyl, alkylsulfonylalkyl, aralkylthioalkyl, heteroaralkylthioalkyl, alkoxyalkyl, heteroaryloxyalkyl, alkenyloxyalkyl, alkylthioalkyl, arylthioalkyl, cycloalkyl, cycloalkylalkyl, cycloalkylalkenyl, cycloalkenyl, cycloalkenylalkyl, haloalkyl, haloalkenyl, halocycloalkyl, halocycloalkenyl, haloalkoxy, haloalkoxyalkyl, haloalkenyloxyalkyl, halocycloalkoxy, halocycloalkoxyalkyl, halocycloalkenyloxyalkyl, perhaloaryl, perhaloaralkyl, perhaloaryloxyalkyl, heteroaryl, heteroarylalkyl, heteroarylthioalkyl, heteroaralkylthioalkyl, monocarboalkoxyalkyl, dicarboalkoxyalkyl, monocyanoalkyl, dicyanoalkyl, carboalkoxycyanoalkyl, alkylsulfinyl, alkylsulfonyl, haloalkylsulfinyl, haloalkylsulfonyl, arylsulfinyl, arylsulfinylalkyl, arylsulfonyl, arylsulfonylalkyl, aralkylsulfinyl, aralkylsulfonyl, cycloalkylsulfinyl, cycloalkylsulfonyl, cycloalkylsulfinylalkyl, cycloalkylsufonylalkyl, heteroarylsulfonylalkyl, heteroarylsulfinyl, heteroarylsulfonyl, heteroarylsulfinylalkyl, aralkylsulfinylalkyl, aralkylsulfonylalkyl, carboxy, carboxyalkyl, carboalkoxy, carboxamide, carboxamidoalkyl, carboaralkoxy, dialkoxyphosphono, diaralkoxyphosphono, dialkoxyphosphonoalkyl, diaralkoxyphosphonoalkyl, a spacer selected from a moiety having a chain length of 3 to 6 atoms connected to the point of bonding selected from the group consisting of R_{XIV-4} and R_{XIV-8} to form a ring selected from the group consisting of a cycloalkenyl ring having from 5 through 8 contiguous members and a heterocyclyl ring having from 5 through 8 contiguous members, and a spacer selected from a moiety having a chain length of 2 to 5 atoms connected to the point of bonding selected from the group consisting of R_{XIV-9} and R_{XIV-13} to form a heterocyclyl having from 5 through 8 contiguous members;

R_{XIV-15} and R_{XIV-15}, when bonded to the different atoms, are taken together to form a group selected from the group consisting of a covalent bond, alkylene, haloalkylene, and a spacer selected from a group consisting of a moiety having a chain length of 2 to 5 atoms connected to form a ring selected from the group of a saturated cycloalkyl having from 5 through 8 contiguous members, a cycloalkenyl having from 5 through 8 contiguous members, and a heterocyclyl having from 5 through 8 contiguous members;

R_{XIV-15} and R_{XIV-15}, when bonded to the same atom are taken together to form a group selected from the group consisting of oxo, thiono, alkylene, haloalkylene, and a spacer selected from the group consisting of a moiety having a chain length of 3 to 7 atoms connected to form a ring selected from the group consisting of a cycloalkyl having from 4 through 8 contiguous members, a cycloalkenyl having from 4 through 8 contiguous members, and a heterocyclyl having from 4 through 8 contiguous members;

R_{XIV-15} is independently selected, when Z_{XIV} is (CH(R_{XIV-15}))_{jXIV}—W—(CH(R_{XIV-15}))_{kXIV} wherein _{jXIV} and _{kXIV} are integers independently selected from 0 and 1, from the group consisting of hydrido, halo, cyano, aryloxy, carboxyl, acyl, aroyl, heteroaroyl, hydroxyalkyl, heteroaryloxyalkyl, acylamido, alkoxy, alkylthio, arylthio, alkyl, alkenyl, alkynyl, aryl, aralkyl, aryloxyalkyl, alkoxyalkyl, heteroaryloxyalkyl, aralkoxyalkyl, heteroaralkoxyalkyl, alkylsulfonylalkyl, alkylsulfinylalkyl, alkenyloxyalkyl, alkylthioalkyl, arylthioalkyl, cycloalkyl, cycloalkylalkyl, cycloalkylalkenyl, cycloalkenyl, cycloalkenylalkyl, haloalkyl, haloalkenyl, halocycloalkyl, halocycloalkenyl, haloalkoxy, haloalkoxyalkyl, haloalkenyloxyalkyl, halocycloalkoxy, halocycloalkoxyalkyl, halocycloalkenyloxyalkyl, perhaloaryl, perhaloaralkyl, perhaloaryloxyalkyl, heteroaryl, heteroarylalkyl, heteroarylthioalkyl, heteroaralkylthioalkyl, monocarboalkoxyalkyl, dicarboalkoxyalkyl, monocyanoalkyl, dicyanoalkyl, carboalkoxycyanoalkyl, alkylsulfinyl, alkylsulfonyl, haloalkylsulfinyl, haloalkylsulfonyl, arylsulfinyl, arylsulfinylalkyl, arylsulfonyl, arylsulfonylalkyl, aralkylsulfinyl, aralkylsulfonyl, cycloalkylsulfinyl, cycloalkylsulfonyl, cycloalkylsulfinylalkyl, cycloalkylsufonylalkyl, heteroarylsulfonylalkyl, heteroarylsulfinyl, heteroarylsulfonyl, heteroarylsulfinylalkyl, aralkylsulfinylalkyl, aralkylsulfonylalkyl, carboxyalkyl, carboalkoxy, carboxamide, carboxamidoalkyl, carboaralkoxy, dialkoxyphosphonoalkyl, diaralkoxyphosphonoalkyl, a spacer selected from a linear moiety having a chain length of 3 to 6 atoms connected to the point of bonding selected from the group consisting of R_{XIV-4} and R_{XIV-8} to form a ring selected from the group consisting of a cycloalkenyl ring having from 5 through 8 contiguous members and a heterocyclyl ring having from 5 through 8 contiguous members, and a spacer selected from a linear moiety having a chain length of 2 to 5 atoms connected to the point of bonding selected from the group consisting of R_{XIV-9} and R_{XIV-13} to form a heterocyclyl ring having from 5 through 8 contiguous members;

R_{XIV-4}, R_{XIV-5}, R_{XIV-6}, R_{XIV-7}, R_{XIV-8}, R_{XIV-9}, R_{XIV-10}, R_{XIV-11}, R_{XIV-12}, and R_{XIV-13} are independently selected from the group consisting of perhaloaryloxy, alkanoylalkyl, alkanoylalkoxy, alkanoyloxy, N-aryl-N-alkylamino, heterocyclylalkoxy, heterocyclylthio, hydroxyalkoxy, carboxamidoalkoxy, alkoxycarbonylalkoxy, alkoxycarbonylalkenyloxy, aralkanoylalkoxy, aralkenoyl, N-alkylcarboxamido, N-haloalkylcarboxamido, N-cycloalkylcarboxamido, N-arylcarboxamidoalkoxy, cycloalkylcarbonyl, cyanoalkoxy, heterocyclylcarbonyl, hydrido, carboxy, heteroaralkylthio, heteroaralkoxy, cycloalkylamino, acylalkyl, acylalkoxy, aroylalkoxy, heterocyclyloxy, aralkylaryl, aralkyl, aralkenyl, aralkynyl, heterocyclyl, perhaloaralkyl, aralkylsulfonyl, aralkylsulfonylalkyl, aralkylsulfinyl, aralkylsulfinylalkyl, halocycloalkyl, halocycloalkenyl, cycloalkylsulfinyl, cycloalkylsulfinylalkyl, cycloalkylsulfonyl, cycloalkylsulfonylalkyl, heteroarylamino, N-heteroarylamino-N-alkylamino, heteroarylaminoalkyl, haloalkylthio, alkanoyloxy, alkoxy, alkoxyalkyl, haloalkoxylalkyl, heteroaralkoxy, cycloalkoxy, cycloalkenyloxy, cycloalkoxyalkyl, cycloalkylalkoxy, cycloalkenyloxyalkyl, cycloalkylenedioxy, halocycloalkoxy, halocycloalkoxyalkyl, halocycloalkenyloxy, halocycloalkenyloxyalkyl, hydroxy, amino, thio, nitro, lower alkylamino, alkylthio, alkylthioalkyl, arylamino, aralkylamino, arylthio, arylthioalkyl, heteroaralkoxyalkyl, alkylsulfinyl, alkylsulfinylalkyl, arylsulfinylalkyl, arylsulfonylalkyl, heteroarylsulfinylalkyl, heteroarylsulfonylalkyl, alkylsulfonyl, alkylsulfonylalkyl, haloalkylsulfinylalkyl, haloalkylsulfonylalkyl, alkylsulfonamido, alkylaminosulfonyl, amidosulfonyl, monoalkyl, amidosulfonyl, dialkyl amidosulfonyl, monoarylamidosulfonyl, arylsulfonamido, diarylamidosulfonyl, monoalkyl monoaryl amidosulfonyl, arylsulfinyl, arylsulfonyl, heteroarylthio, heteroarylsulfinyl, heteroarylsulfonyl, heterocyclylsulfonyl, heterocyclylthio, alkanoyl, alkenoyl, aroyl, heteroaroyl, aralkanoyl, heteroaralkanoyl, haloalkanoyl, alkyl, alkenyl, alkynyl, alkenyloxy, alkenyloxyalky, alkylenedioxy, haloalkylenedioxy, cycloalkyl, cycloalkylalkanoyl, cycloalkenyl, lower cycloalkylalkyl, lower cycloalkenylalkyl, halo, haloalkyl; haloalkenyl, haloalkoxy, hydroxyhaloalkyl, hydroxyaralkyl, hydroxyaikyl, hydoxyheteroaralkyl, haloalkoxyalkyl, aryl, heteroaralkynyl, aryloxy, aralkoxy, aryloxyalkyl, saturated heterocyclyl, partially saturated heterocyclyl, heteroaryl, heteroaryloxy, heteroaryloxyalkyl, arylalkenyl, heteroarylalkenyl, carboxyalkyl, carboalkoxy, alkoxycarboxamido, alkylamidocarbonylamido, arylamidocarbonylamido, carboalkoxyalkyl, carboalkoxyalkenyl, carboaralkoxy, carboxamido, carboxamidoalkyl, cyano, carbohaloalkoxy, phosphono, phosphonoalkyl, diaralkoxyphosphono, and diaralkoxyphosphonoalkyl with the proviso that there are one to five non-hydrido ring substituents R_{XIV-4}, R_{XIV-5}, R_{XIV-6}, R_{XIV-7}, and R_{XIV-8} present, that there are one to five non-hydrido ring substituents R_{XIV-9}, R_{XIV-10}, R_{XIV-11}, R_{XIV-12}, and R_{XIV-13} present, and R_{XIV-4}, R_{XIV-5}, R_{XIV-6}, R_{XIV-7}, R_{XIV-8} R_{XIV-9}, R_{XIV-10}, R_{XIV-11}, R_{XIV-12}, and R_{XIV-13} are each independently selected to maintain the tetravalent nature of carbon, trivalent nature of nitrogen, the divalent nature of sulfur, and the divalent nature of oxygen;

R_{XIV-4} and R_{XIV-5}, R_{XIV-5} and R_{XIV-6}, R_{XIV-6} and R_{XIV-7}, R_{XIV-7} and R_{XIV-8}, R_{XIV-8} and R_{XIV-9}, R_{XIV-9} and R_{XIV-10}, R_{XIV-10} and R_{XIV-11}, R_{XIV-11} and R_{XIV-12}, and R_{XIV-12} and R_{XIV-13} are independently selected to form spacer pairs wherein a spacer pair is taken together to form a linear moiety having from 3 through 6 contiguous atoms connecting the points of bonding of said spacer pair members to form a ring selected from the group consisting of a cycloalkenyl ring having 5 through 8 contiguous members, a partially saturated heterocyclyl ring having 5 through 8 contiguous members, a heteroaryl ring having 5 through 6 contiguous members, and an aryl with the provisos that no more than one of the group consisting of spacer pairs R_{XIV-4} and R_{XIV-5}, R_{XIV-5} and R_{XIV-6}, R_{XIV-6} and R_{XIV-7}, and R_{XIV-7} and R_{XIV-8} are used at the same time and that no more than one of the group consisting of spacer pairs R_{XIV-9} and R_{XIV-10}, R_{XIV-10} and R_{XIV-11}, R_{XIV-11} and R_{XIV-12}, and R_{XIV-12} and R_{XIV-13} are used at the same time;

R_{XIV-4} and R_{XIV-9}, R_{XIV-4} and R_{XIV-13}, R_{XIV-8} and R_{XIV-9}, and R_{XIV-8} and R_{XIV-13} are independently selected to form a spacer pair wherein said spacer pair is taken together to form a linear moiety wherein said linear moiety forms a ring selected from the group consisting of a partially saturated heterocyclyl ring having from 5 through 8 contiguous members and a heteroaryl ring having from 5 through 6 contiguous members with the proviso that no more than one of the group consisting of spacer pairs R_{XIV-4} and R_{XIV-9}, R_{XIV-4} and R_{XIV-13}, R_{XIV-8} and R_{XIV-9}, and R_{XIV-8} and R_{XIV-13} is used at the same time.

Compounds of Formula XIV are disclosed in WO 00/1 8721, the entire disclosure of which is incorporated by reference.

In a preferred embodiment, the CETP inhibitor is selected from the following compounds of Formula XIV:

3-[[3-(3-trifluoromethoxyphenoxy)phenyl][[3-(1,1,2,2-tetrafluoroethoxy)-phenyl]methyl]amino]-1,1,1-trifluoro-2-propanol;

3-[[3-(3-isopropylphenoxy)phenyl][[3-(1,1,2,2-tetrafluoroethoxy)phenyl]-methyl]amino]-1,1,1-trifluoro-2-propanol;

3-[[3-(3-cyclopropylphenoxy)phenyl][[3-(1,1,2,2-tetrafluoroethoxy)phenyl]-methyl]amino]-1,1,1-trifluoro-2-propanol;

3-[[3-(3-(2-furyl)phenoxy)phenyl][[3-(1,1,2,2-tetrafluoroethoxy)phenyl]-methyl]amino]1,1,1-trifluoro-2-propanol;

3-[[3-(2,3-dichlorophenoxy)phenyl][[3-(1,1,2,2-tetrafluoroethoxy)phenyl]-methyl]amino]-1,1,1-trifluoro-2-propanol;

3-[[3-(4-fluorophenoxy)phenyl][[3-(1,1,2,2-tetrafluoroethoxy)phenyl]-methyl]amino]-1,1,1-trifluoro-2-propanol;

3-[[3-(4-methlylphenoxy)phenyl][[3-(1,1,2,2-tetrafluoroethoxy)phenyl]-methyl]amino]-1,1,1-trifluoro-2-propanol;

3-[[3-(2-fluoro-5-bromophenoxy)phenyl][[3-(1,1,2,2-tetrafluoroethoxy)phenyl]-methyl]amino]-1,1,1-trifluoro-2-propanol;

3-[[3-(4-chloro-3-ethylphenoxy)phenyl][[3-(1,1,2,2-tetrafluoroethoxy)phenyl]-methyl]amino]-1,1,1-trifluoro-2-propanol;

3-[[3-[3-(1,1,2,2-tetrafluoroethoxy)phenoxy]phenyl][[3-(1,1,2,2-tetrafluoro-ethoxy)phenyl]methyl]amino]-1,1,1-trifluoro-2-propanol;

3-[[3-[3-(pentafluoroethyl)phenoxy]phenyl][[3-(1,1,2,2-tetrafluoroethoxy)-phenyl]methyl]amino]-1,1,1-trifluoro-2-propanol;

3-[[3-(3,5-dimethylphenoxy)phenyl][[3-(1,1,2,2-tetrafluoroethoxy)phenyl]-methyl]amino]-1,1,1-trifluoro-2-propanol;

3-[[3-(3-ethylphenoxy)phenyl][[3-(1,1,2,2-tetrafluoroethoxy)phenyl]-methyl]amino]-1,1,1-trifluoro-2-propanol;

3-[[3-(3-t-butylphenoxy)phenyl][[3-(1,1,2,2-tetrafluoroethoxy)phenyl]-methyl]amino]1,1,1-trifluoro-2-propanol;

3-[(3-(3-methylphenoxy)phenyl][[3-(1,1,2,2-tetrafluoroethoxy)phenyl]-methyl]amino]-1,1,1-trifluoro-2-propanol;

3-[[3-(5,6,7,8-tetrahydro-2-naphthoxy)phenyl][[3-(1,1,2,2-tetrafluoroethoxy)phenyl]methyl]amino]-1,1,1-trifluoro-2-propanol;

3-[[3-(phenoxy)phenyl][[3-(1,1,2,2-tetrafluoroethoxy)phenyl]methyl]amino]-1,1,1-trifluoro-2-propanol;

3-[[3-[3-(N,N-dimethylamino)phenoxy]phenyl][[3-(1,1,2,2-tetrafluoroethoxy)phenyl]methyl]amino]-1,1,1-trifluoro-2-propanol;

3-[[[3-(1,1,2,2-tetrafluoroethoxy)phenyl]methyl][3-[[3-(trifluoromethoxy)-phenyl]methoxy]phenyl]amino]-1,1,1-trifluoro-2-propanol;

3-[[[3-(1,1,2,2-tetrafluoroethoxy)phenyl]methyl][3-[[3-(trifluoromethyl)-phenyl]methoxy]phenyl]amino]-1,1,1-trifluoro-2-propanol;

3-[[[3-(1,1,2,2-tetrafluoroethoxy)phenyl]methyl][3-[[3,5-dimethylphenyl]-methoxy]phenyl]amino]1,1,1-trifluoro-2-propanol;

3-[[[3-(1,1,2,2-tetrafluoroethoxy)phenyl]methyl][3-[[3-(trifluoromethylthio)-phenyl]methoxy]phenyl]amino]-1,1,-trifluoro-2-propanol;

3-[[[3-(1,1,2,2-tetrafluoroethoxy)phenyl]methyl][3-[[3,5-difluorophenyl]-methoxy]phenyl]amino]-1,1,1-trifluoro-2-propanol;

3-[[[3-(1,1,2,2-tetrafluoroethoxy)phenyl]methyl][3-[cyclohexylmethoxy]-phenyl]amino]-1,1,1-trifluoro-2-propanol;

3-[[3-(2-difluoromethoxy-4-pyridyloxy)phenyl][[3-(1,1,2,2-tetrafluoroethoxy)-phenyl]methyl]amino]-1,1,1-trifluoro-2-propanol;

3-[[3-(2-trifluoromethyl-4-pyridyloxy)phenyl][[3-(1,1,2,2-tetrafluoroethoxy)-phenyl]methyl]amino]-1,1,1-trifluoro-2-propanol;

3-[[3-(3-difluoromethoxyphenoxy)phenyl][[3-(1,1,2,2-tetrafluoroethoxy)-phenyl]methyl]amino]-1,1,1-trifluoro-2-propanol;

3-[[[3-(3-trifluoromethylthio)phenoxy]phenyl][[3-(1,1,2,2-tetrafluoroethoxy)-phenyl]methyl]amino]-1,1,1-trifluoro-2-propanol;

3-[[3-(4-chloro-3-trifluoromethylphenoxy)phenyl][[3-(1,1,2,2-tetrafluoroethoxy)-phenyl]methyl]amino]-1,1,1,-trifluoro-2-propanol;

3-[[3-(3-trifluoromethoxyphenoxy)phenyl][[3-(pentafluoroethymethyl]amino]-1,1,1-trifluoro-2-propanol;

3-[[3-(3-isopropylphenoxy)phenyl][[3-(pentafluoroethyl)phenyl]methyl]-amino]-1,1,1-trifluoro-2-propanol;

3-[[3-(3-cyclopropylphenoxy)phenyl][[3-(pentafluoroethyl)phenyl]methyl]-amino]-1,1,1-trifluoro-2-propanol;

3-[[3-(3-(2-furyl)phenoxy)phenyl][[3-(pentafluoroethyl)phenyl]methyl]-amino]-1,1,1-trifluoro-2-propanol;

3-[[3-(2,3-dichlorophenoxy)phenyl][[3-(pentafluoroethyl)phenyl]methyl]-amino]-1,1,1-trifluoro-2-propanol;

3-[[3-(4-fluorophenoxy)phenyl][[3-(pentafluoroethyl)phenyl]methyl]amino]-1,1,1-trifluoro-2-propanol;

3-[[3-(4-methylphenoxy)phenyl][[3-(pentafluoroethyl)phenyl]methyl]amino]-1,1,1-trifluoro-2-propanol;

3-[[3-(2-fluoro-5-bromophenoxy)phenyl][[3-(pentafluoroethyl)phenyl]methyl]-amino]-1,1,1-trifluoro-2-propanol;

3-[[3-(4-chloro-3-ethylphenoxy)phenyl][[3-(pentafluoroethyl)phenyl]methyl]-amino]-1,1,1-trifluoro-2-propanol;

3-[[3-[3-(1,1,2,2-tetrafluoroethoxy)phenoxy]phenyl][[3-(pentafluoroethyl)-phenyl]methyl]amino]-1,1,1-trifluoro-2-propanol;

3-[[3-[3-(pentafluoroethyl)phenoxy]phenyl][[3-(pentafluoroethyl)phenyl]-methyl]amino]-1,1,1-trifluoro-2-propanol;

3-[[3-(3,5-dimethylphenoxy)phenyl][[3-(pentafluoroethyl)phenyl]methyl]-amino]-1,1,1-trifluoro-2-propanol;

3-[[3-(3-ethylphenoxy)phenyl][[3-(pentafluoroethyl)phenyl]methyl]amino]-1,1,1-trifluoro-2-propanol;

3-[[3-(3-t-butylphenoxy)phenyl][[3-(pentafluoroethyl)phenyl]methyl]amino]-1,1,1-trifluoro-2-propanol;

3-[[3-(3-methylphenoxy)phenyl][[3-pentafluoroethyl)phenyl]methyl]amino]-1,1,1-trifluoro-2-propanol;

3-[[3-(5,6,7,8-tetrahydro-2-naphthoxy)phenyl][[3-(pentafluoroethyl)phenyl]-methyl]amino]-1,1,1-trifluoro-2-propanol;

3-[[3-(phenoxy)phenyl][[3-(pentafluoroethyl)phenyl]methyl]amino]-1,1,1-trifluoro-2-propanol;

3-[[3-[3-(N,N-dimethylamino)phenoxy]phenyl][[3-(pentafluoroethyl)phenyl]-methyl]amino]-1,1,1-trifluoro-2-propanol;

3-[[[3-(pentafluoroethyl)phenyl]methyl][3-[[3-(trifluoromethoxy)phenyl]-methoxy]phenyl]amino]-1,1,1-trifluoro-2-propanol;

3-[[[3-(pentafluoroethyl)phenyl]methyl][3-[[3-(trifluoromethyl)phenyl]-methoxy]phenyl]amino]-1,1,1-trifluoro-2-propanol;

3-[[[3-(pentafluoroethyl)phenyl]methyl][3-[[3,5-dimethylphenyl]methoxy]-phenyl]amino]-1,1,1-trifluoro-2-propanol;

3-[[[3-(pentafluoroethyl)phenyl]methyl][3-[[3-(trifluoromethylthio)phenyl]-methoxy]phenyl]amino]-1,1,1-trifluoro-2-propanol;

3-[[[3-(pentafluoroethyl)phenyl]methyl][3-[[3,5-difluorophenyl]methoxy]-phenyl]amino]-1,1,1-trifluoro-2-propanol;

3-[[[3-(pentafluoroethyl)phenyl]methyl][3-[cyclohexylmethoxy]phenyl]-amino]-1,1,1-trifluoro-2-propanol;

3-[[3-(2-difluoromethoxy-4-pyridyloxy)phenyl][[3-(pentafluoroethyl)phenyl]-methyl]amino]-1,1,1-trifluoro-2-propanol;

3-[[3-(2-trifluoromethyl-4-pyridyloxy)phenyl][[3-(pentafluoroethyl)phenyl]-methyl]amino]-1,1,1-trifluoro-2-propanol;

3-[[3-(3-difluoromethoxyphenoxy)phenyl][[3-(pentafluoroethyl)phenyl]-methyl]amino]-1,1,1-trifluoro-2-propanol;

3-[[[3-(3-trifluoromethylthio)phenoxy]phenyl][[3-(pentafluoroethyl)phenyl]-methyl]amino]-1,1,1-trifluoro-2-propanol;

3-[[3-(4-chloro-3-trifluoromethylphenoxy)phenyl][[3-(pentafluoroethyl)-phenyl]methyl]amino]-1,1,1-trifluoro-2-propanol;

3-[[3-(3-trifluoromethoxyphenoxy)phenyl][[3-(heptafluoropropyl)phenyl]-methyl]amino]-1,1,1-trifluoro-2-propanol;

3-[[3-(3-isopropylphenoxy)phenyl][[3-(heptafluoropropyl)phenyl]methyl]-amino]-1,1,1-trifluoro-2-propanol;

3-[[3-(3-cyclopropylphenoxy)phenyl][[3-(heptafluoropropyl)phenyl]methyl]-amino]-1,1,1-trifluoro-2-propanol;

3-[[3-(3-(2-furyl)phenoxy)phenyl][[3-(heptafluoropropyl)phenyl]methyl]-amino]-1,1,1-trifluoro-2-propanol;

3-[[3-(2,3-dichlorophenoxy)phenyl][[3-(heptafluoropropyl)phenyl]methyl]-amino]-1,1,1-trifluoro-2-propanol;

3-[[3-(4-fluorophenoxy)phenyl][[3-(heptafluoropropyl)phenyl]methyl]amino]-1,1,1-trifluoro-2-propanol;

3-[[3-(4-methylphenoxy)phenyl][[3-(heptafluoropropyl)phenyl]methyl]amino]-1,1,1-trifluoro-2-propanol;

3-[[3-(2-fluoro-5-bromophenoxy)phenyl][[3-(heptafluoropropyl)phenyl]-methyl]amino]-1,1,1-trifluoro-2-propanol;

3-[[3-(4-chloro-3-ethylphenoxy)phenyl][[3-(heptafluoropropyl)phenyl]methyl]-amino]-1,1,1-trifluoro-2-propanol;

3-[[3-[3-(1,1,2,2-tetrafluoroethoxy)phenoxy]phenyl][[3-(heptafluoropropyl)-phenyl]methyl]amino]-1,1,1-trifluoro-2-propanol;

3-[[3-[3-(pentafluoroethyl)phenoxy]phenyl][[3-(heptafluoropropyl)phenyl]-methyl]amino]-1,1,1-trifluoro-2-propanol;

3-[[3-(3,5-dimethylphenoxy)phenyl][[3-(heptafluoropropyl)phenyl]methyl]-amino]-1,1,1-trifluoro-2-propanol;

3-[[3-(3-ethylphenoxy)phenyl][[3-(heptafluoropropyl)phenyl]methyl]amino]-1,1,1-trifluoro-2-propanol;

3-[[3-(3-t-butylphenoxy)phenyl][[3-(heptafluoropropyl)phenyl]methyl]amino]-1,1,1-trifluoro-2-propanol;

3-[[3-(3-methylphenoxy)phenyl][[3-(heptafluoropropyl)phenyl]methyl]amino]-1,1,1-trifluoro-2-propanol;

3-[(3-(5,6,7,8-tetrahydro-2-naphthoxy)phenyl][[3-(heptafluoropropyl)phenyl]-methyl]amino]-1,1,1-trifluoro-2-propanol;

3-[[3-(phenoxy)phenyl][[3-(heptafluoropropyl)phenyl]methyl]amino]-1,1,1-trifluoro-2-propanol;

3-[[3-[3-(N,N-dimethylamino)phenoxy]phenyl][[3-(heptafluoropropyl)phenyl]-methyl]amino]-1,1,1-trifluoro-2-propanol;

3-[[[3-(heptafluoropropyl)phenyl]methyl][3-[[3-(trifluoromethoxy)phenyl]-methoxy]phenyl]amino]-1,1,1-trifluoro-2-propanol;

3-[[[3-(heptafluoropropyl)phenyl]methyl][3-[[3-(trifluoromethyl)phenyl]-methoxy]phenyl]amino]-1,1,1-trifluoro-2-propanol;

3-[[[3-(heptafluoropropyl)phenyl]methyl][3-[[3,5-dimethylphenyl]methoxy]-phenyl]amino]-1,1,1-trifluoro-2-propanol;

3-[[[3-(heptafluoropropyl)phenyl]methyl][3-[[3-(trifluoromethylthio)phenyl]-methoxy]phenyl]amino]-1,1,1-trifluoro-2-propanol;

3-[[[3-(heptafluoropropyl)phenyl]methyl][3-[[3,5-difluorophenyl]methoxy]-phenyl]amino]-1,1,1-trifluoro-2-propanol;

3-[[[3-(heptafluoropropyl)phenyl]methyl][3-[cyclohexylmethoxy]phenyl]-amino]-1,1,1-trifluoro-2-propanol;

3-[[3-(2-difluoromethoxy-4-pyridyloxy)phenyl][[3-(heptafluoropropyl)phenyl]-methyl]amino]-1,1,1-trifluoro-2-propanol;

3-[[3-(2-trifluoromethyl-4-pyridyloxy)phenyl][[3-(heptafluoropropyl)phenyl]-methyl]amino]-1,1,1-trifluoro-2-propanol;

3-[[3-(3-difluoromethoxyphenoxy)phenyl][[3-(heptafluoropropyl)phenyl]-methyl]amino]-1,1,1-trifluoro-2-propanol;

3-[[[3-(3-trifluoromethylthio)phenoxy]phenyl][[3-(heptafluoropropyl)phenyl]-methyl]amino]-1,1,1-trifluoro-2-propanol;

3-[[3-(4-chloro-3-trifluoromethylphenoxy)phenyl][[3-(heptafluoropropyl)-phenyl]-methyl]amino]-1,1,1-trifluoro-2-propanol;

3-[[3-(3-trifluoromethoxyphenoxy)phenyl][[2-fluoro-5-(trifluoromethyl)-phenyl]-methyl]amino]-1,1,1-trifluoro-2-propanol;

3-[[3-(3-isopropylphenoxy)phenyl][[2-fluoro-5-(trifluoromethyl)phenyl]-methyl]amino]-1,1,1-trifluoro-2-propanol;

3-[[3-(3-cyclopropylphenoxy)phenyl][[2-fluoro-5-(trifluoromethyl)phenyl]-methyl]amino]-1,1,1-trifluoro-2-propanol;

3-[[3-(3-(2-furyl)phenoxy)phenyl][[2-fluoro-5-(trifluoromethyl)phenyl]-methyl]amino]-1,1,1-trifluoro-2-propanol;

3-[[3-(2,3-dichlorophenoxy)phenyl][[2-fluoro-5-(trifluoromethyl)phenyl]-methyl]amino]-1,1,1-trifluoro-2-propanol;

3-[[3-(4-fluorophenoxy)phenyl][[2-fluoro-5-(trifluoromethyl)phenyl]-methyl]amino]-1,1,1-trifluoro-2-propanol;

3-[[3-(4-methylphenoxy)phenyl][[2-fluoro-5-(trifluoromethyl)phenyl]-methyl]amino]-1,1,1-trifluoro-2-propanol;

3-[[3-(2-fluoro-5-bromophenoxy)phenyl][[2-fluoro-5-(trifluoromethyl)-phenyl]methyl]amino]-1,1,1-trifluoro-2-propanol;

3-[[3-(4-chloro-3-ethylphenoxy)phenyl][[2-fluoro-5-(trifluoromethyl)-phenyl]methyl]amino]-1,1,1-trifluoro-2-propanol;

3-[[3-[3-(1,1,2,2-tetrafluoroethoxy)phenoxy]phenyl][[2-fluoro-5-(trifluoro-methyl)phenyl]methyl]amino]-1,1,1-trifluoro-2-propanol;

3-[[3-[3-(pentafluoroethyl)phenoxy]phenyl][[2-fluoro-5-(trifluoromethyl)-phenyl]-methyl]amino]-1,1,1-trifluoro-2-propanol;

3-[[3-(3,5-dimethylphenoxy)phenyl][[2-fluoro-5-(trifluoromethyl)phenyl]-methyl]amino]-1,1,1-trifluoro-2-propanol;

3-[[3-(3-ethylphenoxy)phenyl][[2-fluoro-5-(trifluoromethyl)phenyl]methyl]-amino]-1,1,1-trifluoro-2-propanol;

3-[[3-(3-t-butylphenoxy)phenyl][[2-fluoro-5-(trifluoromethyl)phenyl]methyl]-amino]-1,1,1-trifluoro-2-propanol;

3-[[3-(3-methylphenoxy)phenyl][[2-fluoro-5-(trifluoromethyl)phenyl]methyl]-amino]-1,1,1-trifluoro-2-propanol;

3-[[3-(5,6,7,8-tetrahydro-2-naphthoxy)phenyl][[2-fluoro-5-(trifluoromethyl)-phenyl]methyl]amino]-1,1,1-trifluoro-2-propanol;

3-[[3-(phenoxy)phenyl][[2-fluoro-5-(trifluoromethyl)phenyl]methyl]amino]-1,1,1-trifluoro-2-propanol;

3-[[3-[3-(N,N-dimethylamino)phenoxy]phenyl][[2-fluoro-5-(trifluoromethyl)-phenyl]methyl]amino]-1,1,1-trifluoro-2-propanol;

3-[[[2-fluoro-5-(trifluoromethyl)phenyl]methyl][3-[[3-(trifluoromethoxy)-phenyl]methoxy]phenyl]amino]-1,1,1-trifluoro-2-propanol;

3-[[[2-fluoro-5-(trifluoromethyl)phenyl]methyl][3-[[3-(trifluoromethyl)-phenyl]methoxy]phenyl]amino]-1,1,1-trifluoro-2-propanol;

3-[[[2-fluoro-5-(trifluoromethyl)phenyl]methyl][3-[[3,5-dimethylphenyl]-methoxy]phenyl]amino]-1,1,1-trifluoro-2-propanol;

3-[[[2-fluoro-5-(trifluoromethyl)phenyl]methyl][3-[[3-(trifluoromethylthio)-phenyl]methoxy]phenyl]amino]-1,1,1-trifluoro-2-propanol;

3-[[[2-fluoro-5-(trifluoromethyl)phenyl]methyl][3-[[3,5-difluorophenyl]-methoxy]phenyl]amino]-1,1,1-trifluoro-2-propanol;

3-[[[2-fluoro-5-(trifluoromethyl)phenyl]methyl][3-[cyclohexylmethoxy]-phenyl]amino]-1,1,1-trifluoro-2-propanol;

3-[[3-(2-difluoromethoxy-4-pyridyloxy)phenyl][[2-fluoro-5-(trifluoromethyl)-phenyl]methyl]amino]-1,1,1-trifluoro-2-propanol;

3-[[3-(2-trifluoromethyl-4-pyridyloxy)phenyl][[2-fluoro-5-(trifluoromethyl)-phenyl]methyl]amino]-1,1,1-trifluoro-2-propanol;

3-[[3-(3-difluoromethoxyphenoxy)phenyl][[2-fluoro-5-(trifluoromethyl)-phenyl]methyl]amino]-1,1,1-trifluoro-2-propanol;

3-[[[3-(3-trifluoromethylthio)phenoxy]phenyl][[2-fluoro-5-(trifluoromethyl)-phenyl]methyl]amino]-1,1,1-trifluoro-2-propanol;

3-[[3-(4-chloro-3-trifluoromethylphenoxy)phenyl][[2-fluoro-5-(trifluoromethyl)phenyl]methyl]amino]-1,1,1-trifluoro-2-propanol;

3-[[3-(3-trifluoromethoxyphenoxy)phenyl][[2-fluoro-4-(trifluoromethyl)-phenyl]methyl]amino]-1,1,1-trifluoro-2-propanol;

3-[[3-(3-isopropylphenoxy)phenyl][[2-fluoro-4-(trifluoromethyl)phenyl]-methyl]amino]-1,1,1-trifluoro-2-propanol;

3-[[3-(3-cyclopropylphenoxy)phenyl][[2-fluoro-4-(trifluoromethyl)phenyl]-methyl]amino]-1,1,1-trifluoro-2-propanol;

3-[[3-(3-(2-furyl)phenoxy)phenyl][[2-fluoro-4-(trifluoromethyl)phenyl]-methyl]amino]-1,1,1-trifluoro-2-propanol;

3-[[3-(2,3-dichlorophenoxy)phenyl][[2-fluoro-4-(trifluoromethyl)phenyl]-methyl]amino]-1,1,1-trifluoro-2-propanol;

3-[[3-(4-fluorophenoxy)phenyl][[2-fluoro-4-(trifluoromethyl)phenyl]-methyl]amino]-1,1,1-trifluoro-2-propanol;

3-[[3-(4-methylphenoxy)phenyl][[2-fluoro-4-(trifluoromethyl)phenyl]-methyl]amino]-1,1,1-trifluoro-2-propanol;

3-[[3-(2-fluoro-5-bromophenoxy)phenyl][[2-fluoro-4-(trifluoromethyl)-phenyl]methyl]amino]-1,1,1-trifluoro-2-propanol;

3-[[3-(4-chloro-3-ethylphenoxy)phenyl][[2-fluoro-4-(trifluoromethyl)-phenyl]methyl]amino]-1,1,1-trifluoro-2-propanol;

3-[[3-[3-(1,1,2,2-tetrafluoroethoxy)phenoxy]phenyl][[2-fluoro-4-(trifluoro-methyl)phenyl]methyl]amino]-1,1,1-trifluoro-2-propanol;

3-[[3-[3-(pentafluoroethyl)phenoxy]phenyl][[2-fluoro-4-(trifluoromethyl)-phenyl]methyl]amino]-1,1,1-trifluoro-2-propanol;

3-[[3-(3,5-dimethylphenoxy)phenyl][[2-fluoro-4-(trifluoromethyl)phenyl]-methyl]amino]-1,1,1-trifluoro-2-propanol;

3-[[3-(3-ethylphenoxy)phenyl][[2-fluoro-4-(trifluoromethyl)phenyl]methyl]-amino]-1,1,1-trifluoro-2-propanol;

3-[[3-(3-t-butylphenoxy)phenyl][[2-fluoro-4-(trifluoromethyl)phenyl]methyl]-amino]-1,1,1-trifluoro-2-propanol;

3-[[3-(3-methylphenoxy)phenyl][[2-fluoro-4-(trifluoromethyl)phenyl]methyl]-amino]-1,1,1-trifluoro-2-propanol;

3-[[3-(5,6,7,8-tetrahydro-2-naphthoxy)phenyl][[2-fluoro-4-(trifluoromethyl)-phenyl]methyl]amino]-1,1,1-trifluoro-2-propanol;

3-[[3-(phenoxy)phenyl][[2-fluoro-4-(trifluoromethyl)phenyl]methyl]amino]-1,1,1-trifluoro-2-propanol;

3-[[3-[3-(N,N-dimethylamino)phenoxy]phenyl][[2-fluoro-4-(trifluoromethyl)-phenyl]methyl]amino]-1,1,1-trifluoro-2-propanol;

3-[[[2-fluoro-4-(trifluoromethyl)phenyl]methyl][3-[[3-(trifluoromethoxy)-phenyl]methoxy]phenyl]amino]-1,1,1-trifluoro-2-propanol;

3-[[[2-fluoro-4-(trifluoromethyl)phenyl]methyl][3-[[3-(trifluoromethyl)-phenyl]methoxy]phenyl]amino]-1,1,1-trifluoro-2-propanol;

3-[[[2-fluoro-4-(trifluoromethyl)phenyl]methyl][3-[[3,5-dimethylphenyl]-methoxy]phenyl]amino]-1,1,1-trifluoro-2-propanol;

3-[[[2-fluoro-4-(trifluoromethyl)phenyl]methyl][3-[[3-(trifluoromethylthio)-phenyl]methoxy]phenyl]amino]-1,1,1-trifluoro-2-propanol;

3-[[[2-fluoro-4-(trifluoromethyl)phenyl]methyl][3-[[3,5-difluorophenyl]-methoxy]phenyl]amino]-1,1 1-trifluoro-2-propanol;

3-[[[2-fluoro-4-(trifluoromethyl)phenyl]methyl][3-[cyclohexylmethoxy]-phenyl]amino]-1,1,1-trifluoro-2-propanol;

3-[[3-(2-difluoromethoxy-4-pyridyloxy)phenyl][[2-fluoro-4-(trifluoromethyl)-phenyl]methyl]amino]-1,1,1-trifluoro-2-propanol;

3-[[3-(2-trifluoromethyl-4-pyridyloxy)phenyl][[2-fluoro-4-(trifluoromethyl)-phenyl]methyl]amino]-1,1,1-trifluoro-2-propanol;

3-[[3-(3-difluoromethoxyphenoxy)phenyl][[2-fluoro-4-(trifluoromethyl)-phenyl]methyl]amino]-1,1,1-trifluoro-2-propanol;

3-[[[3-(3-trifluoromethylthio)phenoxy]phenyl][[2-fluoro-4-(trifluoromethyl)-phenyl]methyl]amino]-1,1,1-trifluoro-2-propanol; and

3-[[3-(4-chloro-3-trifluoromethylphenoxy)phenyl][[2-fluoro-4-(trifluoro-methyl)phenyl]methyl]amino]-1,1,1-trifluoro-2-propanol.

Another class of CETP inhibitors that finds utility with the present invention consists of substitued N-Aliphatic-N-Aromatic tertiary-Heteroalkylamines having the Formula XV

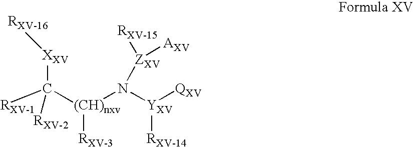

and pharmaceutically acceptable forms thereof, wherein:

n_{XV} is an integer selected from 1 through 2;

A_{XV} and Q_{XV} are independently selected from the group consisting of —CH₂(CR_{XV-37}R_{XV-38})_{vXV}—(CR_{XV-33}R_{XV-34})_{uXV}-T_{XV}-(CR_{XV-35}R_{XV-36})_{wXV-}H,

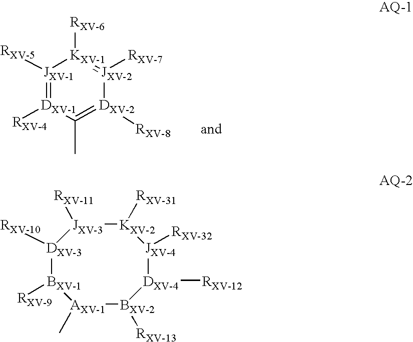

with the provisos that one of A_{XV} and Q_{XV} must be AQ-1 and that one of A_{XV} and Q_{XV} must be selected from the group consisting of AQ-2 and —C₂(CR_{XV-37}R_{XV-38})_{vXV}—(CR_{XV-33}R_{XV-34})_{uXV}-T_{XV}-(CR_{XV-35}R_{XV-36})_{wXV}—H;

T_{XV} is selected from the group consisting of a single covalent bond, O, S, S(O), S(O)₂, C(R_{XV-33})═C(R_{XV-35}), and

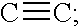

_{vXV} is an integer selected from 0 through 1 with the proviso that _{vXV} is 1 when any one of R_{XV-33}, R_{XV-34}, R_{XV-35}, and R_{XV-36} is aryl or heteroaryl;

_{uXV} and _{wXV} are integers independently selected from 0 through 6;

A_{XV-1} is C(R_{XV-30});

D_{XV-1}, D_{XV-2}, J_{XV-1}, J_{XV-2}, and K_{XV-1} are independently selected from the group consisting of C, N, O, S and a covalent bond with the provisos that no more than one of D_{XV-1}, D_{XV-2}, J_{XV-1}, J_{XV-2}, and K_{XV-1} is a covalent bond, no more than one of D_{XV-1}, D_{XV-2}, J_{XV-1}, J_{XV-2}, and K_{XV-1} is O, no more than one of D_{XV-1}, D_{XV-2}, J_{XV-1}, J_{XV-2}, and K_{XV-1} is S, one of D_{XV-1}, D_{XV-2}, J_{XV-1}, J_{XV-2}, and K_{XV-1} must be a covalent bond when two of D_{XV-1}, D_{XV-2}, J_{XV-1}, J_{XV-2}, and K_{XV-1} are O and S, and no more than four of D_{XV-1}, D_{XV-2}, J_{XV-1}, J_{XV-2}, and K_{XV-1} are N;

B_{XV-1}, B_{XV-2}, D_{XV-3}, D_{XV-4}, J_{XV-3}, J_{XV-4}, and K_{XV-2} are independently selected from the group consisting of C, C(R_{XV-30}), N, O, S and a covalent bond with the provisos that no more than 5 of B_{XV-1}, B_{XV-2}, D_{XV-3}, D_{XV-4}, J_{XV-3}, J_{XV-4}, and K_{XV-2} are a covalent bond, no more than two of B_{XV-1}, B_{XV-2}, D_{XV-3}, D_{XV-4}, J_{XV-3}, J_{XV-4}, and K_{XV-2} are O, no more than two of B_{XV-1}, B_{XV-2}, D_{XV-3}, D_{XV-4}, J_{XV-3}, J_{XV-4}, and K_{XV-2} are S, no more than two of B_{XV-1}, B_{XV-2}, D_{XV-3}, D_{XV-4}, J_{XV-3}, J_{XV-4}, and K_{XV-2} are simultaneously O and S, and no more than two of B_{XV-1}, B_{XV-2}, D_{XV-3}, D_{XV-4}, J_{XV-3}, J_{XV-4}, and K_{XV-2} are N;

B_{XV-1} and D_{XV-3}, D_{XV-3} and J_{XV-3}, J_{XV-3} and K_{XV-2}, K_{XV-2} and J_{XV-4}, J_{XV-4} and D_{XV-4}, and D_{XV-4} and B_{XV-2} are independently selected to form an in-ring spacer pair wherein said spacer pair is selected from the group consisting of C(R_{XV-33})═C(R_{XV-35}) and N═N with the provisos that AQ-2 must be a ring of at least five contiguous members, that no more than two of the group of said spacer pairs are simultaneously C(R_{XV-33})═C(R_{XV-35}) and that no more than one of the group of said spacer pairs can be N═N unless the other spacer pairs are other than C(R_{XV-33})═C(R_{XV-35}), O, N, and S;

R_{XV-1} is selected from the group consisting of haloalkyl and haloalkoxymethyl;

R_{XV-2} is selected from the group consisting of hydrido, aryl, alkyl, alkenyl, haloalkyl, haloalkoxy, haloalkoxyalkyl, perhaloaryl, perhaloaralkyl, perhaloaryloxyalkyl and heteroaryl;

R_{XV-3} is selected from the group consisting of hydrido, aryl, alkyl, alkenyl, haloalkyl, and haloalkoxyalkyl;

Y_{XV} is selected from the group consisting of a covalent single bond, (CH₂)_{q} wherein q is an integer selected from 1 through 2 and (CH₂)ⱼ—O—(CH₂)ₖ wherein j and k are integers independently selected from 0 through 1;

Z_{XV} is selected from the group consisting of covalent single bond, (CH₂)_{q} wherein q is an integer selected from 1 through 2, and (CH₂)ⱼ—O—(CH₂)ₖ wherein j and k are integers independently selected from 0 through 1;

R_{XV-4}, R_{XV-8}, R_{XV-9} and R_{XV-13} are independently selected from the group consisting of hydrido, halo, haloalkyl, and alkyl;

R_{XV-30} is selected from the group consisting of hydrido, alkoxy, alkoxyalkyl, halo, haloalkyl, alkylamino, alkylthio, alkylthioalkyl, alkyl, alkenyl, haloalkoxy, and haloalkoxyalkyl with the proviso that R_{XV-30} is selected to maintain the tetravalent nature of carbon, trivalent nature of nitrogen, the divalent nature of sulfur, and the divalent nature of oxygen;

R_{XV-30}, when bonded to A_{XV-I}, is taken together to form an intra-ring linear spacer connecting the A_{XV-I}-carbon at the point of attachment of R_{XV-30} to the point of bonding of a group selected from the group consisting of R_{XV-10}, R_{XV-11}, R_{XV-12}, R_{XV-31}, and R_{XV-32} wherein said intra-ring linear spacer is selected from the group consisting of a covalent single bond and a spacer moiety having from 1 through 6 contiguous atoms to form a ring selected from the group consisting of a cycloalkyl having from 3 through 10 contiguous members, a cycloalkenyl having from 5 through 10 contiguous members, and a heterocyclyl having from 5 through 10 contiguous members;

R_{XV-30}, when bonded to A_{XV-I}, is taken together to form an intra-ring branched spacer connecting the A_{XV-I}-carbon at the point of attachment of R_{XV-30} to the points of bonding of each member of any one of substituent pairs selected from the group consisting of subsitituent pairs R_{XV-10} and R_{XV-11}, R_{XV-10} and R_{XV-31}, R_{XV-10} and R_{XV-32}, R_{XV-10} and R_{XV-12}, R_{XV-11} and R_{XV-31}, R_{XV-11} and R_{XV-32}, R_{XV-11} and R_{XV-12}, R_{XV-31} and R_{XV-32}, R_{XV-31} and R_{XV-12}, and R_{XV-32} and R_{XV-12} and wherein said intra-ring branched spacer is selected to form two rings selected from the group consisting of cycloalkyl having from 3 through 10 contiguous members, cycloalkenyl having from 5 through 10 contiguous members, and heterocyclyl having from 5 through 10 contiguous members;

R_{XV-4}, R_{XV-5}, R_{XV-6}, R_{XV-7}, R_{XV-8}, R_{XV-9}, R_{XV-10}, R_{XV-11}, R_{XV-12}, R_{XV-13}, R_{XV-31}, R_{XV-32}, R_{XV-33}, R_{XV-34}, R_{XV-35}, and R_{XV-36} are independently selected from the group consisting of hydrido, carboxy, heteroaralkylthio, heteroaralkoxy, cycloalkylamino, acylalkyl, acylalkoxy, aroylalkoxy, heterocyclyloxy, aralkylaryl, aralkyl, aralkenyl, aralkynyl, heterocyclyl, perhaloaralkyl, aralkylsulfonyl, aralkylsulfonylalkyl, aralkylsulfinyl, aralkylsulfinylalkyl, halocycloalkyl, halocycloalkenyl, cycloalkylsulfinyl, cycloalkylsulfinylalkyl, cycloalkylsulfonyl, cycloalkylsulfonylalkyl, heteroarylamino, N-heteroarylamino-N-alkylamino, heteroarylaminoalkyl, haloalkylthio, alkanoyloxy, alkoxy, alkoxyalkyl, haloalkoxylalkyl, heteroaralkoxy, cycloalkoxy, cycloalkenyloxy, cycloalkoxyalkyl, cycloalkylalkoxy, cycloalkenyloxyalkyl, cycloalkylenedioxy, halocycloalkoxy, halocycloalkoxyalkyl, halocycloalkenyloxy, halocycloalkenyloxyalkyl, hydroxy, amino, thio, nitro, lower alkylamino, alkylthio, alkylthioalkyl, arylamino, aralkylamino, arylthio, arylthioalkyl, heteroaralkoxyalkyl, alkylsulfinyl, alkylsulfinylalkyl, arylsulfinylalkyl, arylsulfonylalkyl, heteroarylsulfinylalkyl, heteroarylsulfonylalkyl, alkylsulfonyl, alkylsulfonylalkyl, haloalkylsulfinylalkyl, haloalkylsulfonylalkyl, alkylsulfonamido, alkylaminosulfonyl, amidosulfonyl, monoalkyl amidosulfonyl, dialkyl amidosulfonyl, monoarylamidosulfonyl, arylsulfonamido, diarylamidosulfonyl, monoalkyl monoaryl amidosulfonyl, arylsulfinyl, arylsulfonyl, heteroarylthio, heteroarylsulfinyl, heteroarylsulfonyl, heterocyclylsulfonyl, heterocyclylthio, alkanoyl, alkenoyl, aroyl, heteroaroyl, aralkanoyl, heteroaralkanoyl, haloalkanoyl, alkyl, alkenyl, alkynyl, alkenyloxy, alkenyloxyalky, alkylenedioxy, haloalkylenedioxy, cycloalkyl, cycloalkylalkanoyl, cycloalkenyl, lower cycloalkylalkyl, lower cycloalkenylalkyl, halo, haloalkyl, haloalkenyl, haloalkoxy, hydroxyhaloalkyl, hydroxyaralkyl, hydroxyalkyl, hydoxyheteroaralkyl, haloalkoxyalkyl, aryl, heteroaralkynyl, aryloxy, aralkoxy, aryloxyalkyl, saturated heterocyclyl, partially saturated heterocyclyl, heteroaryl, heteroaryloxy, heteroaryloxyalkyl, arylalkenyl, heteroarylalkenyl, carboxyalkyl, carboalkoxy, alkoxycarboxamido, alkylamidocarbonylamido, alkylamidocarbonylamido, carboalkoxyalkyl, carboalkoxyalkenyl, carboaralkoxy, carboxamido, carboxamidoalkyl, cyano, carbohaloalkoxy, phosphono, phosphonoalkyl, diaralkoxyphosphono, and diaralkoxyphosphonoalkyl with the provisos that R_{XV-4}, R_{XV-5}, R_{V-6}, R_{XV-7}, R_{XV-8}, R_{XV-9}, R_{XV-10}, R_{XV-11}, R_{XV-12}, R_{XV-13}, R_{XV-31}, R_{XV-32}, R_{XV-33}, R_{XV-34}, R_{XV-35}, and R_{XV-36} are each independently selected to maintain the tetravalent nature of carbon, trivalent nature of nitrogen, the divalent nature of sulfur, and the divalent nature of oxygen, that no more than three of the R_{XV-33} and R_{XV-34} substituents are simultaneously selected from other than the group consisting of hydrido and halo, and that no more than three of the R_{XV-35} and R_{XV-36} substituents are simultaneously selected from other than the group consisting of hydrido and halo;

R_{XV-9}, R_{XV-10}, R_{XV-11}, R_{XV-12}, R_{XV-13}, R_{XV-31}, and R_{XV-32} are independently selected to be oxo with the provisos that B_{XV-1}, B_{XV-2}, D_{XV-3}, D_{XV-4}, J_{XV-3}, J_{XV-4}, and K_{XV-2} are independently selected from the group consisting of C and S, no more than two of R_{XV-9}, R_{XV-10}, R_{XV-11}, R_{XV-12}, R_{XV-13}, R_{XV-31}, and R_{XV-32} are simultaneously oxo, and that R_{XV-9}, R_{XV-10}, R_{XV-11}, R_{XV-12}, R_{XV-13}, R_{XV-31}, and R_{XV-32} are each independently selected to maintain the tetravalent nature of carbon, trivalent nature of nitrogen, the divalent nature of sulfur, and the divalent nature of oxygen;

R_{XV-4} and R_{XV-5}, R_{XV-5} and R_{XV-6}, R_{XV-6} and R_{XV-7}, R_{XV-7} and R_{XV-8}, R_{XV-9} and R_{XV-10}, R_{XV-10} and R_{XV-11}, R_{XV-11} and R_{XV-31}, R_{XV-31} and R_{XV-32}, R_{XV-32} and R_{XV-12}, and R_{XV-12} and R_{XV-13} are independently selected to form spacer pairs wherein a spacer pair is taken together to form a linear moiety having from 3 through 6 contiguous atoms connecting the points of bonding of said spacer pair members to form a ring selected from the group consisting of a cycloalkenyl ring having 5 through 8 contiguous members, a partially saturated heterocyclyl ring having 5 through 8 contiguous members, a heteroaryl ring having 5 through 6 contiguous members, and an aryl with the provisos that no more than one of the group consisting of spacer pairs R_{XV-4} and R_{XV-5}, R_{XV-5} and R_{XV-6}, R_{XV-6} and R_{XV-7}, R_{XV-7} and R_{XV-8} is used at the same time and that no more than one of the group consisting of spacer pairs R_{XV-9} and R_{XV-10}, R_{XV-10} and R_{XV-11}, R_{XV-11} and R_{XV-31}, R_{XV-31} and R_{XV-32}, R_{XV-32} and R_{XV-12} and R_{XV-12} and R_{XV-13} are used at the same time;

R_{XV-9} and R_{XV-11}, R_{XV-9} and R_{XV-12}, R_{XV-9} and R_{XV-13} R_{XV-9} and R_{XV-31}, R_{XV-9} and R_{XV-32}, R_{XV-10} and R_{XV-12}, R_{XV-10} and R_{XV-13}, R_{XV-10} and R_{XV-31}, R_{XV-10} and R_{XV-32}, R_{XV-11} and R_{XV-12}, R_{XV-11} and R_{XV-13}, R_{XV-11} and R_{XV-32}, R_{XV-12} and R_{XV-31}, R_{XV-13} and R_{XV-31}, and R_{XV-13} and R_{XV-32} are independently selected to form a spacer pair wherein said spacer pair is taken together to form a linear spacer moiety selected from the group consisting of a covalent single bond and a moiety having from 1 through 3 contiguous atoms to form a ring selected from the group consisting of a cycloalkyl having from 3 through 8 contiguous members, a cycloalkenyl having from 5 through 8 contiguous members, a saturated heterocyclyl having from 5 through 8 contiguous members and a partially saturated heterocyclyl having from 5 through 8 contiguous members with the provisos that no more than one of said group of spacer pairs is used at the same time;

R_{XV-37} and R_{XV-38} are independently selected from the group consisting of hydrido, alkoxy, alkoxyalkyl, hydroxy, amino, thio, halo, haloalkyl, alkylamino, alkylthio, alkylthioalkyl, cyano, alkyl, alkenyl, haloalkoxy, and haloalkoxyalkyl.

Compounds of Formula XV are disclosed in WO 00/18723, the entire disclosure of which is incorporated by reference.

In a preferred embodiment, the CETP inhibitor is selected from the following compounds of Formula XV:

3-[[3-(4-chloro-3-ethylphenoxy)phenyl](cyclohexylmethyl)amino]-1,1,1-trifluoro-2-propanol;

3-[[3-(4-chloro-3-ethylphenoxy)phenyl](cyclopentylmethyl)amino]-1,1,1-trifluoro-2-propanol;

3-[[3-(4-chloro-3-ethylphenoxy)phenyl](cyclopropylmethyl)amino]-1,1,1-trifluoro-2-propanol;

3-[[3-(4-chloro-3-ethylphenoxy)phenyl][(3-trifluoromethyl)cyclohexyl-methyl]amino]-1,1,1-trifluoro-2-propanol;

3-[[3-(4-chloro-3-ethylphenoxy)phenyl][(3-pentafluoroethyl)cyclohexyl-methyl]amino]-1,1,1-trifluoro-2-propanol;

3-[[3-(4-chloro-3-ethylphenoxy)phenyl][(3-trifluoromethoxy)cyclohexyl-methyl]amino]-1,1,1-trifluoro-2-propanol;

3-[[3-(4-chloro-3-ethylphenoxy)phenyl][[3-(1,1,2,2-tetrafluoroethoxy)cyclo-hexylmethyl]amino]-1,1,1-trifluoro-2-propanol;

3-[[3-(3-trifluoromethoxyphenoxy)phenyl](cyclohexylmethyl)amino]-1,1,1-trifluoro-2-propanol;

3-[[3-(3-trifluoromethoxyphenoxy)phenyl](cyclopentylmethyl)amino]-1,1,1-trifluoro-2-propanol;

3-[[3-(3-trifluoromethoxyphenoxy)phenyl](cyclopropylmethyl)amino]-1,1,1-trifluoro-2-propanol;

3-[[3-(3-trifluoromethoxyphenoxy)phenyl][(3-trifluoromethyl)cyclohexyl-methyl]amino]-1,1,1-trifluoro-2-propanol;

3-[[3-(3-trifluoromethoxyphenoxy)phenyl]](3-pentafluoroethyl)cyclohexyl-methyl]amino]-1,1,1-trifluoro-2-propanol;

3-[[3-(3-trifluoromethoxyphenoxy)phenyl][(3-trifluoromethoxy)cyclohexyl-methyl]amino]-1,1,1-trifluoro-2-propanol;

3-[[3-(3-trifluoromethoxyphenoxy)phenyl][[3-(1,1,2,2-tetrafluoroethoxy)cyclohexyl-methyl]amino]-1,1,1-trifluoro-2-propanol;

3-[[3-(3-isopropylphenoxy)phenyl](cyclohexylmethyl]amino]-1,1,1-trifluoro-2-propanol:

3-[[3-(3-isopropylphenoxy)phenyl](cyclopentylmethyl]amino]-1,1,1-trifluoro-2-propanol;

3-[[3-(3-isopropylphenoxy)phenyl](cyclopropylmethyl)amino]-1,1,1-trifluoro-2-propanol;

3-[[3-(3-isopropylphenoxy)phenyl][(3-trifluoromethyl)cyclohexyl-methyl]amino]-1,1,1-trifluoro-2-propanol;

3-[[3-(3-isopropylphenoxy)phenyl][(3-pentafluoroethyl)cyclohexyl-methyl]amino]-1,1,1-trifluoro-2-propanol;

3-[[3-(3-isopropylphenoxy)phenyl][(3-trifluoromethoxy)cyclohexyl-methyl]amino]-1,1,1-trifluoro-2-propanol;

3-[[3-(3-isopropylphenoxy)phenyl][3-(1,1,2,2-tetrafluoroethoxy)cyclohexyl-methyl]amino]-1,1,1-trifluoro-2-propanol;

3-[[3-(2,3-dichlorophenoxy)phenyl](cyclohexylmethyl)amino]-1,1,1-trifluoro-2-propanol;

3-[[3-(2,3-dichlorophenoxy)phenyl](cyclopentylmethyl)amino]-1,1,1-trifluoro-2-propanol;

3-[[3-(2,3-dichlorophenoxy)phenyl](cyclopropylmethy)amino]-1,1,1-trifluoro-2-propanol;

3-[[3-(2,3-dichlorophenoxy)phenyl][(3-trifluoromethyl)cyclohexyl-methyl]amino]-1,1,1-trifluoro-2-propanol;

3-[[3-(2,3-dichlorophenoxy)phenyl][(3-pentafluoroethyl)cyclohexyl-methyl]amino]-1,1,1-trifluoro-2-propanol;

3-[[3-(2,3-dichlorophenoxy)phenyl][(3-trifluoromethoxy)cyclohexyl-methyl]amino]-1,1,1-trifluoro-2-propanol;

3-[[3-(2,3-dichlorophenoxy)phenyl][3-(1,1,2,2-tetrafluoroethoxy)cyclo-hexyl-methyl]amino]-1,1,1-trifluoro-2-propanol;

3-[[3-(4-fluorophenoxy)phenyl](cyclohexylmethyl)amino]-1,1,1-trifluoro-2-propanol;

3-[[3-(4-fluorophenoxy)phenyl](cyclopentylmethyl)amino]-1,1,1-trifluoro-2-propanol;

3-[[3-(4-fluorophenoxy)phenyl](cyclopropylmethyl)amino]-1,1,1-triflouro-2-propanol;

3-[[3-(4-fluorophenoxy)phenyl][(3-trifluoromethyl)cyclohexyl-methyl]amino]-1,1,1-trifluoro-2-propanol;

3-[[3-(4-fluorophenoxy)phenyl][(3-pentafluoroethyl)cyclohexyl-methyl]amino]-1,1,1-trifluoro-2-propanol;

3-[[3-(4-fluorophenoxy)phenyl][(3-trifluoromethoxy)cyclohexyl-methyl]amino]-1,1,1-trifluoro-2-propanol;

3-[[3-(4-fluorophenoxy)phenyl][[3-(1,1,2,2-tetrafluoroethoxy)cyclohexyl-methyl]amino]-1,1,1-trifluoro-2-propanol;

3-[[3-(3-trifluoromethoxybenzyloxy]phenyl](cyclohexylmethyl)amino]-1,1,1-trifluoro-2-propanol;

3-[[3-(3-trifluoromethoxybenzyloxy)phenyl](cyclopentylmethyl)amino]-1,1-trifluoro-2-propanol;

3-[[3-(3-trifluoromethoxybenzyloxy)phenyl](cyclopropylmethyl]amino]-1,1,1-trifluoro-2-propanol;

3-[[3-(3-trifluoromethoxybenzyloxy)phenyl][(3-trifluoromethyl)cyclohexyl-methyl]amino]-1,1,1-trifluoro-2-propanol;

3-[[3-(3-trifluoromethoxybenzyloxy)phenyl][(3-pentafluoroethyl)cyclohexyl-methyl]amino]-1,1,1-trifluoro-2-propanol;

3-[[3-(3-trifluoromethoxybenzyloxy]phenyl][(3-trifluoromethoxy)cyclohexyl-methyl]amino]-1,1,1-trifluoro-2-propanol;

3-[[3-(3-trifluoromethoxybenzyloxy)phenyl][3-(1,1,2,2-tetrafluoroethoxy)-cyclohexylmethyl]amino]-1,1,1-trifluoro-2-propanol;

3-[[3-(3-trifluoromethylbenzyloxy)phenyl](cyclohexylmethyl)amino]-1,1,1-trifluoro-2-propanol;

3-[[3-(3-trifluoromethylbenzyloxy)phenyl](cyclopentylmethyl)amino]-1,1,1-trifluoro-2-propanol;

3-[[3-(3-trifluoromethylbenzyloxy)phenyl](cyclopropylmethyl)amino]-1,1,1-trifluoro-2-propanol;

3-[[3-(3-trifluoromethylbenzyloxy)phenyl][(3-trifluoromethyl)cyclohexyl-methyl]amino]-1,1,1-trifluoro-2-propanol;

3-[[3-(3-trifluoromethylbenzyloxy)phenyl][(3-pentafluoroethyl)cyclohexyl-methyl]amino]-1,1,1-trifluoro-2-propanol;

3-[[3-(3-trifluoromethylbenzyloxy)phenyl][(3-trifluoromethoxy)cyclohexyl-methyl]amino]-1,1,1-trifluoro-2-propanol;

3-[[3-(3-trifluoromethylbenzyloxy)phenyl][3-(1,1,2,2-tetrafluoroethoxy)cyclohexyl-methyl]amino]-1,1,1-trifluoro-2-propanol;

3-[[[(3-trifluoromethyl)phenyl]methyl](cyclohexyl)amino]-1,1,1-trifluoro-2-propanol;

3-[[[(3-pentafluoroethyl)phenyl]methyl](cyclohexyl)amino]-1,1,1-trifluoro-2-propanol;

3-[[[(3-trifluoromethoxy)phenyl]methyl](cyclohexyl)amino]-1,1,1-trifluoro-2-propanol;

3-[[[3-(1,1,2,2-tetrafluoroethoxy)phenyl]methyl](cyclohexyl)amino]-1,1,1-trifluoro-2-propanol;

3-[[[(3-trifluoromethyl)phenyl]methyl](4-methylcyclohexyl)amino]-1,1,1-trifluoro-2-propanol;

3-[[[(3-pentafluoroethyl)phenyl]methyl](4-methylcyclohexyl)amino]-1,1 1-trifluoro-2-propanol;

3-[[[(3-trifluoromethoxy)phenyl]methyl](4-methylcyclohexyl)amino]-1,1,1-trifluoro-2-propanol;

3-[[[3-(1,1,2,2-tetrafluoroethoxy)phenyl]methyl](4-methylcyclohexyl)amino]-1,1,1-trifluoro-2-propanol;

3-[[[(3-trifluoromethyl]phenyl]methyl](3-trifluoromethylcyclohexyl)amino]-1,1,1-trifluoro-2-propanol;

3-[[[(3-pentafluoroethyl)phenyl]methyl](3-trifluoromethylcyclohexyl)amino]-1,1,1-trifluoro-2-propanol;

3-[[[(3-trifluoromethoxy)phenyl]methyl](3-trifluoromethylcyclohexyl)amino]-1,1,1-trifluoro-2-propanol;

3-[[[3-(1,1,2,2-tetrafluoroethoxy)phenyl]methyl](3-trifluoromethylcyclohexyl)amino]-1,1,1-trifluoro-2-propanol;

3-[[[(3-trifluoromethyl)phenyl]methyl][3-(4-chloro-3-ethylphenoxy)cyclo-hexyl]amino]-1,1,1-trifluoro-2-propanol;

3-[[[(3-pentafluoroethyl)phenyl]methyl][3-(4-chloro-3-ethylphenoxy)cyclo-hexyl]amino]-1,1,1-trifluoro-2-propanol;

3-[[[(3-trifluoromethoxy)phenyl]methyl][3-(4-chloro-3-ethylphenoxy)cyclo-hexyl]amino]-1,1,1-trifluoro-2-propanol;

3-[[[3-(1,1,2,2-tetrafluoroethoxy)phenyl]methyl][3-(4-chloro-3-ethylphenoxy)-cyclohexyl]amino]-1,1,1-trifluoro-2-propanol;

3-[[[(3-trifluoromethyl]phenyl]methyl](3-phenoxycyclohexyl)amino]-1,1,1-trifluoro-2-propanol;

3-[[[(3-pentafluoroethyl)phenyl]methyl](3-phenoxycyclohexyl)amino]-1,1,1-trifluoro-2-propanol;

3-[[[(3-trifluoromethoxy)phenyl]methyl](3-phenoxycyclohexyl)amino]-1,1,1-trifluoro-2-propanol;

3-[[[3-(1,1,2,2-tetrafluoroethoxy)phenyl]methyl](3-phenoxycyclohexyl)amino]-1,1,1-trifluoro-2-propanol;

3-[[[(3-trifloromethyl)phenyl]methyl](3-isopropoxycyclohexyl)amino]-1,1,1-trifluoro-2-propanol;

3-[[[(3-pentafluoroethyl)phenyl]methyl](3-isopropoxycyclohexyl)amino]-1,1,1-trifluoro-2-propanol;

3-[[[(3-trifluoromethoxy)phenyl]methyl](3-isopropoxycyclohexyl)amino]-1,1,1-trifluoro-2-propanol;

3-[[[3-(1,1,2,2-tetrafluoroethoxy)phenyl]methyl](3-isopropoxycyclohexyl)-amino]-1,1,1-trifluoro-2-propanol;

3-[[[(3-trifluoromethyl)phenyl]methyl](3-cyclopentyloxycyclohexyl]amino]-1,1,1-trifluoro-2-propanol;

3-[[[(3-pentafluoroethyl]phenyl]methyl](3-cyclopentyloxycyclohexyl)amino]-1,1,1-trifluoro-2-propanol;

3-[[[(3-trifluoromethoxy)phenyl]methyl](3-cyclopentyloxycyclohexyl)amino]-1,1,1-trifluoro-2-propanol;

3-[[[3-(1,1,2,2-tetrafluoroethoxy)phenyl]methyl](3-cyclopentyloxycyclohexyl)-amino]-1,1,1-trifluoro-2-propanol;

3-[[[(2-trifluoromethyl)pyrid-6-yl]methyl](3-isopropoxycyclohexyl)amino]-1,1,1-trifluoro-2-propanol;

3-[[[(2-trifluoromethyl)pyrid-6-yl]methyl](3-cyclopentyloxycyclohexyl)-amino]-1,1,1-trifluoro-2-propanol;

3-[[[(2-trifluoromethyl)pyrid-6-yl]methyl](3-phenoxycyclohexyl)amino]-1,1,1-trifluoro-2-propanol;

3-[[[(2-trifluoromethyl)pyrid-6-yl]methyl](3-trifluoromethylcyclohexyl)amino]-1,1,1-trifluoro-2-propanol;

3-[[[(2-trifluoromethyl)pyrid-6-yl]methyl][3-(4-chloro-3-ethylphenoxy)cyclo-hexyl]amino]-1,1,1-trifluoro-2-propanol;

3-[[[(2-trifluoromethyl)pyrid-6-yl]methyl][3-(1,1,2,2-tetrafluoroethoxy)cyclo-hexyl]amino]-1,1,1-trifluoro-2-propanol;

3-[[[(2-trifluoromethyl)pyrid-6-yl]methyl](3-pentafluoroethylcyclohexyl)-amino]-1,1,1-trifluoro-2-propanol;

3-[[[(2-trifluoromethyl)pyrid-6-yl]methyl](3-trifluoromethoxycyclohexyl)-amino]-1,1,1-trifluoro-2-propanol;

3-[[[(3-trifluoromethyl)phenyl]methyl][3-(4-chloro-3-ethylphenoxy)propyl]-amino]-1,1,1-trifluoro-2-propanol;

3-[[[(3-pentafluoroethyl)phenyl]methyl][3-(4-chloro-3-ethylphenoxy)propyl]-amino]-1,1,1-trifluoro-2-propanol;

3-[[[(3-trifluoromethoxy)phenyl]methyl][3-(4-chloro-3-ethylphenoxy)propyl]-amino]-1,1,1-trifluoro-2-propanol;

3-[[[3-(1,1,2,2-tetrafluoroethoxy)phenyl]methyl][3-(4-chloro-3-ethylphenoxy)-propyl]amino]-1,1,1-trifluoro-2-propanol;

3-[[[(3-trifluoromethyl)phenyl]methyl][3-(4-chloro-3-ethylphenoxy)-2,2,-di-fluropropyl]amino]-1,1,1-trifluoro-2-propanol;

3-[[[(3-pentafluoroethyl)phenyl]methyl][3-(4-chloro-3-ethylphenoxy)-2,2-di-fluropropyl]amino]-1,1,1-trifluoro-2-propanol;

3-[[[(3-trifluoromethoxy)phenyl]methyl][3-(4-chloro-3-ethylphenoxy)-2,2,-di-fluropropyl]amino]-1,1,1-trifluoro-2-propanol;

3-[[[3-(1,1,2,2-tetrafluoroethoxy)phenyl]methyl][3-(4-chloro-3-ethylphenoxy)-2,2,-difluropropyl]amino]-1,1,1-trifluoro-2-propanol;

3-[[[(3-trifluoromethyl)phenyl]methyl][3-(isopropoxy)propyl]amino]-1,1,1-trifluoro-2-propanol;

3-[[[(3-pentafluoroethyl)phenyl]methyl][3-(isopropoxy)propyl]amino]-1,1,1-trifluoro-2-propanol;

3-[[[(3-trifluoromethoxy)phenyl]methyl][3-(isopropoxy)propyl]amino]-1,1,1-trifluoro-2-propanol;

3-[[[3-(1,1,2,2-tetrafluoroethoxy)phenyl]methyl]]3-(isopropoxy)propyl]amino]-1,1,1-trifluoro-2-propanol; and

3-[[[3-(1,1,2,2-tetrafluoroethoxy)phenyl]methyl][3-(phenoxy)propyl]amino]-1,1,1-trifluoro-2-propanol.

Another class of CETP inhibitors that finds utility with the present invention consists of (R)-chiral halogenated 1-substituted amino-(n+1)-alkanols having the Formula XVI

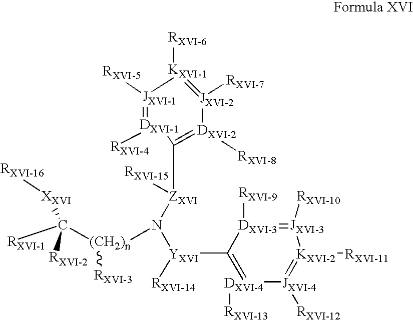

and pharmaceutically acceptable forms thereof, wherein:

n_{XVI} is an integer selected from 1 through 4;

X_{XVI} is oxy;

R_{XV-1}, is selected from the group consisting of haloalkyl, haloalkenyl, haloalkoxymethyl, and haloalkenyloxymethyl with the proviso that R_{XVI-1} has a higher Cahn-lngold-Prelog stereochemical system ranking than both R_{XVI-2} and (CHR_{XVI-3})ₙ—N(A_{XVI})Q_{XVI} wherein A_{XVI} is Formula XVI-(II) and Q is Formula XVI-(III);

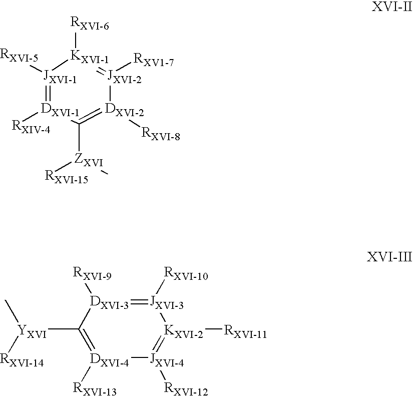

R_{XVI-16} is selected from the group consisting of hydrido, alkyl, acyl, aroyl, heteroaroyl, trialkylsilyl, and a spacer selected from the group consisting of a covalent single bond and a linear spacer moiety having a chain length of 1 to 4 atoms linked to the point of bonding of any aromatic substituent selected from the group consisting of R_{XVI-4}, R_{XVI-8} R_{XVI-9}, and R_{XVI-13} to form a heterocyclyl ring having from 5 through 10 contiguous members;

D_{XVI-1}, D_{XVI-2}, J_{XVI-1}, J_{XVI-2} and K_{XVI-1} are independently selected from the group consisting of C, N, O, S and covalent bond with the provisos that no more than one of D_{XVI-1}, D_{XVI-2}, J_{XVI-1}, J_{XVI-2} and K_{XVI-1} is a covalent bond, no more than one D_{XVI-1}, D_{XVI-2}, J_{XVI-1}, J_{XVI-2} and K_{XVI-1} is be O, no more than one of D_{XVI-1}, D_{XVI-2}, J_{XVI-1}, J_{XVI-2} and K_{XVI-1} is S, one of D_{XVI-1}, D_{XVI-2}, J_{XVI-1}, J_{XVI-2} and K_{XVI-1} must be a covalent bond when two of D_{XVI-1}, D_{XVI-2}, J_{XVI-1}, J_{XVI-2} and K_{XVI-1} are O and S, and no more than four of D_{XVI-1}, D_{XVI-2}, J_{XVI-1}, J_{XVI-2} and K_{XVI-1} is N;

D_{XVI-3}, D_{XVI-4}, J_{XVI-3}, J_{XVI-4} and K_{XVI-2} are independently selected from the group consisting of C, N, O, S and covalent bond with the provisos that no more than one is a covalent bond, no more than one of D_{XVI-3}, D_{XVI-4}, J_{XVI-3}, J_{XVI-4} and K_{XVI-2} is O, no more than one of D_{XVI-3}, D_{XVI-4}, J_{XVI-3}, J_{XVI-4} and K_{XVI-2} is S, no more than two of D_{XVI-3} D_{XVI-4}, J_{XVI-3}, J_{XVI-4} and K_{XVI-2} is 0 and S, one of D_{XVI-3}, D_{XVI-4} J_{XVI-3}, J_{XVI-4} and K_{XVI-2} must be a covalent bond when two of D_{XVI-3}, D_{XVI-4}, J_{XVI-3}, J_{XVI-4} and K_{XVI-2} are O and S, and no more than four of D_{XVI-3} D_{XVI-4}, J_{XVI-3}, J_{XVI-4} and K_{XVI-2} are N;

R_{XVI-2} is selected from the group consisting of hydrido, aryl, aralkyl, alkyl, alkenyl, alkenyloxyalkyl, haloalkyl, haloalkenyl, halocycloalkyl, haloalkoxy, haloalkoxyalkyl, haloalkenyloxyalkyl, halocycloalkoxy, halocycloalkoxyalkyl, perhaloaryl, perhaloaralkyl, perhaloaryloxyalkyl, heteroaryl, dicyanoalkyl, and carboalkoxycyanoalkyl, with the proviso that R_{XVI-2} has a lower Cahn-lngold-Prelog system ranking than both R_{XVI-1} and (CHR_{XVI-3})ₙ—N(A_{XVI})Q_{XVI};

R_{XVI-3} is selected from the group consisting of hydrido, hydroxy, cyano, aryl, aralkyl, acyl, alkoxy, alkyl, alkenyl, alkoxyalkyl, heteroaryl, alkenyloxyalkyl, haloalkyl, haloalkenyl, haloalkoxy, haloalkoxyalkyl, haloalkenyloxyalkyl, monocyanoalkyl, dicyanoalkyl, carboxamide, and carboxamidoalkyl, with the provisos that (CHR_{XVI-3})ₙ—N(A_{XVI})Q_{XVI} has a lower Cahn-lngold-Prelog stereochemical system ranking than R_{XVI-1} and a higher Cahn-lngold-Prelog stereochemical system ranking than R_{XVI-2};

Y_{XVI} is selected from a group consisting of a covalent single bond, (C(R_{XVI-14})₂)_{q} wherein q is an integer selected from 1 and 2 and (CH(R_{XVI-14}))_{g}—W_{XVI}—(CH(R_{XVI-14}))ₚ wherein g and p are integers independently selected from 0 and 1;

R_{XVI-14} is selected from the group consisting of hydrido, hydroxy, cyano, hydroxyalkyl, acyl, alkoxy, alkyl, alkenyl, alkynyl, alkoxyalkyl, haloalkyl, haloalkenyl, haloalkoxy, haloalkoxyalkyl, haloalkenyloxyalkyl, monocarboalkoxyalkyl, monocyanoalkyl, dicyanoalkyl, carboalkoxycyanoalkyl, carboalkoxy, carboxamide, and carboxamidoalkyl;

Z_{XVI} is selected from a group consisting of a covalent single bond, (C(R_{XVI-15})₂)_{q}, wherein q is an integer selected from 1 and 2, and (CH(R_{XVI-15}))ⱼ—W_{XVI}—(CH(R_{XVI-15}))ₖ wherein j and k are integers independently selected from 0 and 1;

W_{XVI} is selected from the group consisting of O, C(O), C(S), C(O)N(R_{XVI-14}), C(S)N(R_{XVI-14}),(R_{XVI-14})NC(O), (R_{XVI-14} )NC(S), S, S(O), S(O)₂, S(O)₂N(R_{XVI-14}), (R_{XVI-14})NS(O)₂, and N(R_{XVI-14}) with the proviso that R_{XVI-14} is other than cyano;

R_{XVI-15} is selected, from the group consisting of hydrido, cyano, hydroxyalkyl, acyl, alkoxy, alkyl, alkenyl, alkynyl, alkoxyalkyl, haloalkyl, haloalkenyl, haloalkoxy, haloalkoxyalkyl, haloalkenyloxyalkyl, monocarboalkoxyalkyl, monocyanoalkyl, dicyanoalkyl, carboalkoxycyanoalkyl, carboalkoxy, carboxamide, and carboxamidoalkyl;

R_{XVI-4}, R_{XVI-5}, R_{XVI-6}, R_{XVI-7}, R_{XVI-8}, R_{XVI-9}, R_{XVI-10}, R_{XVI-11}, R_{XVI-12}, and R_{XV-13} are independently selected from the group consisting of hydrido, carboxy, heteroaralkylthio, heteroaralkoxy, cycloalkylamino, acylalkyl, acylalkoxy, aroylalkoxy, heterocyclyloxy, aralkylaryl, aralkyl, aralkenyl, aralkynyl, heterocyclyl, perhaloaralkyl, aralkylsulfonyl, aralkylsulfonylalkyl, aralkylsulfinyl, aralkylsulfinylalkyl, halocycloalkyl, halocycloalkenyl, cycloalkylsulfinyl, cycloalkylsulfinylalkyl, cycloalkylsulfonyl, cycloalkylsulfonylalkyl, heteroarylamino, N-heteroarylamino-N-alkylamino, heteroaralkyl, heteroarylaminoalkyl, haloalkylthio, alkanoyloxy, alkoxy, alkoxyalkyl, haloalkoxylalkyl, heteroaralkoxy, cycloalkoxy, cycloalkenyloxy, cycloalkoxyalkyl, cycloalkylalkoxy, cycloalkenyloxyalkyl, cycloalkylenedioxy, halocycloalkoxy, halocycloalkoxyalkyl, halocycloalkenyloxy, halocycloalkenyloxyalkyl, hydroxy, amino, thio, nitro, lower alkylamino, alkylthio, alkylthioalkyl, arylamino, aralkylamino, arylthio, arylthioalkyl, heteroaralkoxyalkyl, alkylsulfinyl, alkylsulfinylalkyl, arylsulfinylalkyl, arylsulfonylalkyl, heteroarylsulfinylalkyl, heteroarylsulfonylalkyl, alkylsulfonyl, alkylsulfonylalkyl, haloalkylsulfinylalkyl, haloalkylsulfonylalkyl, alkylsulfonamido, alkylaminosulfonyl, amidosulfonyl, monoalkyl amidosulfonyl, dialkyl, amidosulfonyl, monoarylamidosulfonyl, arylsulfonamido, diarylamidosulfonyl, monoalkyl monoaryl amidosulfonyl, arylsulfinyl, arylsulfonyl, heteroarylthio, heteroarylsulfinyl, heteroarylsulfonyl, heterocyclylsulfonyl, heterocyclylthio, alkanoyl, alkenoyl, aroyl, heteroaroyl, aralkanoyl, heteroaralkanoyl, haloalkanoyl, alkyl, alkenyl, alkynyl, alkenyloxy, alkenyloxyalky, alkylenedioxy, haloalkylenedioxy, cycloalkyl, cycloalkylalkanoyl, cycloalkenyl, lower cycloalkylalkyl, lower cycloalkenylalkyl, halo, haloalkyl, haloalkenyl, haloalkoxy, hydroxyhaloalkyl, hydroxyaralkyl, hydroxyalkyl, hydoxyheteroaralkyl, haloalkoxyalkyl, aryl, heteroaralkynyl, aryloxy, aralkoxy, aryloxyalkyl, saturated heterocyclyl, partially saturated heterocyclyl, heteroaryl, heteroaryloxy, heteroaryloxyalkyl, arylalkenyl, heteroarylalkenyl, carboxyalkyl, carboalkoxy, alkoxycarboxamido, alkylamidocarbonylamido, arylamidocarbonylamido, carboalkoxyalkyl, carboalkoxyalkenyl, carboaralkoxy, carboxamido, carboxamidoalkyl, cyano, carbohaloalkoxy, phosphono, phosphonoalkyl, diaralkoxyphosphono, and diaralkoxyphosphonoalkyl with the proviso that R_{XVI-4}, R_{XVI-5}, R_{XVI-6} R_{XVI-7}, R_{XVI-8}, R_{XVI-9}, R_{XVI-10}, R_{XVI-11}, R_{XVI-12} and R_{XVI-13} are each independently selected to maintain the tetravalent nature of carbon, trivalent nature of nitrogen, the divalent nature of sulfur, and the divalent nature of oxygen;

R_{XVI-4} and R_{XVI-5}, R_{XVI-5} and R_{XVI-6}, R_{XVI-6} and R_{XVI-7} R_{XVI-7} and R_{XVI-8}, R_{XVI-9} and R_{XVI-10}, R_{XVI-10} and R_{XVI-11} R_{XVI-11} and R_{XVI-12} and R_{XVI-12} and R_{XIV-13} are independently selected to form spacer pairs wherein a spacer pair is taken together to form a linear moiety having from 3 through 6 contiguous atoms connecting the points of bonding of said spacer pair members to form a ring selected from the group consisting of a cycloalkenyl ring having 5 through 8 contiguous members, a partially saturated heterocyclyl ring having 5 through 8 contiguous members, a heteroaryl ring having 5 through 6 contiguous members, and an aryl with the provisos that no more than one of the group consisting of spacer pairs R_{XVI-4} and R_{XVI-5} R_{XVI-5} and R_{XVI-6}, R_{XVI-6} and R_{XVI-7}, and R_{XVI-7} and R_{XVI-8} is used at the same time and that no more than one of the group consisting of spacer pairs R_{XIV-9} and R_{XVI-10}, R_{XVI-10} and R_{XVI-11}, R_{XVI-11} and R_{XVI-12,} and R_{XVI-12} and R_{XVI-13} can be used at the same time;

R_{XVI-4} and R_{XVI-9}, R_{XVI-4} and R_{XVI-13}, R_{XV-8} and R_{XVI-9} and R_{XVI-8} and R_{XVI-13} is independently selected to form a spacer pair wherein said spacer pair is taken together to form a linear moiety wherein said linear moiety forms a ring selected from the group consisting of a partially saturated heterocyclyl ring having from 5 through 8 contiguous members and a heteroaryl ring having from 5 through 6 contiguous members with the proviso that no more than one of the group consisting of spacer pairs R_{XVI-4} and R_{XVI-9}, R_{XVI-4} and R_{XVI-13,} R_{XVI-8} and R_{XVI-9} and R_{XVI-8} and R_{XVI-13} is used at the same time.

Compounds of Formula XVI are disclosed in WO 00/18724, the entire disclosure of which is incorporated by reference.

In a preferred embodiment, the CETP inhibitor is selected from the following compounds of Formula XVI:

(2R)-3-[[3-(3-trifluoromethoxyphenoxy)phenyl][[3-(1,1,2,2-tetrafluoroethoxy)phenyl]methyl]amino]-1,1,1-trifluoro-2-propanol;

(2R)-3-[[3-(3-isopropylphenoxy)phenyl][[3-(1,1,2,2-tetrafluoroethoxy)phenyl]-methyl]amino]-1,1,1-trifluoro-2-propanol;

(2R)-3-[[3-(3-cyclopropylphenoxy)phenyl][[3-(1,1,2,2-tetrafluoroethoxy)phenyl]-methyl]amino]-1,1,1-trifluoro-2-propanol;

(2R)-3-[[3-(3-(2-furyl)phenoxy)phenyl][[3-(1,1,2,2-tetrafluoroethoxy)phenyl]-methyl]amino]-1,1,1-trifluoro-2-propanol;

(2R)-3-[[3-(2,3-dichlorophenoxy)phenyl][[3-(1,1,2,2-tetrafluoroethoxy)phenyl]-methyl]amino]-1,1,1-trifluoro-2-propanol;

(2R)-3-[[3-(4-fluorophenoxy)phenyl][[3-(1,1,2,2-tetrafluoroethoxy)phenyl]-methyl]amino]-1,1,1-trifluoro-2-propanol;

(2R)-3-[[3-(4-methylphenoxy)phenyl][[3-(1,1,2,2-tetrafluoroethoxy)phenyl]-methyl]amino]-1,1,1-trifluoro-2-propanol;

(2R)-3-[[3-(2-fluoro-5-bromophenoxy)phenyl][[3-(1,1,2,2-tetrafluoroethoxy)phenyl]-methyl]amino]-1,1,1,1-trifluoro-2-propanol;

(2R)-3-[[3-(4-chloro-3-ethylphenoxy)phenyl][[3-(1,1,2,2-tetrafluoroethoxy)phenyl]-methyl]amino]-1,1,1-trifluoro-2-propanol;

(2R)-3-[[3-[3-(1,1,2,2-tetrafluoroethoxy)phenoxy]phenyl][[3-(1,1,2,2-tetrafluoro-ethoxy)phenyl]methyl]amino]-1,1,1-trifluoro-2-propanol;

(2R)-3-[[3-[3-(pentafluoroethyl)phenoxy]phenyl][[3-(1,1,2,2-tetrafluoroethoxy)-phenyl]methyl]amino]-1,1,1-trifluoro-2-propanol;

(2R)-3-[[3-(3,5-dimethylphenoxy)phenyl][[3-(1,1,2,2-tetrafluoroethoxy)phenyl]-methyl]amino]-1,1,1-trifluoro-2-propanol;

(2R)-3-[[3-(3-ethylphenoxy)phenyl][[3-(1,1,2,2-tetrafluoroethoxy)phenyl]-methyl]amino]-1,1,1-trifluoro-2-propanol;

(2R)-3-[[3-(3-t-butylphenoxy)phenyl][[3-(1,1,2,2-tetrafluoroethoxy)phenyl]-methyl]amino]-1,1,1-trifluoro-2-propanol:

(2R)-3-[[3-(3-methylphenoxy)phenyl][[3-(1,1,2,2-tetrafluoroethoxy)phenyl]-methyl]amino]-1,1,1-trifluoro-2-propanol;

(2R)-3-[[3-(5,6,7,8-tetrahydro-2-naphthoxy)phenyl][[3-(1,1,2,2-tetrafluoro-ethoxy)phenyl]methyl]amino]-1,1,1-trifluoro-2-propanol;

(2R)-3-[[3-(phenoxy)phenyl][[3-(1,1,2,2-tetrafluoroethoxy)phenyl]methyl]amino]-1,1,1-trifluoro-2-propanol;

(2R)-3-[[3-[3-(N,N-dimethylamino)phenoxy]phenyl][[3-(1,1,2,2-tetrafluoro-ethoxy)phenyl]methyl]amino]-1,1,1-trifluoro-2-propanol;

(2R)-3-([[3-(1,1,2,2,-tetrafluoroethoxy)phenyl]methyl][3-[[3-(trifluoromethoxy)-phenyl]methoxy]phenyl]amino]-1,1,1-trifluoro-2-propanol;

(2R)-3-[[[3-(1,1,2,2-tetrafluoroethoxy)phenyl]methyl][3-[[3-(trifluoro-methyl)phenyl]methoxy]phenyl]amino]-1,1,1-trifluoro-2-propanol;

(2R)-3-[[[3-(1,1,2,2-tetrafluoroethoxy)phenyl]methyl][3-[[3,5-dimethylphenyl]-methoxy]phenyl]amino]-1,1,1-trifluoro-2-propanol;

(2R)-3-[[[3-(1,1,2,2-tetrafluoroethoxy)phenyl]methyl][3-[[3-(trifluoromethylthio)-phenyl]methoxy]phenyl]amino]-1,1,1-trifluoro-2-propanol;

(2R)-3-[[[3-(1,1,2,2-tetrafluoroethoxy)phenyl]methyl][3-[[3,5-difluorophenyl]-methoxy]phenyl]amino]-1,1,1-trifluoro-2-propanol;

(2R)-3-[[[3-(1,1,2,2-tetrafluoroethoxy)phenyl]methyl][3-[cyclohexylmethoxy]-phenyl]amino]-1,1,1-trifluoro-2-propanol;

(2R)-3-[[3-(2-difluoromethoxy-4-pyridyloxy)phenyl][[3-(1,1,2,2-tetrafluoroethoxy)-phenyl]methyl]amino]-1,1,1-trifluoro-2-propanol;

(2R)-3-[[3-(2-trifluoromethyl-4-pyridyloxy)phenyl][[3-(1,1,2,2-tetrafluoroethoxy)-phenyl]methyl]amino]-1,1,1-trifluoro-2-propanol;

(2R)-3-[[3-(3-difluoromethoxyphenoxy)phenyl][[3-(1,1,2,2-tetrafluoroethoxy)-phenyl]methyl]amino]-1,1,1-trifluoro-2-propanol;

(2R)-3-[[[3-(3-trifuoromethylthio)phenoxy]phenyl][[3-(1,1,2,2-tetrafluoroethoxy)-phenyl]methyl]amino]-1,1,1-trifluoro-2-propanol;

(2R)-3-[[3-(4-chloro-3-trifluoromethylphenoxy)phenyl][[3-(1,1,2,2-tetrafluoroethoxy)-phenyl]methyl]amino]-1,1,1-trifluoro-2-propanol;

(2R)-3-[[3-(3-trifluoromethoxyphenoxy)phenyl][[3-(pentafluoroethyl)phenyl]-methyl]amino]-1,1,1-trifluoro-2-propanol;

(2R)-3-[[3-(3-isopropylphenoxy)phenyl][[3-(pentafluoroethyl)phenyl]methyl]-amino]-1,1,1-trifluoro-2-propanol;

(2R)-3-[[3-(3-cyclopropylphenoxy)phenyl][[3-(pentafluoroethyl)phenyl]methyl]-amino]-1,1,1-trifluoro-2-propanol;

(2R)-3-[[3-(3-(2-furyl)phenoxy)phenyl][[3-(pentafluoroethyl)phenyl]methyl]-amino]-1,1,1-trifluoro-2-propanol;

(2R)-3-[[3-(2,3-dichlorophenoxy)phenyl][[3-(pentafluoroethyl)phenyl]methyl]- amino]-1,1,1-trifluoro-2-propanol;

(2R)-3-[[3-(4-fluorophenoxy)phenyl][[3-(pentafluoroethyl)phenyl]methyl]amino]-1,1,1-trifluoro-2-propanol;

(2R)-3-[[3-(4-methylphenoxy)phenyl][[3-(pentafluoroethyl)phenyl]methyl]amino]-1,1,1-trifluoro-2-propanol;

(2R)-3-[[3-(2-fluoro-5-bromophenoxy)phenyl][[3-(pentafluoroethyl)phenyl]methyl]-amino]-1,1,1,1-trifluoro-2-propanol;

(2R)-3-[[3-(4-chloro-3-ethylphenoxy)phenyl][[3-(pentafluoroethyl)phenyl]methyl]-amino]-1,1,1,1-trifluoro-2-propanol;

(2R)-3-[[3-[3-(1,1,2,2-tetrafluoroethoxy)phenoxy]phenyl][[3-(pentafluoroethyl)-phenyl]methyl]amino]-1,1,1-trifluoro-2-propanol;

(2R)-3-[[3-[3-(pentafluoroethyl)phenoxy]phenyl][[3-(pentafluoroethyl)phenyl]-methyl]amino]-1,1,1-trifluoro-2-propanol;

(2R)-3-[[3-(3,5-dimethylphenoxy)phenyl][[3-(pentafluoroethyl)phenyl]methyl]-amino]-1,1,1-trifluoro-2-propanol;

(2R)-3-[[3-(3-ethylphenoxy)phenyl][[3-(pentafluoroethyl)phenyl]methyl]amino]-1,1,1-trifluoro-2-propanol;

(2R)-3-[[3-(3-t-butylphenoxy)phenyl][[3-(pentafluoroethyl)phenyl]methyl]amino]-1,1,1-trifluoro-2-propanol;

(2R)-3-[[3-(3-methylphenoxy)phenyl][[3-(pentafluoroethyl) phenyl]methyl]amino]-1,1,1-trifluoro-2-propanol;

(2R)-3-[[3-(5,6,7,8-tetrahydro-2-naphthoxy)phenyl][[3-(pentafluoroethyl)phenyl]-methyl]amino]-1,1,1,-trifluoro-2-propanol;

(2R)-3-[[3-(phenoxy)phenyl][[3(pentafluoroethyl)phenyl]methyl]amino]-1,1,1-trifluoro-2-propanol;

(2R)-3-[[3-[3-(N,N-dimethylamino)phenoxy]phenyl][[3-(pentafluoroethyl)phenyl]-methyl]amino]-1,1,1-trifluoro-2-propanol;

(2R)-3-[[[3-(pentafluoroethyl)phenyl]methyl][3-[[3-(trifluoromethoxy)phenyl]-methoxy]phenyl]amino]-1,1,1-trifluoro-2-propanol;

(2R)-3-[[[3-(pentafluoroethyl)phenyl]methyl][3-[[3-(trifluoromethyl)-phenyl]-methoxy]phenyl]amino]-1,1,1-trifluoro-2-propanol;

(2R)-3-[[[3-(pentafluoroethyl)phenyl]methyl][3-[[3,5-dimethylphenyl]methoxy]-phenyl]amino]-1,1,1-trifluoro-2-propanol;

(2R)-3-[[[3-(pentafluoroethyl)phenyl]methyl][3-[[3-(trifluoromethylthio)phenyl]-methoxy]phenyl]amino]-1,1,1-trifluoro-2-propanol;

(2R)-3-[[[3-(pentafluoroethyl)phenyl]methyl][3-[[3,5-difluorophenyl]methoxy]-phenyl]amino]-1,1,1-trifluoro-2-propanol;

(2R)-3-[[[3-(pentafluoroethyl)phenyl]methyl][3-[cyclohexylmethoxy]phenyl]-amino]-1,1,1-trifluoro-2-propanol;

(2R)-3-[[3-(2-difluoromethoxy-4-pyridyloxy)phenyl][[3-(pentafluoroethyl)phenyl]-methyl]amino]-1,1,1-trifluoro-2-propanol;

(2R)-3-[[3-(2-trifluoromethyl-4-pyridyloxy)phenyl][[3-(pentafluoroethyl)phenyl]-methyl]amino]-1,1,1-trifluoro-2-propanol;

(2R)-3-[(3-(3-difluoromethoxyphenoxy)phenyl][[3-(pentafluoroethyl)phenyl]-methyl]amino]-1,1,1-trifluoro-2-propanol;

(2R)-3-[([3-(3-trifluoromethylthio)phenoxy]phenyl][[3-(pentafluoroethyl)phenyl]-methyl]amino]-1,1,1-trifluoro-2-propanol;

(2R)-3-[[3-(4-chloro-3-trifluoromethylphenoxy)phenyl][[3-(pentafluoroethyl)-phenyl]methyl]amino]-1,1,1-trifluoro-2-propanol;

(2R)-3-[[3-(3-trifluoromethoxyphenoxy)phenyl][[3-(heptafluoropropyl)phenyl]-methyl]amino]-1,1,1-trifluoro-2-propanol;

(2R)-3-[[3-(3-isopropylphenoxy)phenyl][[3-(heptafluoropropyl)phenyl]methyl]-amino]-1,1,1-trifluoro-2-propanol;

(2R)-3-[[3-(3-cyclopropylphenoxy)phenyl][[3-(heptafluoropropyl)phenyl]methyl]-amino]-1,1,1-trifluoro-2-propanol;

(2R)-3-[[3-(3-(2-furyl)phenoxy)phenyl][[3-(heptafluoropropyl)phenyl]methyl]-amino]-1,1,1-trifluoro-2-propanol;

(2R)-3-[[3-(2,3-dichlorophenoxy)phenyl][[3-(heptafluoropropyl)phenyl]methyl]-amino]-1,1,1-trifluoro-2-propanol;

(2R)-3-[[3-(4-fluorophenoxy)phenyl][[3-(heptafluoropropyl) phenyl]methyl]amino]-1,1,1-trifluoro-2-propanol;

(2R)-3-[[3-(4-methylphenoxy)phenyl][[3-(heptafluoropropyl) phenyl]methyl]amino]-1,1,1,-trifluoro-2-propanol;

(2R)-3-[[3-(2-fluoro-5-bromophenoxy)phenyl][[3-(heptafluoropropyl)phenyl]-methyl]amino]-1,1,1-trifluoro-2-propanol;

(2R)-3-[[3-(4-chloro-3-ethylphenoxy)phenyl][[3-(heptafluoropropyl)phenyl]methyl]-amino]-1,1,1-trifluoro-2-propanol;

(2R)-3-[[3-[3-(1,1,2,2-tetrafluoroethoxy)phenoxy]phenyl][[3-(heptafluoropropyl)-phenyl]methyl]amino]-1,1,1-trifluoro-2-propanol;

(2R)-3-[[3-[3-(pentafluoroethyl)phenoxy]phenyl][[3-(heptafluoropropyl)phenyl]-methyl]amino]-1,1,1-trifluoro-2-propanol;

(2R)-3-[[3-(3,5-dimethylphenoxy)phenyl][[3-(heptafluoropropyl)phenyl]methyl]-amino]-1,1,1-trifluoro-2-propanol;

(2R)-3-[[3-(3-ethylphenoxy)phenyl][[3-(heptafluoropropyl)phenyl]methyl]amino]-1,1,1-trifluoro-2-propanol;

(2R)-3-[[3-(3-t-butylphenoxy)phenyl][[3-(heptafluoropropyl) phenyl]methyl]amino]-1,1,1-trifluoro-2-propanol;

(2R)-3-[[3-(3-methylphenoxy)phenyl][[3-(heptafluoropropyl) phenyl]methyl]amino]-1,1,1-trifluoro-2-propanol;

(2R)-3-[[3-(5,6,7,8-tetrahydro-2-naphthoxy)phenyl][[3-(heptafluoropropyl)phenyl]-methyl]amino]-1,1,1-trifluoro-2-propanol;

(2R)-3-[[3-(phenoxy)phenyl][[3-(heptafluoropropyl)phenyl]methyl]amino]-1,1,1-trifluoro-2-propanol;

(2R)-3-[[3-[3-(N,N-dimethylamino)phenoxy]phenyl][[3-(heptafluoropropyl)phenyl]-methyl]amino]-1,1,1-trifluoro-2-propanol;

(2R)-3-[[[3-(heptafluoropropyl)phenyl]methyl][3-[[3-(trifluoromethoxy)phenyl]-methoxy]phenyl]amino]-1,1,1-trifluoro-2-propanol;

(2R)-3-[[[3-(heptafluoropropyl)phenyl]methyl][3-[[3-(trifluoromethyl)phenyl]-methoxy]phenyl]amino]-1,1,1-trifluoro-2-propanol;

(2R)-3-[[[3-(heptafluoropropyl)phenyl]methyl][3-[[3,5-dimethylphenyl]methoxy]-phenyl]amino]-1,1,1-trifluoro-2-propanol;

(2R)-3-[[[3-(heptafluoropropyl)phenyl]methyl][3-[[3-(trifluoromethylthio)phenyl]-methoxy]phenyl]amino]-1,1,1-trifluoro-2-propanol;

(2R)-3-[[[3-(heptafluoropropyl)phenyl]methyl][3-[[3,5-difluorophenyl]methoxy]-phenyl]amino]-1,1,1-trifluoro-2-propanol;

(2R)-3-[[[3-(heptafluoropropyl)phenyl]methyl][3-[cyclohexylmethoxy]phenyl]-amino]-1,1,1-trifluoro-2-propanol;

(2R)-3-[[3-(2-difluoromethoxy-4-pyridyloxy)phenyl][[3-(heptafluoropropyl)phenyl]-methyl]amino]-1,1,1-trifluoro-2-propanol;

(2R)-3-[[3-(2-trifluoromethyl-4-pyridyloxy)phenyl][[3-(heptafluoropropyl)phenyl]-methyl]amino]-1,1,1-trifluoro-2-propanol;

(2R)-3-[[3-(3-difluoromethoxyphenoxy)phenyl][[3-(heptafluoropropyl)phenyl]-methyl]amino]-1,1,1-trifluoro-2-propanol;

(2R)-3-[[[3-(3-trifluoromethylthio)phenoxy]phenyl][[3-(heptafluoropropyl)phenyl]-methyl]amino]-1,1,1-trifluoro-2-propanol;

(2R)-3-[[3-(4-chloro-3-trifluoromethylphenoxy)phenyl][[3-(heptafluoropropyl)-phenyl]methyl]amino]-1,1,1-trifluoro-2-propanol;

(2R)-3-[[3-(3-trifluoromethoxyphenoxy)phenyl][[2-fluoro-5-(trifluoromethyl)-phenyl]methyl]amino]-1,1,1-trifluoro-2-propanol;

(2R)-3-[[3-(3-isopropylphenoxy)phenyl][[2-fluoro-5-(trifluoromethyl )phenyl]-methyl]amino]-1,1,1-trifluoro-2-propanol;

(2R)-3-[[3-(3-cyclopropylphenoxy)phenyl][[2-fluoro-5-(trifluoromethyl)phenyl]-methyl]amino]-1,1,1-trifluoro-2-propanol;

(2R)-3-[[3-(3-(2-furyl)phenoxy)phenyl][[2-fluoro-5-(trifluoromethyl)phenyl]-methyl]amino]-1,1,1-trifluoro-2-propanol;

(2R)-3-[[3-(2,3-dichlorophenoxy)phenyl][[2-fluoro-5-(trifluoromethyl)phenyl]-methyl]amino]-1,1,1-trifluoro-2-propanol;

(2R)-3-[[3-(4-fluorophenoxy)phenyl][[2-fluoro-5-(trifluoromethyl)phenyl]-methyl]amino]-1,1,1-trifluoro-3-propanol;

(2R)-3-[[3-(4-methylphenoxy)phenyl][[2-fluoro-5-(trifluoromethyl)phenyl]-methyl]amino]-1,1,1-trifluoro-2-propanol;

(2R)-3-[[3-(2-fluoro-5-bromophenoxy)phenyl][[2-fluoro-5-(trifluoromethyl)-phenyl]methyl]amino]-1,1,1-trifluoro-2-propanol;

(2R)-3[[3-(4-chloro-3-ethylphenoxy)phenyl][[2-fluoro-5-(trifluoromethyl)-phenyl]methyl]amino]-1,1,1-trifluoro-2-propanol;

(2R)-3-[[3-[3-(1,1,2,2-tetrafluoroethoxy)phenoxy]phenyl][[2-fluoro-5-(trifluoro-methyl)phenyl]methyl]amino]-1,1,1-trifluoro-2-propanol;

(2R)-3-[[3-[3-(pentafluoroethyl)phenoxy]phenyl][[2-fluoro-5-(trifluoromethyl)-phenyl]methyl]amino]-1,1,1-trifluoro-2-propanol;

(2R)-3-[[3-(3,5-dimethylphenoxy)phenyl][[2-fluoro-5-(trifluoromethyl)phenyl]-methyl]amino]-1,1,1-trifluoro-2-propanol;

(2R)-3-[[3-(3-ethylphenoxy)phenyl][[2-fluoro-5-(trifluoromethyl)phenyl]methyl]-amino]-1,1,1-trifluoro-2-propanol;

(2R)-3-[[3-(3-t-butylphenoxy)phenyl][[2-fluoro-5-(trifluoromethyl)phenyl]methyl]-amino]-1,1,1-trifluoro-2-propanol;

(2R)-3-[[3-(3-methylphenoxy)phenyl][[2-fluoro-5-(trifluoromethyl)phenyl]methyl]-amino]-1,1,1-trifluoro-2-propanol;

(2R)-3-[[3-(5,6,7,8-tetrahydro-2-naphthoxy)phenyl][[2-fluoro-5-(trifluoromethyl)-phenyl]methyl]amino]-1,1,1-trifluoro-2-propanol;

(2R)-3-[[3-(phenoxy)phenyl][[2-fluoro-5-(trifluoromethyl)phenyl]methyl]amino]-1,1,1-trifluoro-2-propanol;

(2R)-3-[[3-[3-(N,N-dimethylamino,phenoxy]phenyl][[2-fluoro-5-(trifluoromethyl)-phenyl]methyl]amino]-1,1,1-trifluoro-2-propanol;

(2R)-3-[[[2-fluoro-5-(trifluoromethyl)phenyl]methyl][3-[[3-(trifluoromethoxy)-phenyl]methoxy]phenyl]amino]-1,1,1-trifluoro-3-propanol;

(2R)-3-[[[2-fluoro-5-(trifluoromethyl)phenyl]methyl][3-[[3-(trifluoromethyl)-phenyl]methoxy]phenyl]amino]-1,1,1-trifluoro-2-propanol;

(2R)-3-[[[2-fluoro-5-(trifluoromethyl)phenyl]methyl][3-[[3,5-dimethylphenyl]-methoxy]phenyl]amino]-1,1,1-trifluoro-2-propanol;

(2R)-3-[[[2-fluoro-5-(trifluoromethyl)phenyl]methyl][3-[[3-(trifluoromethylthio)-phenyl]methoxy]phenyl]amino]-1,1,1-trifluoro-2-propanol;

(2R)-3-[[[2-fluoro-5-(trifluoromethyl)phenyl]methyl][3-[[3,5-difluorophenyl]-methoxy]phenyl]amino]-1,1,1-trifluoro-2-propanol;

(2R)-3-[[[2-fluoro-5-(trifluoromethyl)phenyl]methyl][3-[cyclohexylmethoxyl-phenyl]amino]-1,1,1-trifluoro-2-propanol;

(2R)-3-[[3-(2-difluoromethoxy-4-pyridyloxy)phenyl][[2-fluoro-5-(trifluoromethyl)-phenyl]methyl]amino]-1,1,1-trifluoro-2-propanol;

(2R)-3-[[3-(2-trifluoromethyl-4-pyridyloxy)phenyl][[2-fluoro-5-(trifluoromethyl)-phenyl]methyl]amino]-1,1,1-trifluoro-2-propanol;

(2R)-3-[[3-(3-difluoromethoxyphenoxy)phenyl][[2-fluoro-5-(trifluoromethyl)-phenyl]methyl]amino]-1,1,1-trifluoro-2-propanol;

(2R)-3-[[[3-(3-trifluoromethylthio)phenoxy]phenyl][[2-fluoro-5-(trifluoromethyl)-phenyl]methyl]amino]-1,1,1-trifluoro-2-propanol;

(2R)-3-[[3-(4-chloro-3-trifluoromethylphenoxy)phenyl][[2-fluoro-5-(trifluoro-methyl)phenyl]methyl]amino]-1,1,1-trifluoro-2-propanol;

(2R)-3-[[3-(3-trifluoromethoxyphenoxy)phenyl][[2-fluoro-4-(trifluoromethyl)-phenyl]methyl]amino]-1,1,1-trifluoro-2-propanol;

(2R)-3-[[3-(3-isopropylphenoxy)phenyl][[2-fluoro-4-(trifluoromethyl)phenyl]-methyl]amino]-1,1,1-trifluoro-2-propanol;

(2R)-3-[[3-(3-cyclopropylphenoxy)phenyl][[2-flouro-4-(trifluoromethyl)phenyl]-methyl]amino]-1,1,1-trifluoro-2-propanol;

(2R)-3-[[3-(3-(2-furyl)phenoxy)phenyl][[2-fluoro-4-(trifluoromethyl)phenyl]-methyl]amino]-1,1,1-trifluoro-2-propanol;

(2R)-3-[[3-(2,3-dichlorophenoxy)phenyl][[2-fluoro4-(trifluoromethyl)phenyl]-methyl]amino]-1,1,1-trifluoro-2-propanol;

(2R)-3-[[3-(4-fluorophenoxy)phenyl][[2-fluoro-4-(trifluoromethyl)phenyl]-methyl]amino]-1,1,1-trifluoro-2-propanol;

(2R)-3-[[3-(4-methylphenoxy)phenyl][[2-fluoro-4-(trifluoromethyl)phenyl]-methyl]amino]-1,1,1-trifluoro-2-propanol;

(2R)-3-[[3-(2-fluoro-5-bromophenoxy)phenyl][[2-fluoro-4-(trifluoromethyl)-phenyl]methyl]amino]-1,1,1-trifluoro-2-propanol;

(2R)-3-[[3-(4-chloro-3-ethylphenoxy)phenyl][[2-fluoro-4-(trifluoromethyl)-phenyl]methyl]amino]-1,1,1-trifluoro-2-propanol;

(2R)-3-[[3-[3-(1,1,2,2-tetrafluoroethoxy)phenoxy]phenyl][[2-fluoro-4-(trifluoromethyl)phenyl]methyl]amino]-1,1,1-trifluoro-2-propanol;

(2R)-3-[[3-[3-(pentafluoroethyl)phenoxy]phenyl][[2-fluoro-4-(trifluoromethyl)-phenyl]methyl]amino]-1,1,1-trifluoro-2-propanol;

(2R)-3-[[3-(3,5-dimethylphenoxy)phenyl][[2-fluoro-4-(trifluoromethyl)phenyl]-methyl]aminol-1,1,1-trifluoro-2-propanol;

(2R)-3-[[3-(3-ethylphenoxy)phenyl][[2-fluoro-4-(trifluoromethyl)phenyl]methyl]-amino]-1,1,1-trifluoro-2-propanol;

(2R)-3-[[3-(3-t-butylphenoxy)phenyl][[2-fluoro-4-(trifluoromethyl)phenyl]methyl]-amino]-1,1,1-trifluoro-2-propanol;

(2R)-3-[[3-(3-methylphenoxy)phenyl][[2-fluoro-4-(trifluoromethyl)phenyl]methyl]-amino]-1,1,1-trifluoro-2-propanol;

(2R)-3-[[3-(5,6,7,8-tetrahydro-2-naphthoxy)phenyl][[2-fluoro-4-(trifluoromethyl)-phenyl]methyl]amino]-1,1,1-trifluoro-2-propanol;

(2R)-3-[[3-(phenoxy)phenyl][[2-fluoro-4-(trifluoromethyl)phenyl]methyl]amino]-1,1,1-trifluoro-2-propanol;

(2R)-3-[[3-[3-(N,N-dimethylamino)phenoxy]phenyl][[2-fluoro-4-(trifluoromethyl)-phenyl]methyl]amino]-1,1,1-trifluoro-2-propanol;

(2R)-3-[[[2-fluoro-4-(trifluoromethyl)phenyl]methyl][3-[[3-(trifluoromethoxy)phenyl]methoxy]phenyl]amino]-1,1,1-trifluoro-2-propanol;

(3R)-3-[[[2-fluoro-4-(trifluoromethyl)phenyl]methyl][3-[[3-(trifluoromethyl)phenyl]methoxy]phenyl]amino]-1,1,1-trifluoro-2-propanol;

(2R)-3-[[[2-fluoro-4-(trifluoromethyl)phenyl]methyl][3-[[3,5-dimethylphenyl]-methoxy]phenyl]amino]-1,1,1-trifluoro-2-propanol;

(2R)-3-[[[2-fluoro-4-(trifluoromethyl)phenyl]methyl][3-[[3-(trifluoromethylthio)-phenyl]methoxy]phenyl]amino]-1,1,1-trifluoro-2-propanol;

(2R)-3-[[[2-fluoro-4-(trifluoromethyl)phenyl]methyl][3-[[3,5-difluorophenyl]-methoxy]phenyl]amino]-1,1,1-trifluoro-2-propanol;

(2R)-3-[[[2-fluoro-4-(trifluoromethyl)phenyl]methyl][3-[cyclohexylmethoxy]-phenyl]amino]-1,1,1-trifluoro-2-propanol;

(2R)-3-[[3-(2-difluoromethoxy-4-pyridyloxy)phenyl][[2-fluoro-4-(trifluoromethyl)-phenyl]methyl]amino]-1,1,1-trifluoro-2-propanol;

(2R)-3-[[3-(2-trifluoromethyl-4-pyridyloxy)phenyl][[2-fluoro-4-(trifluoromethyl)-phenyl]methyl]amino]-1,1,1-trifluoro-2-propanol;

(2R)-3-[[3-(3-difluoromethoxyphenoxy)phenyl][[2-fluoro-4-(trifluoromethyl)-phenyl]methyl]amino]-1,1,1-trifluoro-2-propanol;

(2R)-3-[[[3-(3-trifluoromethylthio)phenoxy]phenyl][[2-fluoro-4-(trifluoromethyl)-phenyl]methyl]amino]-1,1,1-trifluoro-2-propanol; and

(2R)-3-[[3-(4-chloro-3-trifluoromethylphenoxy)phenyl][[2-fluoro-4-(trifluoromethyl)phenyl]methyl]amino]-1,1,1-trifluoro-2-propanol.

Another class of CETP inhibitors that finds utility with the present invention consists of quinolines of Formula XVII

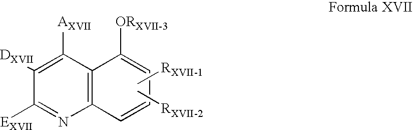

and pharmaceutically acceptable forms thereof, wherein:

A_{XVII} denotes an aryl containing 6 to 10 carbon atoms, which is optionally substituted with up to five identical or different substituents in the form of a halogen, nitro, hydroxyl, trifluoromethyl, trifluoromethoxy or a straight-chain or branched alkyl, acyl, hydroxyalkyl or alkoxy containing up to 7 carbon atoms each, or in the form of a group according to the formula —NR_{XVII-4}R_{XVII-5}, wherein

R_{XVII-4} and R_{XVII-5} are identical or different and denote a hydrogen, phenyl or a straight-chain or branched alkyl containing up to 6 carbon atoms,

D_{XVII} denotes an aryl containing 6 to 10 carbon atoms, which is optionally substituted with a phenyl, nitro, halogen, trifluoromethyl or trifluoromethoxy, or a radical according to the formula

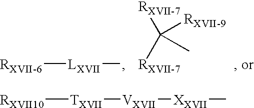

wherein

R_{XVII}, R_{XVII-1} R_{XVII-10} denote, independently from one another, a cycloalkyl containing 3 to 6 carbon atoms, or an aryl containing 6 to 10 carbon atom or a 5- to 7-membered, optionally benzo-condensed, saturated or unsaturated, mono-, bi- or tricyclic heterocycle containing up to 4 heteroatoms from the series of S, N and/or O, wherein the rings are optionally substituted, in the case of the nitrogen-containing rings also via the N function, with up to five identical or different substituents in the form of a halogen, trifluoromethyl, nitro, hydroxyl, cyano, carboxyl, trifluoromethoxy, a straight-chain or branched acyl, alkyl, alkylthio, alkylalkoxy, alkoxy or alkoxycarbonyl containing up to 6 carbon atoms each, an aryl or trifluoromethyl-substituted aryl containing 6 to 10 carbon atoms each, or an optionally benzo-condensed, aromatic 5- to 7-membered heterocycle containing up to 3 heteoatoms from the series of S, N and/or O, and/or in the form of a group according to the formula —OR_{XVII-11}, —SR_{XVII-12}, —SO₂R_{XVII-13}, or —NR_{XVII-14}R_{XVII-15};

R_{XVII-11}, R_{XVII-12}, and R_{XVII-13} denote, independently from one another, an aryl containing 6 to 10 carbon atoms, which is in turn substituted with up to two identical or different substituents in the form of a phenyl, halogen or a straight-chain or branched alkyl containing up to 6 carbon atoms,

R_{XVII-14} and R_{XVII-15} are identical or different and have the meaning of R_{XVII-4} and R_{XVII-5} given above, or

R_{XVII-6} and/or R_{XVII-7} denote a radical according to the formula

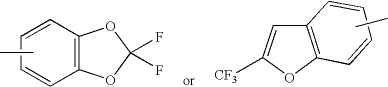

R_{XVII-8} denotes a hydrogen or halogen, and

R_{XVII-9} denotes a hydrogen, halogen, azido, trifluoromethyl, hydroxyl, trifluoromethoxy, a straight-chain or branched alkoxy or alkyl containing up to 6 carbon atoms each, or a radical according to the formula NR_{XVII-16}R_{XVII-17};

R_{XVII-16} and R_{XVII-17} are identical or different and have the meaning of R_{XVII}₄ and R_{XVII-5} above; or

R_{XVII-8} and R_{XVII-9} together form a radical according to the formula ═O or ═NR_{XVII-18};

R_{XVII-18} denotes a hydrogen or a straight-chain or branched alkyl, alkoxy or acyl containing up to 6 carbon atoms each;

L_{XVII} denotes a straight-chain or branched alkylene or alkenylene chain containing up to 8 carbon atoms each, which are optionally substituted with up to two hydroxyl groups;

T_{XVII} and X_{XVII} are identical or different and denote a straight-chain or branched alkylene chain containing up to 8 carbon atoms; or

T_{XVII} and X_{XVII} denotes a bond;

V_{XVII} denotes an oxygen or sulfur atom or —NR_{XVII-19};

R_{XVII-19} denotes a hydrogen or a straight-chain or branched alkyl containing up to 6 carbon atoms or a phenyl;

E_{XVII} denotes a cycloalkyl containing 3 to 8 carbon atoms, or a straight-chain or branched alkyl containing up to 8 carbon atoms, which is optionally substituted with a cycloalkyl containing 3 to 8 carbon atoms or a hydroxyl, or a phenyl, which is optionally substituted with a halogen or trifluoromethyl;

R_{XVII-1} and R_{XVII-2} are identical or different and denote a cycloalkyl containing 3 to 8 carbon atoms, hydrogen, nitro, halogen, trifluoromethyl, trifluoromethoxy, carboxy, hydroxy, cyano, a straight-chain or branched acyl, alkoxycarbonyl or alkoxy with up to 6 carbon atoms, or NR_{XVII-20}R_{XVII-21};

R_{XVII-20} and R_{XVII-21} are identical or different and denote hydrogen, phenyl, or a straight-chain or branched alkyl with up to 6 carbon atoms; and or

R_{XVII-1} and/or R_{XVII-2} are straight-chain or branched alkyl with up to 6 carbon atoms, optionally substituted with halogen, trifluoromethoxy, hydroxy, or a straight-chain or branched alkoxy with up to 4 carbon atoms, aryl containing 6-10 carbon atoms optionally substituted with up to five of the same or different substituents selected from halogen, cyano, hydroxy, trifluoromethyl, trifluoromethoxy, nitro, straight-chain or branched alkyl, acyl, hydroxyalkyl, alkoxy with up to 7 carbon atoms and NR_{XVII-2}R_{XVII-23};

R_{XVII-22} and R_{XVII-23} are identical or different and denote hydrogen, phenyl or a straight-chain or branched akyl up to 6 carbon atoms; and/or R_{XVII-1} and R_{XVII-2} taken together form a straight-chain or branched alkene or alkane with up to 6 carbon atoms optionally substituted with halogen, trifluoromethyl, hydroxy or straight-chain or branched alkoxy with up to 5 carbon atoms;

R_{XVII-3} denotes hydrogen, a straight-chain or branched acyl with up to 20 carbon atoms, a benzoyl optionally substituted with halogen, trifluoromethyl, nitro or trifluoromethoxy, a straight-chained or branched fluoroacyl with up to 8 carbon atoms and 7 fluoro atoms, a cycloalkyl with 3 to 7 carbon atoms, a straight chained or branched alkyl with up to 8 carbon atoms optionally substituted with hydroxyl, a straight-chained or branched alkoxy with up to 6 carbon atoms optionally substituted with phenyl which may in turn be substituted with halogen, nitro, trifluoromethyl, trifluoromethoxy, or phenyl or a tetrazol substitued phenyl, and/or an alkyl that is optionally substituted with a group according to the formula —OR_{XVII-24};

R_{XVII-24} is a straight-chained or branched acyl with up to 4 carbon atoms or benzyl.

Compounds of Formula XVII are disclosed in WO 98/39299, the entire disclosure is incorporated by reference.

Another class of CETP inhibitors that finds utility with the present invention consists of 4-Phenyltetrahydroquinolines of Formula XVIII

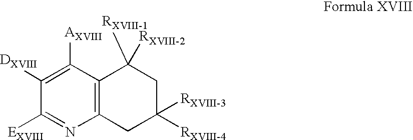

N oxides thereof, and pharmaceutically acceptable forms thereof, wherein:

A_{XVIII} denotes a phenyl optionally substituted with up to two identical or different substituents in the form of halogen, trifluoromethyl or a straight-chain or branched alkyl or alkoxy containing up to three carbon atoms;

D_{XVIII} denotes the formula

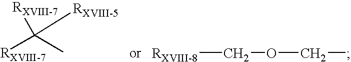

R_{XVIII-5} and R_{XVIII-6} are taken together to form ═O; or

R_{XVIII-5} denotes hydrogen and R_{XVIII-6} denotes halogen or hydrogen; or

R_{XVIII-5} and R_{XVIII-6} denote hydrogen;

R_{XVIII-7} and R_{XVIII-8} are identical or different and denote phenyl, naphthyl, benzothiazolyl, quinolinyl, pyrimidyl or pyridyl with up to four identical or different substituents in the form of halogen, trifluoromethyl, nitro, cyano, trifluoromethoxy, —SO₂—CH₃ or NR_{XVIII-9}R_{XVIII-10};

R_{XVIII-9} and R_{XVII-10} are identical or different and denote hydrogen or a straight-chained or branched alkyl of up to three carbon atoms;

E_{XVIII} denotes a cycloalkyl of from three to six carbon atoms or a straight-chained or branched alkyl of up to eight carbon atoms;

R_{XVIII-1} denotes hydroxy;

R_{XVIII-2} denotes hydrogen or methyl;

R_{XVIII-3} and R_{XVIII-4} are identical or different and denote straight-chained or branched alkyl of up to three carbon atoms; or

R_{XVIII-3} and R_{XVIII-4} taken together form an alkenylene made up of between two and four carbon atoms.

Compounds of Formula XVIII are disclosed in WO 99/15504, the entire disclosure of which is incorporated by reference.

Another class of CETP inhibitors that finds utility with the present invention consists of aminoethanol derivatives of Formula XIX

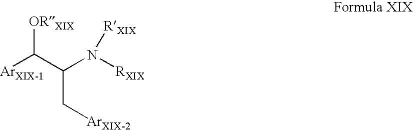

and pharmaceutically acceptable forms thereof, wherein:

Ar_{XIX-1} denotes an aromatic ring group that may contain a substituting group;

Ar_{XIX-2} denotes an aromatic ring group that may contain a substituting group;

R_{XIX} denotes an acyl group;

R′_{XIX} denotes a hydrogen atom or hydrocarbon group that may contain a substituting group; and

OR″_{XIX} denotes a hydroxyl group that may be protected.

Compounds of Formula XIX are disclosed in WO 2002/059077, the entire disclosure of which is incorporated by reference.

In a preferred embodiment, the CETP inhibitor is selected from the following compounds of Formula XIX or their salts:

N-[(1RS,2SR)-2-(4-fluorophenyl)-2-hydroxy-1-[4-(trifluoromethyl)benzyl]ethyl]-6,7-dihydro-5H-benzo[a]cyclopentene-1-carboxamide,

4-fluoro-N-((1R,2S)-2-(4-fluorophenyl)-2-hydroxy-1-((4-(trifluoromethyl)phenyl)methyl)ethyl)-1-naphthalene carboxamide;

N-[(1R,2S)-2-(4-fluorophenyl)-2-hydroxy-1-[3-(1,1,2,2-tetrafluoroethoxy)benzyl]ethyl]-6,7-dihydro-5H-benzo[a]cyclopentene-1-carboxamide;

N-[(1RS,2SR)-2-(4-fluorophenyl)-2-hydroxy-1-[3-(1,1,2,2-tetrafluoroethoxy)benzyl]ethyl]-5,6-dihydronaphthalene-1-carboxamide;

N-[(1RS,2SR)-2-(4-fluorophenyl)-2-hydroxy-1-[3-(1,1,2,2-tetrafluoroethoxy)benzyl]ethyl]-6,7,8,9-tetrahydro-5H-benzo[a]cycloheptene-1-carboxamide;

4-fluoro-N-[(1R,2S)-2-(4-fluorophenyl)-2-hydroxy-1-[3-(1,1,2,2-tetrafluoroethoxy)benzyl]ethyl]naphthalene-1-carboxamide;

N-[(1RS,2SR)-2-(4-fluorophenyl)-2-hydroxy-1-[3-(1,1,2,2-tetrafluoroethoxy)benzyl]ethyl]-5,6,7,8-tetrahydrobenzo[a]cyclooctene-1-carboxamide;

N-[(1RS,2SR)-2-(4-fluorophenyl)-2-hydroxy-1-(4-isopropylbenzyl)ethyl]-6,7-dihydro-5H-benzo[a]cycloheptene-1-carboxamide;

N-((1RS,2SR)-2-(3-fluorophenyl)-2-hydroxy-1-((4-(trifluoromethyl)phenyl)methyl)ethyl)-6,7-dihydro-5H-benzo[a]cycloheptene-1-carboxamide;

N-((1RS,2SR)-2-hydroxy-2-(4-phenoxyphenyl)-1-((4-(trifluoromethyl)phenyl)methyl)ethyl)-6,7-dihydro-5H-benzo[a]cycloheptene-1-carboxamide;

N-[(1RS,2SR)-2-(4-chlorophenyl)-2-hydroxy-1-[3-(1,1,2,2-tetrafluoroethoxy)benzyl)ethyl)-6,7-dihydro-5H-benzo[a]cycloheptene-1-carboxamide;

N-((1RS,2SR)-2-hydroxy-2-(4-phenyloxy)phenyl)-1-((3-((1,1,2,2-tetrafluoroethyl)oxy)phenyl)methyl)ethyl)-6,7-dihydro-5H-benzo[a]cycloheptene-1-carboxamide;

N-((1RS,2SR)-2-(4-((4-chloro-3-ethylphenyl)oxy)phenyl)-2-hydroxy-1-((3-((1,1,2,2-tetrafluoroethyl)oxy)phenyl)methyl)ethyl)-6,7-dihydro-5H-benzo[a]cycloheptene-1-carboxamide;

N-((1RS,2SR)-2-(2-fluoropyridine-4-yl)-2-hydroxy-1-((3-((1,1,2,2-tetrafluoroethoxy)phenyl)methyl)ethyl)-6,7-dihydro-5H-benzo[a]cycloheptene-1-carboxamide;

N-((1RS,2RS)-2-(6-fluoropyridine-2-yl)-2-hydroxy-1-((3-((1,1,2,2-tetrafluoroethoxy)phenyl)methyl)ethyl)-6,7-dihydro-5H-benzo[a]cycloheptene-1-carboxamide;

N-[(1RS,2SR)-1-(4-tert-butylbenzyl)-2-(3-chlorophenyl)-2-hydroxyethyl]-5-chloro-1-napthoamide;

4-fluoro-N-{(1RS,2SR)-2-(4-fluorophenyl)-2-hydroxy-1-[(2,2,3,3-tetrafluoro-2,3-dihydro-1,4-benzodioxin-6-yl)methyl]ethyl}-1-naphthoamide.

In a preferred embodiment, the CETP inhibitor is [2R,4S]-4-[(3,5-bis-trifluoromethyl-benzyl)-methoxycarbonyl-amino]-2-ethyl-6-trifluoromethyl-3,4-dihydro-2H-quinoline-1-carboxylic acid ethyl ester also known as torcetrapib. Torcetrapib is shown by the following Formula

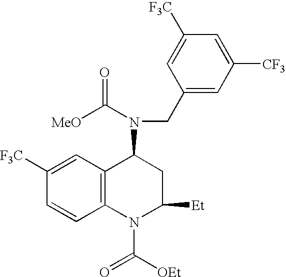

CETP inhibitors, in particular torcetrapib, and methods for preparing such compounds are disclosed in detail in U.S. Pat. Nos. 6,197,786 and 6,313,142, in PCT Application Nos. WO 01/40190A1, WO 02/088085A2, and WO 02/088069A2, the disclosures of which are herein incorporated by reference. Torcetrapib has an unusually low solubility in aqueous environments such as the lumenal fluid of the human GI tract. The aqueous solubility of torcetrapib is less than about 0.04 μg/ml. Torcetrapib must be presented to the GI tract in a solubility-improved form in order to achieve a sufficient drug concentration in the GI tract in order to achieve sufficient absorption into the blood to elicit the desired therapeutic effect.

### Solid Amorphous Adsorbates

The CETP inhibitor is present in the form of a solid amorphous adsorbate comprising the CETP inhibitor and a substrate. The solid amorphous adsorbates resulting from the various preparation techniques, described below, are solid materials comprising about 5 wt % to 90 wt % CETP inhibitor. When doses of the CETP inhibitor are greater than about 20 mg, it is generally preferred that the solid amorphous adsorbates comprise at least 10 wt % CETP inhibitor in order to reduce the total mass of adsorbate that must be delivered.

At least a major portion of the drug in the solid amorphous adsorbate is amorphous. The term “amorphous” indicates simply that the drug is not crystalline as indicated by any conventional method, such as by powder X-ray diffraction (PXRD) analysis in which the sharp scattering lines associated with the crystal forms of the drug are absent or reduced in magnitude or the absence of an endothermic transition at the melting point of the crystalline drug when subjected to thermal analysis. The term “a major portion” of the drug means that at least 60% of the drug is in amorphous form, rather than a crystalline form. Preferably, the drug in the adsorbate is substantially amorphous. As used herein, “substantially amorphous” means that the amount of the drug in amorphous form is at least 80%. More preferably, the drug in the adsorbate is “almost completely amorphous” meaning that the amount of drug in the amorphous form is at least 90% as measured by powder X-ray diffraction or differential scanning calorimetry (“DSC”), or any other standard quantitative measurement.

The solid amorphous adsorbate is capable of supersaturating the CETP inhibitor, at least temporarily, in an aqueous use environment by a factor of about 1.25-fold or more, relative to a control composition consisting essentially of crystalline CETP inhibitor alone. That is, the solid amorphous adsorbate provides a maximum dissolved drug concentration (MDC) of the CETP inhibitor in a use environment that is at least 1.25-fold the equilibrium drug concentration provided by the unadsorbed, crystalline form of the CETP inhibitor alone. The control composition is conventionally the lowest-energy crystalline form of the CETP inhibitor alone. It is to be understood that the control composition is free from solubilizers or other components that would materially affect the solubility of the CETP inhibitor, and that the CETP inhibitor is in solid crystalline form in the control composition. Preferably, the solid amorphous adsorbate increases the MDC of the CETP inhibitor in aqueous solution by at least 2-fold relative to the control composition, more preferably by at least 3-fold, and most preferably by at least 5-fold. Surprisingly, the solid amorphous adsorbate may achieve extremely large enhancements in aqueous concentration. In some cases, especially when formulated with a concentration-enhancing polymer as discussed below, the MDC of CETP inhibitor provided by the solid amorphous adsorbate is at least 10-fold, at least 50-fold, at least 200-fold, at least 500-fold, to more than 1000-fold the equilibrium concentration provided by the crystalline control.

When the crystalline form of the CETP inhibitor is not known, the control composition consists essentially of the lowest-energy amorphous form of the CETP inhibitor. In such cases, the solid amorphous adsorbate may not provide supersaturation relative to the amorphous drug alone, but rather, provides a greatly enhanced dissolution rate such that the aqueous drug concentration reaches the solubility of the amorphous drug much more rapidly than the amorphous control. Methods for determining the dissolution rate of a solid amorphous adsorbate are discussed in detail below.

Because the solid amorphous adsorbate provides rapid dissolution of the CETP inhibitor, the solid amorphous adsorbate provides an area under the CETP inhibitor concentration versus time curve (AUC) in the use environment that may be at least 1.25-fold that provided by a control composition. (The calculation of an AUC is a well-known procedure in the pharmaceutical arts and is described, for example, in Welling, “Pharmacokinetics Processes and Mathematics,” ACS Monograph 185 (1986).) More specifically, in the environment of use, the CETP inhibitor in solid amorphous adsorbate form provides an AUC for any 90-minute period of from about 0 to about 270 minutes following introduction to the use environment that is at least 1.25-fold that of a control composition. The control composition is conventionally the lowest-energy crystalline form of the CETP inhibitor alone without any solubilizing additives, as described above, or the lowest-energy amorphous form of the CETP inhibitor alone. Preferably, the AUC provided by the solid amorphous adsorbate is at least 2-fold, more preferably at least 3-fold that of the control composition. For some CETP inhibitors, the solid amorphous adsorbate may provide an AUC value that is at least 5-fold, at least 25-fold, at least 100-fold, and even more than 250-fold that of the control described above.

The aqueous use environment can be either the in vivo environment, such as the GI tract of an animal, particularly a human, or the in vitro environment of a test solution, such as phosphate buffered saline (PBS) solution or Model Fasted Duodenal (MFD) solution. Concentration enhancement may be determined through either in vivo tests or through in vitro dissolution tests. A composition of the present invention meets the concentration enhancement criteria in at least one of the above test environments.

Where the use environment is the GI tract of an animal, dissolved drug concentration may be determined by any conventional method known in the art. One method is a deconvolution method. In this method, the serum or plasma drug concentration is plotted along the ordinate (y-axis) against the blood sample time along the abscissa (x-axis). The data may then be analyzed to determine drug release rates in the GI tract using any conventional analysis, such as the Wagner-Nelson or Loo-Riegelman analysis. See also Welling, “Pharmacokinetics: Processes and Mathematics” (ACS Monograph 185, Amer. Chem. Soc., Washington, D. C., 1986). Treatment of the data in this manner yields an apparent in vivo drug release profile. Another method is to intubate the patient and periodically sample the GI tract directly.

The solid amorphous adsorbates of CETP inhibitor used in the inventive compositions provide enhanced concentration of the dissolved CETP inhibitor in in vitro dissolution tests. It has been determined that enhanced drug concentration in in vitro dissolution tests in MFD solution or in PBS solution is a good indicator of in vivo performance and bioavailability. An appropriate PBS solution is an aqueous solution comprising 20 mM Na₂HPO₄, 47 mM KH₂PO₄, 87 mM NaCl, and 0.2 mM KCl, adjusted to pH 6.5 with NaOH. An appropriate MFD solution is the same PBS solution wherein there is also present 7.3 mM sodium taurocholic acid and 1.4 mM of 1-palmitoyl-2-oleyl-sn-glycero-3-phosphocholine. In particular, solid amorphous adsorbates of CETP inhibitor can be dissolution-tested by adding it to MFD or PBS solution and agitating to promote dissolution.

An in vitro test to evaluate enhanced CETP inhibitor concentration in aqueous solution can be conducted by (1) adding with agitation a sufficient quantity of control composition, i.e., the CETP inhibitor in unadsorbed form alone, to the in vitro test medium, such as an MFD or a PBS solution, to achieve equilibrium concentration of the CETP inhibitor; (2) in a separate vessel, adding with agitation a sufficient quantity of test composition (e.g., the CETP inhibitor in solid amorphous adsorbate form) in the same test medium, such that if all the CETP inhibitor dissolved, the theoretical concentration of CETP inhibitor would exceed the equilibrium concentration of the CETP inhibitor by a factor of at least 2, and preferably by a factor of at least 10; and (3) comparing the measured MDC and/or aqueous AUC of the test composition in the test medium with the equilibrium concentration, and/or with the aqueous AUC of the control composition. In conducting such a dissolution test, the amount of test composition or control composition used is an amount such that if all of the CETP inhibitor dissolved the CETP inhibitor concentration would be at least 2-fold, and preferably at least lOO-fold that of the equilibrium concentration. Indeed, for some extremely insoluble CETP inhibitors, in order to identify the MDC achieved it may be necessary to use an amount of test composition such that if all of the CETP inhibitor dissolved, the CETP inhibitor concentration would be 1000-fold or even more, that of the equilibrium concentration of the CETP inhibitor.

The concentration of dissolved CETP inhibitor is typically measured as a function of time by sampling the test medium and plotting CETP inhibitor concentration in the test medium vs. time so that the MDC can be ascertained. The MDC is taken to be the maximum value of dissolved CETP inhibitor measured over the duration of the test. The aqueous AUC is calculated by integrating the concentration versus time curve over any 90-minute time period between the time of introduction of the composition into the aqueous use environment (when time equals zero) and 270 minutes following introduction to the use environment (when time equals 270 minutes). Typically, when the composition reaches its MDC rapidly, in say less than about 30 minutes, the time interval used to calculate AUC is from time equals zero to time equals 90 minutes. However, if the AUC of a composition over any 90-minute time period described above meets the criterion of this invention, then the composition formed is considered to be within the scope of this invention.

To avoid large CETP inhibitor particulates that would give an erroneous determination, the test solution is either filtered or centrifuged. “Dissolved drug” is typically taken as that material that either passes a 0.45 μm syringe filter or, alternatively, the material that remains in the supernatant following centrifugation. Filtration can be conducted using a 13 mm, 0.45 μm polyvinylidine difluoride syringe filter sold by Scientific Resources under the trademark TITAN®. Centrifugation is typically carried out in a polypropylene microcentrifuge tube by centrifuging at 13,000 G for 60 seconds. Other similar filtration or centrifugation methods can be employed and useful results obtained. For example, using other types of microfilters may yield values somewhat higher or lower (±10-40%) than that obtained with the filter specified above but will still allow identification of preferred compositions.

Alternatively, an in vivo test may be used to determine whether a composition is within the scope of the present invention. However, due to the inherent difficulties and complexity of the in vivo procedure, it is preferred that in vitro procedures be used to evaluate compositions even though the ultimate use environment is often the human GI tract. The in vitro tests described above are expected to approximate in vivo behavior, and a composition that meets the in vitro release rates described herein are within the scope of the invention.

Thus, the CETP inhibitor in solid amorphous adsorbate form, when dosed orally to a human or other animal in the fasted state, provides an AUC in CETP inhibitor concentration in the blood (serum or plasma) that is at least about 1.25-fold, preferably at least about 2-fold, preferably at least about 3-fold, preferably at least about 4-fold, preferably at least about 6-fold, preferably at least about 10-fold, and even more preferably at least about 20-fold that observed when a control composition consisting of an equivalent quantity of CETP inhibitor in unadsorbed form is dosed to a subject in the fasted state. It is noted that such compositions can also be said to have a relative bioavailability of from about 1.25-fold to about 20-fold that of the control composition.

Alternatively, the CETP inhibitor in solid amorphous adsorbate form, when dosed orally to a human or other animal in the fasted state, provides a maximum CETP inhibitor concentration in the blood, Cₘₐₓ (serum or plasma), that is at least about 1.25-fold, preferably at least about 2-fold, preferably at least about 3-fold, preferably at least about 4-fold, preferably at least about 6-fold, preferably at least about 10-fold, and even more preferably at least about 20-fold that observed when a control composition consisting of an equivalent quantity of CETP inhibitor in unadsorbed form is dosed to a subject in the fasted state.

Relative bioavailability of CETP inhibitors in solid amorphous adsorbate form can be tested in vivo in animals or humans using conventional methods for making such a determination. An in vivo test, such as a crossover study, may be used to determine whether a composition of CETP inhibitor in solid amorphous adsorbate form provides an enhanced relative bioavailability compared with a control composition as described above. In an in vivo crossover study a test composition of a CETP inhibitor in solid amorphous adsorbate form is dosed to half a group of test subjects and, after an appropriate washout period (e.g., one week) the same subjects are dosed with a control composition that consists of an equivalent quantity of crystalline CETP inhibitor as the test composition. The other half of the group is dosed with the control composition first, followed by the test composition. The relative bioavailability is measured as the concentration in the blood (serum or plasma) versus time area under the curve (AUC) determined for the test group divided by the AUC in the blood provided by the control composition. Preferably, this test/control ratio is determined for each subject, and then the ratios are averaged over all subjects in the study. In vivo determinations of AUC can be made by plotting the serum or plasma concentration of drug along the ordinate (y-axis) against time along the abscissa (x-axis). To facilitate dosing, a dosing vehicle may be used to administer the dose. The dosing vehicle is preferably water, but may also contain materials for suspending the test or control composition, provided these materials do not dissolve the composition or change the drug solubility in vivo.

When performing such tests, the subject is preferably in the fasted state. By “fasted state” is meant that the subject has not eaten for at least eight hours, typically overnight, prior to ingestion of the composition or dosage form.

The solid amorphous adsorbate also comprises a substrate. The substrate may be any material that is inert, meaning that the substrate does not adversely interact with the drug to an unacceptably high degree and which is pharmaceutically acceptable. Exemplary materials which are suitable for the substrate include inorganic oxides, such as SiO₂, TiO₂, ZnO₂, ZnO, Al₂O₃, magnesium aluminum silicates, calcium silicates, AIOH₂, magnesium hydroxide, magnesium oxide, magnesium trisilicate, talc, and dibasic calcium phosphate; zeolites, and other inorganic molecular sieves; clays, such as kaolin (hydrated aluminum silicate), bentonite (hydrated aluminum silicate), hectorite, and Veegum®; Na—, Al—, and Fe-montmorillonite; water insoluble polymers, such as cross-linked cellulose acetate phthalate, cross-linked hydroxypropyl methyl cellulose acetate succinate, cross-linked polyvinyl pyrrolidinone (also known as cross povidone), microcrystalline cellulose, polyethylene/polyvinyl alcohol copolymer, polyethylene polyvinyl pyrrolidone copolymer, cross-linked carboxymethyl cellulose, sodium starch glycolate, and cross-linked polystyrene divinyl benzene; and activated carbons, including those made by carbonization of polymers such as polyimides, polyacrylonitrile, phenolic resins, cellulose acetate, regenerated cellulose, and rayon. Preferably, the substrate is selected from the group consisting of inorganic oxides, clays, and water-insoluble polymers. Most preferably, the substrate is SiO₂.

The surface of the substrate may be modified with various substituents to achieve particular interactions of the drug with the substrate. For example, the substrate may have a hydrophobic or hydrophilic surface. By varying the terminating groups of substituents attached to the substrate, the interaction between the drug and substrate may be influenced. For example, where the drug is hydrophobic, it may be desired to select a substrate having hydrophobic substituents to improve the binding of the drug to the substrate.

Generally, the interaction of drug with the substrate should be sufficiently high such that mobility of the drug in the drug/substrate adsorbate is sufficiently decreased such that the composition maintains the amorphous form of the CETP inhibitor, as described herein. However, the drug/substrate interaction should be sufficiently low such that the drug can readily desorb from the adsorbate when it is introduced to a use environment, resulting in a high concentration of drug in solution.

In one embodiment, the solid amorphous adsorbate comprises a CETP inhibitor adsorbed onto a substrate, the substrate having a surface area of at least 20 m²/g, and wherein at least a major portion of the CETP inhibitor in the solid adsorbate is amorphous. The solid adsorbate may optionally comprise a concentration-enhancing polymer. The solid adsorbate may also be mixed with a concentration-enhancing polymer. Such solid adsorbates are disclosed in commonly assigned copending U.S. patent application Ser. No. 10/173,987, filed Jun. 17, 2002, which is incorporated in its entirety by reference.

The substrate has a high surface area, meaning that the substrate has a surface area of at least 20 m²/g, preferably at least 50 m²/g, more preferably at least 100 m²/g, and most preferably at least 180 m²/g. The surface area of the substrate may be measured using standard procedures. One exemplary method is by low-temperature nitrogen adsorption, based on the Brunauer, Emmett, and Teller (BET) method, well known in the art. As discussed below, the higher the surface area of the substrate, the higher the drug-to-substrate ratio that can be achieved and still maintain high concentration-enhancements. Thus, effective substrates can have surface areas of up to 200 m²/g, up to 400 m²/g and up to 600 m²/g or more. The substrate is preferably in the form of small particles ranging in size of from 10 nm to 1 μm, preferably ranging in size from 20 nm to 100 nm. These particles may in turn form agglomerates ranging in size from 10 nm to 100 μm. The substrate is preferably insoluble in the process environment used to form the adsorbate. That is, where the adsorbate is formed by solvent processing, the substrate does not dissolve in the solvent. Where the adsorbate is formed by a melt or thermal process, the substrate has a sufficiently high melting point that it does not melt.

The adsorbates are formed so as to form a thin layer of amorphous drug on the surface of the substrate. By “thin layer” is meant a layer that ranges in average thickness from less than one drug molecule to as many as 10 molecules. When the average drug layer thickness, based on the ratio of the mass of drug-to-substrate surface area, is about the dimensions of one molecule or less, the drug layer is generally termed a “monolayer.” For such monolayers, most drug molecules are in direct contact with the substrate.

The adsorption of drug to the substrate may be characterized by a shift in the infra red (IR) spectra of the drug, indicating interaction of the drug with the substrate. Such interactions are generally due to London dispersion forces, dipole-dipole interactions, hydrogen bonding, electron donor-electron acceptor interactions or ionic interactions. Such interactions usually only have a substantial effect on the IR spectrum when the drug is in direct contact with the substrate. Thus, as the number of layers of molecules on the substrate increases, the average shift of the IR absorption decreases. That is, the IR spectrum will show a composite of those molecules that are in contact with the substrate surface as well as those that are further away from the surface.

The inventors have discovered that if the adsorbate contains too many layers of amorphous drug, the physical stability of the adsorbate may be compromised. Thus, crystallization of the drug molecules on a thick adsorbed layer may occur more rapidly than that observed for a thin adsorbed layer. In general, the acceptable thickness of the amorphous drug layer that has sufficient physical stability is inversely related to the melting point of the drug. Without wishing to be bound by any particular theory or mechanism of action, it is believed that as the melting point of the drug decreases, the driving force for crystallization of the drug decreases. Nucleation theory for drug in a supercooled melt shows that the free energy of the drug is based on two terms: the surface free energy and the volume free energy. The free energy of a nucleating crystal is maximized at a critical radius for the nucleus. A nucleating crystal that is larger than this critical radius will preferentially grow because further growth decreases the total free energy of the system. A nucleating crystal that is smaller than this critical radius will usually re-dissolve because re-dissolution results in a decrease in the total free energy of the system. This critical radius is inversely related to the melting temperature of the drug. Thus, a drug with a lower melting temperature will result in a larger critical radius. The inventors have discovered that in general, a solid amorphous adsorbate with a drug layer thickness that is smaller than the size of the critical radius will be physically stable in the amorphous state for long periods of time. For many CETP inhibitors that have melting points of about 150° C. or less, the average adsorbed layer thickness can be up to 5 to 10 molecules and still have good physical stability. For substrates such as SiO₂ with surface areas of about 200 m²/g (such as CAB-O-SIL M-5P), this corresponds to drug loadings of about 30 to 60 wt %.

One exemplary method for forming the solid amorphous adsorbates of the present invention is “solvent processing.” Solvent processing consists of dissolution of the drug in a solvent containing the substrate followed by rapid removal of the solvent. The term “solvent” is used broadly and includes mixtures of solvents. In general, the substrate will not significantly dissolve in the solvent and remains solid throughout the process.

First, the substrate is added to a solvent that is capable of dissolving the drug. Since it is generally desirable to form adsorbate particles that are small, preferably less than about 1 to 10 μm, the solution is agitated to form a suspension of small particles of substrate suspended in the solvent. Agitation of the solution may be performed by any method that is capable of imparting sufficient energy to the solution to break up agglomerations of substrate particles. A preferred method is sonication. Other methods that may be used to break up the particles to form a suspension of substrate in the solvent include high speed mixing, and high shear mechanical mixing. The solution is agitated for a sufficient length of time so that the substrate remains suspended in the solution for at least a few minutes. Often, to ease processing, it is desirable that the substrate remain suspended for at least 60 minutes without agglomeration. However, this is not required for practice of the invention. The solvent/substrate suspension may be continuously agitated during processing to ensure the substrate remains suspended in the solvent.

The drug is added to the solvent and dissolved. The amount of drug and substrate present in the solution is chosen to yield an adsorbate having the desired ratio of drug to substrate. In general, good results may be obtained where the solution comprises from 0.1 to 2 wt % drug and from 0.1 to 5 wt % substrate. In general, it is desired to maintain the amount of solids in the solution at less than about 10 wt %, as the substrate when present at higher concentrations may clog or stick to the surfaces of the apparatus used to form the adsorbate. The weight ratio of drug to substrate is chosen such that the desired drug-layer thickness is obtained. Generally, better dissolution performance is obtained at lower drug-to-substrate ratios. However, higher drug-to-substrate weight ratios provide good performance when the substrate surface area is high. Typically, drug-to-substrate weight ratios are about 3.0 or less, about 1.0 or less, and often about 0.25 or less to obtain preferred dissolution performance.

After the substrate has been agitated and the drug has been dissolved, the solvent is rapidly removed by evaporation or by mixing with a non-solvent. Exemplary processes are spray-drying, spray-coating (pan-coating, fluidized bed coating, etc.), and precipitation by rapid mixing of the solution with CO₂, hexane, heptane, water of appropriate pH, or some other non-solvent. Preferably, removal of the solvent results in a solid adsorbate. To achieve this end, it is generally desirable to rapidly remove the solvent from the solution such as in a process where the solution is atomized and the drug rapidly solidifies on the substrate.

The solid amorphous adsorbates formed by such processes that rapidly “quench” the material, that is, bring the material from the dissolved state to the solid state very rapidly are generally preferred as they result in a material with superior physical structure and performance.

In one embodiment, the solvent is removed through the process of spray-drying. The term spray-drying is used conventionally and broadly refers to processes involving breaking up liquid mixtures into small droplets (atomization) and rapidly removing solvent from the mixture in a container (spray-drying apparatus) where there is a strong driving force for evaporation of solvent from the droplets. The strong driving force for solvent evaporation is generally provided by maintaining the partial pressure of solvent in the spray-drying apparatus well below the vapor pressure of the solvent at the temperature of the drying droplets. This is accomplished by either (1) maintaining the pressure in the spray-drying apparatus at a partial vacuum (e.g., 0.01 to 0.50 atm); (2) mixing the liquid droplets with a warm drying gas; or (3) both. In addition, at least a portion of the heat required for evaporation of solvent may be provided by heating the spray solution.

Solvents suitable for spray drying can be any compound or mixture of compounds in which the drug has a high solubility and the substrate has a low solubility. Preferably, the solvent is also volatile with a boiling point of about 150° C. or less. In addition, the solvent should have relatively low toxicity and be removed from the adsorbate to a level that is acceptable according to The International Committee on Harmonization (ICH) guidelines. Removal of solvent to this level may require a processing step such as tray-drying subsequent to the spray-drying or spray-coating process. Preferred solvents include alcohols such as methanol, ethanol, n-propanol, isopropanol, and butanol; ketones such as acetone, methyl ethyl ketone and methyl iso-butyl ketone; esters such as ethyl acetate and propylacetate; and various other solvents such as acetonitrile, methylene chloride, toluene, tetrahydrofuran, and 1,1,1-trichloroethane. Mixtures, particularly mixtures of an organic solvent such as methanol, ethanol or acetone and water are often desirable. Lower volatility solvents such as dimethyl acetamide or dimethylsulfoxide can also be used. Mixtures of solvents, such as 50% methanol and 50% acetone, can also be used, as can mixtures with water as long as the drug is sufficiently soluble to make the spray-drying process practicable.

The solvent-bearing feed, comprising the CETP inhibitor and the substrate, can be spray-dried under a wide variety of conditions and yet still yield solid amorphous adsorbates with acceptable properties. For example, various types of nozzles can be used to atomize the spray solution, thereby introducing the spray solution into the spray-dry chamber as a collection of small droplets. Essentially any type of nozzle may be used to spray the solution as long as the droplets that are formed are sufficiently small that they dry sufficiently (due to evaporation of solvent) that they do not stick to or coat the spray-drying chamber wall.

Although the maximum droplet size varies widely as a function of the size, shape and flow pattern within the spray-dryer, generally droplets should be less than about 500 μm in diameter when they exit the nozzle. Examples of types of nozzles that may be used to form the solid amorphous dispersions include the two-fluid nozzle, the fountain-type nozzle, the flat fan-type nozzle, the pressure nozzle and the rotary atomizer. In a preferred embodiment, a pressure nozzle is used, as disclosed in commonly assigned copending U.S. Provisional Application No.60/353,986, the disclosure of which is incorporated herein by reference.

Generally, the temperature and flow rate of the drying gas is chosen so that the droplets containing the adsorbate are dry enough by the time they reach the wall of the apparatus that they are essentially solid, and so that they form a fine powder and do not stick to the apparatus wall. The actual length of time to achieve this level of dryness depends on the size of the droplets. Droplet sizes generally range from 1 μm to 500 μm in diameter, with 5 to 150 μm being more typical. The large surface-to-volume ratio of the droplets and the large driving force for evaporation of solvent leads to actual drying times of a few seconds or less, and more typically less than 0.1 second. Solidification times should be less than 100 seconds, preferably less than a few seconds, and more preferably less than 1 second. In a preferred embodiment, the height and volume of the spray-dryer are adjusted to provide sufficient time for the droplets to dry prior to impinging on an internal surface of the spray-dryer, as described in detail in commonly assigned, copending U.S. Provisional Application No. 60/354,080, incorporated herein by reference. In general, to achieve this rapid solidification of the solution, it is preferred that the size of droplets formed during the spray-drying process be less than about 150 μm in diameter. The resultant solid particles thus formed are generally less than about 150 μm in diameter.

Following solidification, the solid powder typically stays in the spray-drying chamber for about 5 to 60 seconds, further evaporating solvent from the solid powder. The final solvent content of the solid adsorbate as it exits the dryer should be low, since this reduces the mobility of drug molecules in the adsorbate, thereby improving its stability. Generally, the solvent content of the adsorbate as it leaves the spray-drying chamber should be less than 10 wt % and preferably less than 2 wt %. Following spray-drying, the adsorbate may be dried in a solvent drier, such as a tray-dryer or a fluidized-bed dryer to remove residual solvents.

Spray-drying processes and spray-drying equipment are described generally in Perry's Chemical Engineers' Handbook, Sixth Edition (R. H. Perry, D. W. Green, J. O. Maloney, eds.) McGraw-Hill Book Co. 1984, pages 20-54 to 20-57. More details on spray-drying processes and equipment are reviewed by Marshall “Atomization and Spray-Drying,” 50 Chem. Eng. Prog. Monogr. Series 2 (1954).

As mentioned above, preferred adsorbates of the present invention are made by processes such as spray-drying that rapidly bring the drug from the dissolved state to the solid adsorbed state. Such adsorbates have a unique physical structure and have greater physical stability and dissolution performance relative to those made by processes that slowly remove solvent.

Another method to produce solid amorphous adsorbates is a thermal process. Here, the drug is melted and then coated onto the surface of substrates using, for example, a twin-screw extruder. In one exemplary technique the drug is first uniformly blended with the substrate. The blend may be prepared using methods well known in the art for obtaining powdered mixtures with high content uniformity. For example, the drug and substrate may first be independently milled to obtain a small particle size (e.g., less than about 100 μm) and then added to a V blender and blended for 20 minutes. This blend may then be milled to break up any agglomerates, and then blended in a V blender for an additional period of time to obtain a uniform preblend of drug and substrate.

This preblend of drug and substrate is fed into an extruder. By “extruder” is meant a device or collection of devices that creates a molten extrudate by heat and/or shear forces and/or produces a uniformly mixed extrudate. Such devices include, but are not limited to single-screw extruders; twin-screw extruders, including co-rotating, counter-rotating, intermeshing, and non-intermeshing extruders; multiple screw extruders; ram extruders, consisting of a heated cylinder and a piston for extruding the molten extrudate; gear-pump extruders, consisting of a heated gear pump, generally counter-rotating, that simultaneously heats and pumps the molten feed; and conveyer extruders. Conveyer extruders comprise a conveyer means for transporting solid and/or powdered feeds, such, such as a screw conveyer or pneumatic conveyer, and a pump. At least a portion of the conveyer means is heated to a sufficiently high temperature to produce the extrudate. Optionally, an in-line mixer may be used before or after the pump to ensure the extrudate is substantially homogeneous. In each of these extruders the composition is mixed to form a uniformly mixed extrudate. Such mixing may be accomplished by various mechanical and processing means, including mixing elements, kneading elements, and shear mixing by backflow.

In the case of a twin-screw extruder, the screw configuration and mixing paddles are set so as to provide a high degree of fill of the screw sections for efficient heat transfer from the barrel and avoidance of excessive flow restriction. The screw configuration is also selected such that there is sufficient mechanical energy (i.e., shear) to break apart any aggregated substrate still remaining after the preblend step and to uniformly mix the drug and substrates. The barrel temperature should be ramped from approximately room temperature at the feed area to slightly above the melting temperature of the drug in the last barrel zone (discharge end). This technique is applicable for any drug with a melting temperature low enough to melt in the extruder (<400° C.), and for drugs with acceptable chemical stability at the elevated temperatures. Thermal processes such as melt-extrusion processes and equipment are described generally in Encyclopedia of Chemical Technology, 4th Edition (John Wiley & Sons, 1991).

A processing aid may optionally be blended with such drug/substrate mixtures to form a three-component (or more)preblend that is fed to the extruder. One object of such additives is to lower the temperature required for liquefaction of the drug. Thus, the additive typically has a melt point below that of the drug and the drug is typically soluble in the molten additive. The additive may be a volatile material such as water that evaporates from the composition or it may have a high boiling point, such as a mono- or di-glyceride such that it remains part of the composition following processing.

Analogous to the solvent processing method described above, it is preferred to rapidly “quench” the molten material as it exits (is discharged from) the extruder. Any method that results in rapid solidification of the drug as a solid adsorbed layer on the substrate is suitable. Exemplary methods are contact with a cooling fluid such as a cold gas or liquid. Alternatively, the material may enter a cooled mill where heat is transferred from the material at the same time as it is milled into a fine powder with granule sizes from about 100 nm to 100 μm.

Alternatively, a liquid, such as water, can be added to the preblend fed to a twin screw extruder. The screw configuration is designed so that there is sufficient pressure in the extruder to prevent vaporization of the liquid at the temperatures required to melt the drug. When the extrudate exits the extruder, the sudden decrease in pressure causes rapid vaporization of the liquid, leading to rapid cooling and congealing of the adsorbate material. Any residual liquid in the composition can be removed using conventional drying technology such as a tray drier or a fluidized-bed drier.

In another embodiment, the solid amorphous adsorbate comprises a CETP inhibitor absorbed into a water-swellable but insoluble cross-linked polymer. An example of such a solid amorphous adsorbate is disclosed in U.S. Pat. No. 5,569,469, the disclosure of which is incorporated by reference. The drug may be incorporated into a water-swellable but water-insoluble crosslinked polymer (or mixture of two or more such polymers) by any known method such as any of the following:

(a) the drug is dissolved in a suitable solvent and a certain volume of the solution is sprayed onto a given quantity of polymer with the weight ratio of solution to polymer being chosen on the basis of the polymer swelling capacity and on the basis of the concentration of the drug in the solution. The spraying can be carried out in any apparatus used for that purpose, such as in a continuously stirred reactor, in a rotary evaporator under continuous rotation, in a vacuum granulator under constant mixing, in a mortar under light mixing with a pestle, or in a fluidized bed with the polymer kept suspended in an air stream. The product obtained is then dried in the aforesaid apparatuses or in other suitable apparatuses.

(b) the drug is dissolved in a suitable solvent and a quantity of a water-swellable but water-insoluble crosslinked polymer (or a mixture of two or more such polymers) is suspended in an excess of the solution obtained. The suspension is kept stirred until the polymer particles swell. The suspension is then filtered or separated by other suitable means and the product is recovered and dried.

(c) the drug in powder form and the water-swellable but water-insoluble crosslinked polymer (or mixture of two or more such polymers) in powder form are homogeneously mixed together and then ground together in a suitable apparatus such as a ball mill, high-energy vibratory mill, air jet mill etc.

(d) the drug in powder form and the water-swellable but water-insoluble crosslinked polymer (or mixture of two or more such polymers) in powder form are mixed homogeneously and then heated together to the drug melting point in an apparatus such as an oven, rotary evaporator, reaction vessel, oil bath etc. until the drug has melted and has been absorbed by the polymer.

The weight ratio of the drug to water-swellable but water-insoluble crosslinked polymer (or mixture of two or more polymers) is preferably between 0.1 and 1000 parts by weight of drug per 100 parts by weight of polymer and preferably between 10 and 100 parts by weight of drug per 100 parts by weight of polymer.

Examples of water-swellable but water-insoluble crosslinked polymers suitable for use as the substrate (singly or in combinations of two or more than two) are: crosslinked polyvinylpyrrolidone (also known as crospovidone); crosslinked sodium carboxymethylcellulose; crosslinked β-cyclodextrin polymer; and crosslinked dextran. Other polymers suitable to form the crosslinked polymer should have a hydrophilic polymer lattice allowing high swellability in water, and a water insolubility as determined by the nature of the polymer lattice.

Another embodiment of this drug form can be found in U.S. Pat. No. 4,769,236, herein incorporated by reference. In general, this embodiment is obtained by spray-drying the amorphous form of the drug in the presence of a stabilizer and an agent that inhibits crystal formation. The resulting drug form is absorbed onto a crosslinked polymer to prevent recrystalization.

Other embodiments of the drug form can be found in U.S. Pat. Nos. 5,008,114, 5,225,192, 5,275,824, 5,354,560, 5,449,521, and 5,569,469, all of which are hereby incorporated by reference.

Preferably, the solid amorphous adsorbates of the present invention are made by any process that rapidly solidifies (that is, quenches) the material by solvent removal, precipitation with a nonsolvent, cooling, or other means. Such materials, termed “rapidly quenched solid amorphous adsorbates,” have superior properties to adsorbates made by other methods.

In particular, when such “rapidly quenched adsorbates” are delivered to an aqueous use environment, they provide enhanced drug concentrations as described herein. Specifically, such rapidly quenched adsorbates provide a higher maximum free drug concentration or a higher maximum total dissolved drug concentration than that provided by a control, termed a “slow-evaporation control composition,” formed by evaporating the solvent from a suspension of the same substrate in a solution of drug over a period of 30 minutes or more.

Such rapidly quenched adsorbates may also show improved physical stability, slower crystallization rates and superior thermal properties relative to the slow-evaporation control composition.

The solid amorphous adsorbates are typically agglomerates of particles, the agglomerates having a mean diameter ranging from 10 nm to 100 μm. The agglomerates typically retain the fine particulate nature of the starting substrate. In the case of high surface area silicon dioxide substrates, these consist of branched chains composed of many particles with mean diameters of about 10 to 30 nm, or agglomerates of very small spheres (<10 μm).

For adsorbates in which the substrate has a surface area of approximately 200 m²/g, it is believed that for low drug loadings (under about 12 wt %), the drug is present primarily as drug molecules directly adsorbed onto the substrate surface. For such high surface area substrates, there is sufficient surface area for all drug to be directly adsorbed to the substrate up to a drug-to-substrate weight ratio of about 8. Drug adsorbed onto such substrates can be considered a mono layer. Drug adsorbed in this way is noncrystalline and thus may be considered amorphous. However, the interaction of the drug and substrate surface give the drug substantially different physical properties than bulk amorphous drug alone. At greater drug loadings in the adsorbate, it is believed that the drug forms additional layers of amorphous drug on top of the initial monolayer. While not wishing to be bound by any particular theory, it is believed that the interaction of the thin layer(s) of the drug with the substrate improves the physical stability of the drug by decreasing the mobility of the drug on the substrate relative to the mobility of drug in a bulk amorphous material. This may result in improved physical stability by hindering diffusion of drug, and thus inhibiting crystal formation. In addition, as discussed above, if the thickness of the amorphous layer is less than the critical thickness, the amorphous drug on the substrate will be physically stable. The critical thickness is inversely related to the melting point of the CETP inhibitor.

As the surface area of the substrate increases, the amount of drug that can be incorporated into the adsorbate while maintaining a monolayer (or less) of drug also increases. For example, if the substrate has a surface area of 400 m²/g, the drug loading that leads to a monolayer is approximately 21 wt %, while if the substrate has a surface area of 600 m²/g, the drug loading can be about 29% while maintaining a monolayer of drug on the substrate. Thus, it is desirable to use a substrate with as high a surface area as possible to obtain high drug loadings. Such values for the relationship of “drug loading” to substrate surface area are only approximate and depend on the specific size, shape, and orientation of each specific drug.

As indicated above, the solid amorphous adsorbates of the present invention provide concentration enhancement of the CETP inhibitor in an aqueous environment of use. One reason for this concentration enhancement is that solid amorphous adsorbates provide a faster dissolution rate of the drug from the adsorbate than the dissolution rate of particles of crystalline or amorphous drug. This faster dissolution rate results in an increased area under the concentration versus time curve in the use environment, leading to improved bioavailability.

Without wishing to be bound by any particular theory or mechanism of action, it is believed that one reason for the low oral bioavailabilities of many CETP inhibitors, and in particular, hydrophobic CETP inhibitors, is that they have very low dissolution rates in the GI tract. The rate of dissolution of crystalline drug or small particles of amorphous drug is related to the surface area of the drug-containing particle and to the concentration driving force for dissolution, specifically, the difference between the solubility of the solid form of the CETP inhibitor in the aqueous use environment and the bulk solution. The low dissolution rate of CETP inhibitors is believed to be caused by (1) the low solubility of the CETP inhibitors, which results in a very low driving force for dissolution, and (2) the small surface area of the drug-containing particles. While the dissolution rate of a CETP inhibitor can be increased by decreasing the size of the particle, for example, by jet milling the drug particle, the dissolution rate is typically still too low to achieve high bioavailability.

In contrast, the inventors have discovered that the dissolution rates of the solid amorphous adsorbates are much higher than that of crystalline drug or small particles of amorphous drug. The inventors believe that this faster dissolution rate is due in part to the high solubility of the amorphous drug in the adsorbate, but primarily due to the extremely high surface areas achievable with the solid amorphous adsorbates, in some cases about 200 m²/g or more. It is believed that for CETP inhibitors with low solubility, high bioavailability can be achieved by using a solid amorphous adsorbate with a high dissolution rate.

The dissolution rate of a solid amorphous adsorbate is characterized by a first order “dissolution rate constant,” k, obtained by fitting the concentration-versus-time data obtained in the in vitro test previously described to the following first-order exponential equation:

[D]ₜ=[D]ₒ(1−e^{-kt})

where [D]ₜ is the concentration of drug dissolved at any time t, and [D]ₒ is the solubility of the drug in adsorbate form. Although this equation assumes that at long times [D], will approach the solubility of the drug in adsorbate form ([D]ₒ) in practice, the concentration of drug will often reach a maximum value and then start to decrease. This decrease is generally due to the drug beginning to precipitate as a lower solubility form (such as crystalline drug). In such cases, only the upward part of the curve (that is, where

$$\frac{ⅆ {[ D ]}_{t}}{ⅆ t}$$

is positive) is fit to determine the value of k. The dissolution rate constant, k, is typically reported in units of min⁻¹. The inventors have found that there is often a correlation between the dissolution rate constant and the bioavailability of a solid amorphous adsorbate for low-solubility CETP inhibitors. In general, the higher the dissolution rate constant (that is, the faster the dissolution rate), the higher the oral bioavailability will be until the dissolution rate is no longer rate limiting. Thus, in a preferred embodiment the dissolution rate constant for the solid amorphous adsorbate is at least about 0.005 min⁻¹, preferably at least about 0.01 min⁻¹, and most preferably at least about 0.02 min⁻¹. The dissolution rate constant is measured by conducting an in vitro dissolution test as described above with a sufficient amount of adsorbate so that the concentration of CETP inhibitor, if all of the drug dissolved, is at least about 50 μg/ml (where the CETP inhibitor has a solubility of less than 10 μg/ml).

Generally, the dissolution rate constant increases with (1) decreasing drug loading on the substrate, (2) decreasing particle size of the solid amorphous adsorbate, and (3) increasing surface area of the substrate. Thus, to achieve a high dissolution rate, it is preferred that the solid amorphous adsorbate have (1) a low drug loading, generally about 60 wt % or less, preferably about 50 wt % or less; (2) a small particle size, generally less than about 100 μm, preferably less than about 10 μm, and more preferably less than about 1 μm; and (3) a high surface area, preferably about 20 m²/g or greater, more preferably about 50 m²/g or greater, even more preferably about 100 m²/g or greater, and most preferably about 180 m²/g or greater.

The inventors have also found that a dissolution-enhancing agent may be included in the solid amorphous adsorbate to increase the dissolution rate constant. Generally, a dissolution-enhancing agent is a material that, when present in the solid amorphous adsorbate, increases the rate of dissolution of drug relative to an adsorbate that does not include the agent. The dissolution-enhancing agent is preferably water soluble. Exemplary dissolution-enhancing agents include polymers, such as polyvinylpyrrolidone, poloxamers (also known as polyoxyethylene-polyoxypropylene copolymers), polyethylene glycols with molecular weights of less than about 10,000 daltons, polyoxyethylene sorbitan fatty acid esters, polyoxyethylene stearates, polyvinylalcohol; surfactants, such as sodium lauryl sulfate; and phospholipids, such as egg lecithin, soybean lecithin, vegetable lecithin, and 1,2-diacyl-sn-glycero-3-phosphocholines, such as 1-palmitoyl-2-oleyl-sn-glycero-3-phosphocoline, 1,2-dipalmitoyl-sn-glycero-3-phosphocholine, 1,2-distearoyl-sn-glycero-3-phosphocholine, 1-plamitoyl-2-stearoyl-sn-glycero-3-phosphocholine, and other natural or synthetic phosphatidyl cholines. Preferred dissolution-enhancing agents include polyvinylpyrrolidone (PVP) and poloxamers.

The dissolution-enhancing agent is preferably co-adsorbed onto the substrate with the CETP inhibitor. This can be accomplished by any method that results in a thin layer of amorphous drug and dissolution-enhancing agent adsorbed onto the surface of the substrate. One method is to use a solvent process as described above. In that case, the dissolution-enhancing agent and CETP inhibitor are dissolved in a common solvent to which the substrate had been added. By “common solvent” is meant a solvent capable of dissolving both the drug and the dissolution-enhancing agent.

The solid amorphous adsorbate may also include optional additional components, in addition to the processing aids described above, such as surfactants, pH modifiers, disintegrants, binders, lubricants, etc. These materials may help improve processing, performance, or help in preparing dosage forms containing the adsorbates, as discussed below.

A particularly preferred optional additional component is a concentration-enhancing polymer. While the solid amorphous adsorbate provides enhanced concentration of drug in a use environment relative to crystalline drug alone, the inclusion of a concentration-enhancing polymer in the adsorbate may improve the observed enhancement and/or allow for sustaining the enhanced concentration for a longer period of time.

The compositions of the present invention containing concentration-enhancing polymers may be prepared through a variety of methods. The concentration-enhancing polymer may be co-adsorbed onto the substrate with the drug. Alternatively, the concentration-enhancing polymer may be combined with the drug/substrate adsorbate in a mixture.

In one preferred method for combining the solid amorphous adsorbate and concentration-enhancing polymer, the concentration-enhancing polymer is co-adsorbed with the drug onto the substrate. The concentration-enhancing polymer may be co-adsorbed with the drug on the substrate using any method that results in a thin layer of amorphous drug and polymer adsorbed onto the surface of the substrate. The layer may range in thickness from a complete or discontinuous layer of drug and polymer molecules adsorbed directly to the substrate surface, up to a layer of drug and polymer up to a thickness of about the size of 5 to 10 polymer or drug molecules. At least a major portion of the drug present in the adsorbate is amorphous. Preferably, the drug in the adsorbate is substantially amorphous, and more preferably, the drug is almost completely amorphous. While the drug and polymer adsorbed onto the substrate may have drug-rich domains and polymer-rich domains, in one embodiment the drug and polymer are in the form of a solid dispersion adsorbed to the substrate. Preferably, the dispersion is substantially homogeneous, meaning that the amount of the drug present in drug-rich amorphous domains within the dispersion is less than 20%. Often, for such materials the dispersion is “completely homogeneous,” meaning that the amount of drug in drug-rich domains is less than 10%.

One method for adsorbing the concentration-enhancing polymer onto the substrate with the drug is to form the adsorbate using a solvent process as described above. In that case, the concentration-enhancing polymer and drug are dissolved in a common solvent to which the substrate had been added. By “common solvent” is meant a solvent capable of dissolving both the drug and the concentration-enhancing polymer.

In one exemplary method, the substrate is first added to the common solvent and sonicated. The concentration-enhancing polymer is then added to the solution and dissolved. The drug is then added to the solvent and dissolved. The solvent is then rapidly removed from the resulting solution of dissolved drug, dissolved polymer and suspended substrate. The resulting particles of adsorbate are then collected and dried.

An alternative method to co-adsorb drug and polymer onto a substrate is using a thermal process as described above. In one exemplary method, drug, concentration-enhancing polymer, and substrate are preblended and fed to an extruder. The extruder is designed to melt the drug and polymer, resulting in adsorption onto the substrate. The composition is then rapidly cooled to form a rapidly quenched adsorbate, as described above. Additives, such as water, solvents, low-melting-point solids, or plasticizers may be added to the preblend to reduce the melting point of the polymer and allow for lower processing temperatures.

The resulting drug/polymer/substrate adsorbates may comprise from 2 wt % to 90 wt % drug, from 2 to 90 wt % substrate, and from 5 wt % to 95 wt % concentration-enhancing polymer. The mean diameter of the drug/polymer/substrate adsorbates ranges from 10 nm to 100 μm, and the adsorbates are typically agglomerates of particles having mean diameters of 10 nm to 50 nm.

### Concentration-Enhancing Polymers

Concentration-enhancing polymers suitable for use in the various aspects of the present invention should be pharmaceutically acceptable, and should have at least some solubility in aqueous solution at physiologically relevant pHs (e.g. 1-8). Almost any neutral or ionizable polymer that has an aqueous-solubility of at least 0.1 mg/mL over at least a portion of the pH range of 1-8 may be suitable.

It is preferred that the concentration-enhancing polymers be “amphiphilic” in nature, meaning that the polymer has hydrophobic and hydrophilic portions. Amphiphilic polymers are preferred because it is believed that such polymers tend to have relatively strong interactions with the drug and may promote the formation of various types of polymer/drug assemblies in solution. A particularly preferred class of amphiphilic polymers are those that are ionizable, the ionizable portions of such polymers, when ionized, constituting at least a portion of the hydrophilic portions of the polymer. For example, while not wishing to be bound by a particular theory, such polymer/drug assemblies may comprise hydrophobic drug clusters surrounded by the concentration-enhancing polymer with the polymer's hydrophobic regions turned inward towards the drug and the hydrophilic regions of the polymer turned outward toward the aqueous environment. Alternatively, depending on the specific chemical nature of the drug, the ionized functional groups of the polymer may associate, for example, via ion pairing or hydrogen bonds, with ionic or polar groups of the drug. In the case of ionizable polymers, the hydrophilic regions of the polymer would include the ionized functional groups. In addition, the repulsion of the like charges of the ionized groups of such polymers (where the polymer is ionizable) may serve to limit the size of the polymer/drug assemblies to the nanometer or submicron scale. Such drug/concentration-enhancing polymer assemblies in solution may well resemble charged polymeric micellar-like structures. In any case, regardless of the mechanism of action, the inventors have observed that such amphiphilic polymers, particularly ionizable cellulosic polymers such as those listed below, have been shown to interact with drug so as to maintain a higher concentration of drug in an aqueous use environment.

One class of polymers suitable for use with the present invention comprises neutral non-cellulosic polymers. Exemplary polymers include: vinyl polymers and copolymers having at least one substituent selected from the group comprising hydroxyl, alkylacyloxy, and cyclicamido; vinyl copolymers of at least one hydrophilic, hydroxyl-containing repeat unit and at least one hydrophobic, alkyl- or aryl-containing repeat unit; polyvinyl alcohols that have at least a portion of their repeat units in the unhydrolyzed (vinyl acetate) form; polyvinyl alcohol polyvinyl acetate copolymers; polyvinyl pyrrolidone; polyethylene polyvinyl alcohol copolymers, and polyoxyethylene-polyoxypropylene block copolymers (also referred to as poloxamers).

Another class of polymers suitable for use with the present invention comprises ionizable non-cellulosic polymers. Exemplary polymers include: carboxylic acid-functionalized vinyl polymers, such as the carboxylic acid functionalized polymethacrylates and carboxylic acid functionalized polyacrylates such as the EUDRAGITS® manufactured by Rohm Tech Inc., of Malden, Mass.; amine-functionalized polyacrylates and polymethacrylates; high molecular weight proteins such as gelatin and albumin; and carboxylic acid functionalized starches such as starch glycolate.

Non-cellulosic polymers that are amphiphilic are copolymers of a relatively hydrophilic and a relatively hydrophobic monomer. Examples include acrylate and methacrylate copolymers. Exemplary commercial grades of such copolymers include the EUDRAGITS, which are copolymers of methacrylates and acrylates.

A preferred class of polymers comprises ionizable and neutral (or non-ionizable) cellulosic polymers. By “cellulosic” is meant a cellulose polymer that has been modified by reaction of at least a portion of the hydroxyl groups on the saccharide repeat units with a compound to form an ester or an ether substituent. Preferably, the cellulosic polymer has at least one ester- and/or ether- linked substituent in which the polymer has a degree of substitution of at least 0.05 for each substituent. It should be noted that in the polymer nomenclature used herein, ether-linked substituents are recited prior to “cellulose” as the moiety attached to the ether group; for example, “ethylbenzoic acid cellulose” has ethoxybenzoic acid substituents. Analogously, ester-linked substituents are recited after “cellulose” as the carboxylate; for example, “cellulose phthalate” has one carboxylic acid of each phthalate moiety ester-linked to the polymer and the other carboxylic acid unreacted.

It should also be noted that a polymer name such as “cellulose acetate phthalate” (CAP) refers to any of the family of cellulosic polymers that have acetate and phthalate substituents attached via ester linkages to a significant fraction of the cellulosic polymer's hydroxyl groups. Generally, the degree of substitution of each substituent can range from 0.05 to 2.9 as long as the other criteria of the polymer are met. “Degree of substitution” refers to the average number of the three hydroxyls per saccharide repeat unit on the cellulose chain that have been substituted. For example, if all of the hydroxyls on the cellulose chain have been phthalate substituted, the phthalate degree of substitution is 3. Also included within each polymer family type are cellulosic polymers that have additional substituents added in relatively small amounts that do not substantially alter the performance of the polymer.

Amphiphilic cellulosics comprise polymers in which the parent cellulose polymer has been substituted at any or all of the 3 hydroxyl groups present on each saccharide repeat unit with at least one relatively hydrophobic substituent. Hydrophobic substituents may be essentially any substituent that, if substituted to a high enough level or degree of substitution, can render the cellulosic polymer essentially aqueous insoluble. Examples of hydrophobic substituents include ether-linked alkyl groups such as methyl, ethyl, propyl, butyl, etc.; or ester-linked alkyl groups such as acetate, propionate, butyrate, etc.; and ether- and/or ester-linked aryl groups such as phenyl, benzoate, or phenylate. Hydrophilic regions of the polymer can be either those portions that are relatively unsubstituted, since the unsubstituted hydroxyls are themselves relatively hydrophilic, or those regions that are substituted with hydrophilic substituents. Hydrophilic substituents include ether- or ester-linked nonionizable groups such as the hydroxy alkyl substituents hydroxyethyl, hydroxypropyl, and the alkyl ether groups such as ethoxyethoxy or methoxyethoxy. Particularly preferred hydrophilic substituents are those that are ether- or ester-linked ionizable groups such as carboxylic acids, thiocarboxylic acids, substituted phenoxy groups, amines, phosphates or sulfonates.

One class of cellulosic polymers comprises neutral polymers, meaning that the polymers are substantially non-ionizable in aqueous solution. Such polymers contain non-ionizable substituents, which may be either ether-linked or ester-linked. Exemplary ether-linked non-ionizable substituents include: alkyl groups, such as methyl, ethyl, propyl, butyl, etc.; hydroxy alkyl groups such as hydroxymethyl, hydroxyethyl, hydroxypropyl, etc.; and aryl groups such as phenyl. Exemplary ester-linked non-ionizable substituents include: alkyl groups, such as acetate, propionate, butyrate, etc.; and aryl groups such as phenylate. However, when aryl groups are included, the polymer may need to include a sufficient amount of a hydrophilic substituent so that the polymer has at least some water solubility at any physiologically relevant pH of from 1 to 8.

Exemplary non-ionizable cellulosic polymers that may be used as the polymer include: hydroxypropyl methyl cellulose acetate, hydroxypropyl methyl cellulose, hydroxypropyl cellulose, methyl cellulose, hydroxyethyl methyl cellulose, hydroxyethyl cellulose acetate, and hydroxyethyl ethyl cellulose.

A preferred set of non-ionizable (neutral) cellulosic polymers are those that are amphiphilic. Exemplary polymers include hydroxypropyl methyl cellulose and hydroxypropyl cellulose acetate, where cellulosic repeat units that have relatively high numbers of methyl or acetate substituents relative to the unsubstituted hydroxyl or hydroxypropyl substituents constitute hydrophobic regions relative to other repeat units on the polymer.

A preferred class of cellulosic polymers comprises polymers that are at least partially ionizable at physiologically relevant pH and include at least one ionizable substituent, which may be either ether-linked or ester-linked. Exemplary ether-linked ionizable substituents include: carboxylic acids, such as acetic acid, propionic acid, benzoic acid, salicylic acid, alkoxybenzoic acids such as ethoxybenzoic acid or propoxybenzoic acid, the various isomers of alkoxyphthalic acid such as ethoxyphthalic acid and ethoxyisophthalic acid, the various isomers of alkoxynicotinic acid such as ethoxynicotinic acid, and the various isomers of picolinic acid such as ethoxypicolinic acid, etc.; thiocarboxylic acids, such as thioacetic acid; substituted phenoxy groups, such as hydroxyphenoxy, etc.; amines, such as aminoethoxy, diethylaminoethoxy, trimethylaminoethoxy, etc.; phosphates, such as phosphate ethoxy; and sulfonates, such as sulphonate ethoxy. Exemplary ester linked ionizable substituents include: carboxylic acids, such as succinate, citrate, phthalate, terephthalate, isophthalate, trimellitate, and the various isomers of pyridinedicarboxylic acid, etc.; thiocarboxylic acids, such as thiosuccinate; substituted phenoxy groups, such as amino salicylic acid; amines, such as natural or synthetic amino acids, such as alanine or phenylalanine; phosphates, such as acetyl phosphate; and sulfonates, such as acetyl sulfonate. For aromatic-substituted polymers to also have the requisite aqueous solubility, it is also desirable that sufficient hydrophilic groups such as hydroxypropyl or carboxylic acid functional groups be attached to the polymer to render the polymer aqueous soluble at least at pH values where any ionizable groups are ionized. In some cases, the aromatic substituent may itself be ionizable, such as phthalate or trimellitate substituents.

Exemplary cellulosic polymers that are at least partially ionized at physiologically relevant pHs include: hydroxypropyl methyl cellulose acetate succinate (HPMCAS), hydroxypropyl methyl cellulose succinate, hydroxypropyl cellulose acetate succinate, hydroxyethyl methyl cellulose succinate, hydroxyethyl cellulose acetate succinate, hydroxypropyl methyl cellulose phthalate (HPMCP), hydroxyethyl methyl cellulose acetate succinate, hydroxyethyl methyl cellulose acetate phthalate, carboxyethyl cellulose, ethylcarboxymethyl cellulose (also referred to as carboxymethylethyl cellulose or CMEC), carboxymethyl cellulose, cellulose acetate phthalate (CAP), methyl cellulose acetate phthalate, ethyl cellulose acetate phthalate, hydroxypropyl cellulose acetate phthalate, hydroxypropyl methyl cellulose acetate phthalate, hydroxypropyl cellulose acetate phthalate succinate, hydroxypropyl methyl cellulose acetate succinate phthalate, hydroxypropyl methyl cellulose succinate phthalate, cellulose propionate phthalate, hydroxypropyl cellulose butyrate phthalate, cellulose acetate trimellitate (CAT), methyl cellulose acetate trimellitate, ethyl cellulose acetate trimellitate, hydroxypropyl cellulose acetate trimellitate, hydroxypropyl methyl cellulose acetate trimellitate, hydroxypropyl cellulose acetate trimellitate succinate, cellulose propionate trimellitate, cellulose butyrate trimellitate, cellulose acetate terephthalate, cellulose acetate isophthalate, cellulose acetate pyridinedicarboxylate, salicylic acid cellulose acetate, hydroxypropyl salicylic acid cellulose acetate, ethylbenzoic acid cellulose acetate, hydroxypropyl ethylbenzoic acid cellulose acetate, ethyl phthalic acid cellulose acetate, ethyl nicotinic acid cellulose acetate, and ethyl picolinic acid cellulose acetate. Of these cellulosic polymers that are at least partially ionized at physiologically relevant pHs, those that the inventors have found to be most preferred are HPMCAS, HPMCP, CAP, CAT, carboxyethyl cellulose, carboxymethyl cellulose, and CMEC.

One class of concentration-enhancing polymers is acidic polymers. By “acidic polymer” is meant any polymer that possesses a significant number of acidic moieties. In general, a significant number of acidic moieties would be greater than or equal to about 0.1 milliequivalents of acidic moieties per gram of polymer. “Acidic moieties” include any functional groups that are sufficiently acidic that, in contact with or dissolved in water, can at least partially donate a hydrogen cation to water and thus increase the hydrogen-ion concentration. This definition includes any functional group or “substituent,” as it is termed when the functional group is covalently attached to a polymer that has a pKa of less than about 10. Here, the term pKₐ is used in its traditional form, the pKₐ being the negative logarithm of the acid ionization constant. The pKₐ will be influenced by such factors as solvent, temperature, water content, and ionic strength of the media or matrix in which the acid resides. Unless otherwise noted, the pKₐ is assumed to be measured in distilled water at 25° C. Preferably, the pKₐ of the functional groups on the polymer are less than about 7, and even more preferably less than about 6. Exemplary classes of functional groups that are included in the above description include carboxylic acids, thiocarboxylic acids, phosphates, phenolic groups, and sulfonates. Such functional groups may make up the primary structure of the polymer such as for polyacrylic acid, but more generally are covalently attached to the backbone of the parent polymer and thus are termed “substituents.” A preferred set of acidic polymers that are at least partially ionized at physiologically relevant pHs, include hydroxypropyl methyl cellulose acetate succinate, hydroxypropyl methyl cellulose phthalate, cellulose acetate phthalate, cellulose acetate trimellitate and carboxymethyl ethyl cellulose. The most preferred is hydroxypropyl methyl cellulose acetate succinate (HPMCAS).

Another preferred class of polymers consists of neutralized acidic polymers. By “neutralized acidic polymer” is meant any acidic polymer for which a significant fraction of the “acidic moieties” or “acidic substituents” have been “neutralized“; that is, exist in their deprotonated form. Neutralized acidic polymers are described in more detail in commonly assigned U.S. patent application U.S. Ser. No. 10/175,566 entitled “Pharmaceutical Compositions of Drugs and Neutralized Acidic Polymers” filed Jun. 17, 2002, the relevant disclosure of which is incorporated by reference.

While specific polymers have been discussed as being suitable for use in the compositions of the present invention, blends of such polymers may also be suitable. Thus the term “concentration-enhancing polymer” is intended to include blends of polymers in addition to a single species of polymer.

### HMG-CoA Reductase Inhibitors

The HMG-CoA reductase inhibitor may be any HMG-CoA reductase inhibitor capable of lower plasma concentrations of low-density lipoprotein, total cholesterol, or both. In one aspect, the HMG-CoA reductase inhibitor is from a class of therapeutics commonly called statins. Examples of HMG-CoA reductase inhibitors that may be used include but are not limited to lovastatin (MEVACOR®; see U.S. Pat. Nos. 4,231,938; 4,294,926; 4,319,039), simvastatin (ZOCOR®; see U.S. Pat. Nos. 4,444,784; 4,450,171, 4,820,850; 4,916,239), pravastatin (PRAVACHOL®; see U.S. Pat. Nos. 4,346,227; 4,537,859; 4,410,629; 5,030,447 and 5,180,589), lactones of pravastatin (see U.S. Pat. No. 4,448,979), fluvastatin (LESCOL®; see U.S. Pat. Nos. 5,354,772; 4,911,165; 4,739,073; 4,929,437; 5,189,164; 5,118,853; 5,290,946; 5,356,896), lactones of fluvastatin, atorvastatin (LIPITOR®; see U.S. Pat. Nos. 5,273,995; 4,681,893; 5,489,691; 5,342,952), lactones of atorvastatin, cerivastatin (also known as rivastatin and BAYCHOL®; see U.S. Pat. No. 5,177,080, and European Application No. EP-491226A), lactones of cerivastatin, rosuvastatin (Crestor®; see U.S. Pat. Nos. 5,260,440 and RE37314, and European Patent No. EP521471), lactones of rosuvastatin, itavastatin, nisvastatin, visastatin, atavastatin, bervastatin, compactin, dihydrocompactin, dalvastatin, fluindostatin, pitivastatin, mevastatin (see U.S. Pat. No. 3,983,140), and velostatin (also referred to as synvinolin). Other examples of HMG-CoA reductase inhibitors are described in U.S. Pat. Nos. 5,217,992; 5,196,440; 5,189,180; 5,166,364; 5,157,134; 5,110,940; 5,106,992; 5,099,035; 5,081,136; 5,049,696; 5,049,577; 5,025,017; 5,011,947; 5,010,105; 4,970,221; 4,940,800; 4,866,058; 4,686,237; 4,647,576; European Application Nos. 0142146A2 and 0221025A1; and PCT Application Nos. WO 86/03488 and WO 86/07054. Also included are pharmaceutically acceptable forms of the above. All of the above references are incorporated herein by reference. Preferably the HMG-CoA reductase inhibitor is selected from the group consisting of fluvastatin, lovastatin, pravastatin, atorvastatin, simvastatin, cerivastatin, rivastatin, mevastatin, velostatin, compactin, dalvastatin, fluindostatin, rosuvastatin, pitivastatin, dihydrocompactin, and pharmaceutically acceptable forms thereof. By “pharmaceutically acceptable forms” is meant any pharmaceutically acceptable derivative or variation, including stereoisomers, stereoisomer mixtures, enantiomers, solvates, hydrates, isomorphs, polymorphs, pseudomorphs, salt forms and prodrugs.

In one embodiment, the HMG-CoA reductase inhibitor is selected from the group consisting of trans-6-[2-(3 or 4-carboxamido-substituted pyrrol-1-yl)alkyl]-4-hydroxypyran-2-ones and corresponding pyran ring-opened hydroxy acids derived therefrom. These compounds have been described in U.S. Pat. No. 4,681,893, which is herewith incorporated by reference in the present specification. The pyran ring-opened hydroxy acids that are intermediates in the synthesis of the lactone compounds can be used as free acids or as pharmaceutically acceptable metal or amine salts. In particular, these compounds can be represented by the following structure:

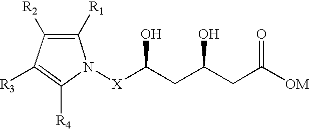

wherein X is —CH₂—, —CH₂CH₂—, —CH₂CH₂CH₂— or —CH₂CH(CH₃)—; R₁ is 1-naphthyl; 2-naphthyl; cyclohexyl, norbornenyl; 2-,3-, or 4-pyridinyl; phenyl; phenyl substituted with fluorine, chlorine, bromine, hydroxyl, trifluoromethyl, alkyl of from one to four carbon atoms, alkoxy of from one to four carbon atoms, or alkanoylalkoxy of from two to eight carbon atoms; either R₂ or R₃ is —CONR₅R₆ where R₅ and R₆ are independently hydrogen; alkyl of from one to six carbon atoms; 2-,3-, or 4-pyridinyl; phenyl; phenyl substituted with fluorine, chlorine, bromine, cyano, trifluoromethyl, or carboalkoxy of from three to eight carbon atoms; and the other of R₂ or R₃ is hydrogen; alkyl of from one to six carbon atoms; cyclopropyl; cyclobutyl; cyclopentyl; cyclohexyl; phenyl; or phenyl substituted with fluorine, chlorine, bromine, hydroxyl, trifluoromethyl, alkyl of from one to four carbon atoms, alkoxy of from one to four carbon atoms, or alkanoyloxy of from two to eight carbon atoms; R₄ is alkyl of from one to six carbon atoms; cyclopropyl; cyclobutyl; cyclopentyl; cyclohexyl; or trifluoromethyl; and M is a pharmaceutically acceptable salt (e.g., counter ion), which includes a pharmaceutically acceptable metal salt or a pharmaceutically acceptable amine salt.

Among the stereo-specific isomers, one preferred HMG-CoA reductase inhibitor is atorvastatin trihydrate hemicalcium salt. This preferred compound is the ring-opened form of (2R-trans)-5-(4-fluorophenyl)-2-(1 methylethyl)-N,4-diphenyl-1-[2-(tetrahydro-4-hydroxy-6-oxo-2H-pyran-2-yl)ethyl]-1H-pyrrole-3-carboxamide, namely, the enantiomer [R-(R*,R*)]-2-(4-fluorophenyl-β,δ-dihydroxy-5-(1-methylethyl)-3-phenyl-4-[(phenylamino)carbonyl)]-1H-pyrrole-1-heptanoic acid hemicalcium salt. Its chemical structure may be represented by the following structure:

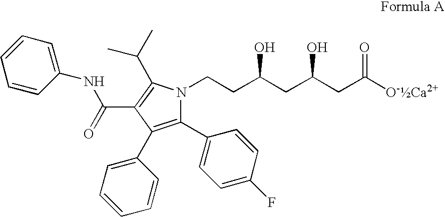

The specific isomer has been described in U.S. Pat. No. 5,273,995, herein incorporated by reference. In a preferred embodiment, the HMG-CoA reductase inhibitor is selected from the group consisting of atorvastatin, the cyclized lactone form of atorvastatin, a 2-hydroxy, 3-hydroxy or 4-hydroxy derivative of such compounds, and a pharmaceutically acceptable forms thereof.

In practice, use of the salt form amounts to use of the acid or lactone form. Appropriate pharmaceutically acceptable salts within the scope of the invention are those derived from bases such as sodium hydroxide, potassium hydroxide, lithium hydroxide, calcium hydroxide, 1-deoxy-2-(methylamino)-D-glucitol, magnesium hydroxide, zinc hydroxide, aluminum hydroxide, ferrous or ferric hydroxide, ammonium hydroxide or organic amines such as N-methylglucamine, choline, arginine and the like. Preferably, the lithium, calcium, magnesium, aluminum and ferrous or ferric salts are prepared from the sodium or potassium salt by adding the appropriate reagent to a solution of the sodium or potassium salt, i.e., addition of calcium chloride to a solution of the sodium or potassium salt of the compound of the formula A will give the calcium salt thereof.

In one embodiment, the HMG-CoA reductase inhibitor is acid-sensitive, meaning that the drug either chemically reacts with or otherwise degrades in the presence of acidic species. Examples of chemical reactions include hydrolysis, lactonization, or transesterification in the presence of acidic species.

### Improved Bioavailability

In one aspect, the compositions of the present invention comprise a solid amorphous adsorbate comprising a CETP inhibitor and a substrate, and an HMG-CoA reductase inhibitor, wherein the CETP inhibitor is present in a sufficient amount such that when the composition is orally administered to an in vivo environment of use it provides at least one of (1) an increase in bioavailability of the HMG-CoA reductase inhibitor relative to a first control composition; (2) an increased maximum drug concentration (Cₘₐₓ) of the HMG-CoA reductase inhibitor in the blood relative to a first control composition; and (3) both (1) and (2). The first control composition consists essentially of the same amount of the HMG-CoA reductase inhibitor but without the CETP inhibitor.

In another aspect, the composition comprises a solid amorphous adsorbate comprising a CETP inhibitor and a substrate and an HMG-CoA reductase inhibitor, wherein the HMG-CoA reductase inhibitor is present in a sufficient amount such that when the composition is orally administered to an in vivo environment of use it provides at least one of (1) an increase in bioavailability of the CETP inhibitor relative to a second control composition; (2) an increased Cₘₐₓ of the CETP inhibitor in the blood relative to a second control composition; and (3) both (1) and (2). The second control composition consists essentially of the same amount of the solid amorphous adsorbate comprising a CETP inhibitor and a substrate but without the HMG-CoA reductase inhibitor.

In yet another aspect, the composition comprises a solid amorphous adsorbate comprising a CETP inhibitor and a substrate and an HMG-CoA reductase inhibitor, wherein the CETP inhibitor is present in a sufficient amount such that when the composition is orally administered to an in vivo environment of use it provides at least one of (1) an increase in bioavailability of the HMG-CoA reductase inhibitor relative to a third control composition; (2) an increased Cₘₐₓ of the HMG-CoA reductase inhibitor in the blood relative to a third control composition; and (3) both (1) and (2). The third control composition consists essentially of the same amount of the HMG-CoA reductase inhibitor and the same amount of the CETP inhibitor, but the CETP inhibitor is not in the form of a solid amorphous adsorbate.

A key to this aspect of the invention is that the CETP inhibitor is in the form of a solid amorphous adsorbate. As described in detail above, the solid amorphous adsorbate comprising a CETP inhibitor and a substrate provides an increased maximum drug concentration (MDC) in an aqueous environment of use relative to a control composition consisting essentially of the CETP inhibitor in unadsorbed form when dosed orally. In vivo, this increased MDC in the GI tract leads to an increased concentration of CETP inhibitor in the blood and an improved area under the concentration versus time curve (AUC) in the blood relative to orally dosing the crystalline control. Thus, when a solid amorphous adsorbate comprising a CETP inhibitor and a substrate is dosed orally to an animal, the concentration of CETP in the GI tract of the animal and in the blood of the animal is improved relative to dosing crystalline drug.

The solid amorphous adsorbate comprising a CETP inhibitor and a substrate results in sufficiently high concentrations of CETP in the GI tract, the epithelial cells of the intestine, or in the blood to achieve a synergistic effect when co-dosed with an HMG-CoA reductase inhibitor. Without wishing to be bound by any theory or mechanism of action, it is believed that the CETP inhibitor may be a substrate for, or may inhibit, P-glycoprotein (PGP), an efflux pump that may slow the rate of absorption of the CETP inhibitor and the HMG-CoA reductase inhibitor. When the CETP inhibitor and HMG-CoA reductase inhibitor are co-dosed, the total amount of CETP inhibitor and HMG-CoA reductase inhibitor that can be effluxed may be reduced relative to dosing of either one individually, resulting in concentration- and bioavailability-enhancement as noted above. Alternatively, the CETP inhibitor may be a substrate or inhibitor for a metabolic enzyme such as the cytochrome P450 3A4 isoenzyme (CYP3A4) that also mediates the metabolism of the HMG-CoA reductase inhibitor. When the CETP inhibitor and HMG-CoA reductase inhibitor are co-administered, the amount of HMG-CoA reductase inhibitor that can be metabolized by CYP3A4 may be reduced, resulting in the observed enhancements. Regardless of the mechanism of action, the compositions of the present invention result in improvements in concentration in the blood or bioavailability as described above.

In addition, the HMG-CoA reductase inhibitor may be a substrate for or inhibit PGP, or a metabolic enzyme, to increase the AUC or Cₘₐₓ of the CETP inhibitor in the blood.

The concentration enhancements in the blood provided by the compositions of the present invention may be tested in vivo in animals or humans using conventional methods for making such a determination. An in vivo test, such as a crossover study, may be used to determine whether a test composition provides enhanced performance compared with the first, second, or third control compositions. In an in vivo crossover study a “test composition” of a solid amorphous adsorbate comprising a CETP inhibitor and a substrate and an HMG-CoA reductase inhibitor is administered to half a group of test subjects and, after an appropriate washout period (e.g., one week) the same subjects are administered a control composition. As described above, the control composition may be either the first control composition, which consists of an equivalent amount of the HMG-CoA reductase inhibitor but without the solid amorphous adsorbate comprising a CETP inhibitor and a substrate, the second control composition, which consists of an equivalent amount of the solid amorphous adsorbate comprising a CETP inhibitor and a substrate but without the HMG-CoA reductase inhibitor, or the third control composition, which consists of an equivalent amount of the HMG-CoA reductase inhibitor and an equivalent amount of the CETP inhibitor, but with the CETP inhibitor not in the form of a solid amorphous adsorbate. The other half of the group is administered the control composition first, followed by the test composition. The concentration of the CETP inhibitor and the HMG-CoA reductase inhibitor in the blood (serum or plasma) is then measured versus time using procedures well known in the art. From these data the maximum concentration of drug in the blood (Cₘₐₓ) and the area under the blood concentration versus time curve (AUC) are determined. The determination of Cₘₐₓ and AUC is a well-known procedure and is described, for example, in Welling, “Pharmacokinetics Processes and Mathematics,” ACS Monograph 185 (1986). Enhancements in Cₘₐₓ and AUC are determined by taking the ratio of the Cₘₐₓ or AUC in the blood for the test group and dividing by the Cₘₐₓ or AUC in the blood for the control group. Preferably, this testcontrol ratio is determined for each subject, and then the ratios are averaged over all subjects in the study.

A preferred embodiment is one in which the compositions of the present invention provide a Cₘₐₓ in the blood for the HMG-CoA reductase inhibitor that is at least 1.25-fold that provided by the first control composition described above. Preferably, the Cₘₐₓ in the blood for the HMG-COA reductase inhibitor is at least 1.5-fold, more preferably at least 2.0-fold that provided by the first control composition.

Another preferred embodiment is one in which the compositions of the present invention provide an AUC in the blood for the HMG-CoA reductase inhibitor that is at least 1.25-fold that provided by the first control composition. Preferably, the AUC in the blood for the HMG-CoA reductase inhibitor is at least 1.5-fold, more preferably at least 2.0-fold that provided by the first control composition. This is the same as saying that the relative bioavailability of the HMG-CoA reductase inhibitor of the composition of the present invention is at least 1.25-fold, preferably at least 1.5-fold, and more preferably at least 2.0-fold relative to the first control composition.

In another separate preferred embodiment, the compositions of the present invention provide a Cₘₐₓ in the blood for the CETP inhibitor that is at least 1.25-fold that provided by the second control composition described above. Preferably, the Cₘₐₓ in the blood for the CETP inhibitor is at least 1.5-fold, more preferably at least 2.0-fold that provided by the second control composition.

In yet another preferred embodiment, the compositions of the present invention provide an AUC in the blood for the CETP inhibitor that is at least 1.25-fold that provided by the second control composition. Preferably, the AUC in the blood for the CETP inhibitor is at least 1.5-fold, more preferably at least 2.0-fold that provided by the second control composition. This is the same as saying that the relative bioavailability of the CETP inhibitor of the composition of the present invention is at least 1.25-fold, preferably at least 1.5-fold, and more preferably at least 2.0-fold relative to the second control composition.

In another separate preferred embodiment, the compositions of the present invention provide a Cₘₐₓ in the blood for the HMG-CoA reductase inhibitor that is at least 1.25-fold that provided by the third control composition described above. Preferably, the Cₘₐₓ in the blood for the HMG-CoA reductase inhibitor is at least 1.5-fold, more preferably at least 2.0-fold that provided by the third control composition.

Another preferred embodiment is one in which the compositions of the present invention provide an AUC in the blood for the HMG-CoA reductase inhibitor that is at least 1.25-fold that provided by the third control composition. Preferably, the AUC in the blood for the HMG-CoA reductase inhibitor is at least 1.5-fold, more preferably at least 2.0-fold that provided by the third control composition. This is the same as saying that the relative bioavailability of the HMG-CoA reductase inhibitor of the composition of the present invention is at least 1.25-fold, preferably at least 1.5-fold, and more preferably at least 2.0-fold relative to the third control composition.

For those embodiments that provide an enhancement in the Cₘₐₓ or bioavailability of the HMG-CoA reductase inhibitor, there must be sufficient CETP inhibitor in the composition to obtain the enhancement. Generally, the greater the amount of CETP inhibitor present in the composition, the greater the enhancement obtained. For example, when the CETP inhibitor is [2R,4S]-4-[(3,5-bis-trifluoromethyl-benzyl)-methoxycarbonyl-amino]-2-ethyl-6-trifluoromethyl-3,4-dihydro-2H-quinoline-1-carboxylic acid ethyl ester (torcetrapib) and the HMG-COA reductase inhibitor is atorvastatin hemicalcium trihydrate, it is preferred that the weight ratio of CETP inhibitor to HMG-CoA reductase inhibitor in the composition be at least about 0.1, more preferably at least about 0.3, and even more preferably at least about 0.5.

For those embodiments that provide an enhancement in the concentration or bioavailability of the CETP inhibitor, there must be sufficient HMG-CoA reductase inhibitor in the composition to obtain the enhancement. Generally, the greater the amount of HMG-COA reductase inhibitor present in the composition, the greater the enhancement obtained. For example, when the CETP inhibitor is torcetrapib and the HMG-CoA reductase inhibitor is atorvastatin hemicalcium trihydrate, it is preferred that the weight ratio of CETP inhibitor to HMG-CoA reductase inhibitor in the composition be no greater than about 36, preferably no greater than about 20, and even more preferably no greater than about 18.

In a specific preferred embodiment, the CETP inhibitor is torcetrapib and the HMG-CoA reductase inhibitor is atorvastatin hemicalcium trihydrate. For these compounds, it is preferred that the weight ratio of CETP inhibitor to HMG-CoA reductase inhibitor in the composition range from about 0.1 to about 36, preferably about 0.3 to about 20, more preferably about 0.5 to about 18.

### Dosage Forms

The compositions of the present invention are generally administered in the form of a pharmaceutical composition comprising at least one of the compounds of this invention together with a pharmaceutically acceptable carrier, vehicle or diluent. Thus, the compounds of this invention can be administered either individually or together in any conventional oral, parenteral or transdermal dosage form.

For oral administration, the composition of the present invention can be formulated into a suitable dosage form, including solutions, suspensions, tablets, pills, capsules, powders, and the like. Tablets containing various excipients such as sodium citrate, calcium carbonate and calcium phosphate are employed along with various disintegrants, together with binding agents such as polyvinylpyrrolidone, sucrose, gelatin and acacia. Additionally, lubricating agents such as magnesium stearate, sodium lauryl sulfate and talc are often very useful for tableting purposes. Solid compositions of a similar type are also employed as fillers in soft and hard-filled gelatin capsules; preferred materials in this connection also include lactose or milk sugar as well as high molecular weight polyethylene glycols. When aqueous suspensions and/or elixirs are desired for oral administration, the compounds of this invention can be combined with various sweetening agents, flavoring agents, coloring agents, emulsifying agents and/or suspending agents, as well as such diluents as water, ethanol, propylene glycol, glycerin and various like combinations thereof.

In one embodiment, the solid amorphous adsorbate comprising a CETP inhibitor and a substrate, and HMG-CoA reductase inhibitor are blended together with optional excipients and then compressed to form the dosage form, such as tablets, caplets, or pills. Virtually any process can be used to blend the materials. For example, the compositions can be blended in rotating shell mixers, fixed-shell mixers, planetary paddle mixers, and twin-shell mixers, all known in the art.

The compressed dosage forms may be formed using any of a wide variety of presses used in the fabrication of pharmaceutical dosage forms. Examples include single-punch presses, rotary tablet presses, and multilayer rotary tablet presses, all well-known in the art. See Remington's Pharmaceutical Sciences (20^{th} Edition, 2000). The compressed dosage form may be of any shape, including round, oval, oblong, cylindrical, or triangular. The upper and lower surfaces of the compressed dosage form may be flat, round, concave, or convex.

The compositions of the present invention can be in the form of a unitary dosage form. By “unitary dosage form” is meant a single dosage form containing both the solid amorphous adsorbate comprising the CETP inhibitor and a substrate and the HMG-CoA reductase inhibitor so that, following administration of the unitary dosage form to a use environment, both the CETP inhibitor and HMG-CoA reductase inhibitor are delivered to the use environment. The term “unitary dosage form” includes a single tablet, caplet, pill, capsule, powder, and the like, as well as a kit comprising one or more tablets, caplets, pills, capsules, sachets, powders, or solutions intended to be taken together.

In one embodiment, the unitary dosage form comprises (1) a CETP inhibitor composition comprising a solid amorphous adsorbate comprising a CETP inhibitor and a substrate, and (2) an HMG-CoA reductase inhibitor composition comprising the HMG-CoA reductase inhibitor. The HMG-CoA reductase inhibitor composition may comprise the HMG-CoA reductase inhibitor alone, or the HMG-CoA reductase inhibitor and optional excipients. The CETP inhibitor composition and the HMG-CoA reductase inhibitor composition may be combined, such as by mixing, granulating, milling, or by other methods known in the art. Alternatively, the two compositions may be associated with each other, meaning the CETP inhibitor composition and the HMG-CoA reductase inhibitor composition may be in separate layers, particles, or granules, in the same dosage form.

In another embodiment, the unitary dosage form comprises (1) a CETP inhibitor composition comprising a solid amorphous adsorbate comprising a CETP inhibitor, an acidic concentration-enhancing polymer, and a substrate, and (2) an HMG-CoA reductase inhibitor composition comprising the HMG-CoA reductase inhibitor. The two compositions are combined such that the solid amorphous adsorbate and the HMG-COA reductase inhibitor are substantially separate from one another in the dosage form. Such unitary dosage forms are disclosed more fully in commonly assigned co-pending Provisional U.S. Patent Application No. 60/435,345, entitled “Dosage Forms Comprising a CETP Inhibitor and an HMG-CoA Reductase Inhibitor,” the disclosure of which is incorporated herein by reference.

By “substantially separate from one another” is meant that a sufficient amount of the HMG-CoA reductase inhibitor is physically separated from the solid amorphous adsorbate so that the acidic concentration-enhancing polymer does not cause an unacceptable level of chemical degradation of the HMG-CoA reductase inhibitor. The HMG-CoA reductase inhibitor thus has improved chemical stability relative to a blended mixture of (1) particles consisting essentially of the solid amorphous adsorbate of the CETP inhibitor, acidic concentration-enhancing polymer, and substrate alone, and (2)particles consisting essentially of the HMG-CoA reductase inhibitor alone. This improved chemical stability of the HMG-CoA reductase inhibitor is believed to be related primarily to reducing the fraction of HMG-CoA reductase inhibitor molecules that are in contact with the solid amorphous adsorbate of CETP inhibitor/acidic concentration-enhancing polymer/substrate. The unitary dosage form limits the fraction of HMG-CoA reductase inhibitor molecules that are in contact with the solid amorphous adsorbate of the CETP inhibitor, acidic concentration-enhancing polymer, and substrate.

For some approaches, the separation is macroscopic in nature; that is, the HMG-CoA reductase inhibitor and the solid amorphous adsorbate may be, for example, in separate layers of the dosage form so that only those HMG-CoA reductase inhibitor molecules present at the interface of the two layers may be in contact with the solid amorphous adsorbate. Further separation between the HMG-CoA reductase inhibitor and the solid amorphous adsorbate may be obtained by providing a third layer that separates the two compositions. Alternatively, the unitary dosage form may be in the form of a kit wherein the HMG-CoA reductase inhibitor and solid amorphous adsorbate are within separate compartments in the dosage form.

For other approaches, the separation is microscopic in nature; that is, the separation may be due to only one or more intervening molecules. For example, the unitary dosage form may comprise the solid amorphous adsorbate and a plurality of relatively large particles or granules comprising the HMG-CoA reductase inhibitor. The HMG-CoA reductase inhibitor molecules located in the interior of the particles or granules are separated from the solid amorphous adsorbate by the molecules on the surface of the particles or granules. Alternatively, the solid amorphous adsorbate may be in the form of relatively large particles or granules, with molecules of the acidic concentration-enhancing polymer in the solid amorphous adsorbate on the interior of the particles of granules being separated from the HMG-CoA reductase inhibitor by the molecules on the surface of the particles or granules. Alternatively, particles or granules of the HMG-CoA reductase inhibitor, particles or granules of the solid amorphous adsorbate, or both may be coated with a protective coating, thus separating the HMG-CoA reductase inhibitor and the solid amorphous adsorbate. In any case, the HMG-CoA reductase inhibitor and the solid amorphous adsorbate are substantially separated from one another so that the acidic concentration-enhancing polymer does not cause an unacceptable level of chemical degradation of the HMG-CoA reductase inhibitor.

When formulated in such manner, the resulting unitary dosage form has improved chemical stability when compared to a control composition where the solid amorphous adsorbate and the HMG-CoA reductase inhibitor are not substantially separate from one another.

In another embodiment, the unitary dosage form comprises (1) a solid amorphous adsorbate comprising a CETP inhibitor, a neutral or neutralized acidic concentration-enhancing polymer, and a substrate, and (2) an HMG-CoA reductase inhibitor. The concentration-enhancing polymer chosen to form the solid amorphous adsorbate should be neutral or a neutralized acidic polymer, so that the concentration-enhancing polymer does not chemically degrade the HMG-CoA reductase inhibitor. The HMG-CoA reductase inhibitor in the resulting unitary dosage form has improved chemical stability when compared to a control dosage form where the concentration-enhancing polymer is an acidic polymer such as hydroxypropyl methyl cellulose acetate succinate (HPMCAS). Such unitary dosage forms are disclosed more fully in commonly assigned co-pending Provisional U.S. Patent Application No. 60/435,298, entitled “Dosage Forms Comprising a CETP Inhibitor and an HMG-CoA Reductase Inhibitor,” the disclosure of which is incorporated herein by reference.

In another embodiment, the solid amorphous adsorbate and the HMG-CoA reductase inhibitor are dissolved or suspended in a liquid or semi-solid vehicle, and encapsulated in a soft or hard gelatin capsule or in a capsule made from some other material, e.g., starch.

In another embodiment, the dosage form may be formed by the following process. First, the HMG-CoA reductase inhibitor may be formed into multiparticulates using processes well known in the art, such as by extrusion spheronization, cryogenic pelletization, spray drying, or melt congealing. See, for example, Remington: The Science and Practice of Pharmacy, 20^{th} Edition (2000). The resulting multiparticulates may then be placed into a capsule along with the solid amorphous adsorbate comprising the CETP inhibitor and substrate. Alternatively, the solid amorphous adsorbate comprising the CETP inhibitor and substrate may first be formed into multiparticulates and placed into a capsule along with the HMG-CoA reductase inhibitor. In another method, the HMG-CoA reductase inhibitor may be formed into multiparticulates and the solid amorphous adsorbate comprising the CETP inhibitor and substrate may be formed into multiparticulates, which are then mixed and placed into a capsule. Alternatively, the multiparticulates may be compressed into a compressed dosage form as previously described.

In addition to the solid amorphous adsorbate and the HMG-CoA reductase inhibitor, dosage forms comprising the compositions of the present invention may include other excipients to aid in formulating the composition into tablets, capsules, suppositories, suspensions, powders for suspension, creams, transdermal patches, depots, and the like. See, for example, Remington: The Science and Practice of Pharmacy (20th ed. 2000)

One very useful class of excipients is disintegrants. The inclusion of a disintegrant into the dosage form promotes rapid dissolution of the dosage form when introduced into an aqueous use environment. Examples of disintegrants include sodium starch glycolate, sodium carboxymethyl cellulose, calcium carboxymethyl cellulose, croscarmellose sodium, crospovidone, polyvinylpolypyrrolidone, methyl cellulose, microcrystalline cellulose, powdered cellulose, lower alkyl-substituted hydroxypropyl cellulose, polacrilin potassium, starch, pregelatinized starch, sodium alginate, and mixtures thereof. Of these, crospovidone, croscarmellose sodium, lower alkyl-substituted hydroxypropyl cellulose, methyl cellulose, polacrilin potassium, and mixtures thereof are preferred.

The dosage forms may also include a porosigen. A “porosigen” is a material that leads to a high porosity and high strength following compression of the blend into a tablet or other compressed dosage form known in the art. In addition, preferred porosigens are soluble in an acidic environment with aqueous solubilities typically greater than 1 mg/mL at a pH less than about 4. Generally, the predominant deformation mechanism for porosigens under compression is brittle fracture rather than plastic flow. Examples of porosigens include acacia, calcium carbonate, calcium sulfate, calcium sulfate dihydrate, compressible sugar, dibasic calcium phosphate (anhydrous and dihydrate), tribasic calcium phosphate, monobasic sodium phosphate, dibasic sodium phosphate, lactose, magnesium oxide, magnesium carbonate, silicon dioxide, magnesium aluminum silicate, maltodextrin, mannitol, methyl cellulose, microcrystalline cellulose, sorbitol, sucrose, and xylitol. Of these, microcrystalline cellulose and both forms of dibasic calcium phosphate (anhydrous and dihydrate) are preferred.

Another useful class of excipients is surfactants, preferably present from 0 to 10 wt %. Suitable surfactants include fatty acid and alkyl sulfates, such as sodium lauryl sulfate; commercial surfactants such as benzalkonium chloride (HYAMINE® 1622 from Lonza, Inc. of Fairlawn, N.J.); dioctyl sodium sulfosuccinate (DOCUSATE SODIUM from Mallinckrodt Specialty Chemicals of St. Louis, Mo.); polyoxyethylene sorbitan fatty acid esters (TWEEN® from ICI Americas Inc. of Wilmington, Del.; LIPOSORB® O-20 from Lipochem Inc. of Patterson N.J.; CAPMUL® POE-0 from Abitec Corp. of Janesville, Wis.); natural surfactants such as sodium taurocholic acid, 1-palmitoyl-2-oleoyl-sn-glycero-3-phosphocholine, lecithin, and other phospholipids and mono- and diglycerides; and polyoxyethylene-polyoxypropylene. Such materials can advantageously be employed to increase the rate of dissolution by, for example, facilitating wetting, or otherwise increase the rate of drug release from the dosage form.

Inclusion of pH modifiers such as acids, bases, or buffers may also be beneficial in an amount of from 0 to 10 wt %. Since many HMG-CoA reductase inhibitors are acid sensitive, care must be taken when formulating a dosage form containing an acidic pH modifier to keep chemical degradation of the HMG-CoA reductase inhibitor at acceptable levels.

In a preferred embodiment, the dosage form also includes a base. The inclusion of a base can improve the chemical stability of the HMG-CoA reductase inhibitor. The term “base” is used broadly to include not only strong bases such as sodium hydroxide, but also weak bases and buffers that are capable of achieving the desired increase chemical stability. Examples of bases include hydroxides, such as sodium hydroxide, calcium hydroxide, ammonium hydroxide, and choline hydroxide; bicarbonates, such as sodium bicarbonate, potassium bicarbonate, and ammonium bicarbonate; carbonates, such as ammonium carbonate, calcium carbonate, and sodium carbonate; amines, such as tris(hydroxymethyl)amino methane, ethanolamine, diethanolamine, N-methyl glucamine, glucosamine, ethylenediamine, N,N′-dibenzylethylenediamine, N-benzyl-2-phenethylamine, cyclohexylamine, cyclopentylamine, diethylamine, isopropylamine, diisopropylamine, dodecylamine, and triethylamine; proteins, such as gelatin; amino acids such as lysine, arginine, guanine, glycine, and adenine; polymeric amines, such as polyamino methacrylates, such as Eudragit E; conjugate bases of various acids, such as sodium acetate, sodium benzoate, ammonium acetate, disodium phosphate, trisodium phosphate, calcium hydrogen phosphate, sodium phenolate, sodium sulfate, ammonium chloride, and ammonium sulfate; salts of EDTA, such as tetra sodium EDTA; and salts of various acidic polymers such as sodium starch glycolate, sodium carboxymethyl cellulose and sodium polyacrylic acid. Preferably, the base is selected from the group consisting of sodium hydroxide, calcium hydroxide, ammonium hydroxide, sodium bicarbonate, potassium bicarbonate, calcium carbonate, sodium carbonate, gelatin, lysine, sodium acetate, sodium benzoate, disodium phosphate, trisodium phosphate, calcium hydrogen phosphate, sodium sulfate, sodium starch glycolate, sodium carboxymethyl cellulose and sodium polyacrylic acid.

Examples of other matrix materials, fillers, or diluents include dextrose, compressible sugar, hydrous lactose, corn starch, silicic anhydride, polysaccharides, dextrates, dextran, dextrin, dextrose, calcium carbonate, calcium sulfate, poloxamers, and polyethylene oxide.

Another optional excipient is a binder such as methyl cellulose, carboxymethylcellulose, hydroxypropylcellulose, hydroxypropyl methyl cellulose, polyvinylpyrrolidone, polyvinylalcohol or starch.

Examples of drug-complexing agents or solubilizers include polyethylene glycols, caffeine, xanthene, gentisic acid and cylodextrins.

Examples of lubricants include calcium stearate, glyceryl monostearate, glyceryl palmitostearate, hydrogenated vegetable oil, light mineral oil, magnesium stearate, mineral oil, polyethylene glycol, sodium benzoate, sodium lauryl sulfate, sodium stearyl fumarate, stearic acid, talc and zinc stearate.

Examples of glidants include silicon dioxide, talc and cornstarch.

In another embodiment, the solid amorphous adsorbate comprising the CETP inhibitor and a substrate, and the HMG-CoA reductase inhibitor are present in separate dosage forms that are co-administered to the environment of use. By “co-administered” is meant that the two dosage forms are administered separately from, but within the same general time frame as, each other. Thus, a dosage form containing, for example, the solid amorphous adsorbate comprising the CETP inhibitor and a substrate, may be administered at approximately the same time as a dosage form containing the HMG-CoA reductase inhibitor. In one embodiment, the two dosage forms are co-administered within the same general time frame as each other, such as within 60 minutes, preferably within 30 minutes, more preferably within 15 minutes of each other. In another embodiment, the two dosage forms are taken at separate times. For example, the dosage form comprising the solid amorphous adsorbate may be taken at meal time, for example, breakfast, lunch, or dinner, while the dosage form comprising the HMG-COA reductase inhibitor is taken in the evening. Either of these scenarios or variations on these scenarios are considered within the scope of the invention.

When administered separately, the invention also relates to combining the solid amorphous adsorbate comprising the CETP inhibitor and a substrate, and the HMG-CoA reductase inhibitor in kit form. The kit includes two separate pharmaceutical compositions: (1) one containing the solid amorphous adsorbate comprising the CETP inhibitor and a substrate, and (2) one containing the HMG-CoA reductase inhibitor. The kit may include means for containing the separate compositions such as a divided container, such as a bottle, pouch, box, bag, or other container known in the art, or a divided foil packet; however, the separate compositions may also be contained within a single, undivided container. Typically the kit includes directions for the administration of the separate components. The kit form is particularly advantageous when the separate components are preferably administered in different dosage forms (e.g., oral and parenteral), are administered at different dosage intervals, or when titration of the individual components of the combination is desired by the prescribing physician.

### Methods of Treatment

The compositions of the present invention may be used to treat any condition, which is subject to treatment by administering a CETP inhibitor and an HMG-CoA reductase inhibitor, as disclosed in commonly assigned, copending U.S. patent application Ser. No. 2002/0035125A1, the disclosure of which is herein incorporated by reference.

In one aspect, the composition of the present invention is used for antiatherosclerotic treatment.

In another aspect, the composition of the present invention is used for slowing and/or arresting the progression of atherosclerotic plaques.

In another aspect, the composition of the present invention is used for slowing the progression of atherosclerotic plaques in coronary arteries.

In another aspect, the composition of the present invention is used for slowing the progression of atherosclerotic plaques in carotid arteries.

In another aspect, the composition of the present invention is used for slowing the progression of atherosclerotic plaques in the peripheral arterial system.

In another aspect, the composition of the present invention, when used for treatment of atherosclerosis, causes the regression of atherosclerotic plaques.

In another aspect, the composition of the present invention is used for regression of atherosclerotic plaques in coronary arteries.

In another aspect, the composition of the present invention is used for regression of atherosclerotic plaques in carotid arteries.

In another aspect, the composition of the present invention is used for regression of atherosclerotic plaques in the peripheral arterial system.

In another aspect, the composition of the present invention is used for HDL elevation treatment and antihyperlipidemic treatment (including LDL lowering).

In another aspect, the composition of the present invention is used for antianginal treatment.

In another aspect, the composition of the present invention is used for cardiac risk management.

Other features and embodiments of the invention will become apparent from the following examples, which are given for illustration of the invention rather than for limiting its intended scope.

### EXAMPLES

### Solid Amorphous Adsorbate 1

The following process was used to form a solid amorphous adsorbate containing 25 wt % [2R,4S]-4-[(3,5-bis-trifluoromethyl-benzyl)-methoxycarbonyl-amino]-2-ethyl-6-trifluoromethyl-3,4-dihydro-2H-quinoline-1-carboxylic acid ethyl ester (torcetrapib) and 75 wt % fumed silica from Cabot Corporation (Boyertown, Pa.) sold as CAB-O-SIL M-5P, having a surface area of about 200 m²/g. First, a spray solution was formed by dissolving 5 g torcetrapib into 380 g acetone, to which 15 g CAB-O-SIL had been suspended. The spray solution was pumped using a Bran+Luebbe small volume high-pressure pump, to a spray drier (Niro type XP Portable Spray-Dryer with a Liquid-Feed Process Vessel [PSD-1]) equipped with a pressure atomizer (Spraying Systems Pressure Nozzle and Body (SK 80-16)). The PSD-1 was equipped with a 9-inch chamber extension. The spray drier was also equipped with a diffuser plate having a 1% open area. The nozzle sat flush with the diffuser plate during operation. The spray solution was pumped to the spray drier, with an atomization pressure of about 25 barg (350 psig). Drying gas (nitrogen) was circulated through the diffuser plate at an inlet temperature of 125° C. The evaporated solvent and wet drying gas exited the spray drier at a temperature of 62° C. The solid amorphous adsorbate was collected in a cyclone.

The concentration-enhancement provided by the solid amorphous adsorbate was demonstrated in an in vitro dissolution test using a syringe method as follows. An 8.0 mg sample of the adsorbate was added to 40 mL phosphate buffered saline (PBS) at pH 6.5 and 290 mOsm/kg, containing 2 wt % sodium taurocholic acid and 1-palmitoyl-2-oleyl-sn-glycero-3-phosphocholine (NaTC/POPC, with a 4/1 weight ratio). The concentration of drug would have been 50 μg/mL, if all of the drug had dissolved. The test solution was stirred at room temperature in a syringe equipped with a Gelman Acrodisc 13 CR 0.45 μm PTFE filter. At each sample time, about 2 mL of the test solution was pushed through the filter and analyzed using UV at a wavelength of 256 nm to determine the concentration of torcetrapib in solution. Samples were collected at 1, 2, 3, 5, 10, 15, 20, 30, 45, 60, and 90 minutes. The results are shown in Table 1. Crystalline torcetrapib alone is shown as a comparison.

TABLE 1

| | | Torcetrapib | |
|---|---|---|---|
| | Time | Concentration | AUC |
| Sample | (min) | (μg/mL) | (min*μg/mL) |
| Solid | 0 | 0 | 0 |
| Amorphous | 1 | 4.07 | 2 |
| Adsorbate 1 | 2 | 9.31 | 9 |
| | 3 | 13.2 | 20 |
| | 5 | 18.7 | 52 |
| | 10 | 23.3 | 157 |
| | 15 | 28.0 | 285 |
| | 20 | 31.5 | 433 |
| | 30 | 33.5 | 758 |
| | 45 | 34.5 | 1270 |
| | 60 | 35.6 | 1790 |
| | 90 | 35.4 | 2860 |
| Crystalline Drug | 0 | <0.5 | 0 |
| Alone | 1 | <0.5 | 0 |
| | 2 | <0.5 | <1 |
| | 3 | <0.5 | <1 |
| | 5 | <0.5 | <2 |
| | 10 | <0.5 | <5 |
| | 15 | <0.5 | <7 |
| | 20 | <0.5 | <10 |
| | 30 | <0.5 | <15 |
| | 45 | <0.5 | <22 |
| | 60 | <0.5 | <30 |
| | 90 | <0.5 | <45 |

The results of these dissolution tests are summarized in Table 2, which shows the maximum concentration of torcetrapib in solution during the first 90 minutes of the test (MDC_{max,90}), the area under the aqueous concentration versus time curve after 90 minutes (AUC₉₀), and the dissolution rate constant, k. The dissolution rate constant was obtained by performing a least squares fit of the experimental data using the following equation:

[D]ₜ=[D]ₒ(1−e^{-kt})

TABLE 2

| | | | Dissolution |
|---|---|---|---|
| | MDC_{max,90} | AUC₉₀ | Rate Constant, k |
| Sample | (μg/mL) | (min-μg/mL) | (min⁻¹) |
| Solid Amorphous | 35.4 | 2860 | 0.13 |
| Adsorbate 1 | | | |
| Control 1 | <0.5 | <45 | <0.0005 |

The results summarized in Table 2 show that the solid amorphous adsorbate provided concentration enhancement relative to crystalline drug. The adsorbate provided an MDC_{max,90} value that was greater than 70-fold that of crystalline drug, and an AUC₉₀ value that was greater than 64-fold that of crystalline drug. In addition, the dissolution rate constant for the solid amorphous adsorbate was much faster than that of the crystalline control, being more than 260-fold that of crystalline drug.

### Solid Amorphous Adsorbate 2

Solid Amorphous Adsorbate 2 was made containing 25 wt % torcetrapib, 10 wt % of the dissolution-enhancing agent, polyvinylpyrrolidone (PVP) (Povidone K-29/30), and 65 wt % CAB-O-SIL M-5P using the same procedure outlined above, with the following exceptions. The spray solution consisted of 62.5 g torcetrapib and 25 g PVP dissolved in methanol, to which was suspended 162.5 g fumed silica (CAB-O-SIL M-5P). The spray solution was pumped at 170 g/min, and the atomization pressure was about 300 psig. The drying gas was circulated through the diffuser plate at an inlet temperature of 215° C., and the evaporated solvent and wet drying gas exited the spray drier at a temperature of 62° C.

The concentration-enhancement provided by Solid Amorphous Adsorbate 2 was demonstrated in an in vitro dissolution test using a syringe method, as described above. In this test, 7.855 mg of the Solid Amorphous Adsorbate 2 was added to 40 mL phosphate buffered saline (PBS) at pH 6.5 and 290 mOsm/kg, containing 2 wt % NaTC/POPC (the concentration of drug would have been 49 μg/mL, if all of the drug had dissolved). The results are shown in Table 3.

TABLE 3

| | | Torcetrapib | |
|---|---|---|---|
| | Time | Concentration | AUC |
| Sample | (min) | (μg/mL) | (min*μg/mL) |
| Solid | 0 | 0 | 0 |
| Amorphous | 1 | 24.7 | 12 |
| Adsorbate 2 | 2 | 36.7 | 43 |
| | 3 | 35.2 | 79 |
| | 5 | 36.6 | 151 |
| | 10 | 38.4 | 338 |
| | 15 | 38.8 | 531 |
| | 20 | 38.5 | 724 |
| | 30 | 39.0 | 1110 |
| | 45 | 38.7 | 1690 |
| | 60 | 39.3 | 2280 |
| | 90 | 40.4 | 3470 |

The results of these dissolution tests are summarized in Table 4, which shows the maximum concentration of torcetrapib in solution during the first 90 minutes of the test (MDC_{max,90}), the area under the aqueous concentration versus time curve after 90 minutes (AUC₉₀), and the dissolution rate constant, k. The results for Solid Amorphous Adsorbate 1 and for crystalline torcetrapib (from Table 2) are shown again for comparison.

TABLE 4

| | | | Dissolution |
|---|---|---|---|
| | MDC_{max,90} | AUC₉₀ | Rate Constant, k |
| Sample | (μg/mL) | (min-μg/mL) | (min⁻¹) |
| Solid Amorphous | 35.4 | 2860 | 0.13 |
| Adsorbate 1 | | | |
| Solid Amorphous | 40.4 | 3470 | 0.94 |
| Adsorbate 2 | | | |
| Crystalline Torcetrapib | <0.5 | <45 | <0.0005 |

The results summarized in Table 4 show that Solid Amorphous Adsorbate 2 provided concentration enhancement relative to crystalline drug. The adsorbate provided an MDC_{max,90} value that was greater than 80-fold that of crystalline drug, and an AUC₉₀ value that was greater than 77-fold that of the crystalline drug. In addition, the data also show that including PVP in Solid Amorphous Adsorbate 2 resulted in an increased dissolution rate constant.

### HMG-CoA Reductase Inhibitor Composition 1

A granulation of atorvastatin hemicalcium trihydrate was prepared using the following process. The granulation contained 13.9 wt % atorvastatin trihydrate hemicalcium salt, 42.4 wt % calcium carbonate, 17.7 wt % microcrystalline cellulose, 3.8 wt % croscarmellose sodium, 0.5 wt % polysorbate 80, 2.6 wt % hydroxypropyl cellulose, and 19.2 wt % pregelatinized starch. To form the granulation, the atorvastatin, calcium carbonate, microcrystalline cellulose, and starch were charged into a fluidized bed granulation apparatus. A granulating fluid comprising the polysorbate 80 and hydroxypropyl cellulose dissolved in water was sprayed into the fluidized material to form the granules. The weight of water used was equal to half the weight of the granulation. The granulation was then dried in the fluidized bed using air with an inlet temperature of about 45° C. until an end point of less than 2% water loss on drying was achieved. The granules were then milled using a Fitzpatrick M5A mill. The mill was fitted with a ˜0.03-inch rasping plate and a rasping bar operating at about 500 rpm in a knives forward direction (counter-clockwise). The average particle size of the granules was about 105 μm using screen analysis. This composition comprised the HMG-CoA reductase inhibitor composition.

### Example 1

To form Example 1, 14.37 g of Solid Amorphous Adsorbate 1 (85 wt %) and 2.54 g of HMG-CoA Reductase Inhibitor Composition 1 (15 wt %) were mixed together in a Turbula mixer for 20 minutes, pushed through a #20 screen, mixed again for 20 minutes in a Turbula mixer, and then pressed into 150 mg compacts using an F-Press. The resulting compacts each contained about 32 mgA torcetrapib and about 3.2 mgA atorvastatin trihydrate hemicalcium salt.

The compacts of Examples 1 were stored in an environmental chamber at 40° C. and 75% relative humidity for 6 weeks and then analyzed for atorvastatin purity using HPLC. No significant concentrations of impurities were observed in the compacts.

### Example 2

To form Example 2, 25.44 g of the Solid Amorphous Adsorbate 2 and 4.57 g of the HMG-CoA Reductase Inhibitor Composition 1 described above were combined, blended, and compressed into 150-mg compacts as described in Example 1. The resulting compacts each contained about 32 mgA torcetrapib and about 3.2 mgA atorvastatin trihydrate hemicalcium salt.

The compacts of Examples 2 were stored in an environmental chamber at 40° C. and 75% relative humidity for 6 weeks and then analyzed for atorvastatin purity using HPLC. No significant concentrations of impurities were observed in the compacts.

### Solid Amorphous Adsorbate 3

The following process was used to form a solid amorphous adsorbate containing 50 wt % [2R,4S]4-[(3,5-bis-trifluoromethyl-benzyl)-methoxycarbonyl-amino]-2-ethyl-6-trifluoromethyl-3,4-dihydro-2H-quinoline-1-carboxylic acid isopropyl ester, “Drug 2”, and 50 wt % CAB-O-SIL M-5P as a substrate. First, a spray solution was formed containing 200 mg Drug 2, 200 mg CAB-O-SIL M-5P, and 14 g of an 8:2 (w:w) mixture of ethanol:water as follows. CAB-O-SIL was added to the ethanol:water solvent and the mixture was sonicated using a Fisher Scientific SF15 sonicator for 30 minutes to ensure full suspension and homogeneity. Drug 2 was then dissolved in this suspension by stirring for 15 minutes, and then sonicating the mixture for 5 minutes. This suspension was then pumped into a “mini” spray-drying apparatus via a Cole Parmer 74900 series rate-controlling syringe pump at a rate of 1.0 mL/min. The spray-drying apparatus used a Spraying Systems Co. two-fluid nozzle, model number SULA, with nitrogen as the atomizing gas. The nitrogen was pressurized and heated to a temperature of 85° C. at the inlet and had a flow rate of about 1 standard ft³/min (SCFM). The suspension was sprayed from the top of an 11-cm diameter stainless steel chamber. The resulting solid amorphous adsorbate was collected on Whatman 1 filter paper, dried under vacuum, and stored in a desiccator.

### Solid Amorphous Adsorbate 4

A solid amorphous adsorbate consisting of 50 wt % Drug 2, 40 wt % CAB-O-SIL M-5P and 10 wt % of the dissolution-enhancing agent PVP (Povidone K-29/30) was prepared using the procedure outlined for Solid Amorphous Adsorbate 3 with the following exceptions. The spray solution was formed by adding 40 mg PVP and 160 mg CAB-O-SIL M-5P to 14 g of the 8:2 w:w enthanol:water solvent and sonicated for 30 minutes. Drug 2 (200 mg) was then dissolved in this suspension and sonicated for 5 minutes. The resulting solid amorphous adsorbate was collected on Whatman 1 filter paper, dried under vacuum, and stored in a desiccator.

### Concentration Enhancement

The concentration enhancement provided by Solid Amorphous Adsorbates 3 and 4 were demonstrated in an in-vitro test using the procedures outlined for Solid Amorphous Adsorbate 1 except that the samples were analyzed for Drug 2 concentration using UV absorbance at a wavelength of 260 nm. The results are shown in Table 5. Crystalline Drug 2 alone is shown as a comparison. In all cases, a sufficient amount of sample was added so that the concentration of drug would have been 50 μg/mL, if all of the drug had dissolved.

TABLE 5

| | | Drug 2 | |
|---|---|---|---|
| | Time | Concentration | AUC |
| Example | (min) | (μg/mL) | (min*μg/mL) |
| Solid | 0 | 0 | 0 |
| Amorphous | 0.5 | <0.5 | 0 |
| Adsorbate 3 | 1 | 1.4 | 4.2 |
| | 2 | 1.4 | 5.6 |
| | 3 | 3.3 | 7.9 |
| | 5 | 10 | 22 |
| | 10 | 18 | 93 |
| | 15 | 22 | 193 |
| | 20 | 25 | 312 |
| | 30 | 29 | 583 |
| | 45 | 33 | 1050 |
| | 60 | 35 | 1550 |
| | 90 | 39 | 2650 |
| Solid | 0 | 0 | 0 |
| Amorphous | 0.5 | 2.0 | 0 |
| Adsorbate 4 | 1 | 4.5 | 2.1 |
| | 2 | 4.5 | 6.6 |
| | 3 | 9.0 | 13 |
| | 5 | 20 | 42 |
| | 10 | 32 | 172 |
| | 15 | 30 | 326 |
| | 20 | 28 | 470 |
| | 30 | 33 | 776 |
| | 45 | 37 | 1300 |
| | 60 | 33 | 1820 |
| | 90 | 36 | 2850 |
| Crystalline | 0 | 0 | 0 |
| Drug 2 | 0.5 | 0.1 | 0 |
| | 5 | 1.0 | 2.6 |
| | 15 | 1.8 | 17 |
| | 45 | 4.5 | 111 |
| | 90 | 7.6 | 383 |

The results of these tests are summarized in Table 6, which shows the maximum concentration of Drug 2 in solution during the first 90 minutes of the test (MDC₉₀) the area under the aqueous concentration versus time curve after 90 minutes (AUC₉₀), and the dissolution rate constant, k.

TABLE 6

| | | | Dissolution |
|---|---|---|---|
| | MDC₉₀ | AUC₉₀ | Rate Constant, k |
| Sample | (ng/mL) | (min*μg/mL) | (min⁻¹) |
| Solid Amorphous | 39 | 2650 | 0.057 |
| Adsorbate 3 | | | |
| Solid Amorphous | 36 | 2850 | 0.14 |
| Adsorbate 4 | | | |
| Crystalline Drug 2 | 7.6 | 383 | 0.005 |

These results show that the Drug 2 concentrations provided by the solid amorphous adsorbates were much greater than the concentrations provided by unadsorbed Drug 2 alone (e.g., crystalline Drug 2). Solid Amorphous Adsorbate 3 provided a MDC₉₀ that was 5.1-fold that of crystalline Drug 2, while Solid Amorphous Adsorbate 4 provided an MDC₉₀ that was 4.7-fold that of crystalline Drug 2. Solid Amorphous Adsorbate 3 provided an AUC₉₀ that was 6.9-fold that of crystalline Drug 2, while Solid Amorphous Adsorbate 4 provided an AUC₉₀ that was 7.4-fold that of crystalline Drug 2.

The data also show that the dissolution rate constant for the solid amorphous adsorbates was greater than that for crystalline drug, with Solid Amorphous Adsorbate 3 providing a dissolution rate constant that was 11.4-fold that of crystalline drug and Solid Amorphous 4 providing a dissolution rate constant that was 28-fold that of crystalline drug. The data also show that the use of the dissolution-enhancing agent PVP in Solid Amorphous Adsorbate 4 resulted in a higher dissolution rate constant.

### Example 3

A tablet containing about 60 mgA Drug 2 and about 10 mgA atorvastatin trihydrate hemicalcium salt is prepared by combining, blending, and compressing about 120 mg of Solid Amorphous Adsorbate 3 and about 72 mg of HMG-CoA Reductase Inhibitor Composition 1, as described in Example 1.

### Example 4

A tablet containing about 60 mgA Drug 2 and about 20 mgA atorvastatin trihydrate hemicalcium salt is prepared by combining, blending, and compressing about 120 mg of Solid Amorphous Adsorbate 3 and about 144 mg of the HMG-CoA Reductase Inhibitor Composition 1, as described in Example 1.

### Solid Amorphous Adsorbate 5

The following process was used to form a solid amorphous adsorbate containing 50 wt % [2R,4S]4-[acetyl-(3,5-bis-trifluoromethyl-benzyl)-amino]-2-ethyl-6-trifluoromethyl-3,4-dihydro-2H-quinoline-1-carboxylic acid isopropyl ester, “Drug 3”, and 50 wt % CAB-O-SIL M-5P as a substrate. First, a spray solution was formed containing 200 mg Drug 3, 200 mg CAB-O-SIL M-5P, and 14 g of methanol as follows. CAB-O-SIL was added to the solvent and the mixture was sonicated using a Fisher Scientific SF15 sonicator for 30 minutes to ensure full suspension and homogeneity. Drug 3 was then dissolved in this suspension by stirring for 15 minutes, and then sonicating for 5 minutes. This suspension was then pumped into a “mini” spray-drying apparatus via a Cole Parmer 74900 series rate-controlling syringe pump at a rate of 1.0 mL/min. The spray-drying apparatus used a Spraying Systems Co. two-fluid nozzle, model number SU1A, with nitrogen as the atomizing gas. The nitrogen was pressurized and heated to a temperature of 70° C. at the inlet and had a flow rate of about 1 SCFM. The suspension was sprayed from the top of an 11-cm diameter stainless steel chamber. The resulting solid amorphous adsorbate was collected on Whatman 1 filter paper, dried under vacuum, and stored in a desiccator.

### Solid Amorphous Adsorbate 6

A solid amorphous adsorbate consisting of 50 wt % Drug 3, 40 wt % CAB-O-SIL M-5P and 10 wt % of the dissolution-enhancing agent PVP (Povidone K-29/30) was prepared using the procedure outlined for Solid Amorphous Adsorbate 5 with the following exceptions. The spray solution was formed by adding 40 mg PVP and 160 mg CAB-O-SIL M-5P to 14 g methanol and sonicating for 30 minutes. Drug 3 (200 mg) was then dissolved in this suspension and sonicated for 5 minutes. The resulting solid amorphous adsorbate was collected on Whatman 1 filter paper, dried under vacuum, and stored in a desiccator.

### Concentration Enhancement

The concentration enhancement provided by Solid Amorphous Adsorbates 5 and 6 were demonstrated in an in-vitro test using the procedures outlined for Solid Amorphous Adsorbate 1. The results are shown in Table 7. Crystalline Drug 3 alone is shown as a comparison. In all cases, a sufficient amount of sample was added so that the concentration of drug would have been 50 μg/mL, if all of the drug had dissolved.

TABLE 7

| | | Drug 3 | |
|---|---|---|---|
| | Time | Concentration | AUC |
| Example | (min) | (μg/mL) | (min*μg/mL) |
| Solid | 0 | 0 | 0 |
| Amorphous | 0.5 | 1.3 | 0.3 |
| Adsorbate 5 | 1 | 1.9 | 1.1 |
| | 2 | 3.0 | 3.6 |
| | 3 | 6.2 | 8.2 |
| | 5 | 11 | 26 |
| | 10 | 19 | 100 |
| | 15 | 22 | 200 |
| | 20 | 25 | 320 |
| | 30 | 27 | 575 |
| | 45 | 32 | 1020 |
| | 60 | 34 | 1510 |
| | 90 | 38 | 2600 |
| Solid | 0 | 0 | 0 |
| Amorphous | 0.5 | 1.7 | 0.4 |
| Adsorbate 6 | 1 | 3.9 | 1.8 |
| | 2 | 8.7 | 8.1 |
| | 3 | 14 | 20 |
| | 5 | 21 | 55 |
| | 10 | 30 | 180 |
| | 15 | 35 | 340 |
| | 20 | 35 | 515 |
| | 60 | 35 | 1900 |
| | 90 | 36 | 2960 |
| Crystalline | 0 | 0 | 0 |
| Drug 3 | 0.5 | <0.5 | 0 |
| | 1 | <0.5 | 0 |
| | 3 | <0.5 | <0.3 |
| | 5 | 1.6 | 2.3 |
| | 10 | 1.6 | 10 |
| | 15 | 1.2 | 17 |
| | 20 | 2.5 | 26 |
| | 30 | 3.7 | 57 |
| | 45 | 5.2 | 124 |
| | 60 | 7.2 | 217 |
| | 90 | 13 | 524 |

The results of these tests are summarized in Table 8, which shows the maximum concentration of Drug 3 in solution during the first 90 minutes of the test (MDC₉₀) and the area under the aqueous concentration versus time curve after 90 minutes (AUC₉₀).

TABLE 8

| | | | Dissolution |
|---|---|---|---|
| | MDC₉₀ | AUC₉₀ | Rate Constant, k |
| Sample | (μg/mL) | (min*μg/mL) | (min⁻¹) |
| Solid Amorphous | 38 | 2600 | 0.062 |
| Adsorbate 5 | | | |
| Solid Amorphous | 36 | 2960 | 0.167 |
| Adsorbate 6 | | | |
| Crystalline Drug 3 | 13 | 524 | 0.004 |

These results show that the Drug 3 concentrations provided by the solid amorphous adsorbates were much greater than the concentrations provided by unadsorbed Drug 3 alone (e.g., crystalline Drug 3). Solid Amorphous Adsorbate 5 provided a MDC₉₀ that was 2.9-fold that of crystalline Drug 3, while Solid Amorphous Adsorbate 6 provided an MDC₉₀ that was 2.8-fold that of crystalline Drug 3. Solid Amorphous Adsorbate 5 provided an AUCgo that was 5.0-fold that of crystalline Drug 3, while Solid Amorphous Adsorbate 6 provided an AUC₉₀ that was 5.6-fold that of crystalline Drug 3.

The data also show that the dissolution rate constant for the solid amorphous adsorbates was greater than that for crystalline drug, with Solid Amorphous Adsorbate 5 providing a dissolution rate constant that was 15.5-fold that of crystalline drug and Solid Amorphous 6 providing a dissolution rate constant that was 42-fold that of crystalline drug. The data also show that the use of the dissolution-enhancing agent PVP in Solid Amorphous Adsorbate 6 resulted in a higher dissolution rate constant.

### Example 5

A tablet containing about 60 mgA Drug 3 and about 10 mgA atorvastatin trihydrate hemicalcium salt is prepared by combining, blending, and compressing about 120 mg of Solid Amorphous Adsorbate 5 and about 72 mg of HMG-CoA Reductase Inhibitor Composition 1, as described in Example 1.

### Example 6

A tablet containing about 60 mgA Drug 3 and about 20 mgA atorvastatin trihydrate hemicalcium salt is prepared by combining, blending, and compressing about 120 mg of Solid Amorphous Adsorbate 6 and about 144 mg of the HMG-CoA Reductase Inhibitor Composition 1, as described in Example 1.

1. A composition comprising:
(a) a solid amorphous adsorbate, said solid amorphous adsorbate comprising a cholesteryl ester transfer protein inhibitor and a substrate; and
(b) an HMG-CoA reductase inhibitor.

2. The composition of claim 1 wherein said composition further comprises a concentration-enhancing polymer.

3. The composition of claim 2 wherein said solid amorphous adsorbate further comprises said concentration-enhancing polymer.

4. The composition of claims 2 or 3 wherein said concentration-enhancing polymer is selected from the group consisting of neutral non-cellulosic polymers, ionizable non-cellulosic polymers, neutral cellulosic polymers, ionizable cellulosic polymers, acidic polymers, neutralized acidic polymers, and blends thereof.

5. The composition of any one of claims 1-3 wherein said cholesteryl ester transfer protein inhibitor is selected from the group consisting of the compounds of Formula I, Formula II, Formula III, Formula IV, Formula V, Formula VI, Formula VII, Formula VIII, Formula IX, Formula X, Formula XI, Formula XII, Formula XIII, Formula XIV, Formula XV, Formula XVI, Formula XVII, Formula XVIII, and Formula XIX.

6. The composition of any one of claims 1-3 wherein said cholesteryl ester transfer protein inhibitor is selected from the group consisting of [2R,4S]-4-[acetyl-(3,5-bis-trifluoromethyl-benzyl)-amino]-2-ethyl-6-trifluoromethyl-3,4-dihydro-2H-quinoline-1-carboxylic acid isopropyl ester, [2R,4S]-4-[3,5-bis-trifluoromethyl-benzyl)-methoxycarbonyl-amino]-2-ethyl-6-trifluoromethyl-3,4-dihydro-2H-quinoline-1-carboxylic acid ethyl ester, (2R)-3-[[3-(4-chloro-3-ethylphenoxy)phenyl][[3-(1,1,2,2-tetrafluoroethoxy)phenyl]methyl]amino]-1,1,1-trifluoro-2-propanol, and [2R,4S]4-[(3,5-bis-trifluoromethyl-benzyl)-methoxycarbonyl-amino]-2-ethyl-6-trifluoromethyl-3,4-dihydro-2H-quinoline-1-carboxylic acid isopropyl ester.

7. The composition of any one of claims 1-3 wherein said HMG-CoA reductase inhibitor is selected from the group consisting of fluvastatin, lovastatin, pravastatin, atorvastatin, simvastatin, cerivastatin, rivastatin, mevastatin, velostatin, compactin, dalvastatin, fluindostatin, rosuvastatin, pitivastatin, dihydrocompactin and pharmaceutically acceptable forms thereof.

8. The composition of any one of claims 1-3 wherein said HMG-CoA reductase inhibitor is selected from the group consisting of atorvastatin, the cyclized lactone form of atorvastatin, a 2-hydroxy, 3-hydroxy or 4-hydroxy derivative of said compounds, and pharmaceutically acceptable forms thereof.

9. The composition of any one of claims 1-3 comprising torcetrapib and an HMG-CoA reductase inhibitor selected from the group consisting of atorvastatin and pharmaceutically acceptable forms thereof.

10. The composition of any one of claims 1-3 wherein said composition, following administration to an in vivo or in vitro aqueous environment of use, provides at least one of
(a) an improvement in the maximum concentration of said cholesteryl ester transfer protein inhibitor in said use environment of at least 1.25 fold relative to a control composition consisting essentially of said cholesteryl ester transfer protein inhibitor alone;
(b) an area under the concentration of said cholesteryl ester transfer protein inhibitor in said use environment versus time curve for any period of at least 90 minutes between the time of introduction into the use environment and about 270 minutes following introduction to the use environment that is at least 1.25-fold that of a control composition consisting essentially of said cholesteryl ester transfer protein inhibitor alone;
(c) an improvement in the relative bioavailability of said cholesteryl ester transfer protein inhibitor of at least 1.25-fold relative to a control composition consisting essentially of said cholesteryl ester transfer protein inhibitor alone; and
(d) an improvement in the maximum concentration of said cholesteryl ester transfer protein inhibitor in the blood of at least 1.25 fold relative to a control composition consisting essentially of said cholesteryl ester transfer protein inhibitor alone.

11. The composition of any one of claims 1-3 wherein said solid amorphous adsorbate further comprises a dissolution-enhancing agent.

12. The composition of any one of claims 1-3 wherein said solid amorphous adsorbate has a dissolution rate constant of at least 0.005 min⁻¹.

13. The composition of any one of claims 1-3 wherein said substrate has a surface area of about 200 m²/g or more.

14. A dosage form selected from the group consisting of a capsule, pill and tablet comprising the composition of any one of claims 1-3.

15. A method of treating a patient in need of combination therapy of a CETP inhibitor and an HMG-CoA reductase inhibitor comprising administering to said patient a therapeutically effective amount of a composition of any one of claims 1-3.
